(19) United States
(12) Patent Application Publication
(10) Pub. No.: US 2009/0137773 A1
(43) Pub. Date: May 28, 2009

(54) Production Processes and Systems, Compositions, Surfactants, Monomer Units, Metal Complexes, Phosphate Esters, Glycols, Aqueous Film Forming Foams, and Foam Stabilizers

(76) Inventors: Andrew Jackson, West Lafayette, IN (US); Vimal Sharma, West Lafayette, IN (US); Bradley E. Edwards, Lafayette, IN (US); Janet Boggs, Crawfordsville, IN (US); Victoria Hedrick, Brookston, IN (US); Stephan Brandstadter, Indianapolis, IN (US); John Chien, West Lafayette, IN (US); Edward Norman, Chester Springs, PA (US); Robert Kaufman, St, Louis, MO (US); Bruno Ameduri, Montpellier (FR); George K. Kostov, Montpellier (FR)

Correspondence Address: Wells St. John P.S., 601 W. First Ave., Suite 1300, Spokane, WA, 99201, US

(21) Appl. No.: 11/922,980

(22) PCT Filed: Jul. 28, 2006
(86) PCT No.: PCT/US06/29459

§ 371 Date: Dec. 28, 2007

Related U.S. Application Data
(63) Continuation of application No. 11/192,832, filed on Jul. 28, 2005.
(60) Provisional application No. 60/704,168, filed on Jul. 29, 2005.

(51) Int. Cl. C08G 75/20; C07C 317/04; C07C 311/01; C07F 9/09; C07C 31/38; C07C 19/08

(52) U.S. Cl. 528/391; 568/35; 564/96; 570/134; 570/127; 568/842; 556/1; 558/204

(57) ABSTRACT

Compositions and methods for making compositions such as RF(Rτ)nQ are provided. The RF group can include at least two —CF3 groups, the Rτ group can be a group having at least two carbons, n can be at least 1, and the Q group can include one or more atoms of the periodic table of elements. RF-intermediates (RF(RT)nQg); Surfactants (RF(RT)nQs); Foam stabilizers (RF(RT)IIQFS); Metal complexes (RF(RT)IIQMC); Phosphate ester (RF(RT)HQPE); Polymers (RF(RT)IIQMU); Monomers (RF(RT)IIQM); Urethanes (RF(Rτ)nQu); and/or Glycols (RF(RT)IIQH) and methods for making the same are provided.

### CLAIM FOR PRIORITY

This application claims priority to U.S. Provisional Patent Application Ser. No. 60/704,168, entitled Production Processes and Systems, Compositions, Surfactants, Monomer Units, Metal Complexes, Phosphate Esters, Glycols, Aqueous Film Forming Foams, and Foam Stabilizers, filed Jul. 29, 2005, as well as U.S. patent application Ser. No. 11/192,832, entitled Compositions, Halogenated Compositions, Chemical Production and Telomerization Processes, filed Jul. 28, 2005, the entirety of both of which are incorporated by reference herein.

This application also claims priority as a continuation-in-part of international patent applications: PCT/US05/03429, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; PCT/US05/02617, entitled Compositions, Halogenated Compositions, Chemical Production and Telomerization Processes, filed Jan. 28, 2005; PCT/US05/03433, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; PCT/US05/03137, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; and PCT/US05/03138, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005, U.S. patent application Ser. No. 11/192,832, entitled Compositions, Halogenated Compositions, Chemical Production and Telomerization Processes, filed Jul. 28, 2005, the entirety of all of which are incorporated by reference herein.

### TECHNICAL FIELD

The present invention relates to the field of halogenated compositions, processes for manufacturing halogenated compositions, and, more specifically, fluorinated compositions, processes for manufacturing fluorinated compositions and methods for treating substrates with the fluorinated compositions.

### BACKGROUND

Compositions such as surfactants and polymers, for example, have incorporated fluorine to affect the performance of the composition when the composition is used as a treatment for materials and when the composition is used to enhance the performance of materials. For example, surfactants incorporating fluorinated functional groups can be used as fire extinguishants either alone or in formulations such as aqueous film forming foams (AFFF). Traditional fluorosurfactants, such as perfluoro-octyl sulfonate derivatives (PFOS), have linear perfluorinated portions.

Polymers incorporating fluorine have been used to treat materials. Exemplary fluorinated treatments include compositions such as Scotchguard®.

### SUMMARY

Compositions and methods for making compositions such as R_{F}(R_{T})ₙQ are provided. The R_{F} group can include at least two —CF₃ groups, the R_{T} group can be a group having at least two carbons, n can be at least 1, and the Q group can include one or more atoms of the periodic table of elements.

RF-intermediates and methods for making same are also provided such as R_{F}(R_{T})ₙQ_{g}, with the Q_{g} group being one or more atoms of the periodic table of elements.

Surfactants and methods from making same are provided that can include R_{F}(R_{T})ₙQₛ, with the Qₛ group being at least one atom of the periodic table of elements, and at least a portion of the R_{F} and R_{T} groups are hydrophobic relative to the Qₛ group, and at least a portion of the Qₛ group is hydrophilic relative to the R_{F} and R_{T} groups.

Foam stabilizers and methods for making same are provided that can include R_{F}(R_{T})ₙQ_{FS}, with the Q_{FS} group being at least one atom of the periodic table of elements, and at least a portion of the R_{F} and R_{T} groups are hydrophobic relative to the Q_{FS} group, and at least a portion of the Q_{FS} group is hydrophilic relative to the R_{F} and R_{T} groups.

Metal complexes and methods for making same are provided that can include R_{F}(R_{T})ₙQ_{MC}, with the Q_{MC} group being at least one atom of the periodic table of elements.

Phosphate ester and methods of making same are provided that can include R_{F}(R_{T})ₙQ_{PE}, with the Q_{PE} group being a portion of a phosphate ester group.

Polymers and methods of making same are provided that can include R_{F}(R_{T})ₙQ_{MU}, with the Q_{MU} group being a portion of a polymer chain backbone

Monomers and methods of making same are provided that can include R_{F}(R_{T})ₙQ_{M}, with the Q_{M} group being at least one atom of the periodic table of elements.

Urethanes and methods of making same are provided that can include R_{F}(R_{T})ₙQ_{U}, with the Q_{U} group being at least one atom of the periodic table of elements.

Glycols and methods for making the same are provided that can include R_{F}(R_{T})ₙQ_{H}, with the Q_{H} group is a portion of a glycol chain backbone.

### BRIEF DESCRIPTION OF THE DRAWINGS

Embodiments are described below with reference to the following accompanying drawings.

FIG. 1 is a general view of exemplary RF-compositions.

FIG. 2 is an exemplary system for preparing compositions according to an embodiment.

### DETAILED DESCRIPTION

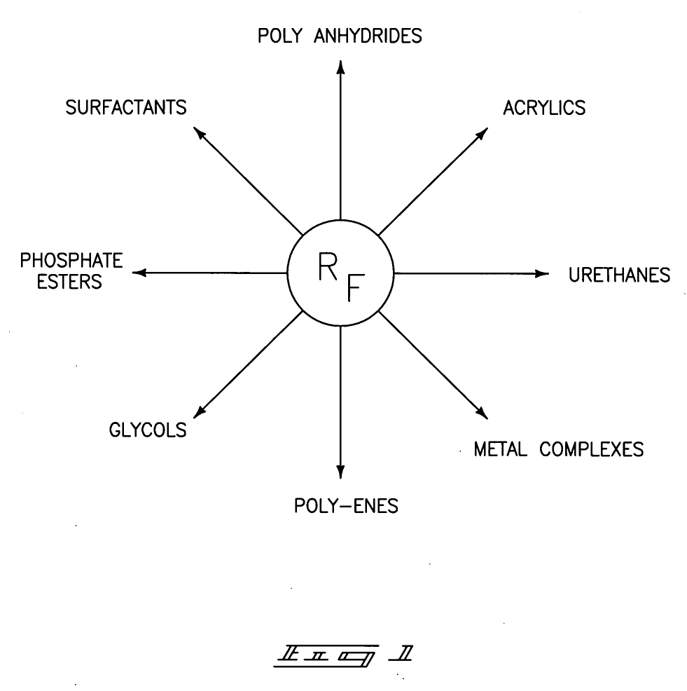

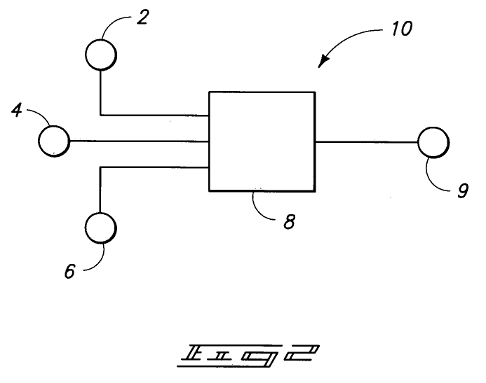

Exemplary R_{F}-compositions and production methods are described with reference to FIGS. 1-2. Starting materials and/or intermediate materials as well as processes for producing the same and/or introducing RF-intermediates compositions into surfactants, polymers, glycols, monomers, monomer units, phosphate esters, metal complexes, and/or foam stabilizers can be described in published International Patent applications: PCT/US05/03429, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; PCT/US05/02617, entitled Compositions, Halogenated Compositions, Chemical Production and Telomerization Processes, filed Jan. 28, 2005; PCT/US05/03433, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; PCT/US05/03137, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; and PCT/US05/03138, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005, the entirety of all of which are incorporated by reference herein (“Published International Applications”).

Referring to FIG. 1, a general view of exemplary R_{F}-compositions is shown. R_{F}-compositions include, but are not limited to, R_{F}-surfactants, R_{F}-monomers, R_{F}-monomer units, R_{F}-metal complexes, R_{F}-phosphate esters, R_{F}-glycols, R_{F}-urethanes, and or R_{F}-foam stabilizers. In exemplary embodiments, poly-anhydrides, acrylics, urethanes, metal complexes, poly-enes, and/or phosphate esters can include R_{F} portions as well.

R_{F}-compositions include compositions that have an R_{F} portion and/or R_{F} portions. The R_{F} portion can be R_{F}-groups, such as pendant groups and/or moieties of compositions. The R_{F} portion can include at least two —CF₃ groups and the —CF₃ groups may be terminal. The R_{F} portion can also include both —CF₃ groups and additional groups containing fluorine, such as —CF₂— groups. In exemplary embodiments, the R_{F} portion can include a ratio of —CF₂— groups to —CF₃ groups that is less than or equal to two, such as (CF₃)₂CF— groups. The R_{F} portion can also include hydrogen. For example, the R_{F} portion can include two —CF₃ groups and hydrogen, such as (CF₃)₂CH— groups. The R_{F} portion can also include two —CF₃ groups and a —CH₂— group, in other embodiments. The R_{F} portion can include at least three —CF₃ groups, such as two (CF₃)₂CF— groups. In exemplary embodiments, the R_{F} portion can include cyclic groups such as aromatic groups. The R_{F} portion can include at least two —CF₃ groups and at least four carbons with, for example, one of the four carbons including a —CH₂— group. According to exemplary embodiments, the RF group can further comprise at least a portion of an (R_{T}) group or groups. In exemplary implementations these R_{T} groups can be incorporated into and form a part of R_{F} groups via processes described herein, such as telomerization processes.

In exemplary implementations, R_{F}-compositions can demonstrate desirable surface energies, affect the surface tension of solutions to which they are exposed, and/or affect the environmental resistance of materials to which they are applied and/or incorporated. Exemplary compositions include, but are not limited to, substrates having R_{F}-compositions thereover and/or liquids having R_{F}-compositions therein. R_{F} portions can be incorporated into compositions such as polymers, acrylate monomers and polymers, glycols, fluorosurfactants, and/or AFFF formulations. These compositions can be used as dispersing agents or to treat substrates such as textile fabric, textile yarns, leather, paper, plastic, sheeting, wood, ceramic clays, as well as, articles of apparel, wallpaper, paper bags, cardboard boxes, porous earthenware, construction materials such as brick, stone, wood, concrete, ceramics, tile, glass, stucco, gypsum, drywall, particle board, chipboard, carpet, drapery, upholstery, automotive, awning fabrics, and rainwear. R_{F}-compositions can be prepared from R_{F}-intermediates.

R_{F} portions can be incorporated into R_{F}-compositions and/or can be starting materials for R_{F}-compositions via R_{F}-intermediates. Exemplary R_{F}-intermediates include an R_{F} portion described above, as well as at least one functional portion that allows for incorporation of the R_{F} portion Into compositions to form R_{F}-compositions. Functional portions can include halogens (e.g., iodine), mercaptan, thiocyanate, sulfonyl chloride, acid, acid halides, hydroxyl, cyano, acetate, allyl, epoxide, acrylic ester, ether, sulfate, thiol, phosphate, and/or amines, for example. Without incorporation and/or reaction, R_{F}-intermediates can include R_{F}-compositions, such as R_{F}-monomers and/or ligands of R_{F}-metal complexes, for example.

R_{F}-intermediates can include R_{F}-Q_{g} with R_{F} representing the R_{F} portion and Q_{g} representing, for example, the functional portion, and/or, as another example, an element of the periodic table of elements. In exemplary embodiments, Q_{g} is not a proton, methyl, and/or a methylene group. Exemplary R_{F}-intermediates include, but are not limited to, those in Table 1 below.

TABLE 1
Exemplary R_{F}-Intermediates

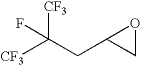

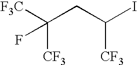

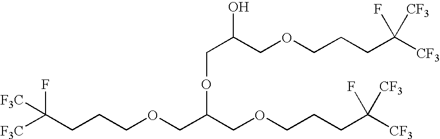

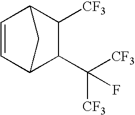

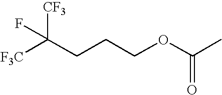

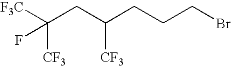

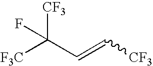

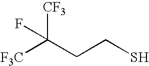

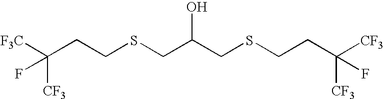

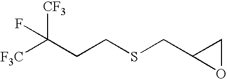

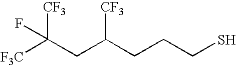

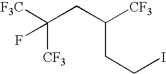

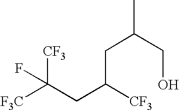

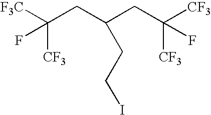

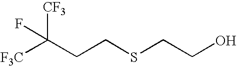

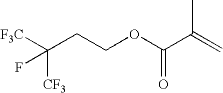

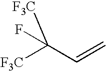

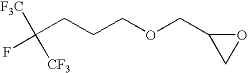

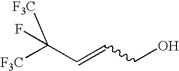

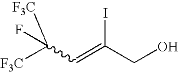

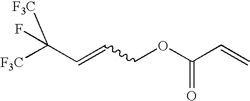

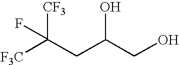

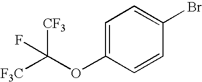

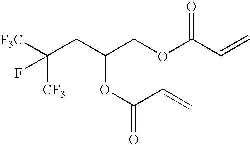

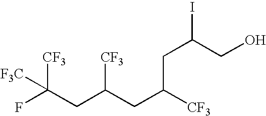

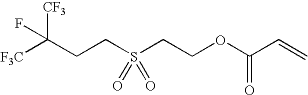

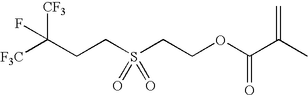

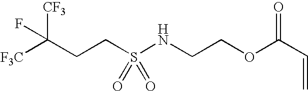

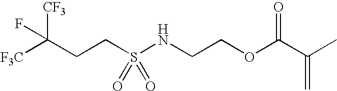

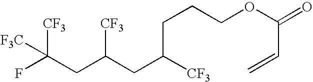

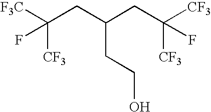

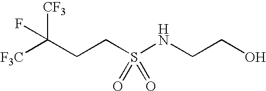

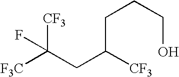

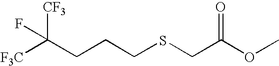

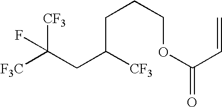

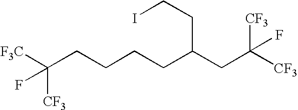

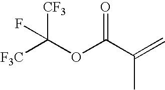

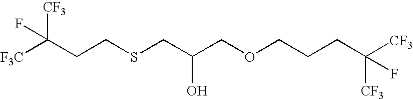

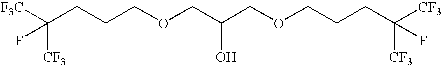

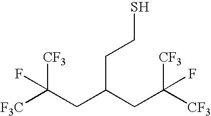

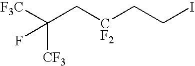

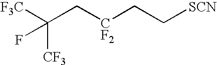

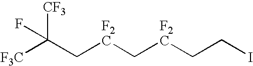

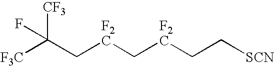

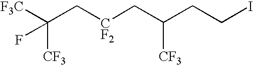

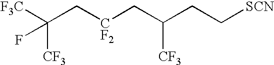

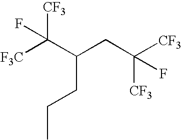

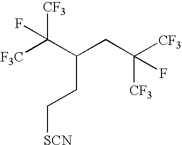

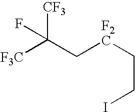

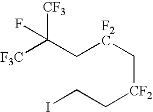

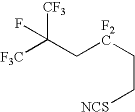

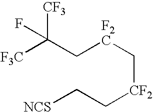

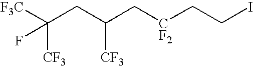

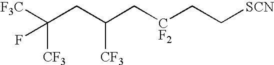

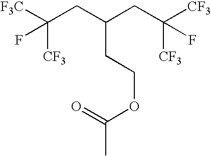

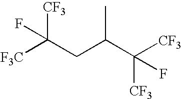

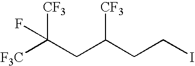

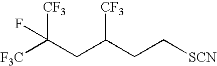

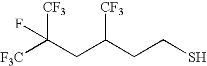

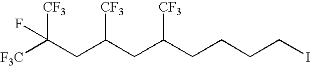

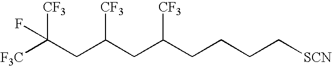

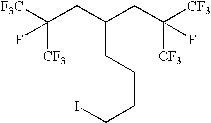

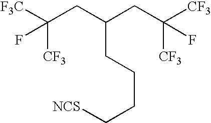

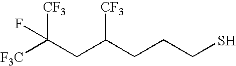

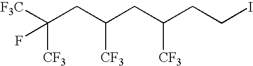

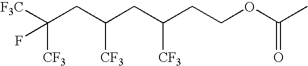

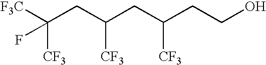

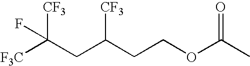

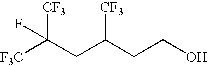

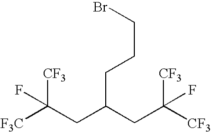

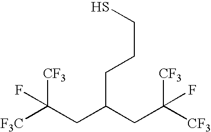

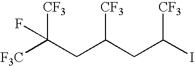

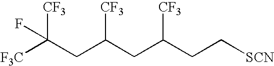

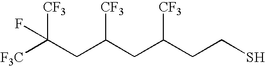

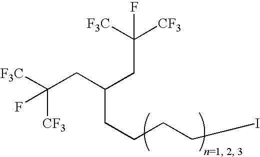

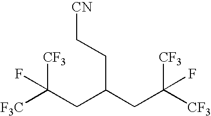

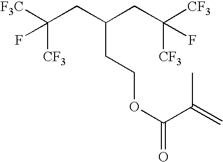

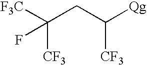

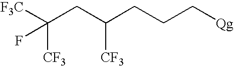

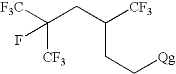

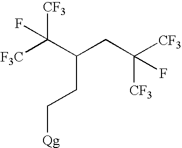

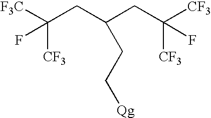

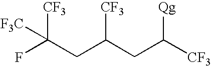

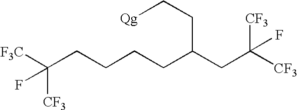

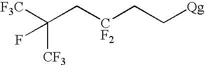

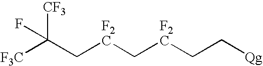

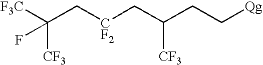

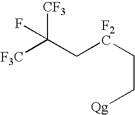

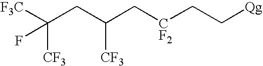

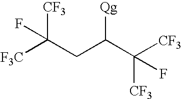

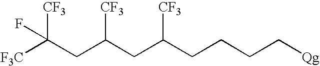

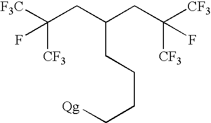

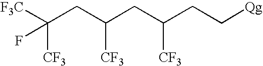

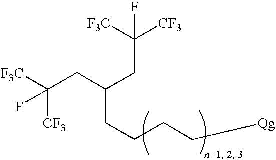

Utilizing the methods and systems to prepare starting materials described in the Published International Applications, novel R_{F}-intermediates can be prepared in accordance with examples 1-27 below.

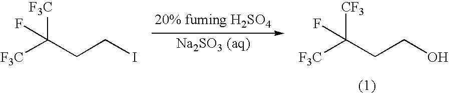

According to scheme (1) above, in a flask that can be equipped with an agitator, thermocouple, reflux condenser, and an addition funnel, 105 ml of 20% fuming sulfuric acid can be placed and cooled to about 10° C. with an ice bath. To the cooled 20% fuming sulfuric acid, 103 grams (0.32 mole) of 1,1,1,2-tetrafluoro-2-(trifluoromethyl)-4-iodobutane (Matrix Scientific)(see, e.g. Published International Applications) can be added slowly over a 15 minute period to form a first mixture whereupon the first mixture became dark. The first mixture can be allowed to warm to from about 18° C. to about 24° C., and/or about 21° C. whereupon an exotherm can be observed by an increase in the first mixture temperature from 17° C. to 45° C. with violent off gassing. Additional ice can be added to the ice bath in order to control the exotherm. In a separate flask that can be equipped with an agitator, thermocouple, a Dean Stark, reflux condenser, and an addition funnel, 50 grams of sodium sulfite and 500 mL of water can be added to form a second mixture. To the second mixture, the entirety of the first mixture can be slowly added such that the temperature can be maintained below about 50° C. to form a reaction mixture. The reaction mixture can be heated to reflux and the condensate collected in the Dean Stark apparatus whereupon an organic phase can be separated from an aqueous phase. The organic phase can be collected in portions throughout the reaction and the aqueous phase allowed to return to the reaction mixture. The combined organic phases can be washed with water to form a multiphase mixture from which an organic phase can be separated from an aqueous phase. The organic phase can be collected to afford 66.2 grams of the 3,4,4,4-tetrafluoro-3-(trifluoromethyl)butan-1-ol having a purity by gas chromatography of 99.6 area percent. The product structure can be confirmed by NMR and/or chromatographic analysis.

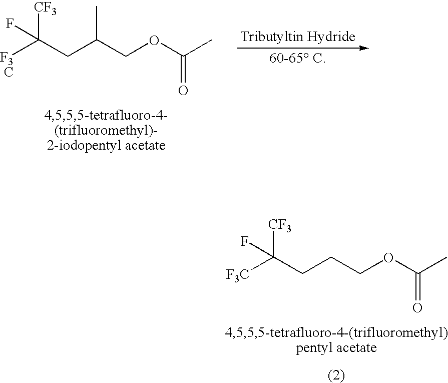

According to scheme (2) above, in a flask that can be equipped with an agitator, thermocouple, reflux condenser, and an addition funnel, 39.1 grams (0.1 mole) of 4,5,5,5-tetrafluoro-4-(trifluoromethyl)-2-iodopentyl acetate (see, e.g. Published International Applications) can be added. The flask can be heated to between about 60° C. to 65° C. then 30.41 grams (0.104 mole) of tributyltin hydride can be added drop wise over about 210 minutes to form a mixture. The mixture can be cooled to from about 18° C. to about 24° C., and/or about 21° C. and held from about 15 hours to about 21 hours, and/or about 18 hours. The mixture can be distilled (66° C. at 27 Torr) to afford about 19.7 grams of 4,5,5,5-tetrafluoro-4-(trifluoromethyl)pentyl acetate product. The product structure can be confirmed by NMR and/or chromatographic analysis.

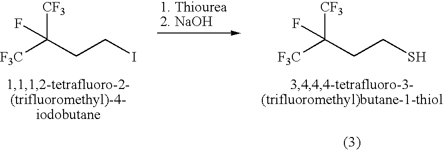

According to scheme (3) above, in a flask that can be equipped with a thermocouple, heating mantle, an agitator, and a reflux condenser, 300 grams (0.926 mole) of 4-iodo-2-(trifluoromethyl)-1,1,1,2-tetrafluorobutane (see, e.g. Published International Applications) can be dissolved in about 2778 mL ethanol to form a mixture. To the mixture, 106 grams (1.39 moles) thiourea can be added to form a reaction mixture. The reaction mixture can be heated to reflux and a transformation from a heterogeneous mixture to a homogeneous mixture can be observed. The reaction mixture can be held at the reflux temperature for from about 42 hours to about 58 hours, and/or about 50 hours. The reaction mixture can then be cooled to from about 18° C. to about 24° C., and/or about 21° C. The cooled reaction mixture can be concentrated in vacuo and a white solid recovered. The white solid can be dissolved in about 1200 mL deionized water to form a solution. To the solution, 156 grams of sodium hydroxide can be added to form a reaction solution, whereupon an exotherm can be observed. The reaction solution can be stirred at from about 18° C. to about 24° C., and/or about 21° C., for about one hour. The reaction solution can be distilled at about 100° C. using a Dean-Stark trap, from which the organic layer can be separated from the aqueous phase; The organic layer can be collected and washed by addition with deionized water to remove residual ethanol to afford 134.4 grams of the 3,4,4,4-tetrafluoro-3-(trifluoromethyl)butane-1-thiol product. The product structure can be confirmed by NMR and/or chromatographic analysis.

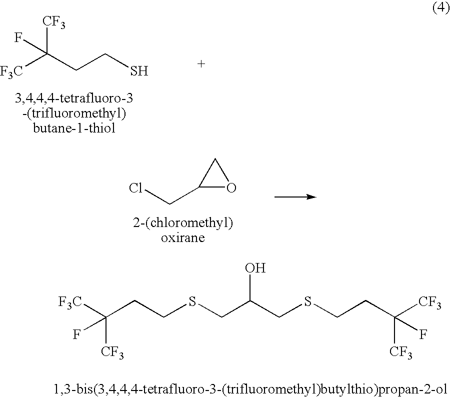

According to scheme (4) above, in a flask that can be equipped with an agitator, thermocouple, reflux condenser, and an addition funnel, about 22 mL of ethanol and 0.5 gram (0.02 mole) of cut sodium metal can be placed to form a mixture. The mixture can be observed to liberate gas and generate an exotherm. The mixture can be allowed to cool to from about 18° C. to about 24° C., and/or about 21° C. followed by the slow addition of 5.0 grams (0.02 mole) of 3,4,4,4-tetrafluoro-3-(trifluoromethyl)butane-1-thiol (see, e.g. Published International Applications) to form a reaction mixture. The reaction mixture can be allowed to stir at from about 18° C. to about 24° C., and/or about 21° C. for about 30 minutes. The reaction mixture can be concentrated to afford what can be observed to be a white crystalline solid. In a separate flask that can be equipped with an agitator, thermocouple, an ice water bath, reflux condenser, and an addition funnel, 1 gram (0.01 mole) of 2-(chloromethyl)oxirane and about 10 mL of anhydrous tetrahydrofuran (THF) can be placed to form a mixture and then chilled to about 3° C. The white crystalline solid can be combined with about 10 mL of anhydrous tetrahydrofuran to form an addition mixture. The addition mixture can be added drop wise to the mixture to form a reaction mixture. The addition rate can be such that the reaction mixture temperature is kept below about 10° C. Following the addition, the reaction mixture can be allowed to warm to from about 18° C. to about 24° C., and/or about 21° C. and held for from about 15 hours to about 21 hours, and/or about 18 hours. In a separate flask that can be equipped with an agitator and a thermocouple, about 22 mL of ethanol and 0.5 gram (0.02 mole) of cut sodium metal can be placed to form a mixture. The mixture can be observed to liberate gas and generate an exotherm. The mixture can be allowed to cool to from about 18° C. to about 24° C., and/or about 21° C. To the mixture, 2.5 grams (0.01 mole) of 3,4,4,4-tetrafluoro-3-(trifluoromethyl)butane-1-thiol can be added to form a new mixture. The new mixture can be held stirring for about 20 minutes, then the ethanol can be removed to afford a salt. The salt can be combined with about 10 mL of THF to form a new addition mixture. The new addition mixture can be slowly added to the reaction mixture at from about 18° C. to about 24° C., and/or about 21° C. The reaction mixture can be observed to generate an exotherm and turn brown in color and can be held stirring for about 30 minutes. To the reaction mixture can be added about 40 mL of water to form a multiphase mixture. The pH of the multiphase mixture can be observed to be about 13, and about 60 mL of ammonium chloride can be added to afford a pH of about 7. The multiphase mixture can be separated and the aqueous layer extracted twice with 60 mL portions of ether. The organic layers can be combined, dried over sodium sulfate, filtered, and concentrated to afford what can be observed as an oil. The oil can be placed on a Kugelrohr distillation apparatus (140° C., 0.03 mmHg, 30 minutes) to afford 3.9 grams of an impure oil containing 1,3-bis(3,4,4,4-tetrafluoro-3-trifluoromethyl-butylsulfanyl)-propan-2-ol product. The product structure can be confirmed by NMR and/or chromatographic analysis.

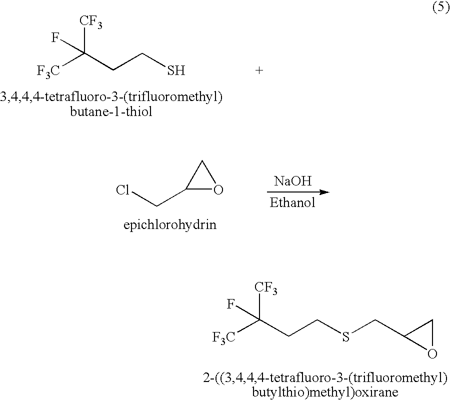

With reference to scheme (5) above, in a flask that can be configured with an agitator, a thermocouple, and an addition funnel, 5.0 gram (0.022 mole) of 3,4,4,4-tetrafluoro-3-(trifluoromethyl)butane-1-thiol (see, e.g. Published International Applications) can be dissolved in about 10 mL of a 40 percent (weight/weight) solution of NaOH in ethanol to form a mixture. To the mixture about 8.04 gram (0.09 mole) epichlorohydrin and about 0.3 gram (8.9×10⁻⁴ mole) of tetrabutylammonium hydrogen sulfate can be added to form a reaction mixture. The reaction mixture can then be held stirring from about 18° C. to about 24° C., and/or at about 21° C., for about one hour. The reaction mixture can be washed by addition with about 40 mL of water to form a multiphase mixture from which an organic layer can be separated from an aqueous layer. The aqueous layer can be treated three times with 30 mL portions of diethyl ether. The ethyl ether portions can be combined with the organic layer, dried over sodium sulfate, filtered, and concentrated in vacuo to afford about 4.09 gram (0.014 mole) of 2-((3,4,4,4-tetrafluoro-3-(trifluoromethyl)butylthio)methyl)oxirane product and an amount of a 1-(3,4,4,4-tetrafluoro-3-(trifluoromethyl)butylthio)-3-chloropropan-2-ol byproduct (not shown above). The product can be 91 percent pure by gas chromatography and can be observed as a colorless oil. The product structure can be confirmed by NMR and/or chromatographic analysis.

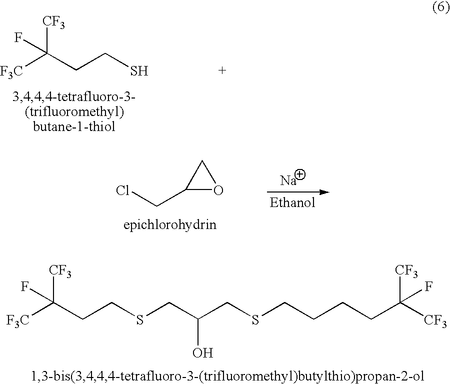

According to scheme (6) above, in a flask that can be equipped with a thermocouple, an agitator, and an addition funnel, 0.5 gram (0.02 mole) of cut sodium metal and about 22 mL of ethanol can be placed to form a mixture wherein an exotherm can be observed. To the mixture can be added drop wise, 5.0 gram (0.02 mole) of 3,4,4,4-tetrafluoro-3-(trifluoromethyl)butane-1-thiol (see, e.g. Published International Applications) at about 18° C. to about 24° C., and/or about 21° C. to form a reaction mixture that can then be stirred for about 30 minutes. The ethanol can then be removed in vacuo and a white crystalline solid recovered. Separately, about 1.0 gram (0.01 mole) of epichlorohydrin and about 10 mL tetrahydrofuran can be combined to form a another mixture, which can be chilled to about 3° C. by employing an ice/acetone bath. In about 10 mL anhydrous tetrahydrofuran, the crystalline white solid can be dissolved and placed into an addition funnel then added drop wise to the mixture wherein the reaction temperature can be kept around 5° C., from about 0° C. to about 10° C. to form another reaction mixture. Following the addition, the reaction mixture can be warmed to about 18° C. to about 24° C., and/or about 21° C. and stirred from about 15 hours to about 21 hours, and/or about 18 hours. To the reaction mixture, about 40 mL of water can be added to form a multiphase mixture having a pH of about 13. To the multiphase mixture, about 60 mL of an ammonium chloride solution can be added and from which an organic layer can be separated from an aqueous layer. The aqueous layer can be washed twice with 60 mL portions of ether and the organic layers combined, dried over sodium sulfate, filtered, and concentrated in vacuo. The concentrated organic can be placed on a Kugelrohr distillation apparatus at about 140° C. and 0.03 mmHg for about 30 minutes, to afford 3.9 gram (0.008 mole) of the 1,3-bis(3,4,4,4-tetrafluoro-3-(trifluoromethyl)butylthio)propan-2-ol product. The product structure can be confirmed by NMR and/or chromatographic analysis.

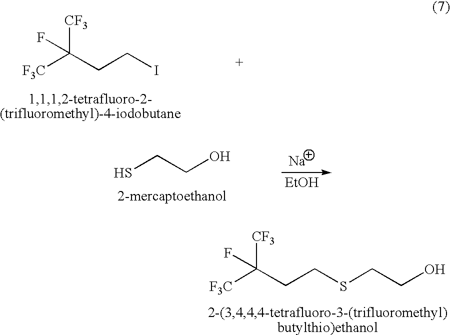

In conformity with scheme (7) above, in a flask that can be equipped with a thermocouple, an agitator, and a reflux condenser, about 30 mL of ethanol and 0.69 gram (0.003 mole) of cut sodium metal can be combined and stirred to form a mixture. To the mixture, 2.4 gram (0.03 mole) of 2-mercaptoethanol and 10.0 gram (0.03 mole) of 1,1,1,2-tetrafluoro-4-iodo-2-trifluoromethylbutane (see, e.g. Published International Applications) can be added separately to form a reaction mixture whereupon a transition of the reaction mixture color from clear to yellow can be observed. The reaction mixture can then be heated to reflux and held for a period of about four hours. To the reaction mixture, 1.0 mL of a 2N HCl solution can be added whereupon the reaction mixture can be observed to turn cloudy and have a pH of about 3. To the reaction mixture, about 40 mL of methylene chloride and 40 mL water can be added to form a multiphase mixture from which an organic phase can be separated from an aqueous phase. The organic phase can be collected, dried over sodium sulfate, filtered, and concentrated in vacuo to afford 8.3 grams of the 2-(3,4,4,4-tetrafluoro-3-trifluoromethyl-butylsulfanyl)-ethanol product that can be observed as a yellow oil. The product structure can be confirmed by NMR and/or chromatographic analysis.

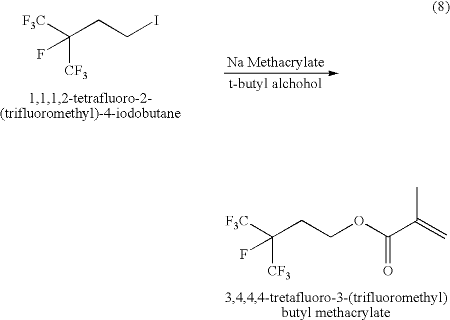

In accordance with scheme (8) above, in a 2 L autoclave that can be equipped with an agitator and a thermocouple, 400 grams (1.71 moles) of 1,1,1,2-tetrafluoro-2-(trifluoromethyl)-4-Iodobutane(see, e.g. Published International Applications), 211 grams (1.95 moles) sodium methacrylate, 4 grams (0.006 mole) 4-tert-butycatachol, and 902 grams of tert-butyl alcohol to form a mixture. The mixture can be stirred and heated to about 170° C. for about 20 hours. The mixture can be cooled to from about 18° C. to about 24° C., and/or about 21° C. The mixture can be washed with water to form a multiphase mixture from which an organic phase can be separated from an aqueous phase to afford 406 grams of crude product mixture having a purity (by gas chromatography) of about 34 (wt/wt) percent. Vacuum distillation can provide the 3,4,4,4-tetrafluoro-3-(trifluoromethyl)butyl methacrylate (b.p. 65° C.-66° C./20 Torr) product. The product structure can be determined by NMR and/or chromatographic analysis.

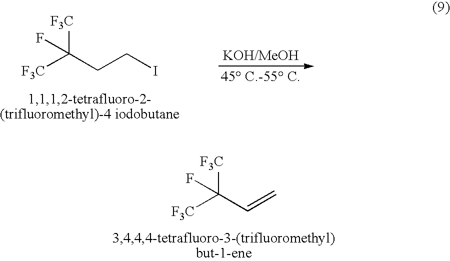

According to scheme (9) above, in a flask that can be equipped with an agitator, thermocouple, cold product trap, and an addition funnel, 64 grams (1.14 moles) of potassium hydroxide and about 240 mL of methanol can be placed to form a mixture. The mixture can be heated to from about 45° C. to about 55° C. followed by the drop wise addition of 244.6 grams (0.75 mole) of 1,1,1,2-tetrafluoro-2-(trifluoromethyl)-4-iodobutane (see, e.g. Published International Applications) to form a reaction mixture. In the cold product trap, 144.8 grams of 3,4,4,4-tetrafluoro-3-(trifluoromethyl)but-1-ene product can be collected having of about 93 percent purity by gas chromatography. The product structure can be confirmed by NMR and/or chromatographic analysis.

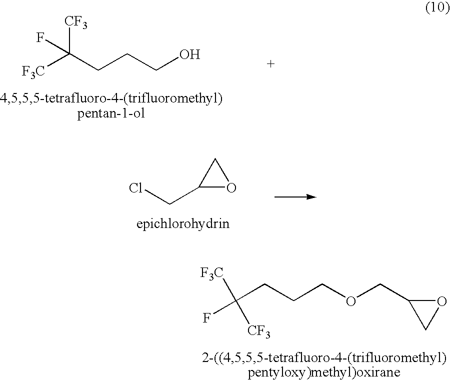

Referring to scheme (10) above, in a flask that can be equipped with agitator, about 15 mL of a 40 (wt/wt) percent solution of NaOH, 10 grams (0.04 mole) of 4,5,5,5-tetrafluoro-4-trifluoromethyl-pentan-1-ol (see, e.g. Published International Applications) 16.2 grams (0.18 mole) of epichlorohydrin, and 0.7 gram (0.002 mole) of tetrabutylammonium hydrogen sulfate can be added to form a mixture. The mixture can be allowed to agitate at from about 18° C. to about 24° C., and/or about 21° C. for from about 15 hours to about 21 hours, and/or about 18 hours. To the mixture, about 30 mL of water can be added to form a multiphase mixture from which an organic phase can be separated from an aqueous phase. The aqueous phase can be extracted with three times with about 30 mL portions of ether. The organic phases can be combined, dried, and concentrated in vacuo to afford what can be observed as an oil. The oil can be further concentrated by placing onto a Kugelrohr distillation apparatus (0.03 mmHg, 21° C., 30 minutes) to afford 6.2 grams of the 2-((4,5,5,5-tetrafluoro-4-(trifluoromethyl)pentyloxy)methyl)oxirane product that can be observed to be a yellowish oil. The product structure can be confirmed by NMR and/or chromatographic analysis.

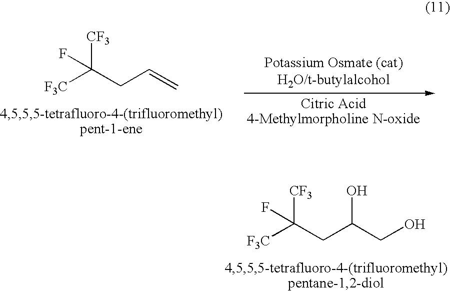

In conformity with scheme (11) above, in flask that can be equipped with an agitator, thermocouple, and heating mantle and controller, 30.4 gram (0.145 mole) of 4,5,5,5-tetrafluoro-4-trifluoromethyl)pent-1-ene (see, e.g. Published International Applications) and 19.7 gram (0.103 mole) of citric acid, 53.4 gram tert-butyl alcohol, 69.4 gram of water, 0.08 gram (0.0002 mole) potassium osmate, and 35.6 gram (0.152 mole) 4-methylmorpholine N-oxide can be added to form a mixture. The mixture can be agitated for from about 4 hours to about 24 hours at from about 18° C. to about 24° C., and/or about 21° C. wherein a change in color of the mixture from a yellowish green to a slight brownish green can be observed. The tert-butyl alcohol can be removed in vacuo providing an aqueous phase that can be acidified with about 100 mL of a 1 molar solution of a hydrochloric acid solution and the aqueous phase can be extracted with about two separate 100 mL ethyl acetate washings. The ethyl acetate can be removed by evaporation to afford about 25.5 gram (0.105 mole) 4,5,5,5-tetrafluoro-4-(trifluoromethyl)pentane-1,2-diol. (m/z: 244 (M⁺), 213 (M⁺ —CH₃O), 193 (M⁺-CH₃OF), 173 (M⁺-CH₃OF₂)).

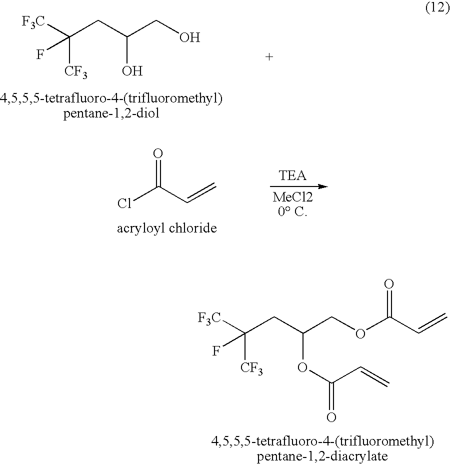

According to scheme (12) above, in a flask that can be equipped with an agitator, thermocouple, reflux condenser, ice water bath, and an addition funnel, 5.128 grams (0.021 mole) of 4,5,5,5-tetrafluoro-4-(trimethyl)pentane-1,2-diol (see scheme 11 above), 5.25 grams (0.052 mole) of triethylamine (TEA) can be added to form a mixture. The mixture can be chilled to from about 0° to about 5° C. using an ice water bath. To the addition funnel, about 20 mL of methylene chloride and 6.6 grams (0.073 mole) of acryloyl chloride can be added to form an addition mixture. The addition mixture can be added drop wise to the mixture to form a reaction mixture. The addition rate of the addition mixture to the mixture can be such that the reaction mixture temperature is maintained at or below about 10° C. The reaction mixture can be warmed to from about 18° C. to about 24° C., and/or about 21° C. and held for from about 15 hours to about 21 hours, and/or about 18 hours. The reaction mixture can then be washed once with about 100 mL of a 2N HCl solution, three times with about 100 mL portions of a saturated sodium bicarbonate solution, once with about 100 mL of saturated KCl solution each time forming a multiphase mixture from which an organic phase can be separated from an aqueous phase. The aqueous phases can be collected and extracted with about 100 mL of methylene chloride, the organic phases combined, dried over magnesium sulfate, filtered, and concentrated in vacuo to afford a viscous oil which can contain the 4,5,5,5-tetrafluoro-4-(trimethyl)pentane-1,2-diacrylate product as well as the hydroxypentylacrylate mono-adduct. m/z: 352 (M⁺), 281 (M⁺-C₃H₃O₂).

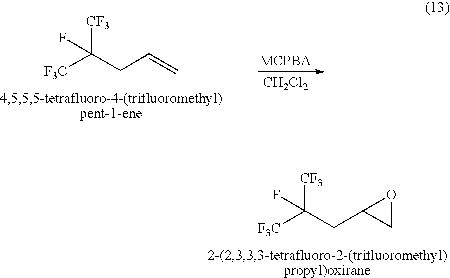

According to scheme (13) above, in a flask that can be equipped with a thermocouple and a heating mantle 2.0 grams (0.01 moles) of 4,5,5,5-tetrafluoro-4-(trifluoromethyl)pent-1-ene (see, e.g. Published International Applications) about 20 mL of chlorobenzene, and 2.5 grams (2.47 mole) of m-chloroperoxybenzoic acid can be added to form a mixture. The mixture can be heated to about 45° C. and held for about 41 hours. To the mixture, 0.5 grams (0.003 mole) of m-chloroperoxybenzoic acid can be added to form a reaction mixture. The reaction mixture can be heated to about 55° C. for about 48 hours. The reaction mixture can be allowed to cool to from about 18° C. to about 24° C., and/or from about 21° C. and allowed to stir for from about 60 hours to about 72 hours, and/or from about 66 hours wherein a white precipitate can be observed to have been formed. The reaction mixture can be filtered and the filtrate washed with about 20 mL saturated sodium bicarbonate solution to form a multiphase mixture from which an organic phase can be separated from an aqueous phase. The organic phase can be dried over sodium sulfate, filtered, and distilled (131° C.-133° C./760 Torr) to afford about 0.6 gram 2-(2,3,3,3-tetrafluoro-2-(trifluoromethyl)propyl)oxirane product. The product structure can be confirmed by NMR and gas chromatographic analysis.

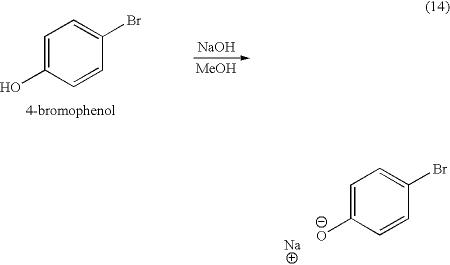

According to scheme (14) above, in a 500 mL flask, 51.8 grams (0.3 mole) of 4-bromophenol, 24.7 grams of a 48.53 percent (wt/wt) NaOH solution, and about 100 mL of methanol can be placed to form a mixture whereupon an exotherm can be observed. The reaction mixture can then be concentrated in vacuo and dried in a vacuum oven to afford 61.7 grams of sodium bromophenoxide as a white solid.

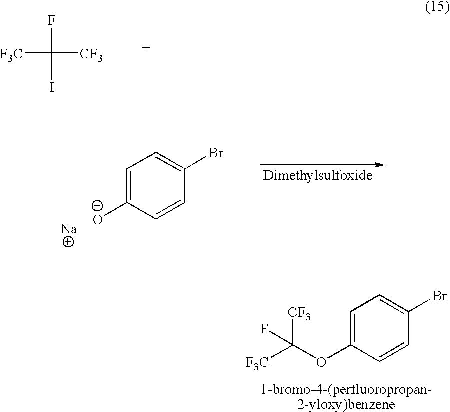

In accordance with scheme (15), into a 500 cc flask that can be equipped with an agitator, thermocouple, reflux condenser, and an addition funnel, 61.7 grams of crude sodium bromophenoxide (refer to scheme (14) above), 330 mL of dimethylsulfoxide can be placed to form a mixture under anhydrous conditions. To the mixture, 95.5 gram (0.32 mole) of 2-iodoheptafluoropropane (see, e.g. Published International Applications) can be added drop wise to form a reaction mixture whereupon an exotherm can be observed. The reaction mixture can be allowed to stir for from about two hours to about four hours at from about 18° C. to about 24°, and/or about 21° C. The reaction mixture can be washed stepwise with water, saturated sodium bicarbonate and with water wherein each step can be observed to form a multiphase mixture from which an organic phase can be separated from an aqueous phase. The aqueous phase can be washed with methylene chloride, all organic phases can be combined, dried over magnesium sulfate, filtered, and concentrated in vacuo to form a concentrated mixture. The concentrated mixture can be distilled under vacuum to provide a mixture of products that include both 1-bromo-4-(1,1,1,3,3,3-hexafluoropropan-2-yloxy)benzene as a viscous colorless oil (m/z: 322(M+)) and 1-bromo-4-(perfluoropropan-2-yloxy)benzene (m/z: 340(M+)). The product structure can be confirmed by NMR and/or chromatographic analysis.

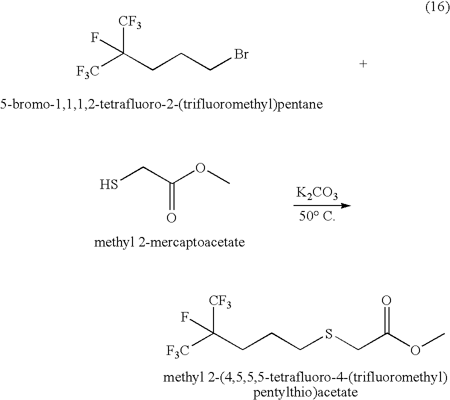

According to scheme (16) above, in a flask that can be equipped with an agitator, thermocouple, and a heating mantle, 5.0 grams (0.017 mole) of 1,1,1,2-tetrafluoro-2-(trifluoromethyl)-5-bromopentane (see, e.g. Published International Applications) 2.37 grams (0.017 mole) of potassium carbonate, 1.82 grams (0.017 mole) of mercapto-acetic acid methylester, and about 20 mL of dimethylformamide (DMF) can be placed to form a reaction mixture. The reaction mixture can be heated to about 50° C. for about three hours and allowed to cool to from about 18° C. to about 24° C., and/or about 21° C. for from about 15 hours to about 21 hours, and/or about 18 hours wherein the reaction mixture can be observed as a yellow slurry. The yellow slurry can be added to about 50 mL water and about 50 mL ethyl acetate to form a multiphase mixture from which an organic phase can be separated from an aqueous phase. The aqueous phase can be collected and washed twice with 50 mL portions of ethyl acetate. The organic phases can be combined, dried over sodium sulfate, filtered, and concentrated in vacuo to afford about 4.4 grams of methyl-2-(4,5,5,5-tetrafluoro-4-(trifluoromethyl)pentylthio)acetate product as a yellow oil. The product structure can be confirmed by NMR and/or chromatographic analysis.

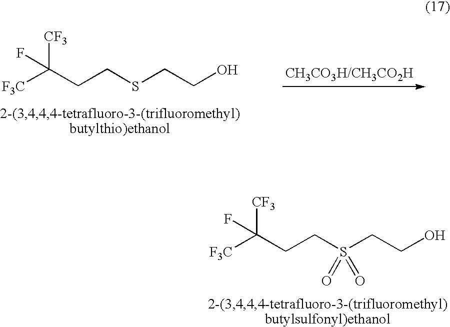

According to scheme (17) above, in a flask that can be configured with a thermocouple, an addition funnel, and an agitator, 5.6 gram (0.02 mole) of 2-(3,4,4,4-tetrafluoro-3-trifluoromethyl-butylsulfanyl)-ethanol (see, e.g. Published International Applications) can be placed and cooled to about 0° C. To the flask, 19.41 gram (0.26 mole) of paracetic acid can be added drop wise to form a mixture at such a rate as to keep the temperature below about 20° C. The mixture can be allowed to stir for about 30 minutes, which can be followed by the addition of about 25 mL water to form a multiphase mixture from which an organic phase can be separated from an aqueous phase. The organic phase, which can be observed to be colorless and can be collected to afford about 3.2 gram of the 2-(3,4,4,4-tetrafluoro-3-(trifluoromethyl)butylsulfonyl)ethanol product. The product structure can be confirmed by NMR and/or chromatographic analysis.

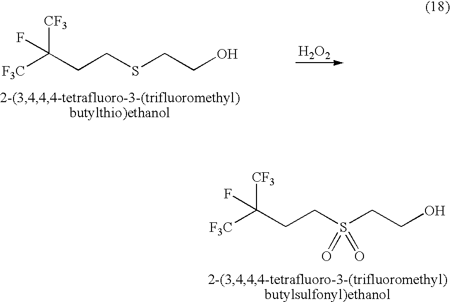

Referring to scheme (18) above, in a flask that can be configured with a thermocouple, an agitator, 200 gram (0.73 mole) of 2-(3,4,4,4-tetrafluoro-3-trifluoromethyl-butylsulfanyl)-ethanol (see, e.g. Published International Applications) can be dissolved in about 275 mL of ethanol and about 44 mL of water to form a mixture. In an addition funnel, 100 mL of a 50 percent (wt/wt) solution of hydrogen peroxide can be placed and added drop wise to the mixture to form a reaction mixture. The reaction mixture can be observed to have an exotherm that can peak at about 83° C. and a color transition from clear to orange to yellow. During the addition, adjustment of the peroxide addition rate and employment of an ice bath can be useful together or separately to control the reaction mixture exotherm. While stirring, the reaction mixture can be allowed to cool to, and maintained at, about 40° C. for about 30 minutes. The reaction mixture can be allowed to cool to from about 18° C. to about 24° C., and/or about 21° C. To the reaction mixture, about 300 mL ethanol and about 100 gram of Norit A (an activated carbon) can be added to form a slurry. The slurry can be allowed to stir for from about 15 hours, from about 10 hours to about 20 hours and then filtered through a suitable media, for example celite. The filter cake can be washed about three times with about 200 mL ethanol. The filtrate can be concentrated in vacuo yielding about 210.9 gram of the 2-(3,4,4,4-tetrafluoro-3-(trifluoromethyl)butylsulfonyl)ethanol product. The product structure can be confirmed by NMR and/or chromatographic analysis.

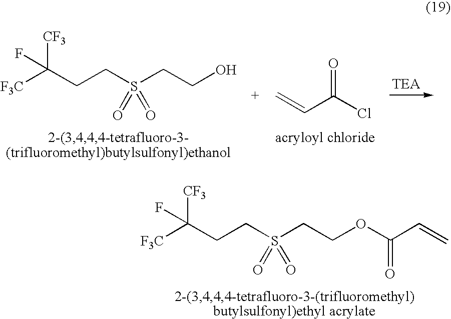

In accordance with scheme (19) above, in a flask under a nitrogen atmosphere, that can be equipped with an addition funnel, a thermocouple, and an ice water bath, 110 grams (0.359 mole) of 2-(3,4,4,4-tetrafluoro-3-(trifluoromethyl)butylsulfonyl)ethanol (refer to scheme (18) above), about 990 mL of methylene chloride, and about 63 mL of triethylamine can be placed to form a mixture and cooled to about 0° C. In the addition funnel that can be under a nitrogen atmosphere, 40 grams (0.45 mole) of acryloyl chloride and about 660 mL of methylene chloride can be placed to form an addition mixture. To the mixture, the addition mixture can be added drop wise to form a reaction mixture. The addition can be completed in about one hour, keeping the reaction mixture temperature below from about 0° C. to about 10° C., and/or about 5° C. The reaction mixture can be allowed to warm to from about 18° C. to about 24° C., and/or about 21° C. and held for about two hours. The reaction mixture can be washed by adding 2 L of a 2N solution of HCl, about 2 L portions of a saturated sodium bicarbonate solution, 2 L of a brine solution wherein each of the aqueous additions above can result in the formation of multiphase mixture from which an organic phase can be separated from an aqueous phase. The organic phase can be collected and dried over sodium sulfate, filtered, and concentrated in vacuo to afford an oil. The oil can be placed on a Kugelrohr distillation apparatus (0.03 mmHg, 70° C., 60 minutes) to afford 92.6 grams of the 2-(3,4,4,4-tetrafluoro-3-(trifluoromethyl)butylsulfonyl)ethyl acrylate product. The product structure can be confirmed by NMR and/or chromatographic analysis.

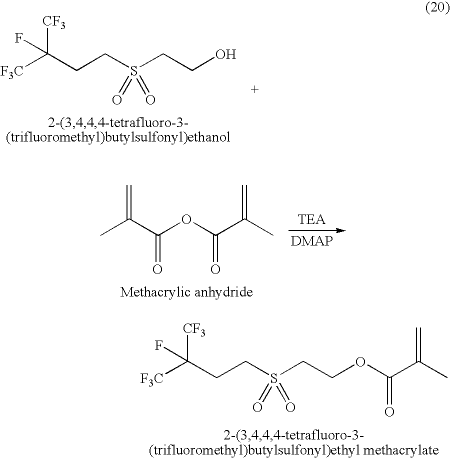

With reference to scheme (20) above, to a flask that can be equipped with a thermocouple, an agitator, an addition funnel, 117.9 gram (0.39 mole) of 2-(3,4,4,4-tetrafluoro-3-trifluoromethyl-butane-1-sulfonyl)-ethanol (refer to scheme (18) above), 4.7 grams (0.039 mole) of 4-dimethylamino pyridine (DMAP), about 67 mL of triethylamine (TEA), and about 450 mL of methylene chloride that can be chilled with an ice/acetone bath to from about 0° C. to about 5° C. to form a reaction mixture can be placed. To the mixture, 74.2 grams (0.48 mole) of methacrylic anhydride, about 300 mL of methylene chloride can be added drop wise to form a reaction mixture wherein the addition rate can be such that the reaction mixture temperature does not exceed about 10° C. The reaction mixture can be allowed to warm from about 18° C. to about 25° C., and/or about 21° C. and washed with about 1 liter of a 0.5N HCl, about three times each with one liter of a saturated sodium bicarbonate solution and then with about one liter of a saturated brine solution wherein each of the aqueous additions above can result in the formation of multiphase mixture from which an organic phase can be separated from an aqueous phase. The organic phase can be collected and dried over sodium sulfate, filtered, and concentrated in vacuo to afford 136.7 grams of the 2-(3,4,4,4-tetrafluoro-3-trifluoromethyl-butane-1-sulfonyl)-ethyl ester product as a yellow oil. The product structure can be confirmed by NMR and/or chromatographic analysis.

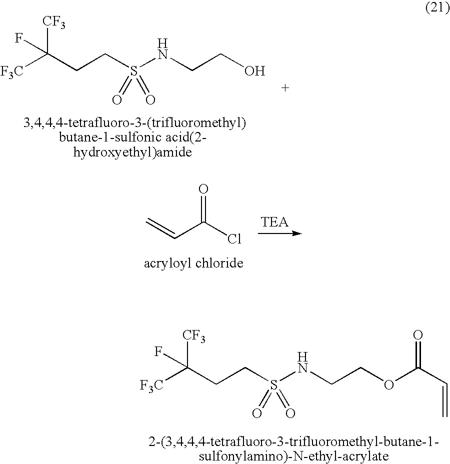

According to scheme (21) above, in a flask under a nitrogen atmosphere that can be equipped with an agitator, an addition funnel, an ice water bath, and a thermocouple, 5 gram (0.016 mole) of 3,4,4,4-tetrafluoro-3-trifluoromethyl-butane-1-sulfonic acid(2-hydroxyethyl)amide (see, e.g. Published International Applications) and about 2.5 mL of trethyl amine can be added to form a mixture. The mixture can be chilled to from about 0° C. to about 10° C., and/or about 5° C. In the addition funnel, 1.6 gram (0.02 mole) of acryloyl chloride and about 30 mL of methylene chloride can be placed to form an addition mixture. To the mixture, the addition mixture can be added drop wise over about 30 minutes to form a reaction mixture. The rate of addition can be such that the temperature remains below about 10° C. The reaction mixture can then be allowed to warm to from about 18° C. to about 24° C., and/or about 21° C. then held at from about 15 hours to about 21 hours, and/or about 18 hours. The reaction mixture can be concentrated to afford what can be observed as a white semisolid. The white semisolid can be dissolved in about 100 mL of methylene chloride then washed with 100 mL of 2N HCl solution, three times with about 100 mL of a saturated sodium bicarbonate solution, and about 100 mL of brine wherein each of the aqueous additions above can result in the formation of multiphase mixture from which an organic phase can be separated from an aqueous phase. The organic phase can be concentrated in vacuo and placed on a Kugelrohr distillation apparatus (0.03 mmHg, 70° C., 20 minutes) to afford an impure mixture containing the product 2-(3,4,4,4-tetrafluoro-3-trifluoromethyl-butane-1-sulfonylamino)-N-ethyl acrylate. The product structure can be confirmed by NMR and/or chromatographic analysis.

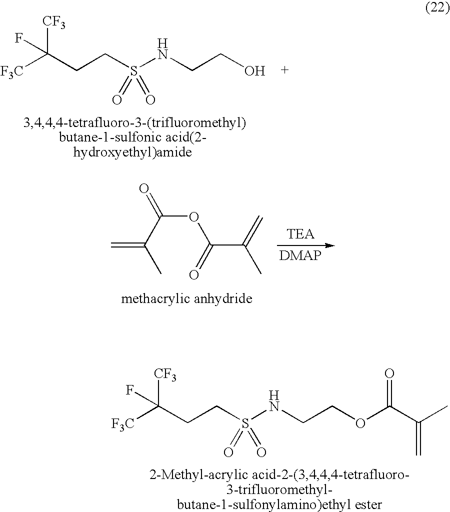

In accordance with scheme (22) above, in a flask that can be equipped with an addition funnel, an agitator, and a thermocouple, under a nitrogen atmosphere, 52.4 grams (0.163 mole) of 3,4,4,4-tetrafluoro-3-trifluoromethylbutane-1-sulfonic acid (2-hydroxyethyl)amide (see, e.g. Published International Applications) can be placed to form a mixture. The mixture can be chilled to from about 0° C. to about 5° C., and/or about 0° using an ice/acetone bath. To the mixture can be added drop wise, 27.66 grams (0.18 mole) of methacrylic anhydride dissolved in about 315 mL of methylene chloride over about 30 minutes to form a reaction mixture. The addition rate can be such that the temperature can be kept below about 10° C. The reaction mixture can be allowed to warm from about 18° to about 24° C., and/or about 21° C., over a period from about 12 hours to about 18 hours, and/or for about 15 hours. The reaction mixture can be washed with about 500 mL of 0.5N HCl, about three times with 700 mL saturated sodium bicarbonate solution, and about 700 mL brine solution wherein each of the aqueous additions above can result in the formation of multiphase mixture from which an organic phase can be separated from an aqueous phase. The organic phase can be collected, dried over sodium sulfate, filtered, and concentrated in vacuo to afford 52 grams of the 2-methylacrylic acid 2-(3,4,4,4-tetrafluoro-3-trifluoromethylbutane-1-sulfonylamino)ethyl ester product as what can be observed as a yellow oil that can solidify upon cooling to about 21° C. The product structure can be confirmed by using NMR and/or chromatographic analysis.

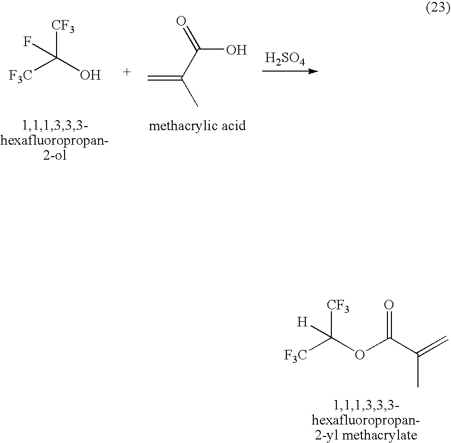

According to scheme (23) above, in a flask that can be equipped with an agitator, thermocouple, and an addition funnel that can be equipped with a dip tube, 300 grams (1.79 moles) of 1,1,1,2,3,3,3-heptafluoropropan-2-ol (see, e.g. Published International Applications) 230.54 grams (2.68 moles) of methacyrlic acid, and 3.0 grams (0.02 mole) of 1,1,1,3,3,3-hexafluoropropan-2-ol can be placed to form a mixture while agitating the mixture at from about 18° C. to about 24° C., and/or about 21° C. To the mixture, 590 grams (5.95 moles) of fuming sulfuric acid can be added drop wise through the dip tube over a period of about 75 minutes to form a multiphase reaction mixture whereupon an exotherm can be observed to afford a peak temperature of about 61.3° C. The reaction mixture can be heated to about 70° C. and held for about three hours wherein some gas evolution can be observed. The multiphase reaction mixture can be observed to contain a clear and colorless liquid phase and a dark orange oily phase. A simple atmospheric distillation at about 280 mmHg can be immediately performed without cooling the reaction flask wherein the reflux condenser can be set at about −12° C. One fraction, about 308.7 grams, can be collected and observed to be clear and colorless and have a boiling point of about 50° C. The fraction can be washed twice with about 220 mL of 1N NaOH for about 15 minutes at from about 18° C. to about 24° C., and/or about 21° C. to form a multiphase mixture from which an organic phase can be separated from an aqueous phase. The organic phase can be collected to afford about 254.6 grams of the 1,1,1,3,3,3-hexafluoropropan-2-yl methacrylate product. To the product, about 25 milligrams of 4-tert butyl catchecol can be added. The product structure can be confirmed by NMR and/or chromatographic analysis.

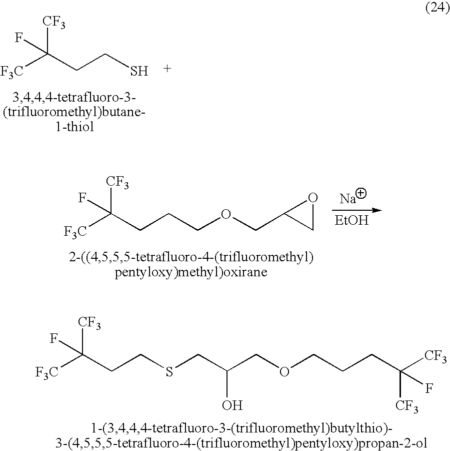

According to scheme (24) above, in a flask that can be equipped with an agitator and an addition funnel, 0.15 gram (0.007 mole) of sodium metal and about 6.6 mL of ethanol can be placed to form a mixture from which an exotherm can be observed. The mixture can be cooled to from about 18° C. to about 24° C., and/or about 21° C., then 1.21 grams (0.005 mole) of 3,4,4,4-tetrafluoro-3-(trifluoromethyl)butane-1-thiol (see, e.g. Published International Applications) can be added to form a pot mixture. The pot mixture can be allowed to stir for about 45 minutes whereupon 1.5 grams (0.005 mole) of 2-(4,5,5,5-tetrafluoro-4-trifluoromethyl-pentyloxymethyl)-oxirane (see, e.g. Published International Applications) can be added drop wise to form a reaction mixture. The reaction mixture can be allowed to agitate for from about 15 hours to about 21 hours, and/or about 18 hours. To the reaction mixture, about 25 mL of water can be added and the pH can be observed to be about 11, about 25 mL of ammonium chloride solution and the pH can be observed to be about 8 wherein each of the aqueous additions above can result in the formation of multiphase mixture from which an organic phase can be separated from an aqueous phase. The aqueous phase can be extracted three times with about 50 mL portions of ether. The organic phase can be combined and washed with about 100 mL of water to form a multiphase mixture from which an organic phase can be separated from an aqueous phase. The organic phase can be collected, dried over sodium sulfate, filtered and concentrated in vacuo to afford what can be observed as a pale yellow oil. The pale oil can be placed onto a Kugelrohr distillation apparatus (0.03 mmHg, 100° C., 30 minutes) to afford 1.8 grams of the 1-(3,4,4,4-tetrafluoro-3-trifluoromethyl-butylsulfanyl-3-(4,5,5,5-tetrafluoro-4-trifluoromethyl-pentyoxy)propan-2-ol product. The product structure can be confirmed by NMR and/or chromatographic analysis.

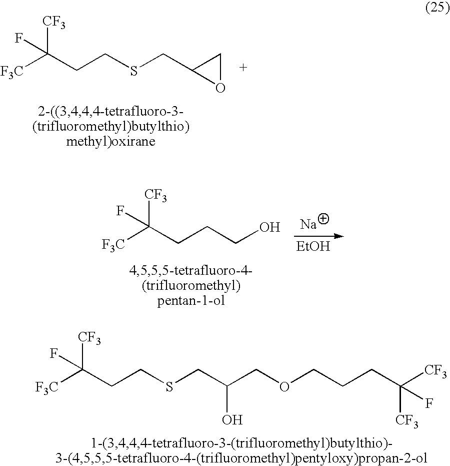

In reference to scheme (25) above, in a flask that can be equipped with an agitator, thermocouple, a nitrogen purge, and an addition funnel, 2.0 grams (0.009 mole) of 4,5,5,5-tetrafluoro-4-trifluoromethyl-pentan-1-ol (see, e.g. Published International Applications) and 0.1 gram (0.0007 mole) of boron trifluoride etherate can be added to form a mixture. The mixture can be heated to about 70° C. then 2.509 grams (0.009 mole) of 2-(3,4,4,4-tetrafluoro-3-trifluoromethyl-butylsulfanylmethyl)-oxirane (see, e.g. Published International Applications) can be slowly added to form a reaction mixture over a period of about 15 minutes wherein the temperature can be maintained at about 70° C. The reaction mixture can be heated to about 75° C. and allowed to stir for about one hour. The reaction mixture can be allowed to cool to from about 18° C. to about 24° C., and/or about 21° C. and held for about one hour. To the reaction mixture, about 25 mL of water can be added to form a multiphase mixture from which an organic phase can be separated from an aqueous phase and the pH can be observed to be about 11. To the organic phase, about 25 mL of ammonium chloride solution can be added to form another multiphase mixture from which an organic phase can be separated from an aqueous phase and the pH can be observed to be about 8. The aqueous phase can be extracted three times with about 50 mL portions of ether. The organic phase can be combined and about 100 mL of water can be added then about 100 mL of ether to form a multiphase mixture from which an organic phase can be separated from an aqueous phase. The organic phase can be dried over sodium sulfate, filtered and stripped of solvent to afford 2.6 grams of the 1-(3,4,4,4-tetrafluoro-3-trifluoromethyl-butylsulfanyl-3-(4,5,5,5-tetrafluoro-4-trifluoromethyl-pentyoxy)propan-2-ol product. The product structure can be confirmed by NMR and/or chromatographic analysis.

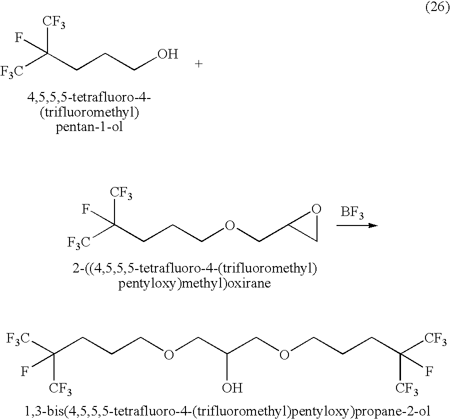

According to scheme (26) above, in a flask that can be under a nitrogen atmosphere and equipped with an agitator, thermocouple, and an addition funnel, 0.682 gram (0.005 mole) of boron trifluoride diethyl etherate and 13.7 grams (0.06 mole) of 4,5,5,5-tetrafluoro-4-(trifluoromethyl)pentan-1-ol (see, e.g. Published International Applications) can be added to form a mixture. The mixture can be heated to about 70° C. and 17 grams (0.06 mole) of 2-((4,5,5,5-tetrafluoro-4-(trifluoromethyl)pentyloxy)methyl)oxirane (see, e.g. Published International Applications) can be slowly added drop wise to form a reaction mixture. The rate of addition can be such that the temperature is maintained at about 70° C. The reaction mixture can be heated to about 75° C. and held for about one hour and allowed to cool to from about 18° C. to about 24° C., and/or about 21° C. and held for about an hour. To the reaction mixture, about 1 L of water can be added to form a multiphase mixture from which an organic phase can be separated from an aqueous phase. The aqueous phase can be extracted with about 1 L of ether. The organic phases can be combined, dried over sodium sulfate, filtered, and concentrated in vacuo to afford what can be observed as a pale-oil. The pale oil can be placed on a Kugelrohr distillation apparatus (0.01 mmHg, 1 hour, 130° C.) to afford about 6.6 grams of the 1,3-bis(4,5,5,5-tetrafluoro-4-(trifluoromethyl)pentyloxy)propan-2-ol as a minor product. The major product can be diadduct 1-(1,3-bis(4,5,5,5-tetrafluoro-4-(trifluoromethyl)pentyloxy)propan-2-yloxy)-3-(4,5,5,5-tetrafluoro-4-(trifluoromethyl)pentyloxy)propan-2-ol. The product structures can be confirmed by NMR and/or chromatographic analysis.

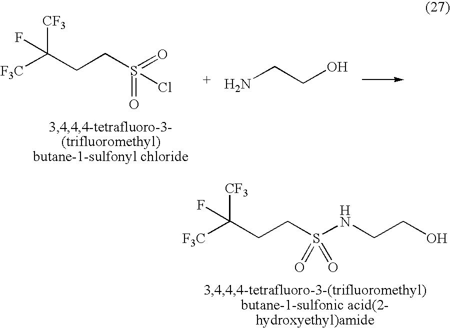

With reference to scheme (27) above, in a flask that can be equipped with an addition funnel, an agitator, and a thermocouple, 92.7 grams (1.52 moles) ethanolamine and about 375 mL methylene chloride can be placed under a nitrogen atmosphere to form a mixture. The mixture can be chilled to about 0° C. using an ice/acetone bath. To the mixture can be added drop wise, 75 grams (0.25 mole) 3,4,4,4-tetrafluoro-3-trifluoromethylbutane-1-sulfonyl chloride (see, e.g. Published International Applications) to form a reaction mixture. The addition rate can be such that the reaction mixture temperature is kept below about 5° C. The reaction mixture can be allowed to warm from about 18° C. to about 24° C., and/or about 21° C., and stirred for about one hour. The reaction mixture can then be diluted with about 750 mL of methylene chloride and washed successively by addition with about 750 mL water, about 750 mL of a 5 percent (wt/wt) HCl solution, and about 750 mL of a saturated sodium bicarbonate solution. The organic layer can be collected and dried over sodium sulfate, filtered and concentrated in vacuo affording 38.38 grams 3,4,4,4-tetrafluoro-3-(trifluoromethyl)butane-1-sulfonic acid (2-hydroxyethyl)amide product that can be observed to be a white solid. The product structure can be confirmed by NMR and/or chromatographic analysis.

R_{F}-Compositions and methods of making R_{F}-compositions are described with reference to FIG. 2. Referring to FIG. 2, a system 10 is shown for preparing halogenated compositions that includes reagents such as a taxogen 2, a telogen 4, and an initiator 6 being provided to reactor 8 to form a product such as a telomer 9. In exemplary embodiments system 10 can perform a telomerization process. According to an embodiment, taxogen 2 can be exposed to telogen 4 to form telomer 9. In accordance with another embodiment, taxogen 2 can be exposed to telogen 4 in the presence of initiator 6. Reactor 8 can also be configured to provide heat to the reagents during the exposing.

Taxogen 2 can include at least one CF₃-comprising compound. The CF₃-comprising compound can have a C-2 group having at least one pendant —CF₃ group. In exemplary embodiments taxogen 2 can comprise an oletin, such as 3,3,3-trifluoropropene (TFP, trifluoropropene), ethene, and/or 1,1,3,3,3-pentafluoropropene (PFP, pentafluoropropene). In exemplary embodiments, taxogen 2 can include trifluoropropene and telogen 44 can include (CF₃)₂CFI, with a mole ratio of taxogen 42 to telogen 44 being from about 0.2:1 to about 10:1, from about 1:1 to about 5:1, and/or from about 2:1 to about 4:1. Taxogen 2 can include 4,5,5,5-tetrafluoro-4-(trifluoromethyl)pen-1-tene and/or 6,7,7,7-tetrafluoro-6-(trifluoromethyl)hept-1-ene, and telogen 4 can include (CF₃)₂CFI, for example.

According to additional embodiments, taxogen 2 can include those compounds shown below in Table 2.

TABLE 2
Exemplary Taxogens

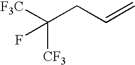

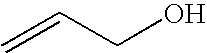

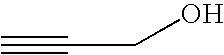

Telogen 4 can include halogens such as fluorine and/or chlorine. Telogen 4 can include at least four fluorine atoms and can be represented as R_{F}Q and/or R_{Cl}Q. The R_{F} group can include at least four fluorine atoms and the Q group can include one or more atoms of the periodic table of elements. Exemplary R_{F} groups can include: ((CF₃)₂CFCH₂)₂CH—; ((CF₃)₂CFCH₂)₂CH₂CH₂—; (CF₃)₂CFCH₂((CF₃)₂CF)CH—; (CF₃)₂CFCH₂CH(CF₃)CH₂CH(CF₃)—; and/or (CF₃)₂CFCH₂CH₂CH₂CH₂((CF₃)₂CFCH)CH—.

R_{F}-Q can be 2-iodofluoropropane, for example. Exemplary telogens can include the halogenated compounds described above, such as (CF₃)₂CFI, C₆F₁₃I, and/or trichloromethane. Additional exemplary telogens can include (CF₃)₂CF₁, C₆F₁₃I, trichloromethane, HP(O)(OEt)₂, BrCFClCF₂Br, R—SH (R being a group having carbon), and/or MeOH. The Q group can be H or I with the R_{F} group being (CF₃)₂CF— and/or —C₆F₁₃, for example. The R_{Cl} group can include at least one —CCl₃ group.

According to additional embodiments, telogen 4 can include those compounds shown below in Table 3. As exemplary implementations are shown in Table 3 below, telogens can be products of telomerizations.

TABLE 3
Exemplary Telogens

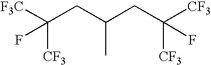

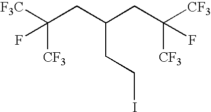

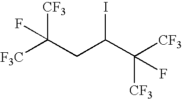

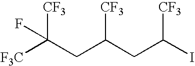

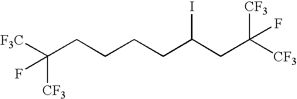

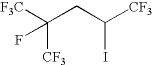

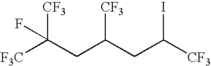

In exemplary embodiments, taxogen 2 can include trifluoropropene and telogen 4 can include (CF₃)₂CFI, with a mole ratio of taxogen 2 to telogen 4 being from about 1:1 to about 1:10, 1:4 to about 4:1, and/or to about 2:1 to about 4:1.

Reactor 8 can be any lab-scale or industrial-scale reactor and, in certain embodiments, reactor 8 can be configured to control the temperature of the reagents therein. According to exemplary embodiments reactor 8 can be used to provide a temperature during the exposing of the reagents: of from about 90° C. to about 180° C.; of from about 60° C. to about 220° C.; and/or of from about 130° C. to about 150° C.

Telomer 9, produced upon exposing taxogen 2 to telogen 4, can include R_{F}(R_{T})ₙQ and/or R_{Cl}(R_{T})ₙH. The R_{T} group can include at least one C-2 group having a pendant —CF₃ group, such as

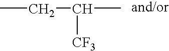

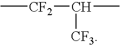

Exemplary products include

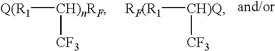

one or both of

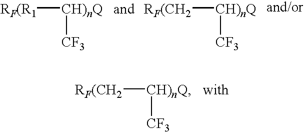

R₁ including at least one carbon atom, such as —CH₂— and/or —CF₂—, for example. R_{T} can also include —CH₂—CF₂—; —CH₂—(CH₂CF(CF₃)₂)CH—; and/or —CH₂—CH₂—. In exemplary embodiments, n can be at least 1 and in other embodiments n can be at least 2 and the product can include one or more

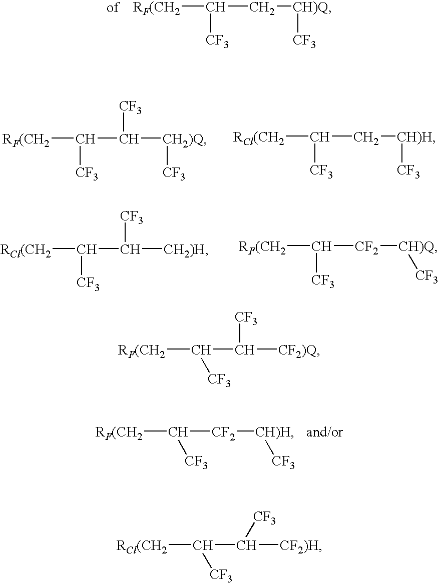

for example. According to other implementations n can be 3 or even at least 4. In exemplary embodiments, n can be at least 1 and in other embodiments n can be at least 2 and the product can include one or more of

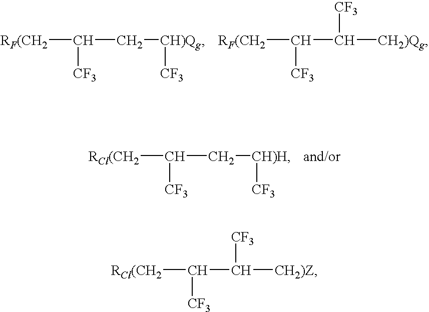

Z being H, Br, and/or Cl, for example.

In an exemplary embodiment, the taxogen trifluoropropene can be exposed to the telogen (CF₃)₂CFI to form the telomer

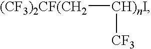

and, by way of another example, trifluoropropene can be exposed to the telogen C₆F₁₃I to form the telomer

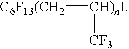

In an exemplary embodiment, the taxogen trifluoropropene can be exposed to the telogen (CF₃)₂CFI to form the telomer

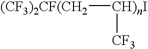

and/or

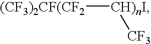

and, by way of another example, trifluoropropene can be exposed to the telogen C₆F₁₃I to form the telomer

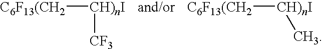

In accordance with another embodiment, the taxogen trifluoropropene can also be exposed to the telogen CCl₃Z, (Z=H, Br, and/or Cl, for example) to form the telomer

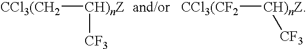

Products having n being at least 2 can be formed when utilizing an excess of the taxogen as compared to the telogen. For example, at least a 2:1 mole ratio of the taxogen to the telogen can be utilized to obtain products having n being at least 2. For example and by way of example only, at least two moles of the taxogen trifluoropropene can be exposed to at least one mole of the telogen (CF₃)₂CFI to form one or both of the telomers

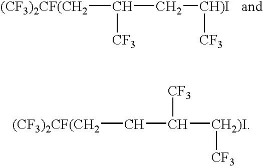

According to exemplary embodiments, telomer 9 can include those compounds shown in Table 4 below. As exemplary implementations are shown in Table 4 below, telomers can also be utilized as telogens.

Heterotelomerization can also be accomplished via cotelomerization and/or oligotelomerization. As an example, at least two different taxogens may be combined with at least one telogen to facilitate the production of at least a cotelomer. As another example, telomers may be produced from a first taxogen and the product telomer may be used in a subsequent telomerization with a second taxogen different from the first taxogen.

TABLE 4
Exemplary Telomers.

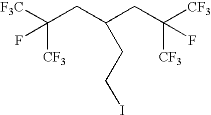

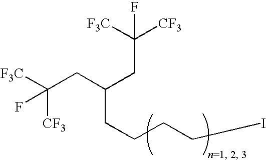

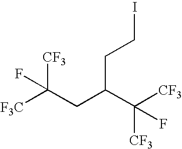

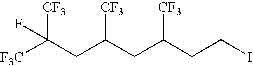

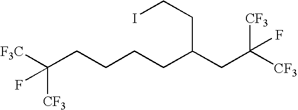

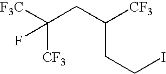

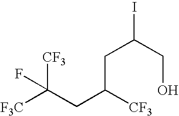

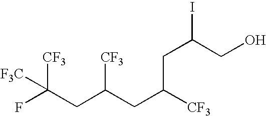

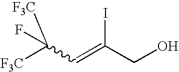

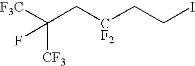

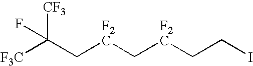

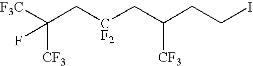

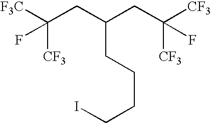

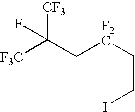

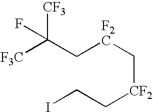

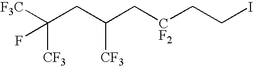

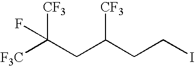

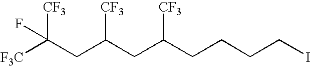

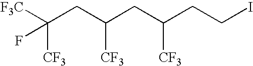

In additional embodiments initiator 6 may be provided to reactor 8 during the exposing of the reagents. Initiator 6 can include thermal, photochemical (UV), radical, and/or metal complexes, for example, including a peroxide such as di-tert-butyl peroxide. Initiator 6 can also include catalysts, such as Cu. Initiator 6 and telogen 4 can be provided to reactor 8 at a mole ratio of initiator 6 to taxogen 2 of from between about 0.001 to about 0.05 and/or from between about 0.01 to about 0.03, for example.

According to exemplary embodiments, various initiators 6 and telogens 4 can be used to telomerize taxogen 2 as referenced in Table 5 below. Telomerizations utilizing photochemical and/or metal-complex initiators 6 can be carried out in batch conditions using Carius tube reactors 8. Telomerizations utilizing thermal and/or peroxide initiators 6 can be carried out in 160 and/or 500 cm³ Hastelloy reactors 8. Telogen 4 (neat and/or as a peroxide solution) can be provided as a gas at a temperature from about 60° C. to about 180° C. and a telogen 4 [T]₀/taxogen 2 [Tx]₀ initial molar ratio R_{O} can be varied from 0.25 to 1.5 and the reaction time from 4 to 24 hrs as dictated in Table 5 below. The product mixture can be analyzed by gas chromatography and/or the product can be distilled into different fractions and analyzed by 1H and 19^{F} NMR and/or 13^{C} NMR. MonoAdduct (n=1) and DiAdduct (n=2) products can be recognized as shown in the Tables below.

TABLE 5
Telomerization of Trifluoropropene Taxogen

| | | | | | | | | | Yield (%) by GC^{c} | | |
|---|---|---|---|---|---|---|---|---|---|---|---|
| | | | | | | P (bars) | | % Conv. of | | MonoAdduct | DiAdduct |
| Run^{a} | Init.^{d} | R₀^{b} | C₀^{b} | T (° C.) | tᵣ(hrs) | max | min | Taxogen | Telogen | (n = 1) | (n = 2) |
| 1 | Therm | 0.50 | — | 160 | 20 | 22 | 17 | 79.2 | 27.6 | 51.9 | 20.5 |
| 2 | Therm | 0.25 | — | 160 | 20 | 39 | 34 | 36.8 | 52.8 | 26.2 | 21 |
| 3 | Therm | 0.50 | — | 180 | 22 | 30 | 11 | 73.4 | 2.4 | 65.9 | 31.2 |
| 4 | Perk | 0.50 | 0.03 | 62 | 20 | 7 | 5 | 79.2 | 23.8 | 35.4 | 40.8 |
| 5 | AIBN | 0.50 | 0.03 | 82 | 18 | 10 | 7 | 79.2 | 17.4 | 38.8 | 42 |
| 6 | TRIG | 0.50 | 0.03 | 134 | 6 | 16 | 0.6 | 89.6 | 3.7 | 19 | 63.8 |
| 7 | DTBP | 0.50 | 0.03 | 140 | 6 | 17 | 0.2 | 97.9 | 3.7 | 19 | 63.8 |
| 8 | DTBP | 0.50 | 0.03 | 143 | 4 | 19 | 0.8 | 94.3 | 9.6 | 21 | 66.6 |
| 9 | DTBP | 1.4 | 0.03 | 150 | 4 | 13 | 1.1 | 95.2 | 22.5 | 54.4 | 15.7 |
| 10 | DTBP | 0.75 | 0.03 | 145 | 4 | 20 | 3.0 | 93.8 | 6.8 | 34.1 | 49.0 |
| 11 | DTBP | 1.2 | 0.03 | 150 | 4 | 20 | 5.0 | 90.0 | 14.9 | 46.3 | 33.4 |
| 12 | DTBP | 1.4 | 0.03 | 150 | 4 | 21 | 3.5 | 95.0 | 12.6 | 54.1 | 28.6 |
| 13 | DTBP | 1.5 | 0.03 | 150 | 4 | 19 | 5.0 | 95.0 | 24.6 | 43.9 | 28.3 |

^{a}Telogen can be C₆F₁₃I in Runs Nos 1-9 and (CF₃)₂CFI in Runs No 10-13
^{b}R₀ = [T]₀/[Tx]₀; C₀ = [In]₀/[Tx]₀
^{c}Heavy TFP telomers (n > 2) can make up remainder of product
^{d}Initiators can be Perk. 16s(t-butyl cyclohexyl dicarbonate); AIBN; Trig.101(2,5-bis-(t-butyl peroxy)-2,5-dimethylhexane); and DTBP

TABLE 6
Telomerization of Pentafluoropropene Taxogen^{f}

| | | | | | | | Yield (%) by GC^{j} | | |
|---|---|---|---|---|---|---|---|---|---|
| | | | | | | % Conv. Of | | MonoAdduct | DiAdduct |
| Run^{g} | Init.^{h} | Roⁱ | Coⁱ | T (° C.) | tᵣ(hrs) | Taxogen | Telogen | (n = 1) | (n = 2) |
| 1 | DTBP | 1.4 | 0.03 | 143 | 4 | <8 | 62.5 | 7.9 | 6.1 |
| 2 | DTBP | 1.4 | 0.03 | 143 | 4 | <5 | 82.8 | 5.1 | 1.1 |
| 3 | TRIG.101 | 1.4 | 0.03 | 150 | 4 | <5 | 85.9 | 6.4 | 3.8 |
| 4 | TRIG.A80 | 1.4 | 0.03 | 180 | 5 | <10 | 63.4 | 4.9 | 1.6 |
| 5 | TRIG.A80 | 1.4 | 0.05 | 200 | 72 | <15 | 44.8 | 6.1 | 3.7 |
| 6 | TRIG.A80 | 1.4 | 0.06 | 220 | 48 | — | 50.7 | 3.2 | 1.4 |
| 7 | TRIG.A80 | 1.0 | 0.07 | 220 | 48 | — | 60.4 | 1.2 | 4.5 |
| 8 | TRIG.A80 | 0.5 | 0.08 | 220 | 48 | — | 41.7 | 1.2 | 2.8 |
| 9 | DIAD | 1.4 | 0.06 | 220 | 48 | — | 42.8 | 0.9 | 2.5 |
| 10 | DIAD | 1.0 | 0.06 | 220 | 48 | — | 42.7 | 0.8 | 1.8 |
| 11 | DIAD | 0.5 | 0.06 | 220 | 48 | — | 45.2 | 0.7 | 1.5 |
| 12 | CuCl | 1.4 | 0.4 | 140 | 48 | — | 20.2 | 0.1 | 0.2 |
| 13 | FeCl₂/benz | 1.4 | 0.4 | 140 | 48 | — | 14.8 | — | — |
| 14 | (PH₃P)₄Pd | 1.4 | 0.4 | 140 | 48 | — | 15.3 | 0.1 | 0.4 |
| 15 | Fe(II)acetate | 1.4 | 0.4 | 140 | 48 | — | 56.6 | 0.1 | 0.1 |

^{f}Telomerization of PFP with Rfl telogens at different reaction conditions (Hastelloy 160 cc reactor for runs 1-5 and 8 cc Carius tube for runs 6-15)
^{g}R_{F} is C₆F₁₅ except for run 2 where it is C₃F₇.
^{h}DTBP-di = tert-butyl peroxide; TRIG.101-2,5-bis(tert-butylperoxy)2,5-dimethylhexane; TRIG A80-tert-butyl hydroxyperoxide; DIAD-diisopropyl azodicoarboxylate
ⁱR₀ = [T]₀/[Tx]₀; C₀ = [In]₀/[Tx],
^{j}The remaining part is I₂ and/or heavy PFP telomers.

TABLE 7
Telomerization of PFP with non-fluorinated telogens (XY)^{k}

| | | | | | Yield (% by GC)ⁿ | | | |
|---|---|---|---|---|---|---|---|---|
| | | | | t_{R} | | n = | n = | n = |
| Run^{l} | Telogen | R₀^{m} | C₀^{m} | (hours) | XY | 1 | 1 | 3 |
| 1 | HP(O)(Oet)₂ | 1.4 | 0.07 | 48 | 34.8 | 16.2 | 8.6 | 3.3 |
| 2 | BrCF₂CHClBr | 1.4 | 0.03 | 48 | 22.7 | 1.8 | 0.8 | — |
| 3 | CBrCl₃ | 1.4 | 0.03 | 48 | 77.8 | 0.3 | 0.3 | — |
| 4 | CHCl₃ | 1.4 | 0.05 | 48 | 18.1 | 27.1 | 12.0 | 6.3 |
| 5 | HS(CH₂)₂OH | 1.4 | 0.05 | 15 | 15.5 | 23.9 | 13.4 | — |

^{k}initiator can be DTBP; solvent CH₃CN at 50% (wt./wt.); Temperature 143° C.;
^{l}runs 1-4 in 8 cc Carious tube, run 5 in Hatelloy reactor
^{m}R₀ = [T]₀/[Tx]₀; C₀ = [In]₀/[Tx]
ⁿfor run No. 5, (% wt by distillation): HSR-18.2; n = 1-50.1, n = 2-28.3

TABLE 8
Cotelomerization of PFP with VDF and TFP^{o}

| | | | | | Conv vs | | | | | | |
|---|---|---|---|---|---|---|---|---|---|---|---|
| | Feed (mol %) | | In cotelomer (mol %) | | SM % | Yield (% by GC) | | | Yield (% by distillation) | | |
| Run | PFP | coM₂ | PFP | coM₂ | (wt./wt.) | R_{f}I | n = 1 | n = 2 | R_{f}I | n = 1 | n = 2 |
| 1 | 85 | VDF-15 | <3 | 98 | 33.2 | 57.8 | 6.3 | 4.7 | 85.3 | 18.5 | 12.8 |
| 2 | 85 | TFP-15 | 39 | 61 | 51.9 | 45.9 | 24.2 | 3.0 | 55.1 | 32.9 | 6.8 |

^{o}Runs performed in 160 cc Hastelloy reactor with DTBP initiator (3 mol %); R_{f}I = C₆F₁₃I; R₀ = 1.0; T = 145° C.; T_{R} = 5 hours

According to exemplary embodiments telomerization processes can be utilized to produce R_{F}-Intermediates that can be incorporated and/or used to produce R_{F}-compositions such as surfactants, foam stabilizers, monomers, monomer units of polymers, urethanes, glycols, metal complexes, and/or phosphate esters. The R_{F}-intermediates can be characterized as R_{F}(R_{T})ₙQ with the R_{F} group including at least two —CF₃ groups, three or even at least four from —CF₃ groups. R_{T} can include a group having at least two carbons as described herein and n can be 1, 2, 3 or at least 4. Q can represent an atom of periodic table of elements such as a halogen. Furthermore, according to exemplary embodiments the R_{F}(R_{T})ₙ portion of the composition can include an R_{S} portion. The R_{T} portion can include the R_{S} portion, for example. According to at least one implementation, the R_{S} portion can be used to provide additional carbon chain length between the Q portion and the R_{F} portion of the composition. An exemplary embodiment of the disclosure includes R_{F}(R_{T})ₙ(R_{S})ₘQ. Like n described above, m can be 1, 2, 3, or at least 4. As just one example, R_{S} can be —CH₂—CH₂— for example and another R_{T} group of the composition can be —CH₂—CF₂— with R_{F} being (CF₃)₂CF— giving one exemplary telomer of (CF₃)₂CFCH₂CF₂CH₂CH₂Q. As described herein Q can also include Qg, for example.

According to exemplary embodiments, preparing R_{F}-compositions via telomerization of multiple taxogens with a single type of telogen can result in the preparation of cotelomers. Exemplary cotelomers can include different R_{T} groups, such as telomers of PFP, TFP, VDF, ethylene, for example. Exemplary schemes 28 through 39 further exemplify telomerizations that can be performed.

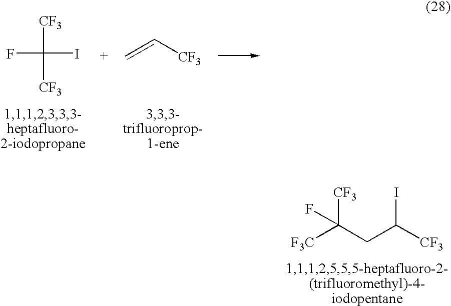

In accordance with scheme (28) above, in a 0.5″ outside diameter Inconel® tube having a volume of 34 cm³, can be packed with carbon, forming a carbon bed, and equipped with two inlet valves, a vaporizer or pre-heater, a thermocouple, a pressure relief valve, dry/ice trap, a pressure gauge, and a 10 (wt/wt) percent KOH scrubber on the outlet. Materials leaving the reactor can be scrubbed and passed through a Drierite® tube and a dry ice/acetone trap. The carbon bed can be dried thoroughly before being used and the tube can be heated until the carbon bed reaches about 300° C. To the heated tube, 3,3,3-trifluoroprop-1-ene at a flow rate of 51.43 cm³ per minute and 1,1,1,2,3,3,3-heptafluoro-2-iodopropane at a flow rate of 19.88 cm³ per minute can be fed simultaneously over the bed yielding a mole ratio of 3,3,3-trifluoroprop-1-ene to 1,1,1,2,3,3,3-heptafluoro-2-iodopropane of 2.86 and a contact time of 13.6 seconds to afford 1.44 grams of 1,1,1,2,5,5,5-heptafluoro-2-(trifluoromethyl)-4-iodopentane, 0.78 grams of 1,1,1,4,5,5,5-heptafluoro-4-(trifluoromethyl)pent-2-ene, and 0.02 grams of 1,1,1,2,7,7,7-heptafluoro-2,4-bis(trifluoromethyl)-6-iodoheptane as analyzed by gas chromatography.

In reference to scheme (28) above, in a 0.5″ outside diameter Inconel® tube having a volume of about 34 cm³, can be packed with carbon, to form a carbon bed, and equipped with two inlet valves, a vaporizer or pre-heater, a thermocouple, a pressure relief valve, dry/ice trap, a pressure gauge, and a 10 (wt/wt) percent KOH scrubber on the outlet. Materials leaving the reactor can be scrubbed and passed through a Drierite® tube and a dry ice/acetone trap. The carbon bed can be dried thoroughly before being used and the tube can be heated so that the bed is about 300° C. To the heated tube, 3,3,3-trifluoroprop-1-ene at a flow rate of about 58.07 cm³ per minute and 1,1,1,2,3,3,3-heptafluoro-2-iodopropane at a flow rate of about 47.72 cm³ per minute can be fed simultaneously over the bed to afford a mole ratio of 3,3,3-trifluoroprop-1-ene to 1,1,1,2,3,3,3-heptafluoro-2-iodopropane of about 1.24 and a contact time of about 9.19 seconds to afford a product mixture containing about 2.8 grams of 1,1,1,2,5,5,5-heptafluoro-2-(trifluoromethyl)-4-iodopentane, 0.3 grams of 1,1,1,4,5,5,5-heptafluoro-4-(trifluoromethyl)pent-2-ene, and 0.43 grams of 1,1,1,2,7,7,7-heptafluoro-2,4-bis(trifluoromethyl)-6-iodoheptane as analyzed by gas chromatography. The product mixture can be confirmed by NMR and/or chromatographic analysis.

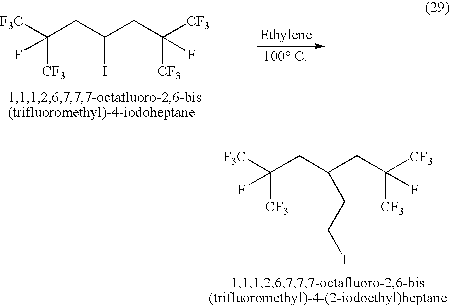

According to scheme (29) above, into a 300 mL autoclave that can be equipped with a dip tube, thermocouple, agitator, pressure gauge, and an attachment to a reservoir containing ethylene gas, 319 grams (0.63 mole) 1,1,1,2,6,7,7,7-octafluoro-2,6-bis(trifluoromethyl)-4-iodoheptane (see, e.g. Published International Applications) and 3 grams (0.012 mole) dibenzoyl peroxide can be added to form a mixture. The autoclave can be sealed, evacuated, and heated to about 100° C. Ethylene gas can be added to the mixture to form a reaction mixture. The reaction mixture can be held at a pressure, generated by ethylene, of about 380 psig for about four hours. The reaction mixture can then be chilled using an ice water bath and degassed. To the reaction mixture, an additional 3.0 grams (0.012 mole) dibenzoyl peroxide can be added to form a new mixture. The autoclave can be sealed, evacuated, and heated to about 100° C. Ethylene gas can be added to the mixture to form a new reaction mixture. The new reaction mixture can be held at a pressure, generated in-part by ethylene, of about 380 psig for about four hours chilled with an ice water bath, degassed, and opened to provide 336.5 grams of 80 (wt/wt) percent pure (by gas chromatography) 1,1,1,2,6,7,7,7-octafluoro-2,6-bis(trifluoromethyl)-4-iodoheptane product. The product can be purified by vacuum distillation (b.p. 53° C./1.3 Torr) the structure confirmed by NMR and/or chromatographic analysis.

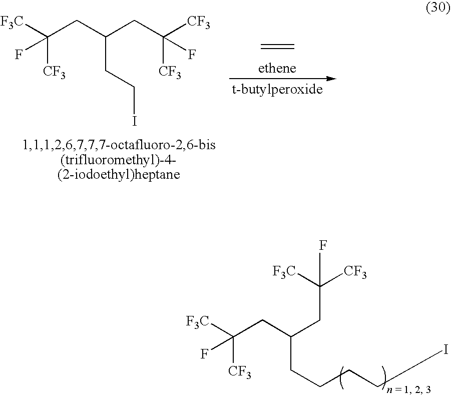

According to scheme (30) above, into a 300 mL autoclave can be added 90.97 grams (0.17 moles) of 1,1,1,2,6,7,7,7-octafluoro-2,6(trifluoromethyl)-4-(2-iodoethyl)heptane (refer to scheme (29) above) and 4.56 grams of t-butyl peroxide can be placed to form a mixture. The mixture can be heated to 135° C. and ethylene gas can be added to form a reaction mixture and give an initial pressure of about 400 psig. As the pressure decreased more ethylene gas can be added to maintain a pressure of between 385 and 410 psig. After about 5 hours the reactor can be allowed to cool and the excess ethylene was slowly vented from the autoclave and the reaction mixture collected to afford a product mixture that can comprise 37% of 1,1,1,2-tetrafluoro-4-(2,3,3,3-tetrafluoro-2-(trifluoromethyl)propyl)-2-(trifluoromethyl)-8-iodooctane and 7.8% of 1,1,1,2-tetrafluoro-4-(2,3,3,3-tetrafluoro-2-(trifluoromethyl)propyl)-2-(trifluoromethyl)-10-iododecane. The product structure can be confirmed by NMR and/or chromatographic analysis.

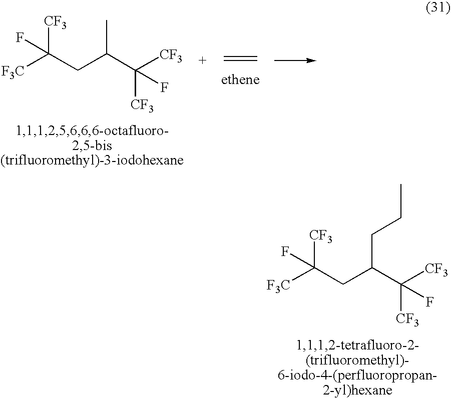

Referring to scheme (31) above, in an autoclave that can be equipped with an agitator, thermocouple, relief valves, sample valves and a pressure gauge, 59 grams of crude 1,1,1,2,5,6,6,6-octafluoro-2,5-bis(trifluoromethyl)-3-iodohexane (50% by go) and 0.59 grams (0.002 mole) of benzoyl peroxide can be placed to form a mixture. The reactor was sealed, chilled with dry ice/acetone and a vacuum imposed. The mixture can be heated to 98° C. and pressurized to about 300 psig with gaseous ethylene to form a reaction mixture. The reaction mixture pressure can be maintained at between about 280 and 320 psig for about 6 hours. The reaction mixture can be sampled to afford the 1,1,1,2-tetrafluoro-2-(trifluoromethyl)-6-iodo-4-(perfluoropropan-2-yl)hexane product (crude yield of 53% by go). The product structure can be confirmed by NMR and/or chromatographic analysis.

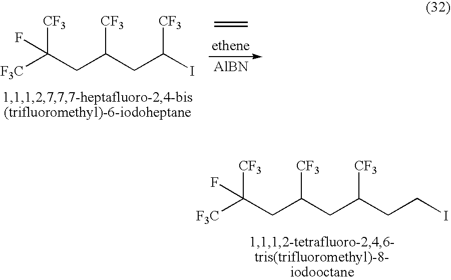

According to scheme (32) above, in a 600 mL autoclave that can be equipped with a dip tube with check valve for feeding ethylene, pressure gauge, rupture disk, venting valve, agitator and a thermocouple, 202.5 grams (0.415 mole) of 1,1,1,2,7,7,7-heptafluoro-2,4-bis(trifluoromethyl)-6-iodoheptane (the diadduct telomer described above) and 1.1 grams (0.007 mole) of 2,2′-Azobisisobutyronitrile (AIBN) can be placed to form a mixture and the autoclave sealed. The mixture can be heated to from about 80° C. to about 140° C. and ethylene fed into the autoclave to form a reaction mixture and maintained for at least about 26 hours. The total amount of ethylene added to the autoclave can be at least about 11.6 grams (0.415 mole). The autoclave can be vented and emptied to afford 175 grams of the 1,1,1,2-tetrafluoro-2,4,6-tris(trifluoromethyl)-8-iodooctane product. The product structure can be confirmed by NMR and/or chromatographic analysis.

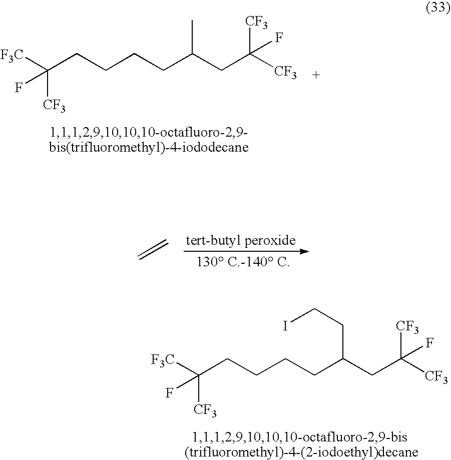

According to scheme (33) above, in a 600 mL stainless steel autoclave that can be equipped with an agitator, thermocouple, a braided stainless steel hose coupled to an ethylene reservoir cylinder, and a dip-tube for supplying the ethylene gas subsurface relative to, starting material, 254 grams (0.46 mole) of 1,1,1,2,9,10,10,10-octafluoro-2,9-bis(trifluoromethyl)-4-iododecane and 4.24 grams (0.03 mole) tert-butyl peroxide can be added to form a mixture. The autoclave can be sealed and the mixture heated to from about 130° C. to about 140° C., then ethylene can be added subsurface until the autoclave pressure reaches from about 100 psig to about 300 psig, and/or from about 150 psig to about 250 psig to form a reaction mixture. Ethylene can be consumed as the reaction proceeds as can be evidenced by a decrease in autoclave pressure. The autoclave pressure can be maintained at the above ranges through use of a regulator or can be added discretely several times throughout the reaction. The total amount of ethylene added can be about 12.9 grams (0.463 mole). The reaction mixture can be held at temperature and pressure for from about six hours to about twelve hours. The autoclave can be cooled and vented then the reaction mixture can be washed three times with 100 mL portions of 30 percent (wt/wt) sodium metabisulfite solution to form a multiphase mixture from which the organic layer can be collected and dried over magnesium sulfate, filtered and concentrated in vacuo to afford the product 1,1,1,2,9,10,10,10-octafluoro-2,9-bis(trifluoromethyl)-4-(2-iodoethyl)decane and a small amount of the diadduct 1,1,1,2-tetrafluoro-7-(2,3,3,3-tetrafluoro-2-(trifluoromethyl)propyl)-2-(trifluoromethyl)-11-iodoundecane. m/z: 449 (M⁺-I), 239 (M⁺-C₆H₆F₇), 225 (M⁺-C₇H₆F₇).

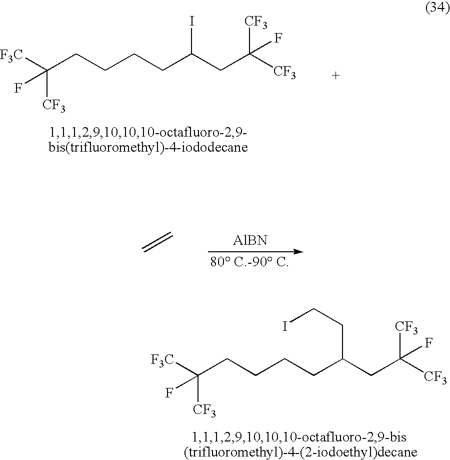

In accordance with scheme (34) above, in a 15 mL stainless steel autoclave that can be equipped with an agitator, thermocouple, pressure gauge, and a needle valve that can be equipped to receive ethylene gas, 5.0 grams (0.009 mole) of 1,1,1,2,9,10,10,10-octafluoro-2,9-bis(trifluoromethyl)-4-iododecane and 0.1 gram (6.1×10⁻⁴ mole) of 2,2′-azobisisobutrylonitrile can be added to form a mixture. The mixture can be heated to from about 65° C. to about 95° C. To the mixture, about 0.26 gram. (0.009 mole) of ethylene can be added to form a reaction mixture. The ethylene addition can be continuous or discrete such that an autoclave pressure is maintained from about 150 psig to about 250 psig. The reaction can be held at temperature for from about four hours to about eight hours to afford the 1,1,1,2,9,10,10,10-octafluoro-2,9-bis(trifluoromethyl)-4-(2-iodoethyl)decane product and a small amount of the diadduct 1,1,1,2-tetrafluoro-7-(2,3,3,3-tetrafluoro-2-(trifluoromethyl)propyl)-2-(trifluoromethyl)-11-iodoundecane. The product structure can be confirmed by NMR and GC/MS analysis.

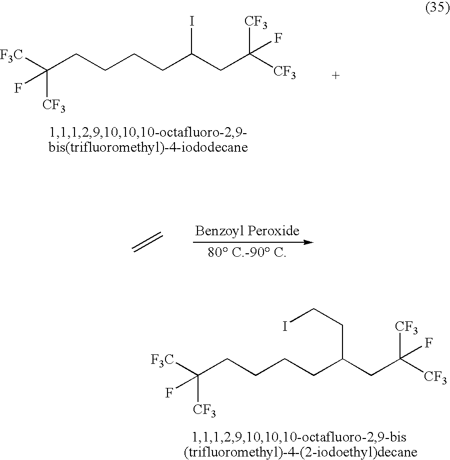

In reference to scheme (35) above, in a 15 mL stainless steel autoclave that can be equipped with an agitator, thermocouple, pressure gauge, and a needle valve that can be equipped to receive ethylene gas, 15.09 grams (0.028 mole) of 1,1,1,2,9,10,10,10-octafluoro-2,9 bis(trifluoromethyl)-4-iododecane and 0.2 gram (0.0008 mole) of benzoyl peroxide can be added to form a mixture. The mixture can be heated to from about 80° C. to about 100° C., and/or about 95° C. then about 0.79 gram (0.028 mole) of ethylene can be added to form a reaction mixture. The ethylene addition can be continuous or discrete such that an autoclave pressure is maintained from about 150 psig to about 300 psig. The reaction mixture can be held at the temperature for from about 5 hours to about 12 hours or until about all of the starting material is converted to the 1,1,1,2,9,10,10,10-octafluoro-2,9-bis(trifluoromethyl)-4-(2-iodoethyl)decane product and a small amount of the diadduct 1,1,1,2-tetrafluoro-7-(2,3,3,3-tetrafluoro-2-(trifluoromethyl)propyl)-2-(trifluoromethyl)-11-iodoundecane. The product structure can be confirmed by NMR and GC/MS analysis.

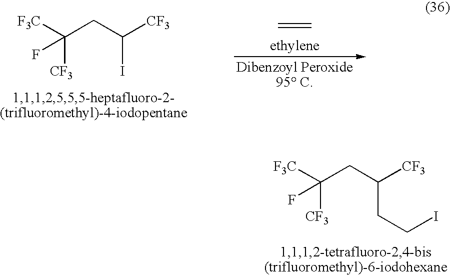

Referring to scheme (36) above, in a 20 mL autoclave that can be equipped with an agitator, a thermocouple, and a pressure gauge, 3.42 grams (0.0087 mole) of 1,1,1,2,5,5,5-heptafluoro-2-(trifluoromethyl)-4-iodopentane and 0.034 gram (1.4×10⁻⁴ mole) of dibenzoyl peroxide to form a mixture. The autoclave can then be sealed and heated to about 95° C. whereupon ethylene gas can be delivered to the autoclave to form a reaction mixture so that a pressure of about 350 psig can be achieved. The autoclave pressure can be observed to decline over the course of the reaction and as such the ethylene gas can be continuously delivered to the autoclave so that an autoclave pressure of about 300 psig can be maintained for about one hour. The reaction mixture can be degassed and analyzed by gas chromatography to afford the product 1,1,1,2-tetrafluoro-2,4-bis(trifluoromethyl)-6-iodohexane having about 81.3 (wt/wt) percent purity. The product structure can be confirmed by NMR and/or chromatographic analysis.

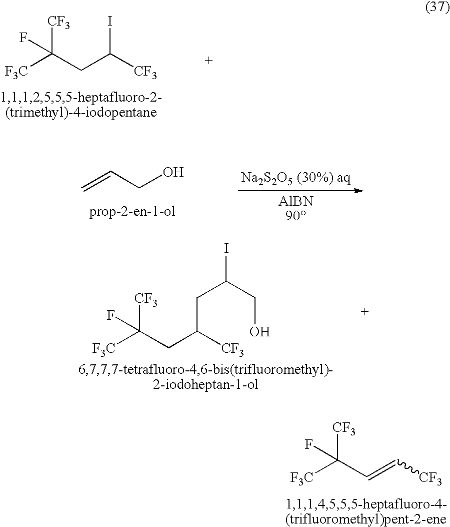

According to scheme (37) above, to a round bottom flask that can be equipped with thermocouple well and thermocouple, agitator, and reflux condenser, 60.41 grams (0.154 mole) of 1,1,1,2,5,5,5-heptafluoro-2-(trifluoromethyl)-4-iodopentane (see scheme (18) above), 9.57 grams (0.165 mole) of prop-2-en-1-ol, 0.292 gram (0.002 mole) of 2,2′-azobisisobutyronitrile, and about 15 gram of a 30 percent (wt/wt) aqueous Na₂S₂O₅ solution can be placed to form a reaction mixture. The reaction mixture can be heated to at least about 80° C., from about 65° C. to about 100° C., and/or about 80° C. to about 90° C. where a reflux can be observed. After about four hours, from about two hours to about six hours, and/or about three hours to about five hours, about 0.25 grams (0.002 mole) 2,2′-azobisisobutyronitrile can be added to the reaction mixture. After about four hours, about 0.28 grams (0.002 mole) of 2,2′-azobisisobutyronitrile can be added to the reaction mixture and held for about four hours at reflux. To the reaction mixture, about 0.23 grams (0.001 mole) of 2,2′-azobisisobutyronitrile can be added and held at reflux for about four hours. The reaction mixture can be concentrated in vacuo to afford the 1,1,1,2,5,5,5-heptafluoro-2-(trifluoromethyl)-4-iodopentane product along with the side product, 1,1,1,4,5,5,5-heptafluoro-4-(trifluoromethyl)pent-2-ene. (m/z: 323 (M⁺-I) 303 (M⁺-IF) 255 (M⁺-CF₃I) 237 (M⁺-CF₄I)).

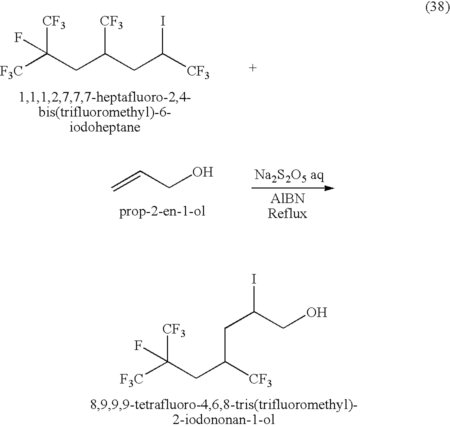

According to scheme (38) above, in a 125 mL round bottom flask that can be configured with a thermocouple, reflux condenser and a 50 mL pressure equalized addition funnel, 11.1 gram (0.191 mole) of propen-1-ol, 4.41 gram (mole) sodium metabisulfite, and 10.81 gram (mole) water can be placed to form a mixture. The mixture can be heated from about 50° C. to about 100° C., 75° C. to about 85° C., and/or about 80° C. To the mixture can be added drop wise, 89.4 gram (0.183 mole) of 1,1,1,2,7,7,7-heptafluoro-2,4-bis(trifluoromethyl)-6-iodoheptane (two isomers) and about 0.32 gram (0.002 mole) 2,2′-azobisisobutyronitrile to form a reaction mixture. The addition rate can be at least about 0.55 milliliters per minute (mL/min) from about 0.30 mL/min to about 0.75 mL/min, and/or about 0.45 mL/min to about 0.65 mL/min. This new mixture can then be held at about 80° C. for about four hours. After said hold period, 0.69 gram (0.004 mole) 2,2′-azobisisobutyronitrile can be added to the reaction and held at about 80° C. for four hours. The organic layer of the reaction mixture can be collected, dried over magnesium sulfate, filtered, to afford 48.8 grams of an isomeric mixture of 8,9,9,9-tetrafluoro-4,6,8-tris(trifluoromethyl)-2-iodononan-1-ol product having a purity of about 68 percent (area percent by gas chromatography). m/z 419 (M⁺-I′), 349 (M⁺-CF₃I′), 335 (M⁺-CF₃IOH′), 127 (I′).

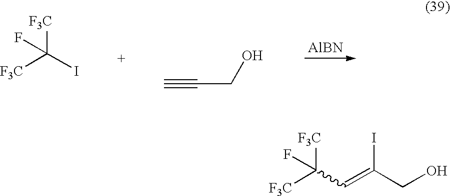

In accordance with scheme (39) above, into a 600 cc Parr reactor that can be equipped with an agitator, a thermocouple, pressure gauge, and feeding dip tube, 193 grams (0.63 moles) of 2-iodoheptafluoropropane, 39.67 grams (0.71 moles) of propargyl alcohol and 1.07 grams of 2,2′-azobisisobutryonitrile (AIBN) can be added to form a mixture. The reactor can be sealed and heated from about 75° C. to about 95° C., and/or about 85° C. for about 24 hours. Analysis of the mixture by gas chromatography can show the formation of 4,5,5,5-tetrafluoro-4-(trifluoromethyl)-2-iodopent-2-en-1-ol isomers of about 48 area percent. To the mixture, 1.2 grams AIBN can be added to form a reaction mixture. The reaction mixture can be heated to from about 75° C. to about 95° C., and/or about 85° C. for about 24 hours. Analysis of the reaction mixture by gas chromatography can show the formation of 4,5,5,5-tetrafluoro-4-(trifluoromethyl)-2-iodopent-2-en-1-ol isomers of about 64 area percent. The product can be further characterized by gas chromatography/mass spectroscopy and NMR.

According to exemplary embodiments, telomers can be used as R_{F}-intermediates directly and/or converted to R_{F}-intermediates. Schemes 40 to 70 are exemplary of R_{F}-intermediate preparations from utilizing telomers as at least one starting material.

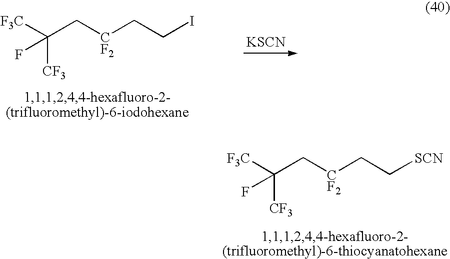

According to scheme (40) above, in a flask that can be equipped with an agitator, thermocouple, and a reflux condenser, 30.7 grams (0.08 mole) of 1,1,1,2,4,4-hexafluoro-2-(trifluoromethyl)-6-iodohexane (i.e., telomer of F7I, VDF, and ethylene) about 100 mL of ethanol 11.8 grams (0.12 mole) of potassium thiocyanate and 0.4 mL of glacial acetic acid to form a mixture. The mixture can be heated to reflux and maintained for about 4.5 hours. The mixture can be concentrated and about 100 mL of water and about 100 mL of ether can be added to form a multiphase mixture from which an organic phase can be separated from an aqueous phase. The phases can be partitioned and the aqueous phase can be once more extracted with about 100 mL of ether. The organic phases can be combined and dried over sodium sulfate, filtered and concentrated to afford 21.2 grams of the 1,1,1,2,4,4-hexafluoro-2-(trifluoromethyl)-6-thiocyanatohexane that can be observed as a yellow oil. The product structure can be confirmed by LCMS and/or NMR analysis.

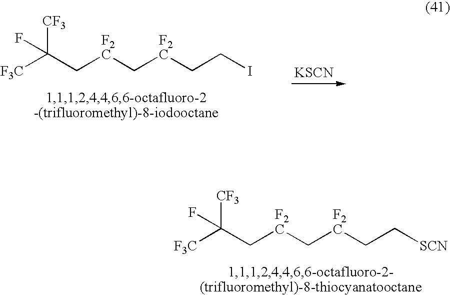

Referring to scheme (41) above, in a flask that can be equipped with an agitator, thermocouple and a reflux condenser, 21.2 grams (0.05 mole of solution of 1,1,1,2,4,4,6,6-octafluoro-2-(trifluoromethyl)-8-iodooctane (i.e., telomer of F7I, VDF, and ethylene), about 50 mL of ethanol, 7.1 grams (0.07 mole) of potassium thiocyanate and 0.3 mL of glacial acetic acid to form a mixture. The mixture can be heated to reflux and maintained for about 5.5 hours. The mixture can be observed as a heterogeneous mixture of white salts and brown liquid. The mixture can be concentrated and about 100 mL of water and about 100 mL of ether can be added to form a multiphase mixture from which an organic phase can be separated from an aqueous phase. The phases can be separated and the aqueous phase once more extracted with about 100 mL of ether. The organic phases can be combined, dried over sodium sulfate, filtered and concentrated to afford 17.7 grams of the 1,1,1,2,4,4,6,6-octafluoro-2-(trifluoromethyl)-8-thiocyanatooctane product which can be observed as a brown oil, which solidified upon standing. The product structure can be confirmed by NMR and/or GCMS analysis.

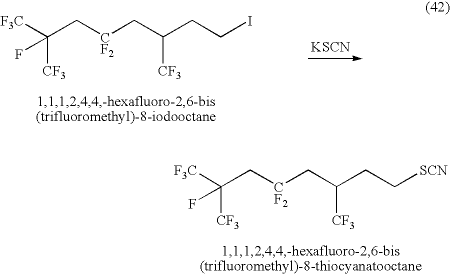

Referring to scheme (42) above, in a flask that can be equipped with an agitator, thermocouple and a reflux condenser, 34 grams (0.07 mole of 1,1,1,2,4,4-hexafluoro-2,6-bis(trifluoromethyl)-8-iodooctane (i.e., telomer of F71, VDF, TFP, and ethylene), about 70 mL of absolute ethanol, 10.24 grams (0.11 mole) of potassium thiocyanate and 0.35 mL of glacial acetic acid to form a mixture. The mixture can be heated to reflux and maintained for about 5 hours. The mixture can be observed as a heterogeneous mixture of white salts and yellow liquid. The mixture can be concentrated and about 100 mL of water and about 100 mL of ether can be added to form a multiphase mixture from which an organic phase can be separated from an aqueous phase. The phases can be separated and the aqueous phase once more extracted with about 100 mL of ether. The organic phases can be combined, dried over sodium sulfate, filtered and concentrated to afford 25.7 grams of the 1,1,1,2,4,4-hexafluoro-2,6-bis(trifluoromethyl)-8-thiocyanatooctane product which can be observed as a brown oil, which solidified upon standing. The product structure can be confirmed by NMR and/or GC analysis.

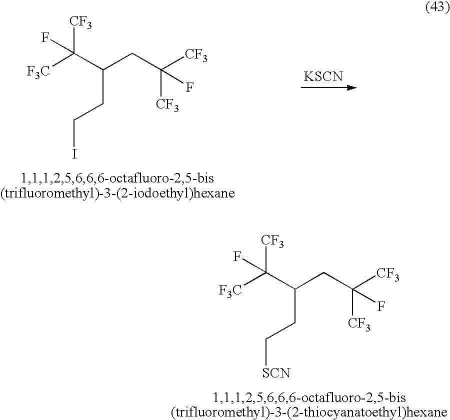

Referring to scheme (43) above, in a flask that can be equipped with an agitator, thermocouple and a reflux condenser, 33.85 grams (0.07 mole of solution of 1,1,1,2,5,6,6,6-octafluoro-2,5-bis(trifluoromethyl)-3-(2-Iodoethyl)hexane (refer to scheme (31) above), about 65 mL of ethanol, 9.5 grams (0.1 mole) of potassium thiocyanate and 0.35 mL of glacial acetic acid to form a mixture. The mixture can be heated to reflux and maintained overnight. The mixture can be observed as a heterogeneous mixture of white salts and brown liquid. The mixture can be concentrated and about 100 mL of water and about 100 mL of ether can be added to form a multiphase mixture from which an organic phase can be separated from an aqueous phase. The phases can be separated and the aqueous phase once more extracted with about 100 mL of ether. The organic phases can be combined, dried over sodium sulfate, filtered and concentrated to afford 26.15 grams of the 1,1,1,2,5,6,6,6-octafluoro-2,5-bis(trifluoromethyl)-3-(2-thiocyanatoethyl)hexane product which can be observed as a brown oil, which solidified upon standing. The product structure can be confirmed by NMR and/or chromatographic analysis.

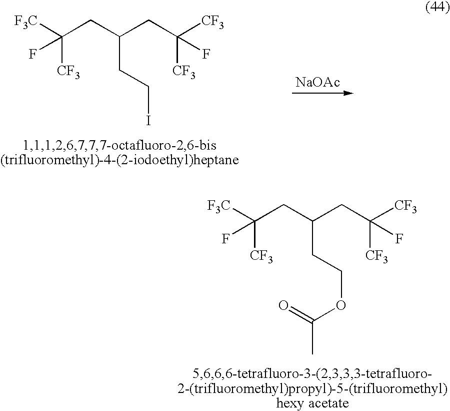

According to scheme (44) above, in a flask that can be equipped with an agitator and a thermocouple, 30 grams 90.056 mole) of 1,1,1,2,6,7,7,7-octafluoro-2,6-bis(trifluoromethyl)-4-(2-iodoethyl)heptane (refer to scheme (29) above), 13.82 grams (0.169 mole) of sodium acetate and about 185 mL of dimethylformamide can be placed to form a mixture. The mixture can be heated to 80° C. and maintained overnight. The mixture can be combined with about 300 mL of water to form a multiphase mixture from which an organic phase can be separated from an aqueous phase. The aqueous phase can be extracted twice with 300 mL portions of ether. The organic phases can be combined and washed with about 300 mL of brine to form a multiphase mixture from which an organic phase can be separated from an aqueous phase. The organic phase can be dried, concentrated and placed on a Kugelrohr apparatus at 40° C. and 0.03 mmHg for a period of about one hour to afford 16.45 grams of the 5,6,6,6-tetrafluoro-3-(2,3,3,3-tetrafluoro-2-5. (trifluoromethyl)propyl)-5-(trifluoromethyl)hexyl acetate product. The product structure can be confirmed by NMR and/or chromatographic analysis.

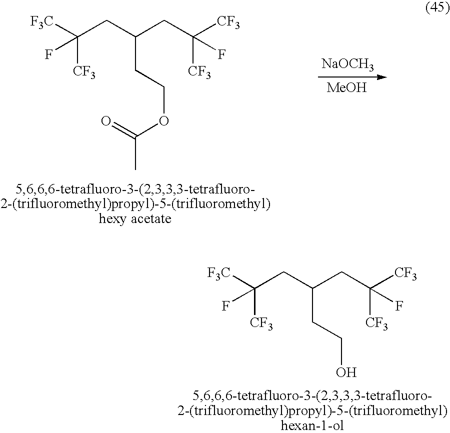

In reference to scheme (45) above, in a flask that can be equipped with an agitator and a thermocouple, 30.3 grams (0.065 mole) of 5,6,6,6-tetrafluoro-3-(2,3,3,3-tetrafluoro-2-(trifluoromethyl)propyl)-5-(trifluoromethyl)hexyl acetate (refer to scheme 44 above), 0.2 grams (0.009 mole) of sodium metal and about 100 mL of methanol can be placed to form a mixture. The mixture can be allowed to stir overnight at room temperature. The mixture can be treated with about 17 mL of a 1N solution of HCl in water, the pH can be observed to be about 5. The mixture can be concentrated and about 100 mL of ether and washed with two 100 mL portions of a saturated bicarbonate solution to form a multiphase mixture from which an organic phase can be separated from an aqueous phase. The organic phase can be dried and concentrated to afford 25 grams of the 5,6,6,6-tetrafluoro-3-(2,3,3,3-tetrafluoro-2-(trifluoromethyl)propyl)-5-(trifluoromethyl)hexan-1-ol product that can be observed as a yellow oil. The product structure can be confirmed by NMR and/or chromatographic analysis.

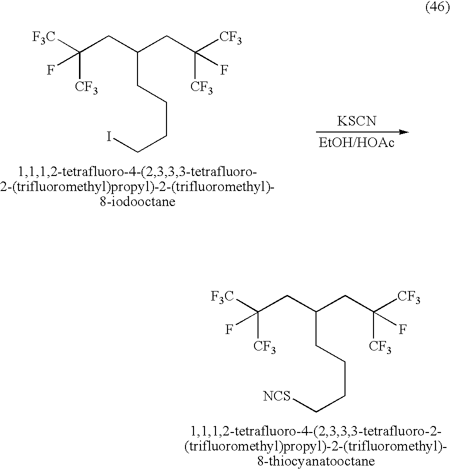

According to scheme (46) above, in a flask that can be equipped with an agitator, thermocouple, reflux condenser, and an addition funnel, 40.0 grams (71.2 mmol) of 1,1,1,2-tetrafluoro-4-(2,3,3,3-tetrafluoro-2-(trifluoromethyl)propyl)-2-(trifluoromethyl)-8-iodooctane (refer to scheme (30) above), 50 ml of absolute ethanol, 10.4 grams (106.7 mmol) of KSCN and 1.5 ml of acetic acid can be added to form a mixture. The mixture can be heated to reflux (84.7° C.), stirred for about 5 hours, cooled to room temperature and stirred and maintained for overnight. The mixture can be heated to reflux and maintained for about four hours. The mixture can be observed as a pale yellow slurry and can be cooled to room temperature and concentrated in vacuo to give what can be observed as a thick yellow slurry. The yellow slurry can be extracted with about 3 liters of diethyl ether, decanted twice and filtered. The wet cake can be washed with three 100 ml portions of diethyl ether. The filtrate can be concentrated in vacuo to afford about 34.66 g (98.8% yield) of the 1,1,1,2-tetrafluoro-4-(2,3,3,3-tetrafluoro-2-(trifluoromethyl)propyl)-2-(trifluoromethyl)-8-thiocyanatooctane product which can be observed as a light yellow oil. The product structure can be confirmed by NMR and/or chromatographic analysis.

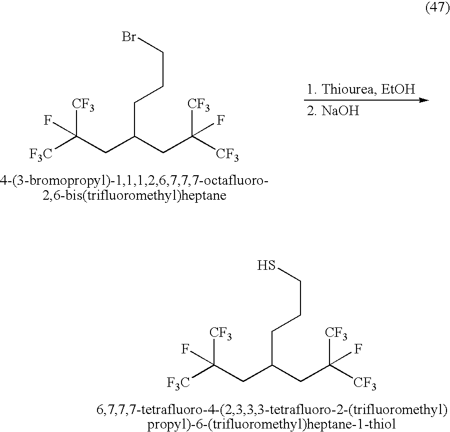

According to scheme (47) above, in a flask that can be equipped with an agitator, thermocouple, and a reflux condenser, 70 grams (0.14 mole) of 4-(3-bromopropyl)-1,1,2,6,7,7,7-octafluoro-2,6 bis(trifluoromethyl)heptane, 15.9 grams (0.21 mole) of thiourea, and 648 mL of ethanol can be placed to form a first mixture. The first mixture can be heated to reflux and held for from about 1 g hours to about 25 hours, and/or about 23 hours. To the first mixture, 5.3 grams (0.069 mole) of thiourea can be placed to form a second mixture. The second mixture can be refluxed for from about 19 hours to about 25 hours, and/or about 23 hours. To the second mixture, 5.3 grams (0.069 mole) of thiourea can be placed to form a reaction mixture. The reaction mixture can be held at reflux for from about 15 hours to about 21 hours, and/or about 18 hours and cooled to from about 18° C. to about 24° C., and/or about 21° C. and concentrated in vacuo to afford what can be observed as a sticky solid. The sticky solid can be placed on a Kugelrohr apparatus (0.1 Torr, 50° C., 60 minutes) to afford a mixture containing the 6,7,7,7-tetrafluoro-4-(2,3,3,3-tetrafluoro-2-(trifluoromethyl)propyl)-6-(trifluoromethyl)heptane-1-thiol product. The product structure can be confirmed by NMR and/or chromatographic analysis.

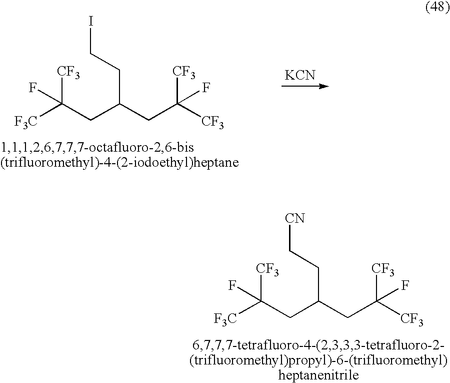

Referring to scheme (48) above, in a flask that can be equipped with an agitator, thermocouple and an addition funnel, 5 grams (0.009 mole) of 1,1,1,2,6,7,7,7-octafluoro-2,6-bis(trifluoromethyl)-4-(2-iodoethyl)heptane (refer to scheme (29) above) and about 20 mL of dimethylformamide can be placed to form a mixture. To the mixture, 1.22 grams (0.019 mole) of potassium cyanide can be added to form a reaction mixture. The reaction mixture can be heated to 80° C. and maintained for about 2 hours, allowed to cool to room temperature and maintained overnight. The reaction mixture can be poured into about 75 mL of water to form a multiphase mixture from which an organic phase can be separated from an aqueous phase. The aqueous phase can be extracted with two 75 mL portions of ether and the resulting organic phases can be combined and dried, filtered and concentrated to afford 1.3 grams of the 6,7,7,7-tetrafluoro-4-(2,3,3,3 tetrafluoro-2-(trifluoromethyl)propyl)-6-trifluoromethyl)heptanenitrile that can be observed as a brown oil. The product structure can be confirmed by NMR and/or GCMS and/or IR analysis.

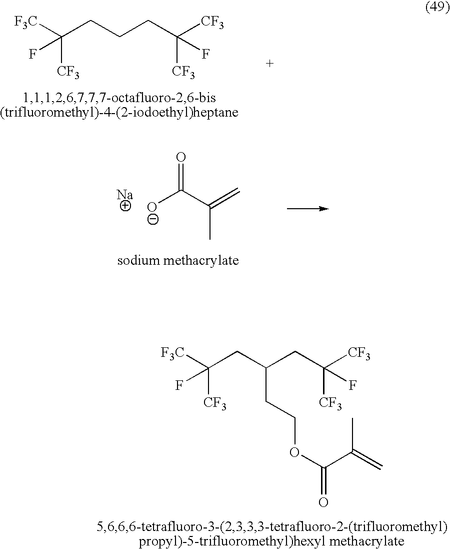

Referring to scheme (49) above, into a 600 mL autoclave, 300 grams of t-butyl alcohol, 200 grams (0.374 moles) of 1,1,2,6,7,7,7-octafluoro-2,6-bis(trifluoromethyl)-4-(2-iodoethyl)heptane (refer to scheme (29) above), 73.5 grams (0.677 moles) of sodium methacrylate and 10 grams of 4-t-butyl cathechol can be placed to form a mixture. The reactor can be sealed and heated to 110° C. and maintained for about 18 hours. The mixture can be heated to 125° C. and maintained for 6 hours. The mixture was washed three times with water to form a multiphase mixture from which an organic phase can be separated from an aqueous phase. The organic phase can be collected to afford 190 grams (67% by GC) that can be dried over MgSO₄ and distilled at 67° C./1.7 Torr to afford the 5,6,6,6-tetrafluoro-3-(2,3,3,3-tetrafluoro-2-(trifluoromethyl)propyl)-5-(trifluoromethyl)hexyl methacrylate. The product structure can be confirmed by NMR and/or chromatographic analysis.

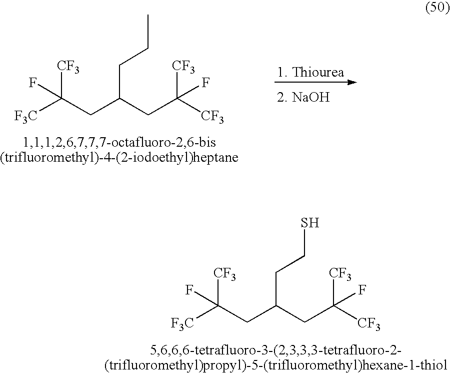

According to scheme (50) above, in a flask that can be equipped with an agitator, thermocouple, and a reflux condenser, 100.5 grams (0.19 mole) of 1,1,1,2,6,7,7,7-octafluoro-2,6-bis(trifluoromethyl)-4-(2-iodoethyl)heptane (refer to scheme (29) above) and about 575 mL of ethanol can be added to form a mixture. To the mixture, 21.5 grams (0.28 mole) of thiourea can be added to form a reaction mixture. The reaction mixture can be heated to reflux temperature and held until the starting material has disappeared. The reaction mixture can be concentrated to afford what can be observed as a white solid. To the white solid, about 245 mL of water can be added, followed by the portion wise addition of 32 grams of NaOH to form a new mixture. The new mixture can be allowed to stir at from about 18° C. to about 24° C., and/or about 21° C. for about one hour. The flask can be equipped with a Dean-Stark apparatus that can contain a reflux condenser set at about −10° C. and a dry ice trap whereupon the organic portion of the mixture can be separated from the new mixture at a pot temperature of about 100° C. to afford about 55.5 grams of distillate. The distillate can be washed with two 100 mL portions of water to form a multiphase mixture from which an organic phase can be separated from an aqueous phase. The organic phase can be collected to afford 49.6 grams of the 5,6,6,6-tetrafluoro-3-(2,3,3,3-tetrafluoro-2-(trifluoromethyl)propyl)-5-(trifluoromethyl)hexane-1-thiol product. The product structure can be confirmed by NMR and/or chromatographic analysis.

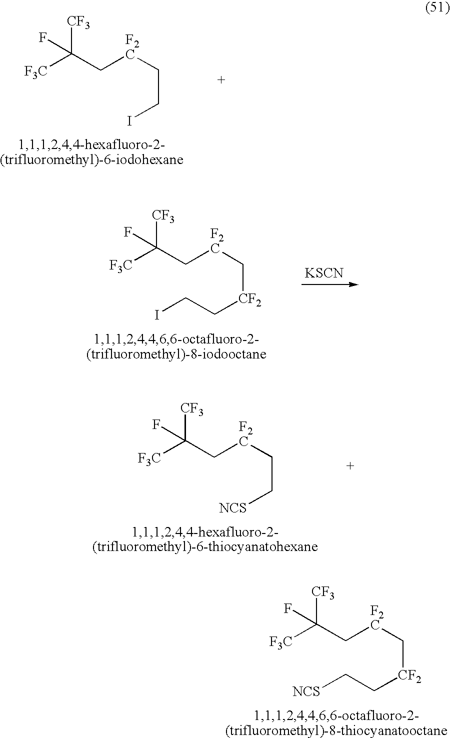

Referring to scheme (51) above, in a flask that can be equipped with an agitator, thermocouple and a reflux condenser, 19.5 grams (0.04 mole of solution of 1,1,1,2,4,4,6,6-octafluoro-2-(trifluoromethyl)-8-iodooctane (i.e., telomers of F71, VDF, and ethylene), 30.6 grams (0.08 mole) of 1,1,1,2,4,4-hexafluoro-2-(trifluoromethyl)-6-iodohexane (i.e., telomers of F71, VDF, and ethylene) about 125 mL of ethanol, 17.8 grams (0.18 mole) of potassium thiocyanate and 0.61 mL of glacial acetic acid to form a mixture. The mixture can be heated to reflux and maintained for about 5 hours. The mixture can be observed as a heterogeneous mixture of white salts and yellow liquid. The mixture can be concentrated and about 200 mL of water and about 200 mL of ether can be added to form a multiphase mixture from which an organic phase can be separated from an aqueous phase. The phases can be separated and the aqueous phase once more extracted with about 100 mL of ether. The organic phases can be combined, dried over sodium sulfate, filtered and concentrated to afford 40.6 grams of the 1,1,1,2,4,4-hexafluoro-2-(trifluoromethyl)-6-thiocyanatohexane and 1,1,1,2,4,4,6,6-octafluoro-2-(trifluoromethyl)-8-thiocyanatooctane product mixture which can be observed as a brown oil, which solidified upon standing. The product structure can be confirmed by NMR and/or chromatographic analysis.

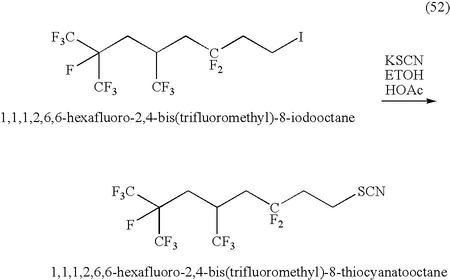

In accordance with scheme (52), in a flask that can be equipped with an agitator, thermocouple and a reflux condenser, 23.3 grams (0.05 mole) of 1,1,1,2,6,6-hexafluoro-2,4-bis(trifluoromethyl)-8-iodooctane (i.e., telomers of F71, VDF, TFP, and ethylene), 50 mL of absolute ethanol, 7.3 grams (0.07 mole) of potassium thiocyanate and 0.3 mL of glacial acetic acid can be placed to form a mixture. The mixture can be heated to reflux and maintained for about 5 hours. The mixture can be observed as a heterogeneous mixture of white salts and yellow liquid. The mixture can be allowed to cool to room temperature and maintained overnight. The ethanol can be removed followed by the addition of 100 mL of water and 100 mL of ether for form a multiphase mixture from which an organic phase can be separated from an aqueous phase. To the aqueous phase, 100 mL of ether can be added and the organic phase collected and dried over sodium sulfate and concentrated to afford 18.4 grams of the 1,1,1,2,6,6-hexafluoro-2,4-is(trifluoromethyl)-8-thiocyanatooctane product that can be observed as a yellow oil. The product structure can be confirmed by NMR and GC/MS analysis.

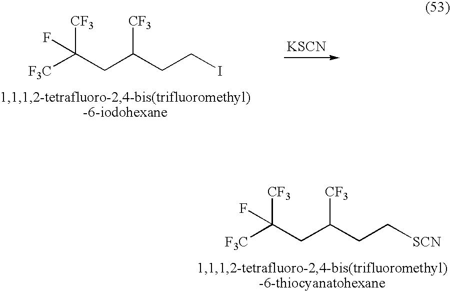

In accordance with scheme (53) above, in a flask that can be equipped with an agitator, thermocouple, reflux condenser, and an addition funnel, 35 grams (0.08 mole) of 1,1,1,2-tetrafluoro-2,4-bis(trifluoromethyl)-6-iodohexane (i.e., telomer of F71, TFP, and ethylene), 85 mL of ethanol, 12.15 grams (0.12 mole) of potassium thiocyanate and 0.5 mL of acetic acid can be placed to form a mixture and heated to reflux and maintained for overnight. The mixture can be cooled and the ethanol removed to afford what can be observed as a heterogeneous mixture of white salts and a liquid. To the heterogeneous mixture, 100 mL of water and 100 mL of ether can be added to form a multiphase mixture from which an organic phase can be separated from an aqueous phase. The aqueous phase can be extracted twice with 100 ml portions of ether and the organic phases combined. The combined organic phase can be dried over sodium sulfate, filtered and concentrated to afford 28.4 grams of the 1,1,1,2-tetrafluoro-2,4-bis(trifluoromethyl)-6-thiocyanatohexane product (97% yd.). The product structure can be confirmed by NMR and/or chromatographic analysis.

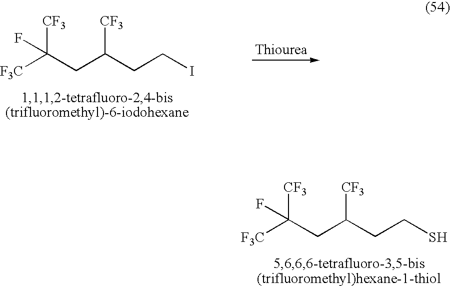

Referring to scheme (54) above, in a flask that can be equipped with an agitator, thermocouple, reflux condenser, dry-ice trap and an addition funnel, 30 grams (0.07 mole) of 1,1,1,2-tetrafluoro-2,4-bis(trifluoromethyl)-6-iodohexane (i.e., telomer of F71, TFP, and ethylene) and 214 mL of ethanol can be placed to form a mixture. To the mixture, 8.2 grams (0.11 mole) of thiourea can be added to form a reaction mixture. The first reaction mixture can be heated to 78° C. and maintained for 24 hours. The reaction mixture can be distilled (156.2 g, 194 mL, 90.7% recovery of ethanol) to afford what can be observed as a slushy white solid in the distillation pot. To the solid, 95 mL of water and 12 grams of sodium hydroxide can be added portion wise at room temperature to afford a multiphase mixture from which an organic phase can be separated from an aqueous phase (maximum temperature can be observed during addition of about 52° C.). The multiphase mixture can be allowed to stir at room temperature for an hour. An atmospheric distillation can be performed to retrieve the product. The distillate can begin to collect when the pot temperature reached about 93° C. Periodically, the temperature can be raised, with a maximum temperature of about 110° C. The product can be separated from the aqueous phase using a Dean Stark trap to afford 20.45 grams of the 5,6,6,6-tetrafluoro-3,5-bis(trifluoromethyl)hexane-1-thiol product and ethanol. The product can be washed with two 20 mL portions of water to remove the remaining ethanol to afford 18.8 grams of the product and can be observed as a clear and colorless liquid (80.7% yd.). The product structure can be confirmed by NMR and/or chromatographic analysis.

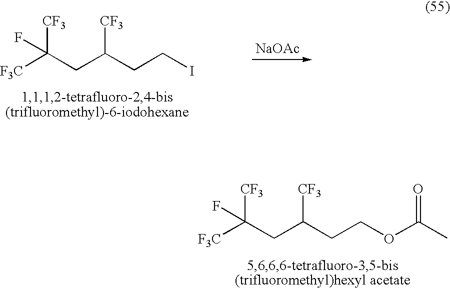

According to scheme (55) above, in a flask that can be equipped with an agitator, thermocouple, reflux condenser, and an addition funnel, 35 grams (0.083 mole) of 1,1,1,2-tetrafluoro-2,4-bis(trifluoromethyl)-6-iodohexane, 20.5 grams (0.25 mole) of sodium acetate and 275 mL of dimethylforamide (DMF) can be placed to form a mixture. The mixture can be heated to 80° C. and maintained for overnight. The mixture can be cooled to room temperature and poured into 300 mL of water and extracted with three 300 mL portions of ether to form a multiphase mixture from which an organic phase can be separated from an aqueous phase. The organic phases can be combined and washed with 500 mL of brine. The organic phase can be collected and dried and stripped of solvent to afford what can be observed as a multiphase oil. The oil can be placed on a Kugelrohr apparatus (40° C., 0.5 hour, 0.03 mmHg) to remove any remaining DMF and can afford 22.3 grams (76.1% yd.) of the 5,6,6,6-tetrafluoro-3,5-bis(trifluoromethyl)hexyl acetate product that can be observed as a oil. The product structure can be confirmed by NMR and/or chromatographic analysis.

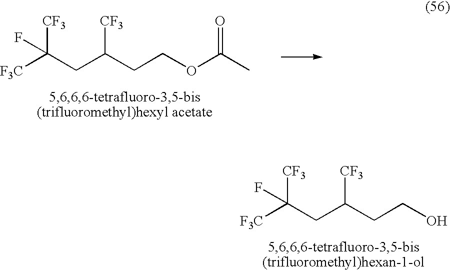

Referring to scheme (56) above, in a flask that can be equipped with an agitator, thermocouple, reflux condenser, and an addition funnel, 0.2 grams of sodium metal, 100 mL of methanol and 22.3 grams of 5,6,6,6 tetrafluoro-3,5-bis(trifluoromethyl)hexyl acetate (see scheme 55 above) can be placed to form a mixture. The mixture can be allowed to stir overnight at room temperature. To the mixture, 17 mL of a 5% (wt/wt) solution of HCl in water can be added and a pH=5 can be observed. To the acidified mixture, 100 mL of ether and two 100 mL portions a saturated bicarbonate solution in water can be added to form a multiphase mixture from which an organic phase can be separated from an aqueous phase. The organic phase can be dried and stripped of solvent to afford 10.9 grams of the 5,6,6,6-tetrafluoro-3,5-bis(trifluoromethyl)hexan-1-ol product that can be observed as a clear and colorless oil (55.6% yd.). The product structure can be confirmed by NMR and/or chromatographic analysis.

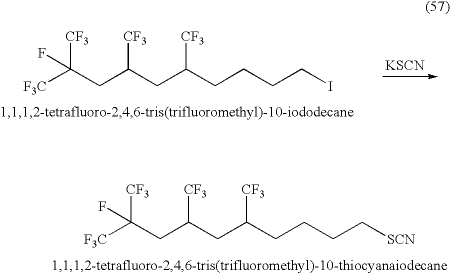

In reference to scheme (57) above, in a flask that can be equipped with an agitator, thermocouple and a reflux condenser, 35 grams (64.3 mmol) of 1,1,1,2-tetrafluoro-2,4,6-tris(trifluoromethyl)-10-iododecane (telomer of F71, TFP, and ethylene), 30 ml of absolute ethanol, 9.5 grams (96.5 mmol) of KSCN and 1.3 ml of acetic acid can be placed to form a mixture. The mixture can be heated to reflux (84.7° C.), stirred and maintained for overnight. The mixture can be cooled to room temperature and concentrated in vacuo to afford what can be observed as a viscous yellow slurry. The slurry can be extracted with 3 liters of diethyl ether, decanted twice, and filtered to produce a wet-cake and a filtrate. The wet cake can be washed three times with 100 ml portions of diethyl ether. The filtrate can be concentrated in vacuo to afford 29.99 grams of 1,1,1,2-tetrafluoro-2,4,6-tris(trifluoromethyl)-10-thiocyanatodecane (97.9% yield) of what can be observed as a light yellow oil. The product structure can be confirmed by NMR and/or chromatographic analysis.

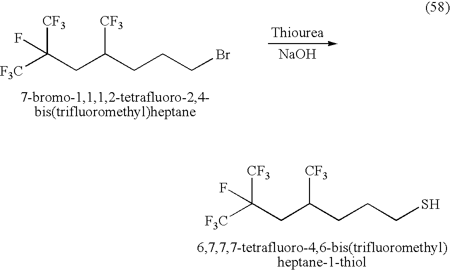

According to scheme (58) above, in a flask that can be equipped with an agitator, thermocouple, reflux condenser, and an addition funnel, 100 grams (0.26 mole) of 7-bromo-1,1,1,2-tetrafluoro-2,4-bis(trifluoromethyl)heptane and about 850 mL of ethanol can be added to form a mixture. To the mixture, 29.5 grams (0.39 mole) of thiourea can be added to form a reaction mixture. The reaction mixture can be heated to reflux and held for from about 42 hours to about 58 hours, and/or about 50 hours. The reaction mixture can be allowed to cool to from about 18° C. to about 24° C., and/or about 21° C. and concentrated in vacuo. To the concentrate, about 360 grams of water and 62.01 grams (1.55 moles) of sodium hydroxide can be added to form a second mixture whereupon an exotherm can be observed. The second mixture can be held at from about 18° C. to about 24° C., and/or about 21° C. for about one hour. The flask can be equipped with a Dean Stark distillation apparatus and the second mixture can be distilled. The distillate can be washed with water to form a multiphase mixture from which an organic phase can be separated from an aqueous phase. The organic phase can be collected, afford the 6,7,7,7-tetrafluoro-4,6-bis(trifluoromethyl)heptane-1-thiol product. The product structure can be confirmed by NMR and/or chromatographic analysis.

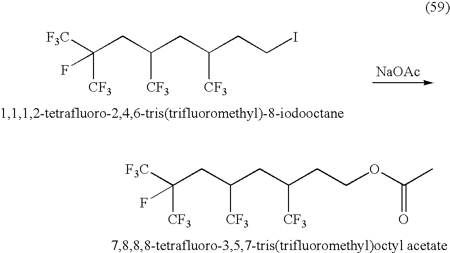

In reference to scheme (59) above, in a flask that can be equipped with an agitator, thermocouple, reflux condenser, and an addition funnel, 35 grams (0.068 mole) of 1,1,1,2-tetrafluoro-2,4,6-tris(trifluoromethyl)-8-iodooctane (i.e., telomer of F71, TFP, and ethylene), 16.69 grams (0.203 mole) of sodium acetate and 223.8 mL of dimethylformamide (DMF) can be placed to form a mixture. The mixture can be heated to 80° C. and maintained for overnight. The reaction mixture can be cooled to room temperature and poured into 300 mL of water to form a multiphase mixture from which an organic phase can be separated from an aqueous phase. The aqueous phase can be extracted with three 300 mL portions of ether. The organic phases can be combined and washed with 500 mL of brine. The organic phase can be dried and stripped of solvent to afford what can be observed as a multiphase oil. The multiphase oil can be placed on a Kugelrohr apparatus (40 C, 1 hour, 0.03 mmHg) to afford 27.25 grams of the 7,8,8,8-tetrafluoro-3,5,7-tris(trifluoromethyl)octyl acetate product (89.6% yd.). The product structure can be confirmed by NMR and/or chromatographic analysis.

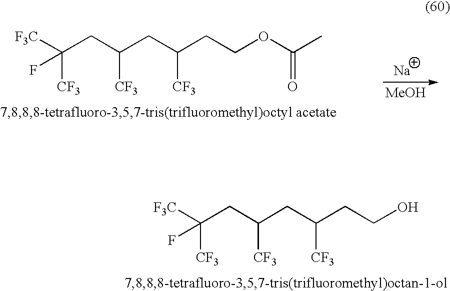

According to scheme (60) above, in a flask that can be equipped with an agitator, thermocouple, reflux condenser, and an addition funnel, 0.2 gram of sodium metal, 100 mL of methanol, and 27.25 grams of 7,8,8,8-tetrafluoro-3,5,7-tris(trifluoromethyl)octyl acetate can be added to form a mixture. The mixture can be allowed to stir for over the weekend at room temperature. The mixture can be treated with 5 ml of a 5% (wt/wt) solution of HCl in water to afford an acidic mixture having a pH of about 5. The acidic mixture can be stripped of methanol and 100 mL of ether can be added to afford a diluent. The diluent can be washed with two 100 mL portions of a saturated solution of sodium bicarbonate in water to form a multiphase mixture from which an organic phase can be separated from an aqueous phase. The organic phase can be dried and stripped of solvent to afford a multiphase oil. The multiphase oil can be placed on a Kugelrohr apparatus (0.03 mmHg, 40 C, 1 hour) to afford 17.6 grams of the 7,8,8,8-tetrafluoro-3,5,7-tris(trifluoromethyl)octan-1-ol product that can be observed as a clear and colorless oil (71.3% % yd.). The product structure can be confirmed by NMR and/or chromatographic analysis.

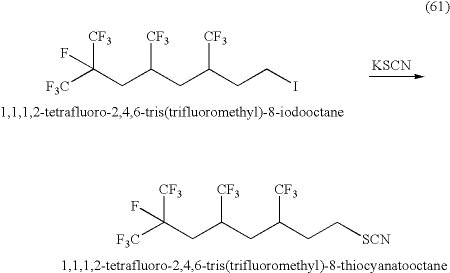

According to scheme (61) above, in a flask that can be equipped with an agitator, thermocouple, reflux condenser, and an addition funnel, 35 grams (0.07 mole) of 1,1,1,2-tetrafluoro-2,4,6-tris(trifluoromethyl)-8-iodooctane and 70 mL of ethanol, 9.9 grams (0.1 mole) of potassium thiocyanate and 0.4 mL of acetic acid can be placed to form a mixture. The mixture can be heated to reflux and maintained for overnight. The mixture can be cooled and the ethanol removed, leaving what can be observed as a heterogeneous mixture of white salts and a liquid. To the heterogeneous mixture, 100 mL of water and 100 mL of ether can be added to form a multiphase mixture from which an organic phase can be separated from an aqueous phase. The aqueous phase can be extracted twice with 100 mL portions of ether the organic phases combined. The combined organic phase can be dried over sodium sulfate, filtered and concentrated to afford 28.8 grams of the 1,1,1,2-tetrafluoro-2,4,6-tris(trifluoromethyl)-8-thiocyanatooctane (95.0% yd.). The product structure can be confirmed by NMR and/or chromatographic analysis.

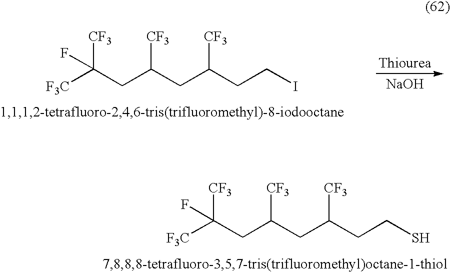

According to scheme (62) above, in a flask that can be equipped with an agitator, thermocouple, reflux condenser, and an dry-ice trap, 30 grams (0.06 mole) of 1,1,1,2-tetrafluoro-2,4,6-tris(trifluoromethyl)-8-iodooctane, 175 mL of ethanol and 6.7 grams (0.09 mole) of thiourea can be added to form a mixture. The mixture can be heated to 78° C. and maintained for 24 hours. The mixture can be concentrated to afford what can be observed as a slushy white solid. To the solid, 75 mL of water can be added to form a multiphase mixture from which an organic phase can be separated from an aqueous phase. To the multiphase mixture, 9.8 grams of sodium hydroxide can be added portion wise at room temperature wherein maximum temperature during addition can be about 48.7° C. The multiphase mixture can be allowed to cool, while stirring, to room temperature and maintained for an hour. The multiphase mixture can be collected via a Dean-Stark wherein the organic phase can be collected to afford 22.4 grams of the 7,8,8,8-tetrafluoro-3,5,7-tris(trifluoromethyl)octane-1-thiol product that can be observed as a clear and colorless liquid. The product can be twice washed with about 25 mL portions of water to remove the remaining ethanol to afford 19.7 grams of the product (80.4% yd.). The product structure can be confirmed by NMR and/or chromatographic analysis.

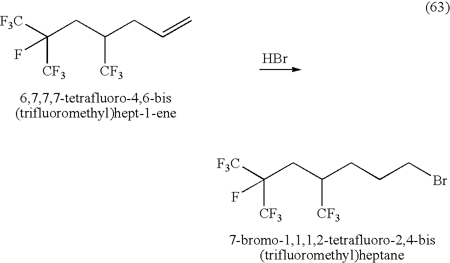

In accordance with scheme (63) above, in a 1 L photochemical reaction vessel that can be equipped with a threaded nylon bushing and an agitator. The threaded nylon bushing can be equipped with a nine inch Pen-Ray® 5.5 watt ultraviolet (UV) lamp with corresponding power supply, pressure gauge, gaseous anhydrous hydrobromic acid feeding tube (feeding tube) set at a depth to feed the gaseous anhydrous hydrobromic acid (HBr) subsurface relative to the olefin, and a venting valve, 708.2 grams (2.314 moles) of 6,7,7,7-tetrafluoro-4,6-bis(trifluoromethyl)hept-1-ene (see scheme 24 above) can be placed. A cylinder of HBr can be connected to the feeding tube and the reaction can be performed by employing the following steps: 1.) While exposing the reaction vessel contents to the UV light, continuously charge the reaction vessel with HBr to achieve and maintain a pressure of about 25 psig to form a mixture that can be held for about eight hours; 2.) Discontinue HBr feed and hold mixture at about 25 psig for from about 15 hours to about 21 hours, and/or about 18 hours. Repeat steps 1 and 2 about four times or until essentially all of the 6,7,7,7-tetrafluoro-4,6-bis(trifluoromethyl)hept-1-ene has been consumed. The mixture can be vacuum distilled to afford the 7-bromo-1,1,1,2-tetrafluoro-2,4-bis(trifluoromethyl)heptane product. (m/z: 307(M⁺-Br) 287(M⁺-BrF) 237(M⁺-CF₃Br) 203(M⁺-C₄H₂F₇))

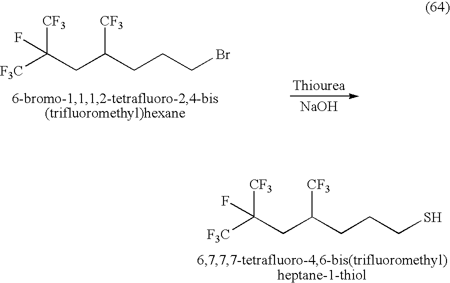

According to scheme (64) above, in a flask that can be equipped with an agitator, thermocouple, reflux condenser, and an addition funnel, 100 grams (0.26 mole) of 6-bromo-1,1,1,2-tetrafluoro-2,4-bis(trifluoromethyl)hexane and about 850 mL of ethanol can be added to form a mixture. To the mixture, 29.5 grams (0.39 mole) of thiourea can be added to form a reaction mixture. The reaction mixture can be heated to reflux and held for from about 42 hours to about 58 hours, and/or about 50 hours. The reaction mixture can be allowed to cool to from about 18° C. to about 24° C., and/or about 21° C. and concentrated in vacuo. To the concentrate, about 360 grams of water and 62.01 grams (1.55 moles) of sodium hydroxide can be added to form a second mixture: whereupon an exotherm can be observed. The second mixture can be held at from about 18° C. to about 24° C., and/or about 21° C. for about one hour. The flask can be equipped with a Dean Stark distillation apparatus and the second mixture can be distilled. The distillate can be washed with water to form a multiphase mixture from which an organic phase can be separated from an aqueous phase. The organic phase can be collected, afford the 6,7,7,7-tetrafluoro-4,6-bis(trifluoromethyl)heptane-1-thiol product. The product structure can be confirmed by NMR and/or chromatographic analysis.

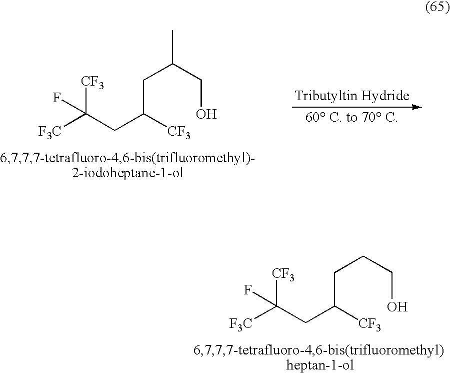

Referring to scheme (65) above, into a 50 mL parallel three neck round bottom flask that can be equipped with a thermocouple, agitator, and a 50 mL pressure equalized addition funnel, 14.48 grams (0.032 mole) of 1,1,1,2,5,5,5-heptafluoro-2-(trifluoromethyl)-4-iodopentane (see scheme—(37), above) and 0.19 gram (0.001 mole) of 2,2′-azobisisobutyronitrile can be placed to form a mixture. The mixture can be heated to from about 60° C. to about 80° C., and/or to about 65° C. To the mixture, 10.06 grams (0.035 mole) of tributyltin hydride can be added drop wise to form a reaction mixture and held at from about 60° C. to about 80° C. for about four hours. The reaction mixture can then be distilled under vacuum to afford the 6,7,7,7-tetrafluoro-4,6-bis(trifluoromethyl)heptan-1-ol product. (m/z: 286(M⁺-F₂) 237(M⁺-CF₄) 226(M⁺-C₃H₈F₂O) High Resolution Mass Spectroscopy: Calculated Mass: 323.0494 Actual Mass: 323.0501 Infrared Spectroscopy: R—OH stretch (w) 3336 cm⁻¹, Csp³-H stretch (w) 2965 cm⁻¹, Csp³-H stretch (w) 2885 cm⁻¹, fingerprint bands 1061 cm⁻¹, 1167 cm⁻¹, 1226 cm⁻¹, 1260 cm⁻¹, 1297 cm⁻¹).

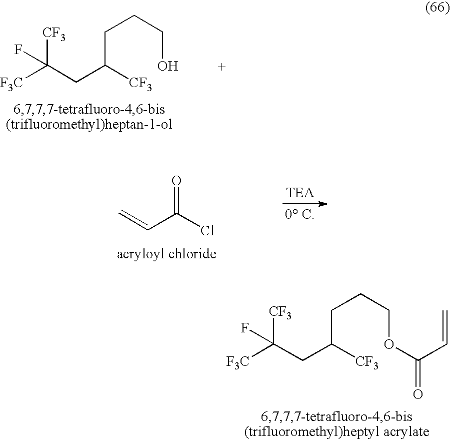

With reference to scheme (66) above, in a flask that can be equipped with an agitator, thermocouple, and an addition funnel, 35 grams (0.11 mole) of 6,7,7,7-tetrafluoro-4,6-bis(trifluoromethyl)heptan-1-ol and 13.5 grams (0.13 mole) of triethylamine can be added to form a mixture. The mixture can be cooled to about 0° C. by employment of an ice-water bath. To the cooled mixture, 11.7 grams (0.13 mole) of acryloyl chloride can be added to form a reaction mixture at a rate such that the reaction mixture is maintained below about 10° C. The reaction mixture can be gradually brought to from about 18° C. to about 24° C., and/or about 21° C. and held stirring for about from about 15 hours to about 21 hours, and/or about 18 hours. The reaction mixture can be washed with a 10 percent (wt/wt) HCl solution at least one time to form a multiphase mixture from which the organic layer can be separated from the aqueous layer and collected, dried over magnesium sulfate and filtered to afford about 39 grams of 97 area percent pure (by gas chromatography) 6,7,7,7-tetrafluoro-4,6-bis(trifluoromethyl)heptyl acrylate product. To the product, 0.012 gram 4-tert-Butylcatechol can be added. (m/z: 379 (M⁺) 332 (M⁺-C₂H₃F) 238 (M⁺-C4H₃F₃O₂) 237 (M⁺-C₄HF₄O)).

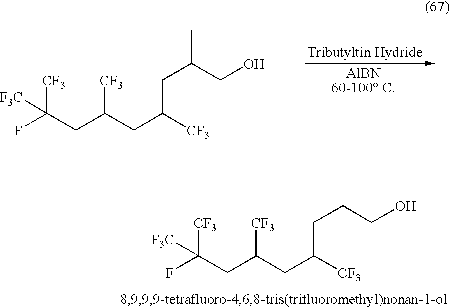

Referring to scheme (67) above, 33.2 gram (0.061 mole) of 8,9,9,9-tetrafluoro-4,6,8-tris(trifluoromethyl)-2-iodononan-1-ol and 4.4 gram (0.03 mole) of 2,2′-azobisisobutyronitrile can be placed into a 125 mL-three neck round bottom flask which can be quipped with an agitator, thermocouple, means of heating, a reflux condenser, and a 50 mL pressure equalizing addition funnel containing about 17.8 gram (0.061 mole) tributyltin hydride (TBTH) to form a mixture. The mixture can be heated to about 65° C., from about 50° C. to about 75° C., and/or about 60° C. to about 65° C. TBTH addition may be carried out over about 90 minutes to form a reaction mixture, whereupon the reaction mixture can change from dark purple-red to a weak orange yellow can be observed. Following the TBTH addition, the reaction mixture can be held at about 65° C. for a period of about four hours. The product 8,9,9,9-tetrafluoro-4,6,8-tris(trifluoromethyl)nonal-1ol can be isolated upon distillation as a viscous colorless oil at about 80° C./8.2 Torr. m/z 419 (M⁺-H′), 382 (M⁺-F₂′), 333 (M⁺-CF₄′), 313 (M⁺-CF₅′), 237 (M⁺-C₄H₂F₇′).

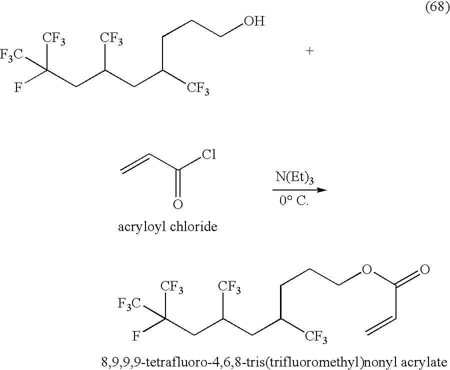

In accordance with scheme (68) above, to a 50 mL three neck round bottom flask that can be equipped with a thermocouple, agitator, ice bath, reflux condenser, and a pressure equalizing funnel which can contain about 2.5 gram (0.03 mole) of acryloyl chloride, about 10.5 gram (0.63 mole) 8,9,9,9-tetrafluoro-4,6,8-tris(trifluoromethyl)nonal-101, about 2.7 gram (0.03 mole) triethylamine, and about 13.6 gram ethyl ether were added to form a mixture. The mixture can be cooled to about 0° C., from about −5° C. to about 5° C., and/or about −2° C. to about 2° C. followed by the slow addition of acryloyl chloride to form a reaction mixture. An immediate exotherm coupled with the mixture changing from a slight brown color to bright pale yellow can be observed. After completion of acryloyl chloride addition the ice bath can be removed allowing the reaction mixture to gradually warm to from about 18° C. to about 24° C., and/or 21° C. for about one hour. The reaction mixture can be washed twice by addition with about 10 mL water to form a multiphase mixture from which an organic phase can be separated from an aqueous phase. The aqueous phase can be further washed twice with about 10 mL portions of ether and an organic phase. The organic layer and the ether extracts can be combined, dried over magnesium sulfate, filtered, and concentrated in vacuo to afford 8,9,9,9-tetrafluoro-4,6,8-tris(trifluoromethyl)nonyl acrylate product that can be observed as a yellow oil. The acrylate product can be a R_{F}-monomer and/or unit as well. About 300 ppm of tert-butylcatechol can be added as a polymerization inhibitor. (m/z 475 (M⁺+H⁺), 434 (M⁺-F₂), 293 (C₈H₇F₁₀′), 209 (C₉H₁₂F₃O₂′), 113 (C₆H₉O₂′))

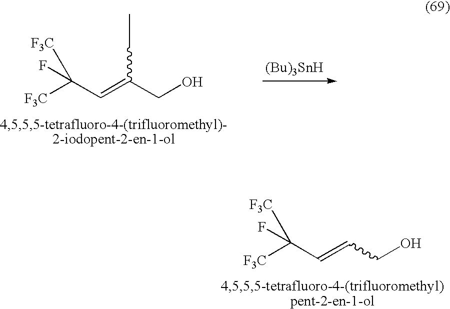

Referring to scheme (69) above, into a flask, that can be equipped with an addition funnel and a thermocouple, 193 grams (0.55 moles) of 4,5,5,5-tetrafluoro-4-(trifluoromethyl)-2-iodopent-2-en-1-ol and 2.0 grams (0.012 mole) of 2,2′-azobisisobutryonitrile (AIBN) can be placed to form a mixture. The mixture can be heated to from about 50° C. to about 75° C., and/or about 64° C. To the mixture, 203.7 grams (0.7 mole) of tributyl tin hydride can be added drop wise to form a reaction mixture. The addition of tributyl tin hydride can be at a rate such that the reaction mixture temperature can be maintained at from about 60° C. to about 70° C., to about 65° C. The reaction mixture can be heated to about 75° C. and maintained for about 1.5 hours. Distillation of the reaction mixture can afford the 4,5,5,5-tetrafluoro-4-(trifluoromethyl)pent-2-en-1-ol product (bp; 63.5° C./26.6 torr) at about 86 percent yield. The product structure can be confirmed by gas chromatography/mass spectroscopy and/or NMR.

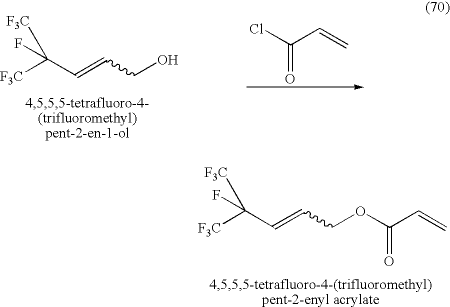

With reference to scheme (70) above, into a 2 liter round bottom flask that can be equipped with an addition funnel, agitator, and a thermocouple can be placed 200 grams (0.885 mole) of 4,5,5,5-tetrafluoro-4-(trifluoromethyl)pent-2-en-1-ol, 106 grams (1.05 moles) of triethyl amine and 500 ml of diethyl ether to form a mixture. The mixture can be chilled in an ice/water bath from about 0° to about 5° C., and/or about 0° C. To the chilled mixture, 112 grams (1.24 moles) of acryloyl chloride can be added to form a reaction mixture. The rate of addition of the acryloyl chloride to the mixture is such that reaction mixture temperature should not exceed about 15° C. The reaction mixture can be maintained at about 4° C. for about 1 hour. To the reaction mixture, about 700 ml of H₂O can be added to form a multiphase mixture from which an organic phase can be separated from an aqueous phase. The aqueous phase can be extracted twice with diethyl ether and combined with the previously separated organic phase, dried over MgSO₄, and filtered. The solvent can be removed under reduced pressure to afford the product isomer mixture (4,5,5,5-tetrafluoro-4-(trifluoromethyl)pent-2-enyl acrylate that can be about 92.2 area (wt/wt) % by gas chromatography. The product isomer mixture can be further characterized by NMR and gas chromatography/mass spectroscopy.

An embodiment of the disclosure provides R_{F}-surfactant compositions that include the R_{F} portions described above. Exemplary R_{F}-surfactant compositions can be referred to as R_{F}-Qₛ. According to exemplary embodiments the RF portion can at least partially include an R_{F}(R_{T})n portion as described above. The R_{F}(R_{T})n portion of the surfactant can also include the Rₛ portion described above. In accordance with exemplary implementations the Rₛ portion can be incorporated to provide additional carbon between the R_{F} and/or R_{F}(R_{T})n portions and the Q_{S} portion of the surfactant. Exemplary Rₛ portions include —CH₂—CH₂—.

In a system having at least two parts, R_{F} can have a greater affinity for a first part of the system than Qₛ, and Qₛ can have a greater affinity for a second part of the system than R_{F}. The system can include liquid/liquid systems, liquid/gas systems, liquid/solid systems, and/or gas/solid systems. Liquid/liquid systems, for example, can include systems having at least one liquid part that includes water and another liquid part that is hydrophobic relative to the part that includes water. Liquid/liquid systems can also include systems of which water is not a part of the system, such as hydrocarbon liquid systems. In exemplary embodiments, R_{F} can be hydrophobic relative to Qₛ and/or Qₛ can be hydrophilic relative to R_{F}. R_{F} can be hydrophobic and Qₛ can be hydrophilic, for example. The hydrophobic portion can be referred to as the tail of the R_{F}-surfactant, and the hydrophilic portion can be referred to as the head of the R_{F}-surfactant. The R_{F}-surfactants can include those surfactants having a tail or hydrophobic portion containing fluorine. The R_{F}-surfactant tail or hydrophobic portion can be referred to as an R_{F} portion, and the R_{F}-surfactant head or hydrophilic portion can be referred to as a Qₛ portion. The R_{F}-surfactants can be produced from R_{F}-intermediates utilizing the methods and systems detailed in Published International Applications. Exemplary R_{F}-surfactants include those in Table 9 below.

TABLE 9
R_{F}-surfactants

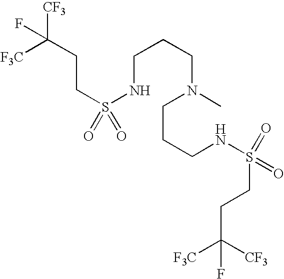

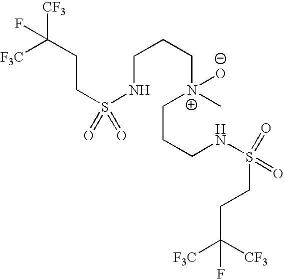

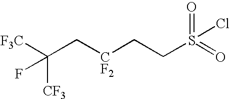

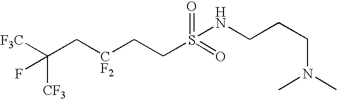

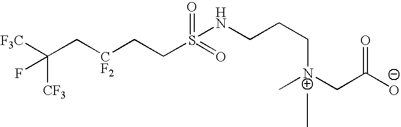

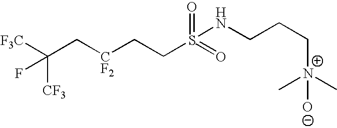

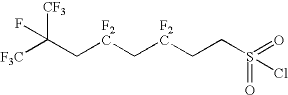

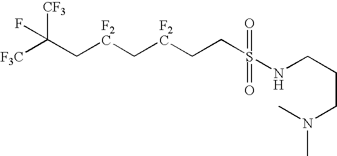

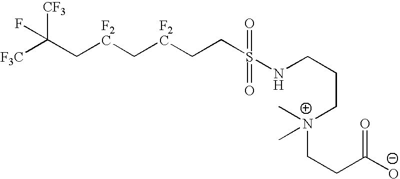

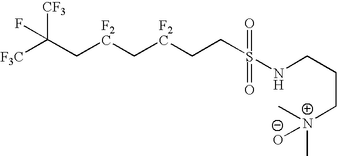

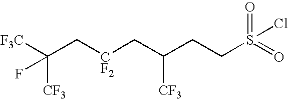

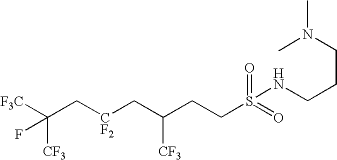

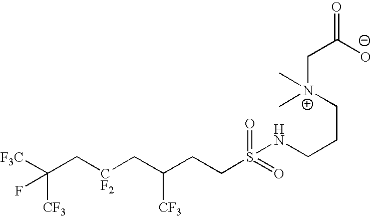

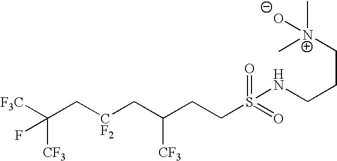

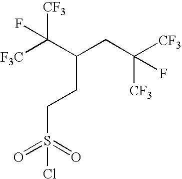

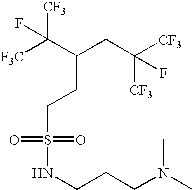

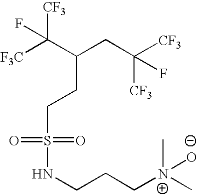

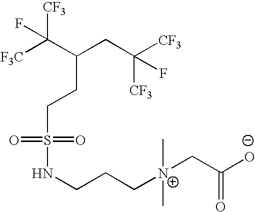

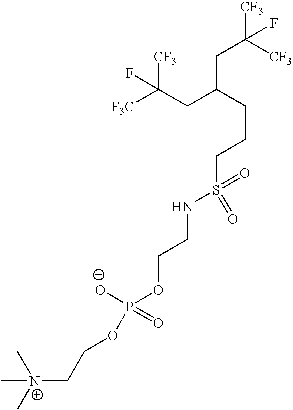

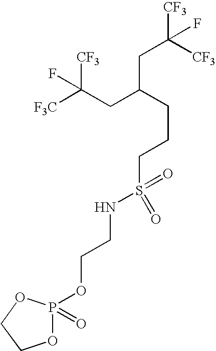

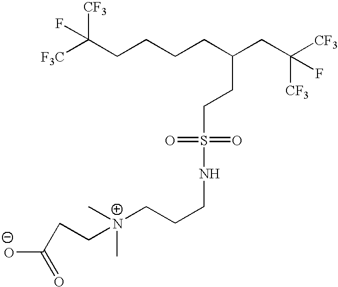

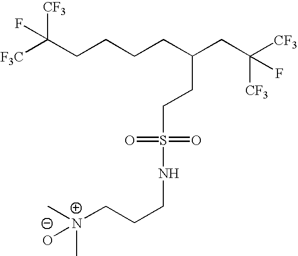

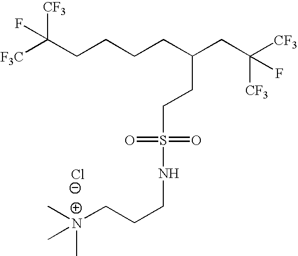

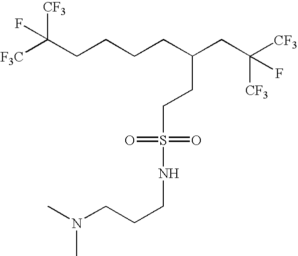

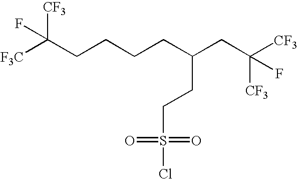

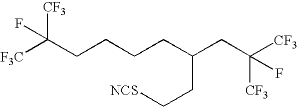

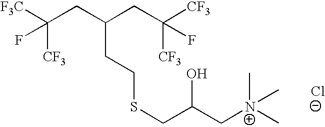

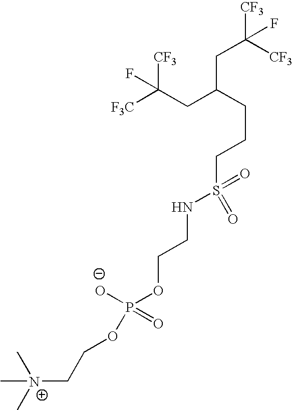

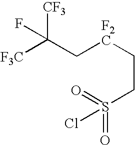

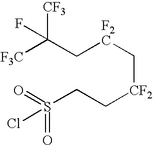

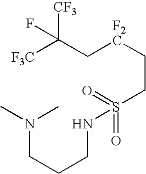

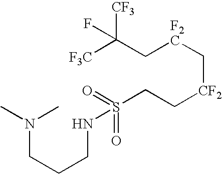

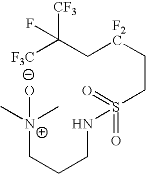

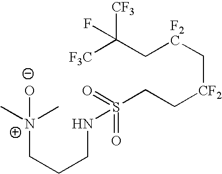

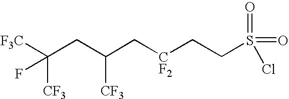

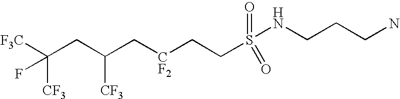

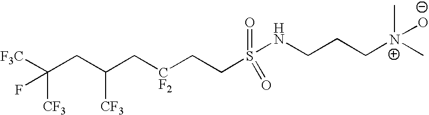

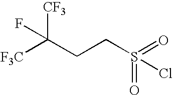

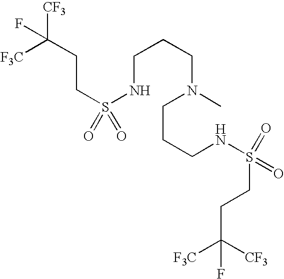

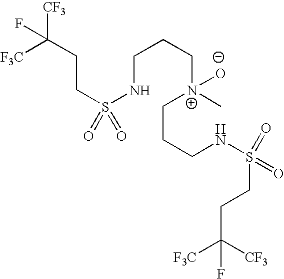

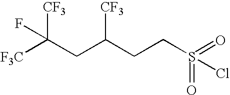

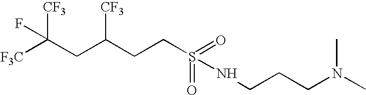

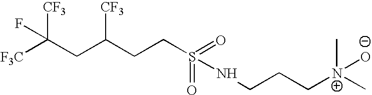

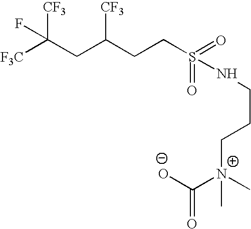

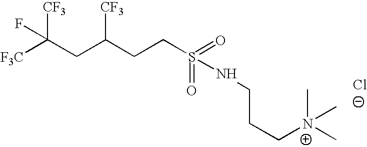

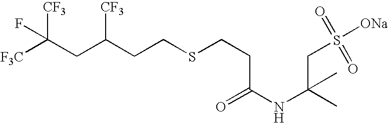

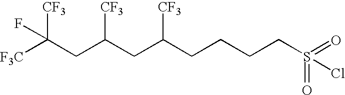

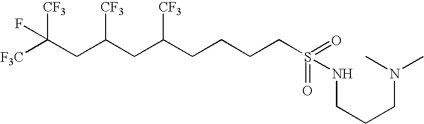

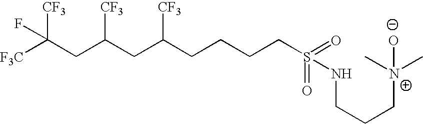

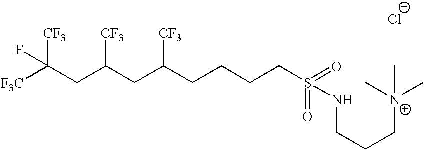

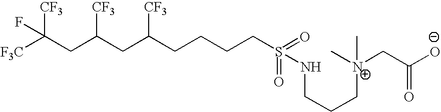

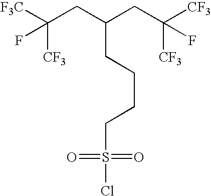

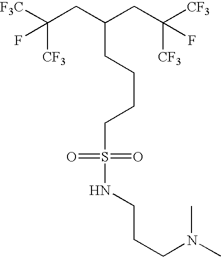

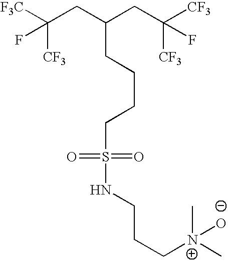

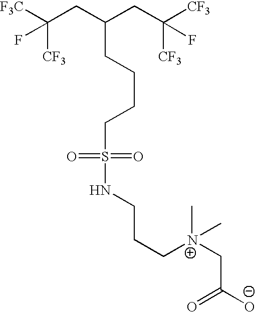

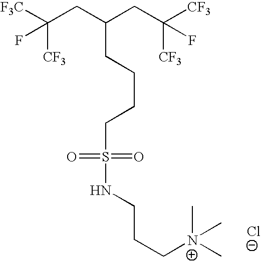

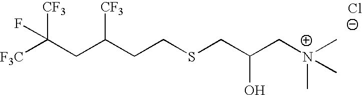

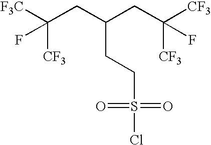

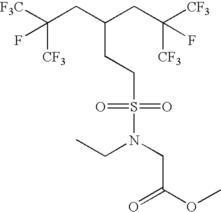

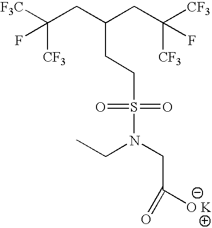

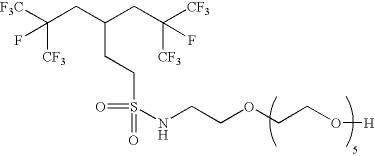

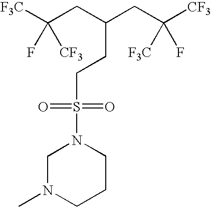

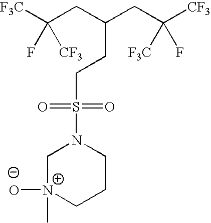

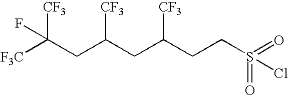

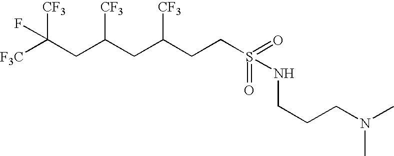

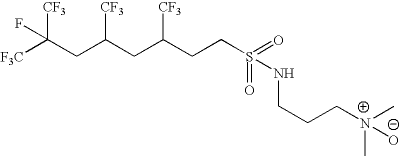

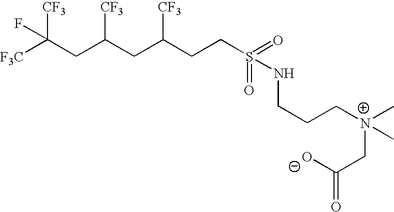

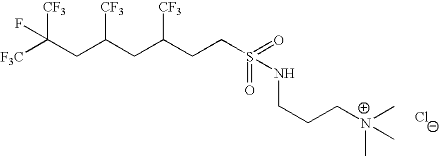

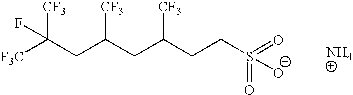

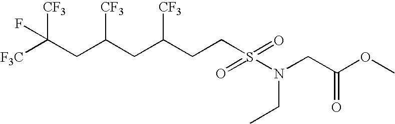

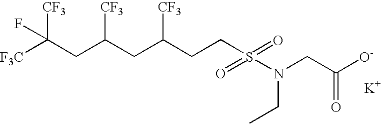

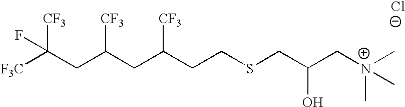

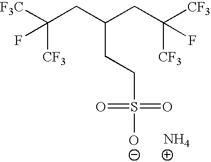

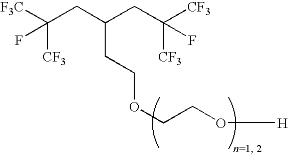

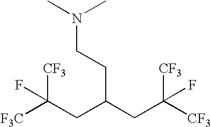

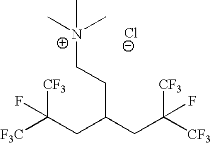

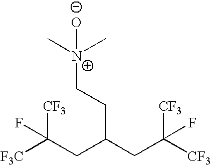

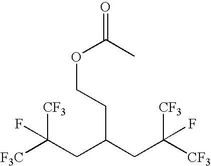

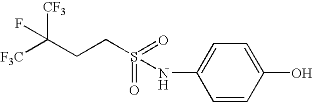

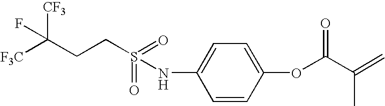

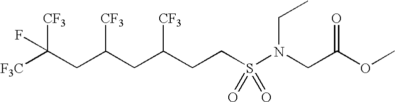

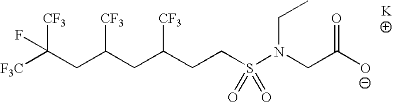

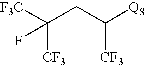

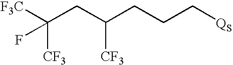

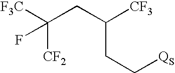

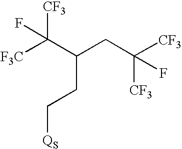

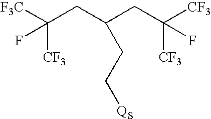

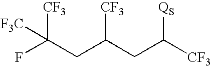

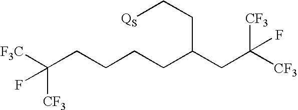

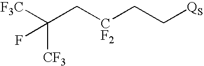

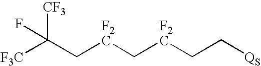

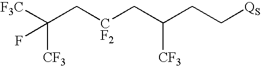

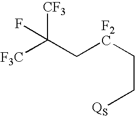

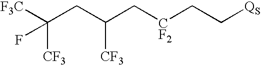

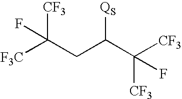

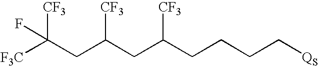

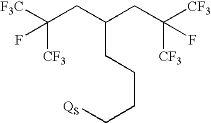

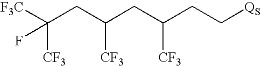

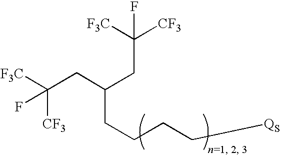

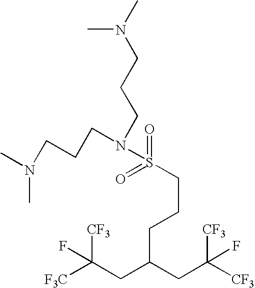

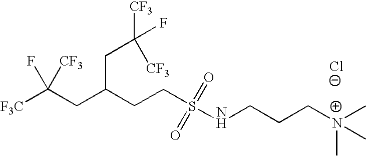

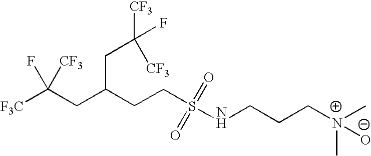

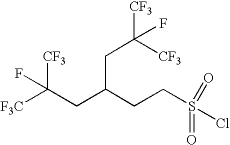

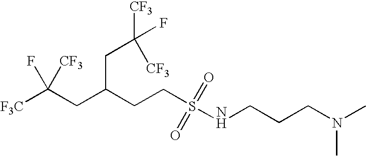

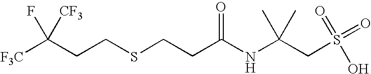

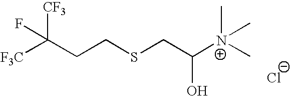

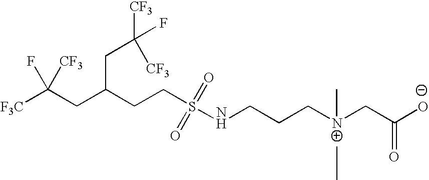

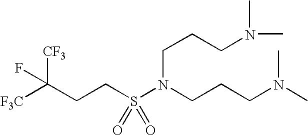

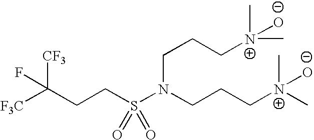

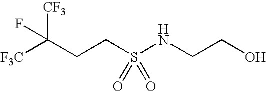

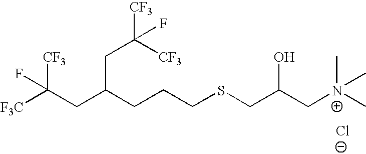

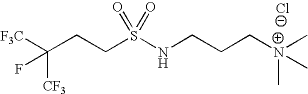

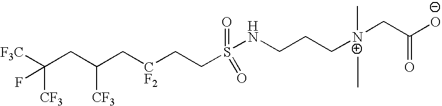

R_{F}-surfactants can also include

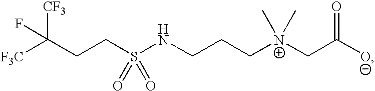

having;

NMR: ¹H (D6-DMSO, 300 MHz) δ 1.8 (m, 2H), 2.6 (m, 2H), 3.0 (m, 2H), 3.1 (bs, 6H), 3.6 (m, 2H), 3.9 (m, 4H), 7.9 (bs, 1H); ¹³C (D6-DMSO, 75 MHz) δ 22.6, 22.9, 23.1, 43.1, 50.0, 60.8, 64.4, 88-93 (ds), 114.5-126.5 (qd); and ¹⁹F (CFCl₃, D6-DMSO, 282 MHz) δ −76.4 (d, 6.95 Hz, 6F), −183.4 (m, ¹F)

Where LC/MS can be used to identify compounds, Table 10 of LC/MS parameters, below, can be used.

TABLE 10
LC-MS Parameters

| | |
|---|---|
| Column Type: | Phenomonex Luna C18 column, 5 micrometer |
| Column Size: | 2 × 50 mm |
| Column Temp: | 25° C. |
| Gradient Pump | Agilent 1100 Quat Pump G1311A |
| Detector: | Agilent Diode Array Detector G13115B |
| Detector Wavelength: | 250 nm (referenced against 360 nm) |
| Mass Detector: | Agilent 1100 MSD G1946C |
| Source: | Electrospray Positive Ion |
| Fragmentor: | 80 |
| Software | ChemStation Rev A.08.03 |
| Conc: | Ca 100 ppm |
| Injector:Rheodyne | 10 microliter |
| Elution Type: | Gradient |
| Flow Rate: | 0.3 mL/min |
| Mobile Phase: | A: Water (JT Baker HPLC grade) w/0.05% |
| | HCO₂H |
| | B: Acetonitrile w/0.05% HCO₂H |
| Gradient Conditions: | 90:10 A:B increase to 100% B in 6 min and |
| | then hold for 4 min at 100% B |

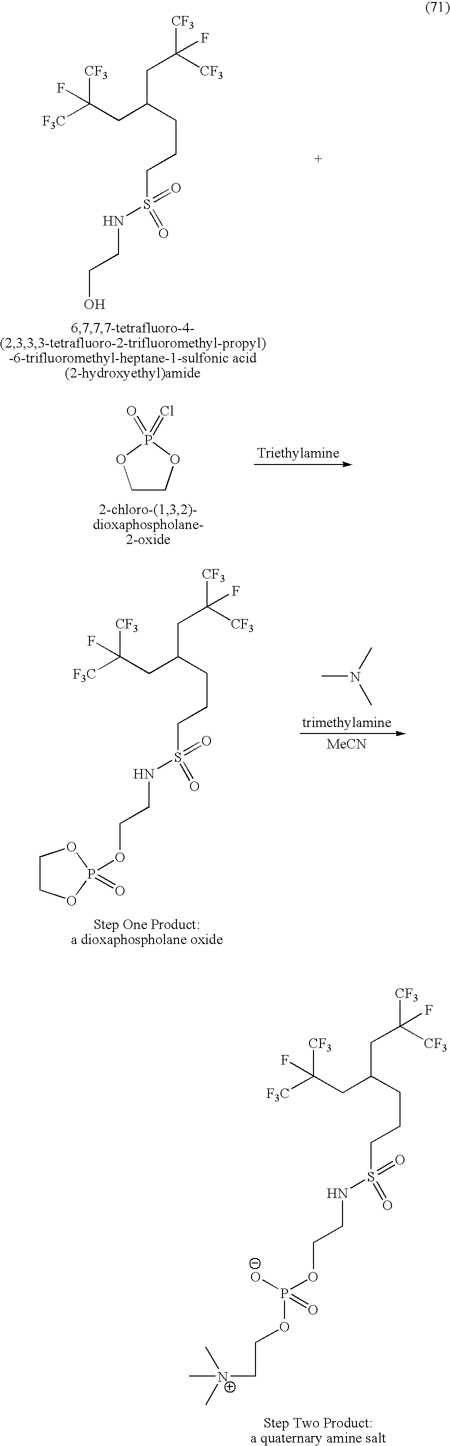

According to scheme (71) above, in a flask that can be equipped with an agitator, thermocouple, ice water bath, and an addition funnel, 11.0 grams (0.02 mole) of 6,7,7,7-tetrafluoro-4-(2,3,3,3-tetrafluoro-2-trifluoromethyl-propyl)-6-trifluoromethyl-heptane-1-sulfonic acid-(2-hydroxyethyl)amide, 2.87 grams (0.02 mole) of 2-chloro-[1,3,2]dioxaphospholane-2-oxide, and about 66 mL of anhydrous ether can be placed to form a mixture. The mixture can be cooled to about 0° C. using an ice water bath. To the mixture, 0.88 grams (0.009 mole) of triethylamine can be added drop wise to form a reaction mixture. A white precipitate can be observed to form immediately upon addition of the triethylamine to the mixture. The reaction mixture can be allowed to warm to from about 18° C. to about 24° C., and/or about 21° C. and held for about four hours. The reaction mixture can then be filtered and concentrated in vacuo to afford crude step one reaction product observed as a pale yellow oil. To remove residual ether, the crude step one product can be placed on a Kugelrohr apparatus (40° C., 0.1 torr, 60 minutes) to afford about 12.8 grams of step one product. The product structure can be confirmed by NMR analysis. In flask that can be equipped with an agitator, thermocouple, and an addition funnel, the step one product can be added and about 130 mL of acetonitrile to form a mixture The mixture can be chilled using a dry ice/acetone bath and 18.45 grams (0.31 mole) of trimethylamine can be added drop wise to form a reaction mixture. The reaction mixture can be allowed to warm to from about 18° C. to about 24° C., and/or about 21° C. followed by heating to about 60° C. for about five hours wherein a white precipitate can be observed to form. The reaction mixture can be chilled to about 0° C. using an ice water bath and held from about 15 hours to about 21 hours, and/or about 18 hours. The white precipitate can be filtered from the reaction mixture and dried from about 15 hours to about 21 hours, and/or about 18 hours in vacuo at about 50° C. to afford 4.36 grams of step two product. The product structure can be confirmed by NMR and/or chromatographic analysis.

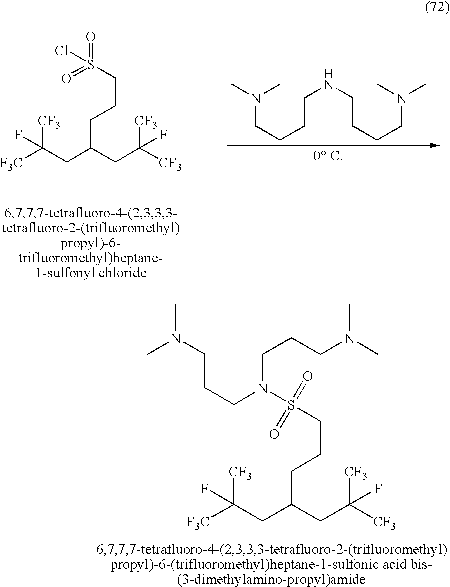

In accordance with scheme (72) above, in a flask that can be equipped with a thermocouple, addition funnel, and an agitator, 10.1 gram (0.054 mole) of 3,3′-iminobis(N,N′-dimethylaminopropylamine), about 45 mL chloroform can be placed to form a mixture and chilled to about 0° C. using an ice/acetone bath. To the mixture, 10.0 gram (0.019 mole) of 6,7,7,7-tetrafluoro-4-(2,3,3,3-tetrafluoro-2-(trifluoromethyl)propyl)-6-(trifluoromethyl)heptane-1-sulfonyl chloride (see, e.g. published International Patent applications: PCT/US05/03429, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; PCT/US05/02617, entitled Compositions, Halogenated Compositions, Chemical Production and Telomerization Processes, filed Jan. 28, 2005; PCT/US05/03433, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; PCT/US05/03137, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005; and PCT/US05/03138, entitled Production Processes and Systems, Compositions, Surfactants, Monomer Units, Metal Complexes, Phosphate Esters, Glycols, Aqueous Film Forming Foams, and Foam Stabilizers, filed Jan. 28, 2005) and about 45 mL of methylene chloride can be added drop wise to form a reaction mixture. The rate of addition can be such that a reaction mixture temperature can be maintained at about 0° C. The reaction mixture can be held at about 0° C. for about one hour. To the reaction mixture, about 90 mL of saturated sodium bicarbonate, about 90 mL water, and about 90 mL brine solution can be added sequentially wherein each step a multiphase mixture can be formed from which an organic phase can be separated from an aqueous phase. The organic phase can be collected and dried over magnesium sulfate, filtered, and concentrated in vacuo to provide about 11.66 gram of the 6,7,7,7-tetrafluoro-4-(2,3,3,3-tetrafluoro-2-(trifluoromethyl)propyl)-6-(trifluoromethyl)heptane-1-sulfonic acid bis-(3-dimethylamino-propyl)amide product as what can be observed as a yellow oil. The product structure can be confirmed by employing NMR and/or chromatographic analysis.

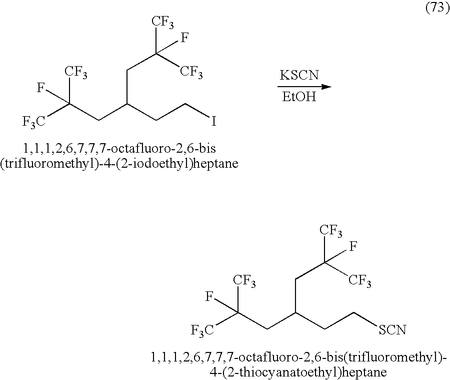

According to scheme (73) above, in a flask that can be equipped with an agitator, thermocouple, and a reflux condenser, 20.0 grams (0.04 mole) of 1,1,1,2,6,7,7,7-octafluoro-4-(2-iodoethyl)-2,6-bis(trifluoromethyl)heptane (refer to scheme (29) above), 4.7 grams (0.05 mole) of potassium thiocyanate, about 75 mL of ethanol, and about 0.2 mL of acetic acid can be placed to form a mixture. The mixture can be heated to reflux and held for from about 15 hours to about 21 hours, and/or about 18 hours. The mixture can be cooled to from about 18° C. to about 24° C., and/or about 21° C. and concentrated in vacuo to form a residue. The residue can be extracted with about 100 mL of ether, filtered, and concentrated in vacuo to afford 17.1 grams of the 1,1,1,2,6,7,7,7-octafluoro-4-(2-thiocyanatoethyl)-2,6-bis(trifluoromethyl))heptane product. The product structure can be confirmed by NMR and/or chromatographic analysis.

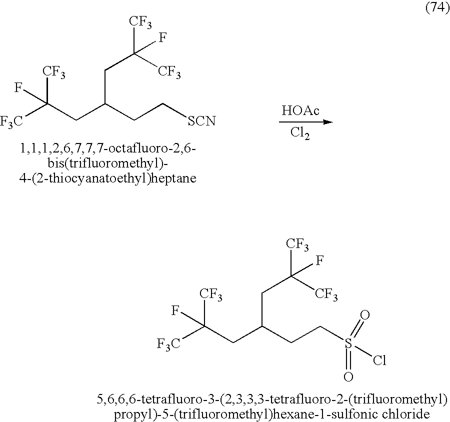

In reference to scheme (74) above, in a flask that can be equipped with an agitator and a thermocouple, 262 grams (0.56 mole) of 1,1,1,2,6,7,7,7-octafluoro-4-(2-thiocyanatoethyl)-2,6-bis(trifluoromethyl))heptane (refer to scheme (73) above) and about 530 mL of acetic acid can be placed to form a mixture. The mixture can be heated to 50° C. and then sparged with chlorine gas to form a reaction mixture. To the reaction mixture can be added about 3.5 mL of water, which can be performed about every four hours during the course of the reaction. An exotherm can be observed during the addition of water to the reaction mixture. The reaction mixture can be held at 50° C. with continuous sparging with chlorine gas for from about 15 hours to about 21 hours, and/or about 18 hours. The reaction mixture can be cooled to from about 18° C. to about 24° C., and/or about 21° C. and about 500 mL of water and about 500 mL of chloroform can be added to form a multiphase mixture from which an organic phase can be separated from an aqueous phase. The organic phase can be collected and rewashed with about 500 mL of water and about 500 mL of a saturated solution of NaHCO₃ and a saturated solution of NaCl wherein in each case above a multiphase mixture can be formed from which an organic phase can be separated from an aqueous phase. The organic phase can be dried and concentrated to afford 270.1 gram of the 5,6,6,6-tetrafluoro-3-(2,3,3,3-tetrafluoro-2-trifluoromethyl-propyl)-5-trifluoromethyl-hexanesulfonyl chloride product that can be observed to be a pale oil. The product structure can be confirmed by NMR and/or chromatographic analysis.

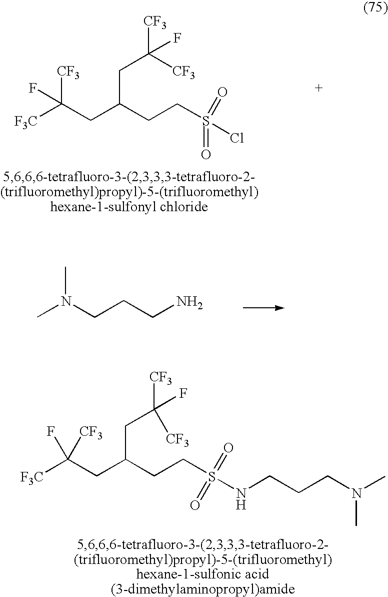

In accordance with scheme (75) above, in a flask that can be equipped with an agitator, thermocouple, an addition funnel, and an ice bath, 152.52 grams (1.49 moles) of dimethylpropylamine and about 705 mL of chloroform can be added to form a mixture. The mixture can be chilled to from about 0° C. to about 5° C., and/or about 2.5° C. In the addition funnel, 270 grams (0.53 mole) of 5,6,6,6-tetrafluoro-3-(2,3,3,3-tetrafluoro-2-trifluoromethyl-propyl)-5-trifluoromethyl-hexanesulfonyl chloride (refer to scheme (74) above) and about 470 mL of chloroform can be added to form an addition mixture. To the chilled mixture the addition mixture can be added drop wise to form a reaction mixture. The rate of the addition can be such that the reaction mixture maintains a temperature below about 5° C. The reaction mixture can be allowed to warm to from about 18° C. to about 24° C., and/or about 21° C. for from about 15 hours to about 21 hours, and/or about 18 hours. The reaction mixture can be washed sequentially three times with 1 L of a saturated solution of sodium bicarbonate, twice with 1 L portions of saturated brine solution, and once with 1 L of water to form a multiphase mixture from which an organic phase can be separated from an aqueous phase. The organic phase can be collected, dried over sodium sulfate, and concentrated to afford 282.5 grams of the 5,6,6,6-tetrafluoro-3-(2,3,3,3-tetrafluoro-2-trifluoromethyl-propyl)-5-trifluoromethyl-hexane-1-sulfonic acid(3-dimethylamino-propyl)amide product that can be observed as a white solid. The product structure can be confirmed by NMR and/or chromatographic analysis.

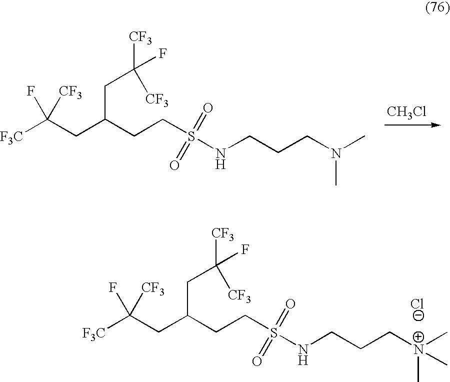

Referring to scheme (76) above, in a sealable flask that can be equipped with a thermocouple and an agitator, 10 grams (0.02 mole) of 5,6,6,6-tetrafluoro-3-(2,3,3,3-tetrafluoro-2-trifluoromethyl-propyl)-5-trifluoromethyl-hexane-1-sulfonic acid(3-dimethylamino-propyl)amide (refer to scheme (75) above) and 17.5 mL of a 1M solution of chloromethane in tert-butyl methyl ether can be added to form a mixture. The mixture can be heated to about 55° C. and held for from about 15 hours to about 21 hours, and/or about 18 hours. The flask can then be vented and the mixture filtered and washed with ether to afford 7.7 grams of 5,6,6,6-tetrafluoro-3-(2,3,3,3-tetrafluoro-2-trifluoromethyl-propyl)-5-trifluoromethyl-hexane-1-sulfonic acid(3-trimethylamino-propyl)amide chloride product that can be observed as a white solid. The product structure can be confirmed by NMR and/or chromatographic analysis.

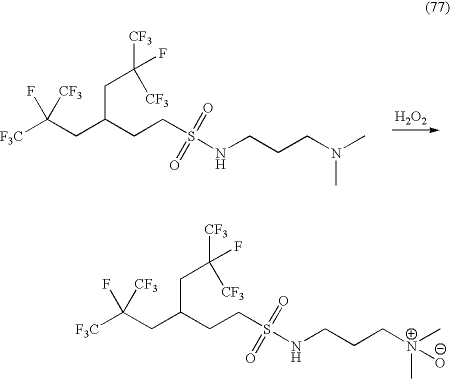

According to scheme (77) above, in a flask that can be equipped with an agitator and a thermocouple, 49 grams (0.09 mole) of 5,6,6,6-tetrafluoro-3-(2,3,3,3-tetrafluoro-2-trifluoromethyl-propyl)-5-trifluoromethyl-hexane-1-sulfonic acid(3-dimethylamino-propyl)amide (refer to scheme (75) above), about 65 mL of ethanol, about 9.7 mL of water, and about 40 mL of a 50 (wt/wt) percent solution of hydrogen peroxide to form a mixture. The mixture can be heated to about 35° C. and held for about 3.5 hours. To the mixture, about 26 grams of carbon can be slowly added to form a slurry. The slurry can be agitated at from about 18° C. to about 24° C., and/or about 21° C. for from about 15 hours to about 21 hours, and/or about 18 hours. The slurry can be filtered through celite and the filter cake can be washed with about 500 mL of ethanol. The filtrate can be observed to be colorless and can be concentrated to afford 49 grams of the 5,6,6,6-tetrafluoro-3-(2,3,3,3-tetrafluoro-2-trifluoromethyl-propyl)-5-trifluoromethyl-hexane-1-sulfonic acid(3-dimethylamino-propyl)amide oxide product that can be observed to be a white solid. The product structure can be confirmed by NMR and/or chromatographic analysis.

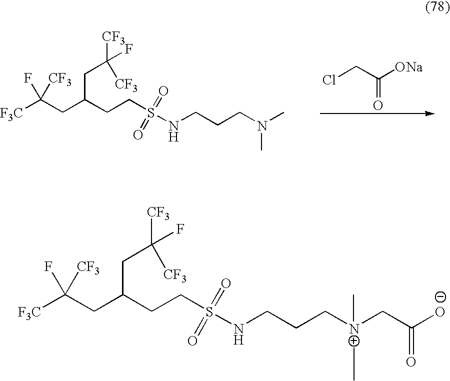

According to scheme (78) above, in a flask that can be equipped with an agitator, thermocouple, and a reflux condenser, 10 grams (0.0175 mole) of 5,6,6,6-tetrafluoro-3-(2,3,3,3-tetrafluoro-2-trifluoromethyl-propyl)-5-trifluoromethyl-hexane-1-sulfonic acid(3-dimethylamino-propyl)amide (refer to scheme (75) above), about 50 mL of ethanol, and 2.03 grams (0.0175 mole) of sodium chloroacetate can be added to form a mixture. The mixture can be heated to reflux and held for from about 66 hours to about 74 hours, and/or about 70 hours. The mixture can be filtered and the filtrate collected and concentrated in vacuo to afford the

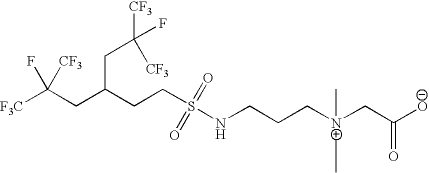

product that can be observed to be an impure pasty solid. The product structure can be confirmed by NMR and/or chromatographic analysis.

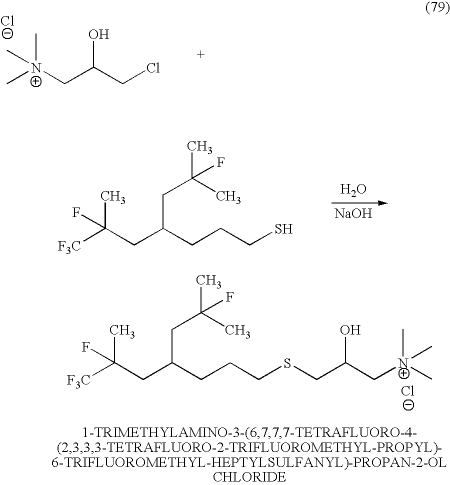

According to scheme (79) above, in a flask that can be equipped with an agitator, about 6 mL of water, 10 grams (0.022 mole) of 6,7,7,7-tetrafluoro-(2,3,3,3-tetrafluoro-2-trifluoromethyl-propyl)-6-trifluoromethyl-heptane-1-thiol, 6.9 grams (0.02 mole) of 3-chloro-2-hydroxypropyl-trimethyl ammonium chloride in 18 grams of water, and 0.88 gram (0.02 mole) of sodium hydroxide can be placed to form a mixture. The mixture can be held at from about 18° C. to about 24° C., and/or about 21° C. for from about 15 hours to about 21 hours, and/or about 18 hours. The mixture can be observed to be a white slurry and can be filtered with the filtrate being collected. The filtrate can be stripped of water by using ethanol followed by chloroform to afford an oil that can be observed as clear and colorless. The oil can be placed on a Kugelrohr apparatus (50° C., 0.03 mmHg, 30 minutes) to afford 15.6 grams of impure 1-trimethylamino-3-[6,7,7,7-tetrafluoro-4-(2,3,3,3-tetrafluoro-2-trifluoromethyl-propyl)-6-trifluoromethyl-heptylsulfanyl]propan-2-ol chloride product. The product can be dissolved in about 50 mL of ethanol to form a mixture and held at from about 18° C. to about 24° C., and/or about 21° C. for from about 54 hours to about 70 hours, and/or about 62 hours. The mixture can be filtered and concentrated to afford 12.3 grams of product that can be observed at a white solid. The product structure can be confirmed by NMR and/or chromatographic analysis.

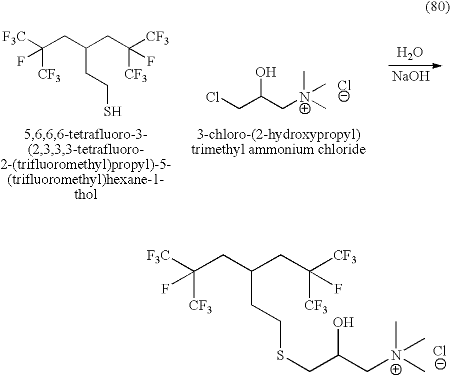

According to scheme (80) above, in a flask that can be equipped with an agitator and a thermocouple, 10 grams (0.023 mole) of 5,6,6,6-tetrafluoro-3-(2,3,3,3-tetrafluoro-2-(trifluoromethyl)propyl)-5-(trifluoromethyl)hexane-1-thiol, 7.12 grams (0.023 mole) of 3-chloro-(2-hydroxypropyl)trimethyl ammonium chloride, and 0.91 grams (0.023 mole) of sodium hydroxide can be placed to form a mixture and held from about 15 hours to about 21 hours, and/or about 18 hours whereupon a white solid can be observed to have formed. The mixture can be filtered and washed three times with 500 mL portions of ethanol and twice with 500 mL portions of chloroform to afford what can be observed as a clear and colorless oil. The oil can be placed on a Kugelrohr apparatus (50° C., 0.03 mmHg, 20 minutes) to afford another oil which can be titrated with four 200 mL portions of ether wherein the ether was decanted each time to afford a solid. The solid can be dried to afford 8.8 grams of the

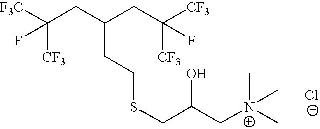

product. The product structure can be confirmed by NMR and/or chromatographic analysis.

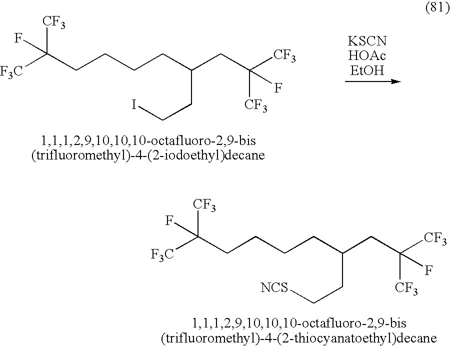

According to scheme (81) above, in a flask that can be equipped with an agitator, thermocouple, and a reflux condenser, 60 grams of a mixture containing about 85 (wt/wt) percent 1,1,1,2,9,10,10,10-octafluoro-2,9-bis(trifluoromethyl-4-(2-iodoethyl)decane (refer to scheme ( ) above) and about 15 (wt/wt) percent of 1,1,1,2,9,10,10,10-octafluoro-2,9-bis(trifluoromethyl-4-(4-iodobutyl)decane, about 75 mL of ethanol, 15.2 grams (0.16 mole) of potassium thiocyanate, and about 1 mL of acetic acid can be placed to form a reaction mixture which can be observed to be a heterogeneous mixture of white salts and brownish liquid. The mixture can be heated to reflux and held for from about 15 hours to about 21 hours, and/or about 18 hours. The ethanol can be removed from the reaction mixture and about 150 mL of water and 150 mL of ether can be added to form a multiphase mixture from which an organic phase can be separated from an aqueous phase. To the aqueous phase, 150 mL of ether can be added to form a separate multiphase mixture from which an organic phase can be separated from an aqueous phase. The organic phases from both multiphase mixtures can be combined, dried over sodium sulfate, filtered, and concentrated. The concentrated organic phase can be placed on a Kugelrohr apparatus (45 minutes, 0.03 mmHg, 150° C.) to afford 44.1 grams of the product mixture containing 1,1,1,2,9,10,10,10-octafluoro-2,9-bis(trifluoromethyl-4-(2-thiocyanatoethyl)decane and 1,1,1,2,9,10,10,10-octafluoro-2,9-bis(trifluoromethyl-4-(4-thiocyanatobutyl)decane which can be observed to be yellow in color. The product structure(s) can be confirmed by NMR and/or chromatographic analysis.

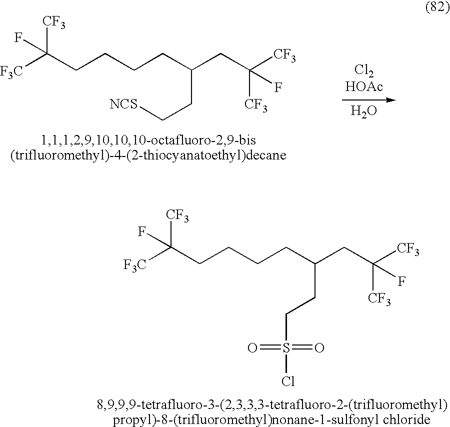

In reference to scheme (82) above, in a flask that can be equipped with an agitator, chlorine gas addition tube, and thermocouple, 44.1 grams of a mixture containing about 85 (wt/wt) percent of 1,1,1,2,9,10,10,10-octafluoro-2,9-bis(trifluoromethyl-4-(2-thiocyanatoethyl)decane (refer to scheme (81) above) and about 15 (wt/wt) percent of 1,1,1,2,9,10,10,10-octafluoro-2,9-bis(trifluoromethyl-4-(4-thiocyanatobutyl)decane and about 850 mL of acetic acid can be placed to form a new mixture. The new mixture can be heated to about 50° C. and chlorine gas can be continuously added for about four hours to form a reaction mixture. To the reaction mixture, about 4 mL of water can be slowly added whereupon a large exotherm can be observed causing the reaction mixture temperature to peak at about 62° C. The reaction mixture can be allowed to cool to from about 18° C. to about 24° C., and/or about 21° C. and about 100 mL of water and 100 mL of chloroform can be added to form a multiphase mixture from which an organic phase can be separated from an aqueous phase. The multiphase mixture can be agitated for about five minutes and allowed to separate. The organic phase can be additionally washed by adding about 100 mL of water, two 100 mL portions of a saturated sodium bicarbonate solution, and 100 mL of brine wherein each washing step can provide a multiphase mixture from which an organic phase can be separated from an aqueous phase and taken to the next washing step. The organic phases can be combined, dried over sodium sulfate, filtered, and concentrated to afford about 47.7 grams of a product mixture containing 8,9,9,9-tetrafluoro-3-(2,3,3,3-tetrafluoro-2-(trifluoromethyl)nonane-1-sulfonyl chloride and 8,9,9,9-tetrafluoro-3-(2,3,3,3-tetrafluoro-2-(trifluoromethyl)undecane-1-sulfonyl chloride. The product structure(s) can be confirmed by NMR and/or chromatographic analysis.

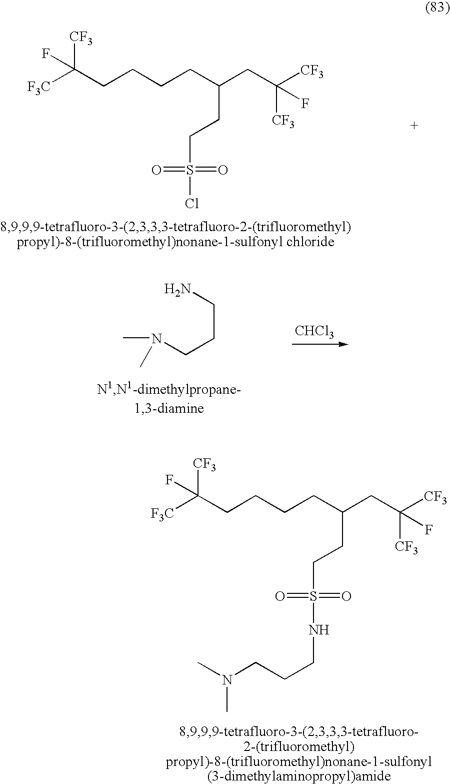

In conformity with scheme (83) above, in a flask that can be equipped with an agitator, thermocouple, reflux condenser, and an addition funnel, 24.5 grams (0.24 mole) of N′,N′-dimethylpropylamine and about 200 mL of chloroform can be added to form a mixture and cooled to about 0° C. using an ice/acetone bath. To the mixture, 47 grams of a mixture containing about 85 (wt/wt) percent of 8,9,9,9-tetrafluoro-3-(2,3,3,3-tetrafluoro-2-(trifluoromethyl)nonane-1-sulfonyl chloride (refer to scheme (82) above) and about 15 (wt/wt) percent of 8,9,9,9-tetrafluoro-3-(2,3,3,3-tetrafluoro-2-(trifluoromethyl)undecane-1-sulfonyl chloride and 100 mL of chloroform can be added drop wise over a period of two hours so that the maximum temperature does not exceed about 5° C. to form a reaction mixture. The reaction mixture can be washed by adding 200 mL of saturated sodium bicarbonate, 200 mL of water, and 200 mL of brine wherein each step can provide a multiphase mixture from which an organic phase can be separated from an aqueous phase and taken to the next washing step. The final organic phase can be dried over sodium sulfate, filtered, and concentrated to afford 57.3 grams of a product mixture containing 8,9,9,9-tetrafluoro-3-(2,3,3,3-tetrafluoro-2-(trifluoromethyl)nonane-1-sulfonyl-(dimethylaminopropyl)amide and 8,9,9,9-tetrafluoro-3-(2,3,3,3-tetrafluoro-2-(trifluoromethyl)undecane-1-sulfonyl-(dimethylaminopropyl)amide that can be observed as a yellowish oil. The product structure(s) can be confirmed by NMR and/or chromatographic analysis.

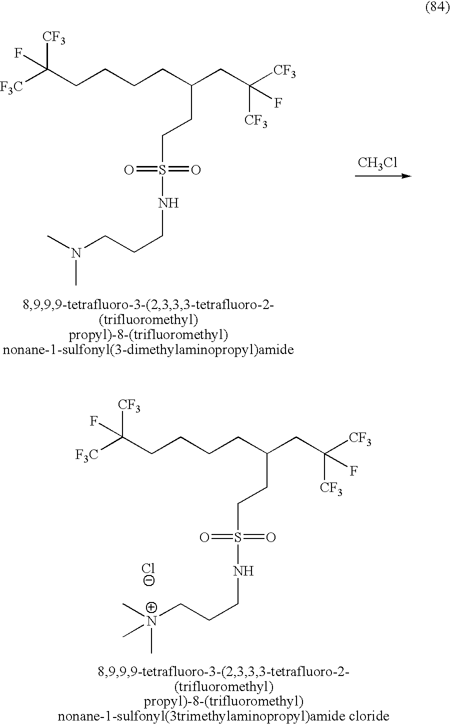

In accordance with scheme (84) above, in a flask that can be equipped with an agitator and a thermocouple, 15 grams of a mixture containing about 85 (wt/wt) percent of 8,9,9,9-tetrafluoro-3-(2,3,3,3-tetrafluoro-2-(trifluoromethyl)nonane-1-sulfonyl-(dimethylaminopropyl)amide (refer to scheme (83) above) and about 15 (wt/wt) percent of 8,9,9,9-tetrafluoro-3-(2,3,3,3-tetrafluoro-2-(trifluoromethyl)undecane-1-sulfonyl-(dimethylaminopropyl)amide and about 25 mL of a 1M solution of chloromethane in tert-butyl methyl ether can be placed and the flask sealed to form a mixture. The mixture can be heated to about 55° C. and held for from about 15 hours to about 21 hours, and/or about 18 hours. The mixture can be cooled and the flask vented and the mixture observed to be clear and yellow. The mixture can be concentrated to afford about 7.2 grams of a product mixture containing 8,9,9,9-tetrafluoro-3-(2,3,3,3-tetrafluoro-2-(trifluoromethyl)nonane-1-sulfonylamide-(trimethylaminopropyl) chloride and 8,9,9,9-tetrafluoro-3-(2,3,3,3-tetrafluoro-2-(trifluoromethyl)undecane-1-sulfonylamide-(trimethylaminopropyl) chloride as a yellow fryable foam. The product structure(s) can be confirmed by NMR and/or chromatographic analysis.

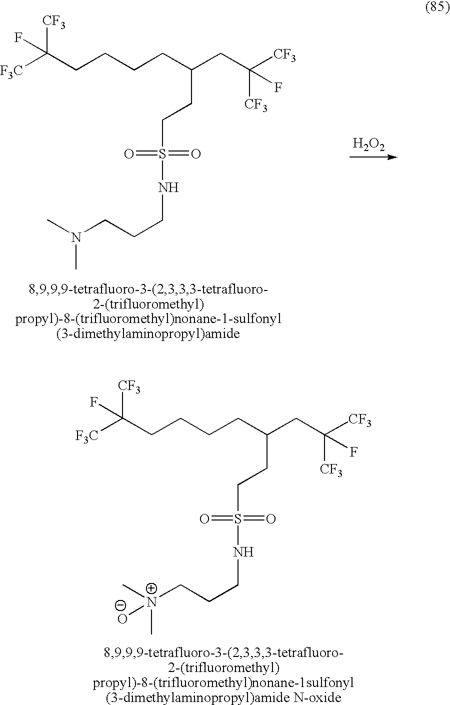

Referring to scheme (85) above, in a flask that can be equipped with an agitator, thermocouple, reflux condenser, and an addition funnel, 15 grams of a mixture containing about 85 (wt/wt) percent of 8,9,9,9-tetrafluoro-3-(2,3,3,3-tetrafluoro-2-(trifluoromethyl)nonane-1-sulfonyl-(dimethylaminopropyl)amide (refer to scheme (83) above) and about 15 (wt/wt) percent of 8,9,9,9-tetrafluoro-3-(2,3,3,3-tetrafluoro-2-(trifluoromethyl)undecane-1-sulfonyl-(dimethylaminopropyl)amide, about 20 mL of ethanol, and about 3 mL of water can be placed to form a mixture and heated to about 30° C. To the mixture, about 11.5 mL of a 50 (wt/wt) percent solution of hydrogen peroxide can be added drop wise over a period of about 30 minutes to form a reaction mixture. The reaction mixture can be heated to about 35° C. and held for about three hours. To the reaction mixture, 7.5 grams of carbon can be added to form a slurry and allowed to cool to from about 18° C. to about 24° C., and/or about 21° C. and held for from about 15 hours to about 21 hours, and/or about 18 hours. The slurry can be filtered through celite and the filter cake washed with about 200 mL ethanol. The filtrate can be concentrated to afford 10.9 grams of a product mixture containing 8,9,9,9-tetrafluoro-3-(2,3,3,3-tetrafluoro-2-(trifluoromethyl)nonane-1-sulfonylamide-(trimethylaminopropyl)oxide and 8,9,9,9-tetrafluoro-3-(2,3,3,3-tetrafluoro-2-(trifluoromethyl)undecane-1-sulfonylamide-(trimethylaminopropyl)oxide as a yellow oil. The product structure(s) can be confirmed by NMR and/or chromatographic analysis.

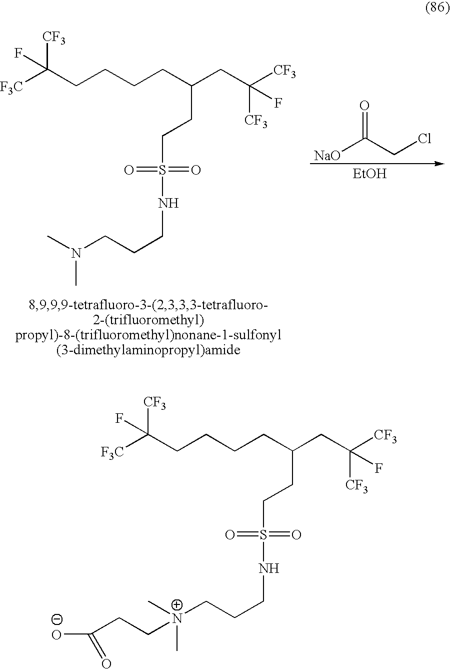

In reference to scheme (86) above, in a flask that can be equipped with an agitator, thermocouple, and a reflux condenser, 15 grams of a mixture containing about 85 (wt/wt) percent of 8,9,9,9-tetrafluoro-3-(2,3,3,3-tetrafluoro-2-(trifluoromethyl)nonane-1-sulfonyl-(dimethylaminopropyl)amide and about 15 (wt/wt) percent of 8,9,9,9-tetrafluoro-3-(2,3,3,3-tetrafluoro-2-(trifluoromethyl)undecane-1-sulfonyl-(dimethylaminopropyl)amide, 2.84 grams (0.024 mole) of sodium chloroacetate, and about 61 mL of ethanol can be placed to form a mixture and heated to reflux and held for from about 42 hours to about 48 hours, and/or about 45 hours. The mixture can be allowed to cool to from about 18° C. to about 24° C., and/or about 21° C. and filtered. The filtrate can be concentrated to afford 13 grams of a product mixture containing

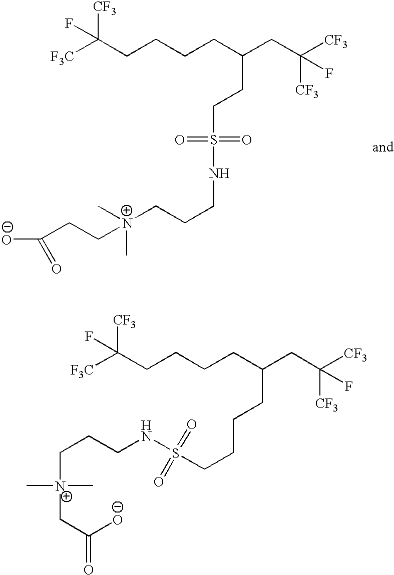

as a fryable foam. The product structure(s) can be confirmed by NMR and/or chromatographic analysis.

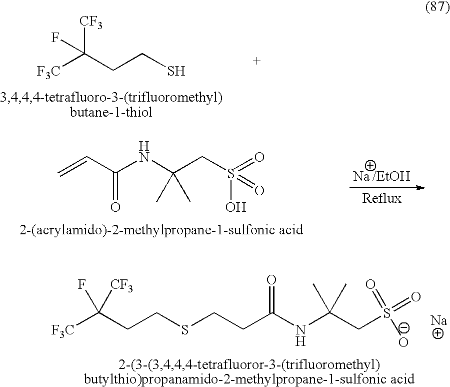

In reference to scheme (87) above, in a flask that can be equipped with an addition funnel, an agitator, a thermocouple, and a reflux condenser, 1.0 gram (0.043 mole) cut sodium metal can be dissolved in about 60 mL of ethanol to form a mixture. To the mixture can be slowly added, 7.5 gram (0.03 mole) 3,4,4,4-tetrafluoro-3-trifluoromethylbutane-1-thiol (see, e.g. Published International Applications) to form a second mixture. To the second mixture, 4.5 grams (0.022 mole) 2-acryloylamino-2-methylpropane-1-sulfonic acid can be added slowly to form a reaction mixture. The reaction mixture can be heated to reflux and held for about three hours, cooled to from about 18° to about 24° C., and/or to about 21° C. and held while stirring for from about 12 hours to about 18 hours, and/or about 15 hours. The reaction mixture can be observed to have taken on an orange color and become a viscous slurry. To the reaction mixture can be added, about 11 mL of 6N HCl solution whereupon the reaction mixture can be observed to transition from orange to yellow in color. The reaction mixture can be filtered and concentrated in vacuo. The concentrated filtrate can then be washed with two separate 50 mL portions of ether and then refiltered and concentrated in vacuo to afford an orange colored oily solid. The oily solid can be dried, affording 6.7 grams of concentrate. The concentrate can then be dissolved in about 65 mL ethanol to form a new mixture. To the new mixture, 0.61 grams (0.015 mole) NaOH can be added and held while stirring for about three hours. The new mixture can be concentrated in vacuo to afford 6 grams of the sodium salt of 3-(3-(3,4,4,4-tetrafluoro-3-(trifluoromethyl)butylthiol)propanamido)-3-methylbutane-1-sulfonic acid product. The product structure can be confirmed by proton NMR and liquid chromatography/mass spectroscopy.

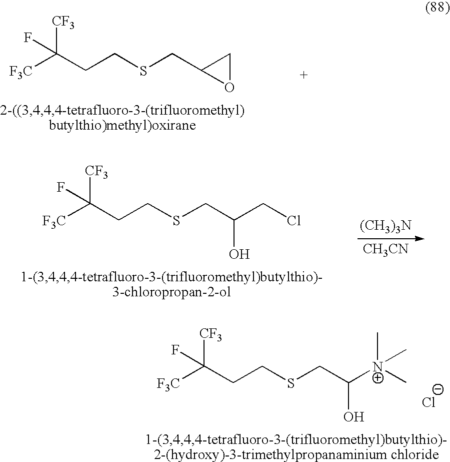

According to scheme (88) above, about 1.2 gram (0.02 mole) of trimethyl amine can be placed into a small flask and chilled in a acetone and ice bath. In a small pressure flask, about 6 mL acetonitrile can be combined with 0.135 gram (4.7×10⁻⁴ mole) of 2-((3,4,4,4-tetrafluoro-3-(trifluoromethyl)butylthio)methyl)oxirane (refer to scheme (5) above) and 0.765 gram (2.4×10⁻³ mole) of 1-(3,4,4,4-tetrafluoro-3-(trifluoromethyl)butylthio)-3-chloropropan-2-ol (refer to scheme (5) above) to form a mixture which can be chilled to about 0° C. using a ice water bath. The chilled trimethyl amine can be added to the mixture to form a reaction mixture. The flask can be sealed and heated to about 60° C. and held for about 4 hours. The reaction mixture can be cooled to from about 18° C. to about 24°, and/or 21° C. and held for from about 15 hours to about 21 hours, and/or from about 18 hours whereupon the reaction mixture can be observed to contain a brownish colored slurry containing a white precipitate. The reaction mixture can be filtered and concentrated in vacuo to provide 2.0 grams of what can be observed as a brown colored oil. The brown colored oil can be dissolved in about 5 mL ethyl acetate and treated with about 6 mL of a 2M HCl solution in ether to form a multiphase mixture that can be observed to be clear and yellow and from which an organic phase can be separated from an aqueous phase. The organic phase can be placed into a Kugelrohr apparatus (0.03 mmHg, 55° C., 20 minutes) to afford 1.7 gram (4.5×10⁻³ mole) of the 1-(3,4,4,4-tetrafluoro-3-(trifluoromethyl)butylthio)-2-(hydroxyl)-3-trimethylpropanaminium chloride product that can be observed as a brown oil. The product structure can be characterized by ¹HNMR analysis and/or LCMS analysis and/or ¹⁹FNMR analysis.

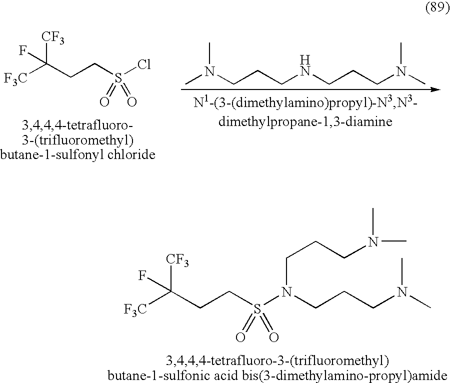

Referring to scheme (89) above, in a flask that can be equipped with a thermocouple, an agitator, and an addition funnel, 17.7 gram (0.095 mole) of N¹-(3-(dimethylamino)propyl)-N³,N³-dimethylpropane-1,3-diamine and about 45 mL of chloroform can be combined to form a mixture and cooled to from about 0° C. to about 5° C., and/or about 0° C. using an ice/acetone bath. To the mixture, 10.0 gram (0.034 mole) of 3,4,4,4-tetrafluoro-3-trifluoromethylbutane-1-sulfonyl chloride (see, e.g., Published International Applications) that can be dissolved in about 45 mL chloroform can be added drop wise over about an hour to form a reaction mixture. The rate of the addition may be such that the reaction mixture temperature is kept at about 0° C. The reaction mixture can be observed to be yellow in color and can be heated to from about 62° to about 72° C., and/or about 67° C. for about one hour. The reaction mixture can be washed successively with about three times with 90 mL saturated sodium bicarbonate solution, about three times with 90 mL deionized water, and about two times with 90 mL brine wherein each step can form a multiphase mixture from which an organic phase can be separated from an aqueous phase. The organic phase can be collected, dried over sodium sulfate, filtered, and concentrated in vacuo to provide 13.9 grams of the 3,4,4,4-tetrafluoro-3-(trifluoromethyl)butane-1-sulfonic acid-bis(3-dimethylaminopropyl)amide product that can be observed as a yellow oil. The product structure can be confirmed with NMR and/or chromatographic analysis.

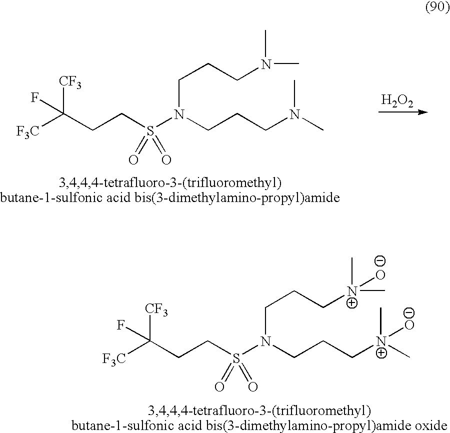

In accordance with scheme (90) above, in a flask that can be equipped with an agitator, a thermocouple, 12.37 grams (0.028 mole) of 3,4,4,4-tetrafluoro-3-trifluoromethyl-butane-1-sulfonic acid bis(3-dimethylaminopropyl)amide (refer to scheme (89) above), about 13.0 mL of a 50 percent (wt/wt) hydrogen peroxide, about 20 mL of ethanol, and about 3.0 mL water to form a reaction mixture. The reaction mixture can be stirred from about 12 hours to about 18 hours, and/or about 15 hours, at a temperature of from about 18° C. to about 24° C. and/or about 21° C. The reaction mixture, about 20 mL ethanol and 8.0 grams of Norit A, an activated carbon, can be added to form a slurry. The slurry can be stirred at from about 18° C. to about 24° C., and/or about 21° C. for from about 62 hours to about 72 hours, and/or about 67 hours. The slurry can be tested for peroxide using a potassium iodide test strip and filtered through celite, washed with ethanol and concentrated in vacuo to afford 12 grams of 91 percent pure by liquid chromatography/mass spectroscopy analysis 3,4,4,4-tetrafluoro-3-trifluoromethyl-butane-1-sulfonic acid bis(3-dimethylaminopropyl)amide oxide product that can be observed as a gummy solid. The product structure can be confirmed by NMR and liquid chromatography/mass spectroscopy (LCMS) analysis.

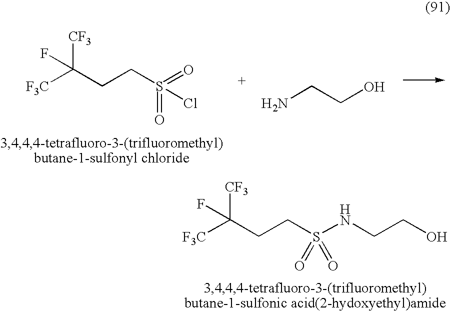

With reference to scheme (91) above, in a flask that can be equipped with an addition funnel, an agitator, and a thermocouple, 92.7 grams (1.52 moles) ethanolamine and about 375 mL methylene chloride can be placed while under a nitrogen atmosphere to form a mixture and chilled to about 0° C. using an ice/acetone bath. To the mixture, 75 grams (0.25 mole) 3,4,4,4-tetrafluoro-3-trifluoromethylbutane-1-sulfonyl chloride (see, e.g., Published International Applications) can be added drop wise to form a reaction mixture. The addition rate can be such that the reaction mixture temperature is kept at or below about 5° C. The reaction mixture can be allowed to warm to from about 18° C. to about 24° C., and/or about 21° C., and stirred for about one hour. The reaction mixture can be diluted with about 750 mL of methylene chloride and washed successively by addition with about 750 mL water, about 750 mL of a 5 percent (wt/wt) HCl solution, and about 750 mL of a saturated sodium bicarbonate solution wherein each step can form a multiphase mixture from which an organic phase can be separated from an aqueous phase. The organic phase can be collected and dried over sodium sulfate, filtered and concentrated in vacuo affording 38.38 grams 3,4,4,4-tetrafluoro-3-(trifluoromethyl)butane-1-sulfonic acid (2-hydroxyethyl)amide product that can be observed to be a white solid. The product structure can be confirmed by NMR and/or chromatographic analysis.

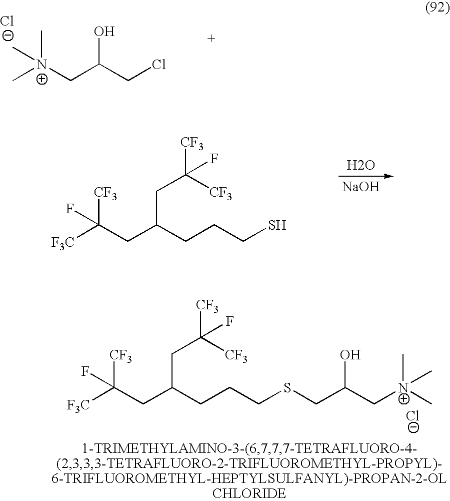

According to scheme (92) above, in a flask that can be equipped with an agitator, about 6 mL of water, 10 grams (0.022 mole) of 6,7,7,7-tetrafluoro-4-(2,3,3,3-tetrafluoro-2-trifluoromethyl-propyl)-6-trifluoromethyl-heptane-1-thiol, 6.9 grams (0.02 mole) of 3-chloro-2-hydroxypropyl-trimethyl ammonium chloride in 18 grams of water, and 0.88 gram (0.02 mole) of sodium hydroxide can be placed to form a mixture. The mixture can be held at from about 18° C. to about 24° C., and/or about 21° C. for from about 15 hours to about 21 hours, and/or about 18 hours. The mixture can be observed as a white slurry and can be filtered with the filtrate being collected. The filtrate can be stripped of water by using ethanol followed by chloroform to afford an oil that can be observed as clear and colorless. The oil can be placed on a Kugelrohr apparatus (50° C., 0.03 mmHg, 30 minutes) to afford 15.6 grams of impure 1-trimethylamino-3-[6,7,7,7-tetrafluoro-4-(2,3,3,3-tetrafluoro-2-trifluoromethyl-propyl)-6-trifluoromethyl-heptylsulfanyl]propan-2-ol chloride product. The product can be dissolved in about 50 mL of ethanol to form a mixture then held at from about 18° C. to about 24° C., and/or about 21° C. for from about 54 hours to about 70 hours, and/or about 62 hours. The mixture can be filtered and concentrated to afford 12.3 grams of product that can be observed as a white solid. The product structure can be confirmed by NMR and/or chromatographic analysis.

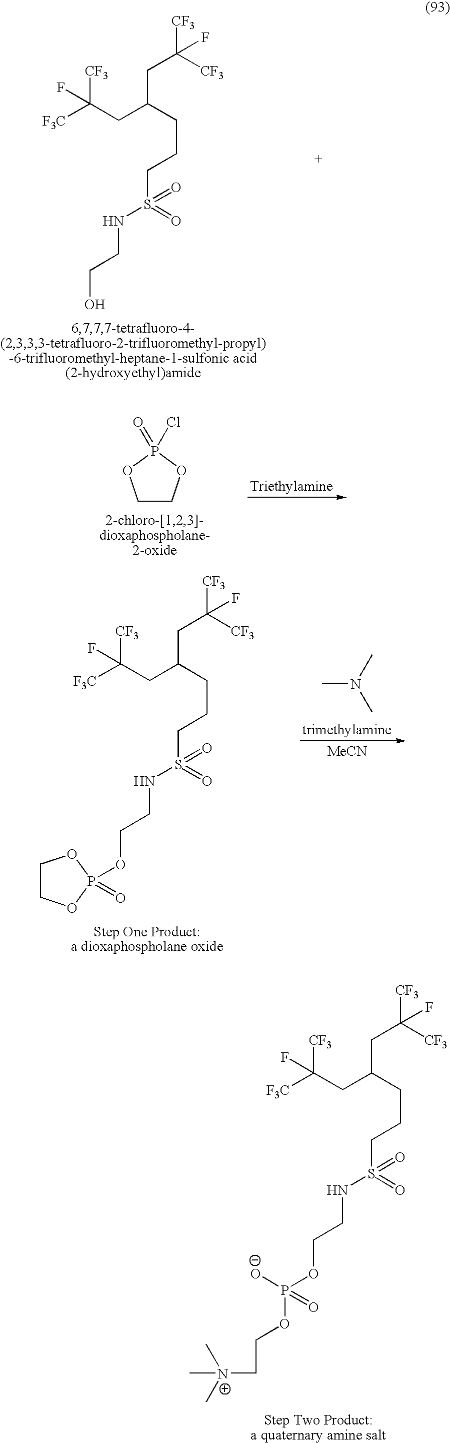

According to scheme (93) above, in a flask that can be equipped with an agitator, thermocouple, ice water bath, and an addition funnel, 11.0 grams (0.02 mole) of 6,7,7,7-tetrafluoro-4-(2,3,3,3-tetrafluoro-2-trifluoromethyl-propyl)-6-trifluoromethyl-heptane-1-sulfonic acid-(2-hydroxyethyl)amide, 2.87 grams (0.02 mole) of 2-chloro-[1,3,2]dioxaphospholane-2-oxide, and about 66 mL of anhydrous ether can be placed to form a mixture. The mixture can be cooled to about 0° C. using an ice water bath. To the mixture, 0.88 grams (0.009 mole) of triethylamine can be added drop wise to form a reaction mixture. A white precipitate can be observed to form immediately upon addition of the triethylamine to the mixture. The reaction mixture can be allowed to warm to from about 18° C. to about 24° C., and/or about 21° C. and held for about four hours. The reaction mixture can be filtered and concentrated in vacuo to afford crude step one reaction product observed as a pale yellow oil. To remove residual ether, the crude step one product can be placed on a Kugelrohr apparatus (40° C., 0.1 torr, 60 minutes) to afford about 12.8 grams of step one product. The product structure can be confirmed by NMR and/or chromatographic analysis. In flask that can be equipped with an agitator, thermocouple, and an addition funnel, the step one product can be added and about 130 mL of acetonitrile to form a mixture The mixture can be chilled using a dry ice/acetone bath and 18.45 grams (0.31 mole) of trimethylamine can be added drop wise to form a reaction mixture. The reaction mixture can be allowed to warm to from about 18° C. to about 24° C., and/or about 21° C. followed by heating to about 60° C. for about five hours wherein a white precipitate can be observed to form. The reaction mixture can be chilled to about 0° using an ice water bath and held from about 15 hours to about 21 hours, and/or about 18 hours. The white precipitate can be filtered from the reaction mixture and dried from about 15 hours to about 21 hours, and/or about 18 hours in vacuo at about 50° C. to afford 4.36 grams of the step two product. The product structure can be confirmed by NMR and/or chromatographic analysis.

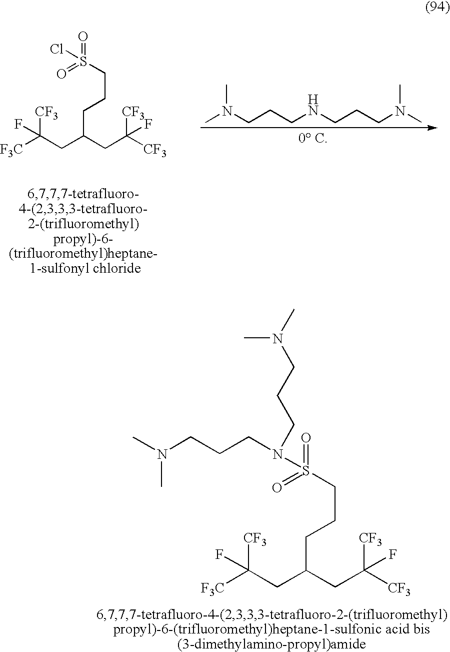

In accordance with scheme (94) above, in a flask that can be equipped with a thermocouple, addition funnel, and an agitator, 10.1 gram (0.054 mole) of 3,3′-iminobis(N,N′-dimethylaminopropylamine) can be dissolved in about 45 mL chloroform can be placed to form a mixture. The mixture can be chilled to about 0° C. using an ice/acetone bath. To the mixture can be added drop wise, 10.0 gram (0.019 mole) of 6,7,7,7-tetrafluoro-4-(2,3,3,3-tetrafluoro-2-(trifluoromethyl)propyl)-6-(trifluoromethyl)heptane-1-sulfonyl chloride dissolved in about 45 mL to form a reaction mixture. The rate of addition can be such that a reaction mixture temperature can be kept at about 0° C. Following the addition, the reaction mixture can be held at about 0° C. for about one hour. The reaction mixture can be washed in the following manner: about 90 mL saturated sodium bicarbonate solution, about 90 mL water, and about 90 mL brine solution. The organic layer can then be collected and dried over magnesium sulfate, filtered, and concentrated in vacuo to provide about 11.66 gram of the 6,7,7,7-tetrafluoro-4-(2,3,3,3-tetrafluoro-2-(trifluoromethyl)propyl)-6-(trifluoromethyl)heptane-1-sulfonic acid bis-(3 dimethylamino-propyl)amide product as a yellow oil. The product structure can be confirmed by employing NMR and/or chromatographic analysis.

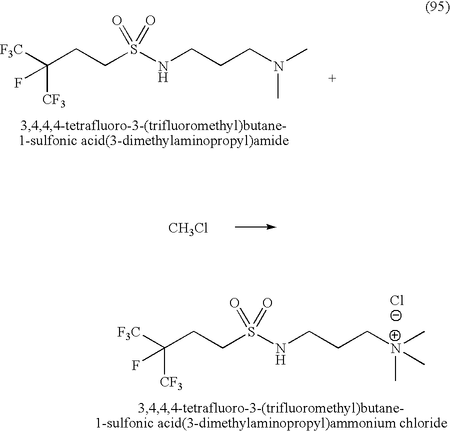

According to scheme (95) above, in a sealed tube about 10 grams (0.03 mole) of 3,4,4,4-tetrafluoro-3-(trifluoromethyl)butane-1-sulfonic acid (3-dimethylamino-propyl)-amide (see, e.g., Published International Applications) can be dissolved in about 28 mL (0.03 mole) of a 1.0M solution of chloromethane in tert-butyl methyl ether to form a mixture. The mixture can be heated to about 55° C. using a hot oil bath and held from about 15 hours to about 21 hours, and/or about 18 hours. The mixture can be cooled from about 18° C. to about 24° C., and/or about 21° C. and vented. The mixture can be filtered and washed with ether to afford about 5.2 grams of the 3,4,4,4-tetrafluoro-3-trifluoromethyl-butane-1-sulfonic acid (3-dimethylamino-propyl)ammonium chloride product. The product structure can be confirmed by NMR and/or chromatographic analysis.

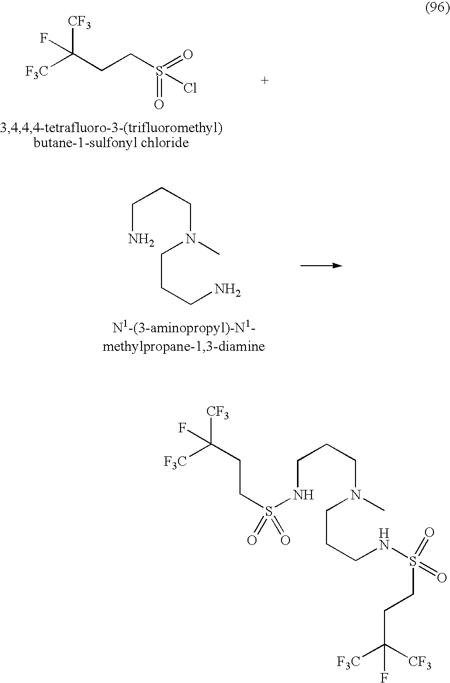

According to scheme (96) above, in a flask that can be equipped with an agitator, thermocouple, and an dry-ice/acetone bath, 10.0 grams (0.069 mole) of 3,3 diamino N methyl dipropylamine and about 60 mL chloroform can be placed to form a first mixture. The first mixture can be chilled to about 0° C. by using the dry-ice/acetone bath. In the addition funnel, 14.6 grams (0.049 mole) of 3,4,4,4-tetrafluoro-3-trifluoromethyl-butane-1-sulfonyl chloride (see, e.g., Published International Applications) and about 40 mL of chloroform can be added to form a second mixture. The second mixture can be added to the first mixture drop wise over a period of about 35 minutes to form a reaction mixture. The reaction mixture can be kept at a temperature at or below about 5° C. The peak temperature during addition can be about −2.5° C. The reaction mixture can be allowed to warm to room temperature and maintained for about two hours. The reaction mixture can be washed with three 100 mL portions of water wherein each can form a multiphase mixture from which an organic phase can be separated from an aqueous phase. The organic phase can be dried and concentrated to afford 16.8 grams of a crude product mixture that contained starting material. The product mixture can be placed on a Kugelrohr apparatus at 80° C. and 0.03 mmHg for about 30 minutes to afford 13.9 grams of a second crude product mixture that contained starting material. The second product mixture can be triturated with two 200 mL portions of water to afford 6.9 grams of the

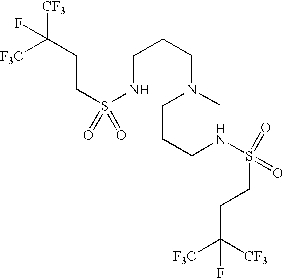

product.

The product structure can be confirmed by NMR and/or LCMS analysis.

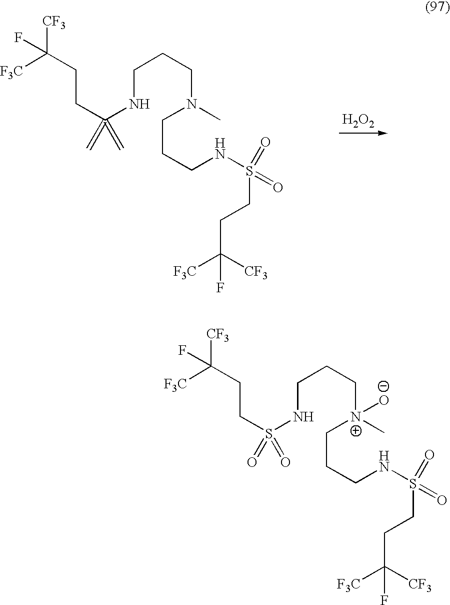

In accordance with scheme (97), in a flask that can be equipped with an agitator, thermocouple and an addition funnel, 7.8 grams (0.012 mole) of

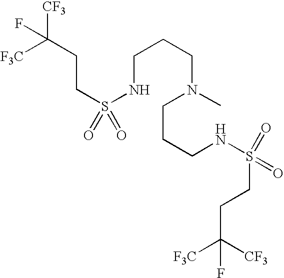

and about 23 mL of ethanol and about 3.6 mL of water can be placed to form a mixture. To the mixture, 6.14 grams of a 50% (wt/wt) solution of hydrogen peroxide in water can be added slowly over a period of 15 minutes at room temperature to form a reaction mixture. The peak temperature of the reaction mixture during addition can be about 20.8° C. The reaction mixture can be observed as a cloudy orange solution which can clarify upon heating. The reaction mixture can be heated to and maintained at about 35° C. for about 3 hours. The reaction mixture can be allowed to cool to room temperature and maintained overnight. The reaction mixture can be heated to and maintained at about 35° C. for about 2 hours. To the reaction mixture, 5 grams of carbon can be added slowly to quench the peroxides over a period of about 20 minutes to form a slurry. To the slurry, about 30 mL of ethanol can be also added to facilitate uniform stirring. The mixture was left to stir overnight at room temperature. The slurry can be heated to about 50° C. for about four hours. The slurry can be filtered through celite and the filter cake washed with about 300 mL of ethanol to provide a filtrate that can be observed as clear and colorless. The filtrate can be concentrated to afford 4.8 grams of the

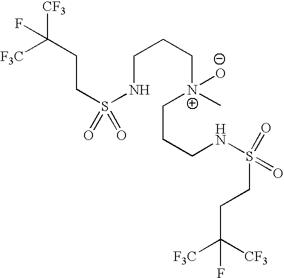

product. The product structure can be confirmed by NMR and/or LCMS analysis.

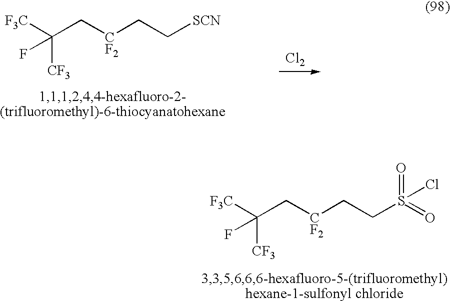

According to scheme (98) above, in a flask that can be equipped with an agitator, thermocouple, and an a sparging apparatus, 21.24 grams (0.07 mole) of 1,1,1,2,4,4-hexafluoro-2-(trifluoromethyl)-6-thiocyanatohexane (refer to scheme (40) above) and about 85 mL of acetic acid can be placed to form a mixture. The mixture can be heated to about 50° C. and vigorously sparged with chlorine gas for about 5 hours to form a reaction mixture. The gas and the heat can be turned off overnight and heating and sparging can be resumed the next day for an additional hour. The reaction mixture can be allowed to cool and about 2.4 mL of water added. To the reaction mixture, about 100 mL of chloroform and about 100 mL of water to form a multiphase mixture from which an organic phase can be separated from an aqueous phase. The phases can be partitioned and the organic phase collected and successively washed with three 100 mL portions of a saturated bicarbonate solution one 100 mL portion of brine. The organic phase can be collected, dried over sodium sulfate, filtered and concentrated to afford 20.4 grams of 3,3,5,6,6,6-hexafluoro-5-(trifluoromethyl)hexane-1-sulfonyl chloride product that can be observed as a pale oil. The product structure can be confirmed by LCMS and/or NMR analysis.

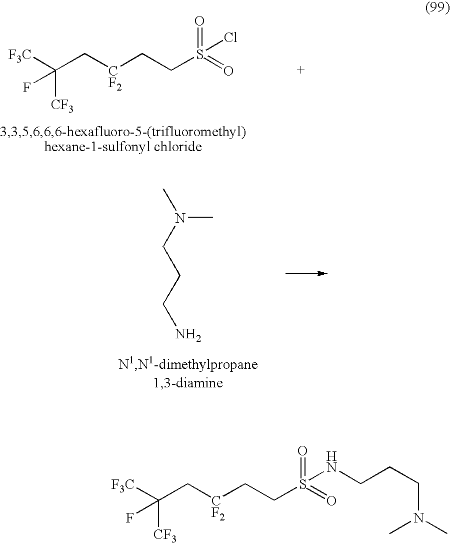

In accordance with scheme (99) above, in a flask that can be equipped with an agitator, thermocouple, an ice water bath, and an addition funnel, 21.5 mL (0.17 mole) of 3-(dimethylamino)propylamine and about 55 mL of chloroform can be placed to form a first mixture. The first mixture can be chilled to about 0° C. using the ice water bath. In the addition funnel, 20.4 grams (0.06 mole) of 3,3,5,6,6,6-hexafluoro-5-(trifluoromethyl)hexanesulfonyl chloride (refer to scheme (98) above) and about 55 mL chloroform can be placed to form a second mixture. The second mixture can be added drop wise to the first mixture over a period of about one hour to form a reaction mixture. The reaction mixture can be maintained at a temperature below 5° C. The peak temperature during addition can be 5.3° C. The reaction mixture can be allowed to warm to room temperature and maintained overnight. The reaction mixture can be successively washed with two 200 mL portions of a saturated NaHCO₃ solution, one 200 mL portion of a saturated NaCl solution and one 200 mL portion of water each step affording a multiphase mixture from which an organic phase can be separated from an aqueous phase. The organic phase can be collected, dried and concentrated to afford 21.3 grams of

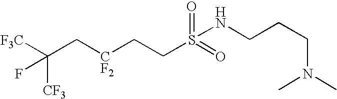

product. The product structure can be confirmed by LCMS and/or NMR analysis.

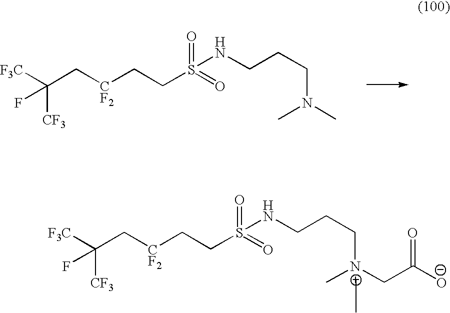

Referring to scheme (100) above, in a flask that can be equipped with an agitator, thermocouple, and a reflux condenser, about 62.2 mL of ethanol, 2.9 grams (0.025 mole) of sodium chloroacetate and 10.6 grams (0.25 mole)

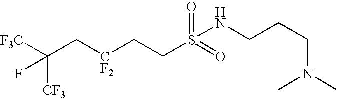

(refer to scheme (99) above) can be placed to form a mixture. The mixture can be to reflux and maintained for about 1.5 days. The mixture can be cooled and filtered through celite to afford a filtrate. The filtrate can be concentrated to afford 7.65 grams of the

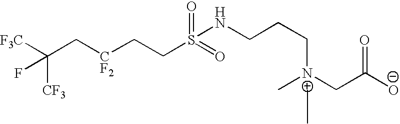

product that can be observed as a fryable foam. The product structure can be confirmed by NMR and LCMS analysis.

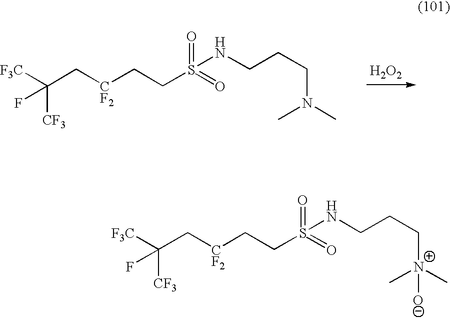

According to scheme (101) above, in a flask that can be equipped with an agitator, thermocouple, and an addition funnel, 10.6 grams (0.025 mole) of

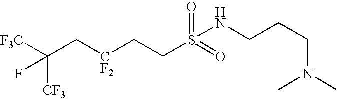

(refer to scheme (99) above), about 25 mL of ethanol and about 3.7 mL of water to form a mixture. To the mixture, about 11.73 grams (0.191 mole) of a 50% wt/wt solution of hydrogen peroxide in water can be added over a period of about 30 minutes to form a first reaction mixture. An exotherm can be observed wherein the peak temperature during the addition can be about 22.9° C. The first reaction mixture can be heated to and maintained at about 35° C. for about 5 hours. To the first reaction mixture, about 25 mL of ethanol and 6.36 grams of decolorizing carbon can be added over a period of about 20 minutes to quench the peroxides and form a first slurry. A slight exotherm can be observed along with some foaming. The first slurry can be held at room temperature for about 3 days. The first slurry can be filtered through celite which and washed with about 100 mL of ethanol to form a first filtrate. The first filtrate, which can be observed as clear and colorless, can be concentrated to afford about 10 grams of a first white solid that upon analysis by proton NMR revealed to contain a significant amount of starting material. In the flask, 10 grams of the first white solid can be placed in about 25 mL of ethanol, about 2 mL of water and about 6 mL of the peroxide solution to form a second reaction mixture. The second reaction mixture can be heated to 35° C. and maintained overnight. To the second reaction mixture, 5.2 grams of decolorizing carbon can be added to form a second slurry. The second slurry can be heated to 45° C. and maintained overnight. The second slurry can be filtered through celite to afford a second filtrate which can be observed as clear and colorless filtrate. The second filtrate can be concentrated to afford a second white solid. To further concentrate the second white solid, the flask was placed on the Kugelrohr apparatus set at 0.03 mmHg, 35° C. and 45 minutes. The contents of the flask can be observed to gum up and turn yellow. The heat can be turned off while the vacuum pump remained on for an additional 2 hours to afford 6.9 grams of the

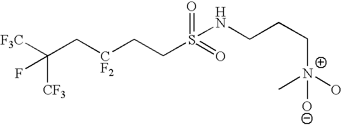

product. The product structure can be confirmed by LCMS and/or NMR analysis.

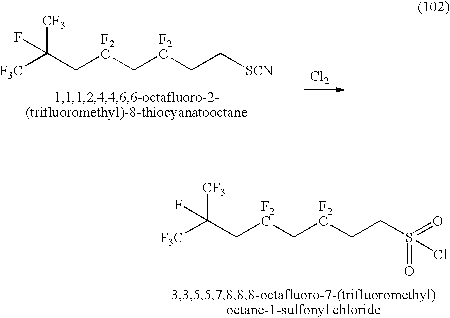

In accordance with scheme (102) above, in a flask that can be equipped with an agitator, thermocouple and a sparging apparatus, 17.7 grams (0.05 mole) of 1,1,1,2,4,4,6,6-octafluoro-2-(trifluoromethyl)-8-thiocyanatooctane (refer to scheme (41) above) and about 58 mL of acetic acid can be placed to form a mixture. The mixture can be heated to about 50° C. and vigorously sparged with chlorine gas for about 4 hours to form a reaction mixture. The chlorine sparging can be discontinued and allowed to cool to room temperature and maintained overnight. To the reaction mixture, about 2 mL of water added. To the reaction mixture, about 100 mL of chloroform and about 100 mL of water to form a multiphase mixture from which an organic phase can be separated from an aqueous phase. The organic phase can be successively washed with three 100 mL portions of a saturated bicarbonate solution one 100 mL portion of a saturated brine solution. The organic phase can be collected and dried over sodium sulfate, filtered and concentrated to afford 16.6 grams of the 3,3,5,5,7,8,8,8-octafluoro-7-(trifluoromethyl)octane-1-sulfonyl chloride product. The product structure can be confirmed by NMR and/or GC/MS and/or GC and/or LCMS (i.e. collectively chromatographic analysis) analysis.

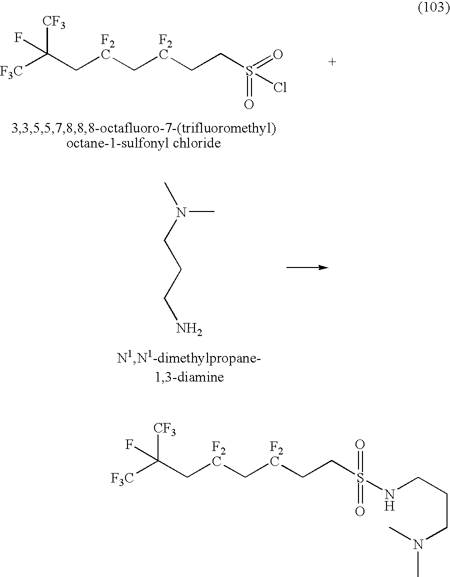

Referring to scheme (103) above, in a flask that can be equipped with an agitator, thermocouple, ice water bath, and an addition funnel, 12 mL (0.12 mole) of 3-(dimethylamino)propylamine and about 40 mL of chloroform can be placed to form a first mixture. The first mixture can be cooled to about 0° C. In the addition funnel, 16.6 grams (0.04 mole) of 3,3,5,5,7,8,8,8-octafluoro-7-(trifluoromethyl)octane-1-sulfonyl chloride (refer to scheme (102) above) and about 40 mL of chloroform can be placed to form a second mixture. The second mixture can be added drop wise to the first mixture over a period of about an hour to form a reaction mixture. The peak temperature during addition can be about 6.6° C. The reaction mixture can be allowed to warm to room temperature and stir overnight. The reaction mixture can be successively washed with two 200 mL portions of a saturated NaHCO₃ solution, one 200 mL portion of a saturated solution of NaCl and one 200 mL portion of water wherein each step can produce a multiphase mixture from which an organic phase can be separated from an aqueous phase and each organic phase can be collected and transferred to the next step. The final organic phase can be dried and concentrated to afford 17.2 grams of the

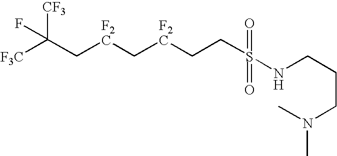

product which can be observed as a viscous yellow oil that solidified upon standing. The product structure can be confirmed by NMR and/or LCMS analysis.

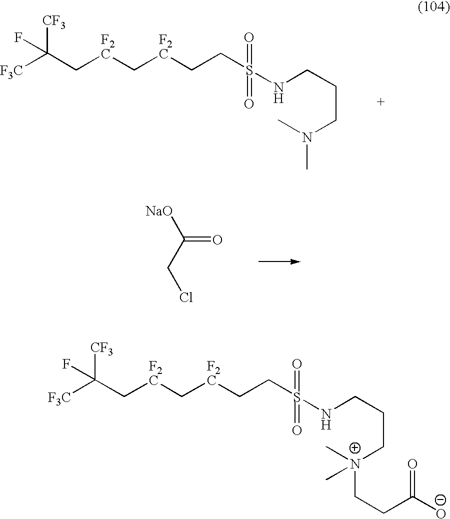

In reference to scheme (104) above, in a flask that can be equipped with an agitator, thermocouple, and a reflux condenser, about 44 mL of ethanol, 2.04 grams (0.018 mole) of sodium chloroacetate and 8.6 grams (0.018 mole) of

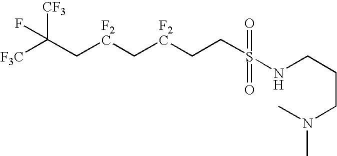

(refer to scheme (103) above) can be placed to form a mixture. The mixture can be heated to reflux for and maintained for about 1.5 days. The mixture can be allowed to cool and filtered through celite to afford a filtrate. The filtrate can be concentrated to afford 4.55 grams of the

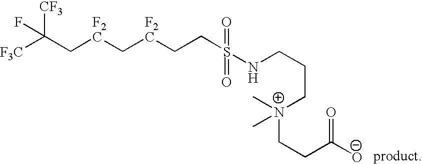

The product structure can be confirmed by NMR and/or LCMS analysis.

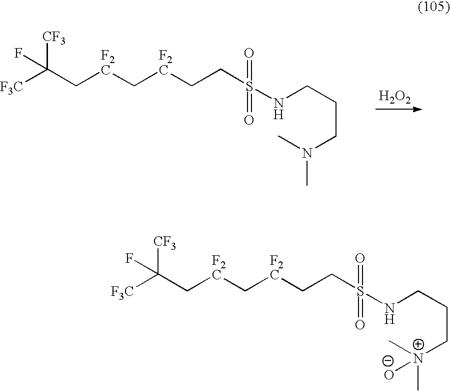

In conformity with scheme (105) above, in a flask that can be equipped with an agitator, thermocouple, ice water bath and an addition funnel, 8.6 grams (0.018 mole) of

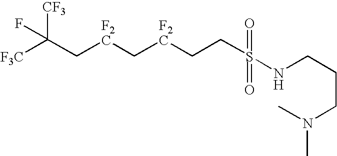

and about 18 mL of ethanol and about 2.6 mL of water to form a mixture. The mixture can be chilled to about 0° C. using the bath. To the mixture, 8.5 mL of a 50% (wt/wt) solution of hydrogen peroxide in water can be added over a period of about 30 minutes to form a reaction mixture. The reaction mixture can be observed to have peak temperature during addition of 22.5° C. The reaction mixture can be heated to and maintained at 35° C. for about 6 hours. To the reaction mixture, about 20 mL of ethanol and 5.2 grams of decolorizing carbon can be added over a period of about 20 minutes to form a slurry. A slight exotherm can be observed along with some foaming during the addition. The slurry can be allowed to cool to room temperature and maintained over the weekend (i.e., from about 54 hours to about 70 hours, and/or about 62 hours). The slurry can be filtered through celite and the filter cake washed with about 100 mL of ethanol to afford a filtrate that can be observed as clear and colorless. The filtrate can be concentrated to afford about 6.5 grams of the

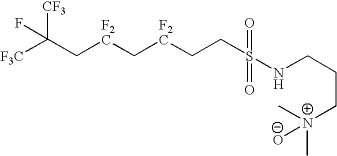

product that can be observed as a white solid. The product structure can be confirmed by NMR and/or LCMS analysis.

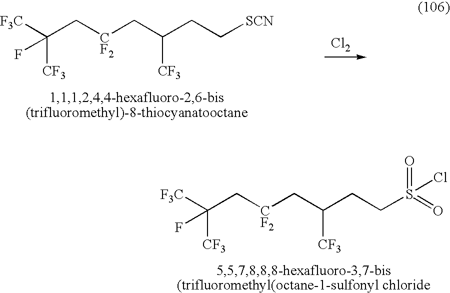

In accordance with scheme (106) above, in a flask that can be equipped with an agitator, thermocouple and a sparging apparatus, (25.7 grams (0.06 mole) of 1,1,1,2,4,4-hexafluoro-2,6-bis(trifluoromethyl)-8-thiocyanatooctane (refer to scheme (42) above) and about 80 mL of acetic acid can be placed to form a mixture. The mixture can be heated to about 50° C. and vigorously sparged with chlorine gas for about 2 days to form a reaction mixture. The reaction mixture can be allowed to cool and about 2.5 mL of water added. To the reaction mixture, about 100 mL of chloroform and about 100 mL of water to form a multiphase mixture from which an organic phase can be separated from an aqueous phase. The organic phase can be successively washed with three 100 mL portions of a saturated bicarbonate solution one 100 mL portion of a saturated brine solution. The organic phase can be collected and dried over sodium sulfate, filtered and concentrated to afford 22.3 grams of the 5,5,7,8,8,8-hexafluoro-3,7-bis(trifluoromethyl)octane-1-sulfonyl chloride product that can be observed as an oil. The product structure can be confirmed by NMR and/or GC/MS and/or GC analysis.

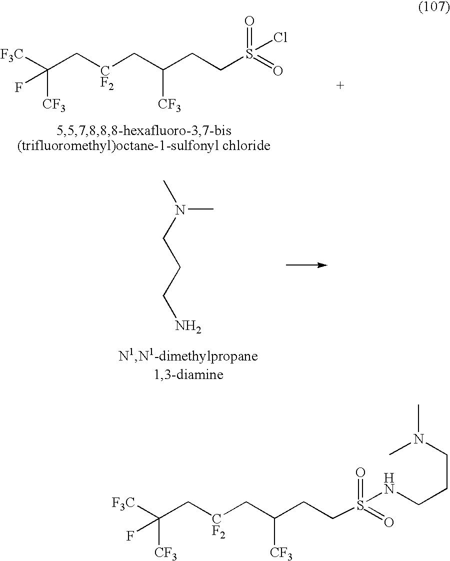

Referring to scheme (107) above, in a flask that can be equipped with an agitator, thermocouple, ice water bath, and an addition funnel, 18.5 mL (0.15 mole) of 3-(dimethylamino)propylamine and about 50 mL of chloroform can be placed to form a first mixture. The first mixture can be cooled to about 0° C. In the addition funnel, 22.3 grams (0.05 mole) of 5,5,7,8,8,8-hexafluoro-3,7-bis(trifluoromethyl)octane-1-sulfonyl chloride (refer to scheme (106) above) and about 50 mL of chloroform can be placed to form a second mixture. The second mixture can be added drop wise to the first mixture over a period of about an hour to form a reaction mixture. The reaction mixture can be maintained at a temperature below 5° C. The peak temperature during addition can be about 2.4° C. The reaction mixture can be allowed to warm to room temperature and stir overnight. The reaction mixture can be successively washed with two 200 mL portions of a saturated NaHCO₃ solution, one 200 mL portion of a saturated solution of NaCl and one 200 mL portion of water wherein each step can produce a multiphase mixture from which an organic phase can be separated from an aqueous phase and each organic phase can be collected and transferred to the next step. The final organic phase can be dried and concentrated to afford 21.5 grams of the

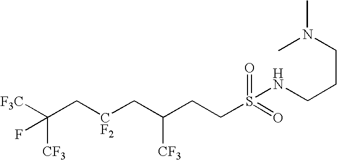

product which can be observed as a yellow solid. The product structure can be confirmed by NMR and/or LCMS analysis.

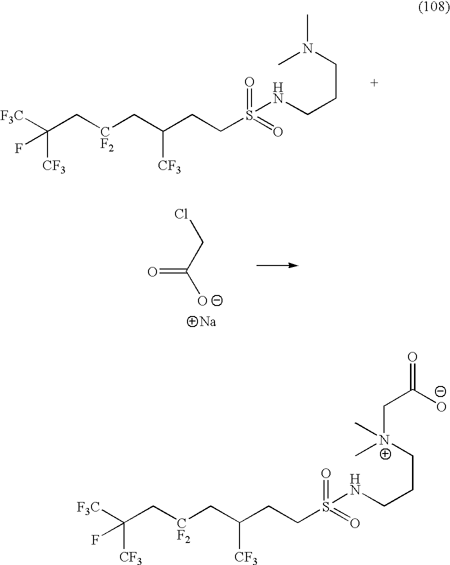

In reference to scheme (108) above, in a flask that can be equipped with an agitator, thermocouple, and a reflux condenser, about 50 mL of ethanol, 2.34 grams (0.02 mole) of sodium chloroacetate and 10.5 grams (0.02 mole) of

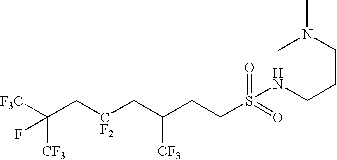

(refer to scheme (107) above) can be placed to form a mixture. The mixture can be heated to reflux for and maintained for about 6 days. The mixture can be allowed to cool and filtered through celite to afford a filtrate. The filtrate can be concentrated to afford 6.75 grams of the

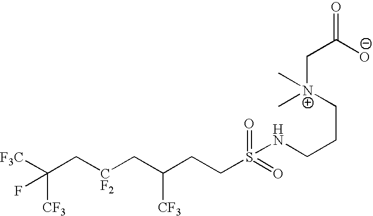

product. The product structure can be confirmed by NMR and/or LCMS analysis.

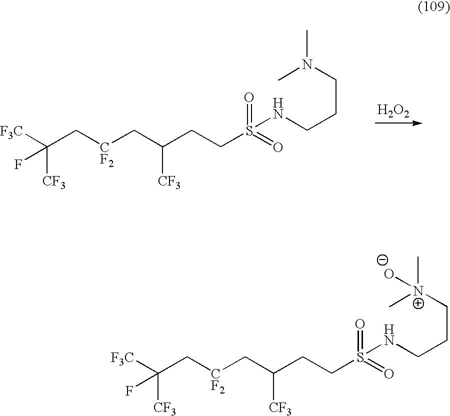

In conformity with scheme (109) above, in a flask that can be equipped with an agitator, thermocouple, ice water bath and an addition funnel, 10.5 grams (0.02 mole) of

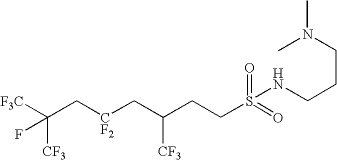

and about 20 mL of ethanol and about 3 mL of water to form a mixture the mixture can be chilled to about 0° C. using the bath. To the mixture, 9.5 mL of a 50% (wt/wt) solution of hydrogen peroxide in water can be added over a period of about 15 minutes to form a reaction mixture. The reaction mixture can be observed to have peak temperature during addition of 2.5° C. The reaction mixture can be heated to and maintained at 35° C. for about 20 hours. To the reaction mixture, about 20 mL of ethanol and 6.3 grams of decolorizing carbon can be added over a period of about 20 minutes to form a slurry. A slight exotherm can be observed along with some foaming during the addition. The slurry can be allowed to cool to room temperature and maintained over the weekend. The slurry can be filtered through celite and the filter cake washed with about 100 mL of ethanol to afford a filtrate that can be observed as clear and colorless. The filtrate can be concentrated to afford 8.5 grams of the

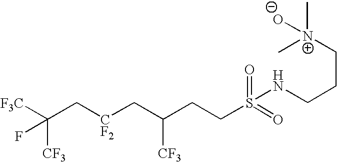

product that can be observed as a white solid. The product structure can be confirmed by NMR and/or LCMS analysis.

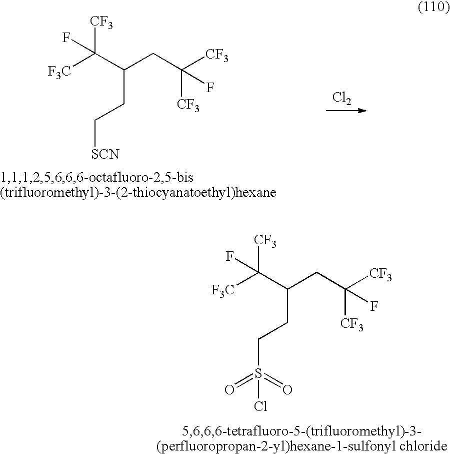

In accordance with scheme (110) above, in a flask that can be equipped with an agitator, thermocouple and a sparging apparatus, (26.15 grams (0.06 mole) of 1,1,1,2,5,6,6,6-octafluoro-2,5-bis(trifluoromethyl)-3-(2-thiocyanatoethyl)hexane (refer to scheme (43) above) and about 75 mL of acetic acid can be placed to form a mixture. The mixture can be heated to about 50° C. and vigorously sparged with chlorine gas for about 5 days to form a reaction mixture. Acetic acid can be replenished as needed to keep the sparging apparatus submerged. The reaction mixture can be allowed to cool and about 2 mL of water added. To the reaction mixture, about 150 mL of chloroform and about 150 mL of water to form a multiphase mixture from which an organic phase can be separated from an aqueous phase. The organic phase can be successively washed with three 150 mL portions of a saturated bicarbonate solution one 150 mL portion of a saturated brine solution. The organic phase can be collected and dried over sodium sulfate over the weekend to form a slurry. The slurry can be filtered and concentrated to afford 20 grams of the 5,6,6,6-tetrafluoro-5-(trifluoromethyl)-3-(perfluoropropan-2-yl)hexane-1-sulfonyl chloride product that can be observed as an oil. The product structure can be confirmed by NMR and/or GC/MS and/or GC analysis.

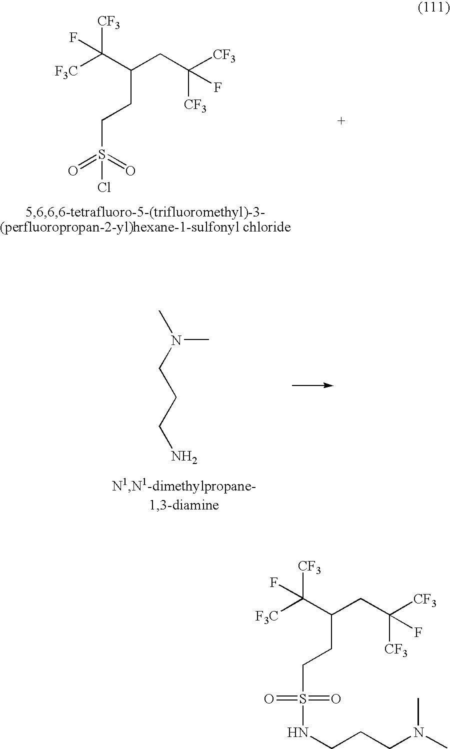

Referring to scheme (111) above, in a flask that can be equipped with an agitator, thermocouple, ice water bath, and an addition funnel, 13.5 mL (0.11 mole) of 3-(dimethylamino)propylamine and about 35 mL of chloroform can be placed to form a first mixture. The first mixture can be cooled to about 0° C. In the addition funnel, 20 grams (0.04 mole) of 5,6,6,6-tetrafluoro-5-(trifluoromethyl)-3-(perfluoropropan-2-yl)hexane-1-sulfonyl chloride (refer to scheme (110) above) and about 35 mL of chloroform can be placed to form a second mixture. The second mixture can be added drop wise to the first mixture over a period of about an hour to form a reaction mixture. The reaction mixture can be maintained at a temperature below 5° C. The peak temperature during addition can be about 1.7° C. The reaction mixture can be allowed to warm to room temperature and stir overnight. The reaction mixture can be successively washed with two 150 mL portions of a saturated NaHCO₃ solution, one 150 mL portion of a saturated solution of NaCl and one 150 mL portion of water wherein each step can produce a multiphase mixture from which an organic phase can be separated from an aqueous phase and each organic phase can be collected and transferred to the next step. The final organic phase can be dried and concentrated to afford 22.7 grams of the

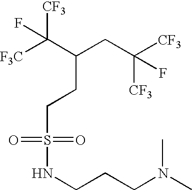

product which can be observed as a brown oil. The product structure can be confirmed by NMR and/or LCMS analysis.

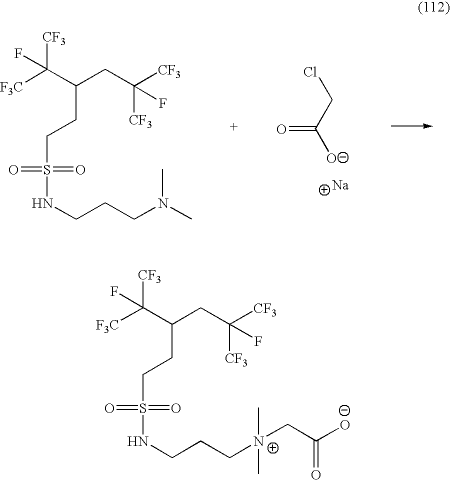

In reference to scheme (112) above, in a flask that can be equipped with an agitator, thermocouple, and a reflux condenser, about 50 mL of ethanol, 2.29 grams (0.02 mole) of sodium chloroacetate and 11 grams (0.02 mole) of

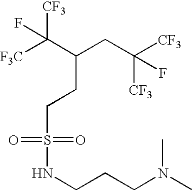

(refer to scheme (111) above) can be placed to form a mixture. The mixture can be heated to reflux for and maintained for about 5 days. The mixture can be allowed to cool and filtered through celite to afford a filtrate. The filtrate can be concentrated to afford the

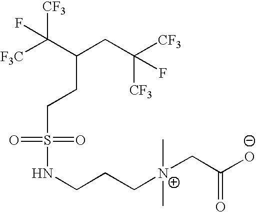

product. The product structure can be confirmed by NMR and/or LCMS analysis.

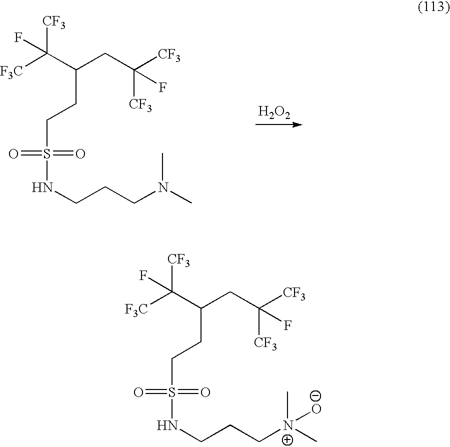

In conformity with scheme (113) above, in a flask that can be equipped with an agitator, thermocouple, ice water bath and an addition funnel, 11 grams (0.02 mole) of

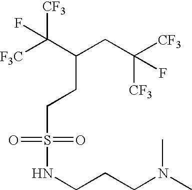

and about 20 mL of ethanol and about 3 mL of water to form a mixture. The mixture can be chilled to about 0° C. using the bath. To the mixture, 9.3 mL of a 50% (wt/wt) solution of hydrogen peroxide in water can be added over a period of about 15 minutes to form a reaction mixture. The reaction mixture can be observed to have peak temperature during addition of 30.3° C. The reaction mixture can be heated to and maintained at 35° C. for about 20 hours. To the reaction mixture, about 20 mL of ethanol and 6.6 grams of decolorizing carbon can be added over a period of about 20 minutes to form a slurry. A slight exotherm can be observed along with some foaming during the addition. The slurry can be allowed to cool to room temperature and maintained over the weekend. The slurry can be filtered through celite and the filter cake washed with about 100 mL of ethanol to afford a filtrate that can be observed as clear and colorless. The filtrate can be concentrated to afford 8.6 grams of the

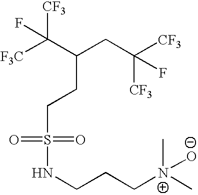

product that can be observed as a white solid. The product structure can be confirmed by NMR and/or LCMS analysis.

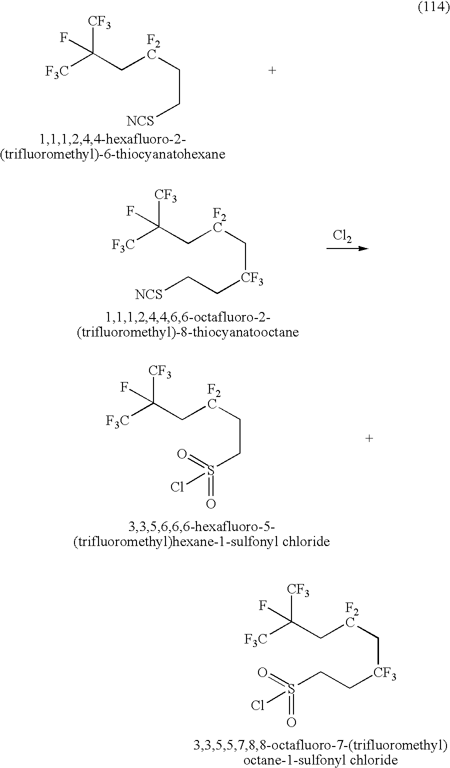

In accordance with scheme (114) above, in a flask that can be equipped with an agitator, thermocouple and a sparging apparatus, 24.68 grams (0.08 mole) of 1,1,1,2,4,4-hexafluoro-2-(trifluoromethyl)-6-thiocyanatohexane and 15.92 (0.04 mole) of 1,1,1,2,4,4,6,6-octafluoro-2-(trifluoromethyl)-8-thiocyanatooctane (refer to scheme (51) above) and about 58 mL of acetic acid can be placed to form a mixture. The mixture can be heated to about 50° C. and vigorously sparged with chlorine gas for about 4 hours to form a reaction mixture. The chlorine sparging can be discontinued and allowed to cool to room temperature and maintained overnight. To the reaction mixture, about 2 mL of water added. To the reaction mixture, about 100 mL of chloroform and about 100 mL of water to form a multiphase mixture from which an organic phase can be separated from an aqueous phase. The organic phase can be successively washed with three 100 mL portions of a saturated bicarbonate solution one 100 mL portion of a saturated brine solution. The organic phase can be collected, dried over sodium sulfate, filtered and concentrated to afford 16.6 grams of the 3,3,5,5,7,8,8,8-octafluoro-7-(trifluoromethyl)octane-1-sulfonyl chloride product. The product structure can be confirmed by NMR and/or GC/MS and/or GC analysis.

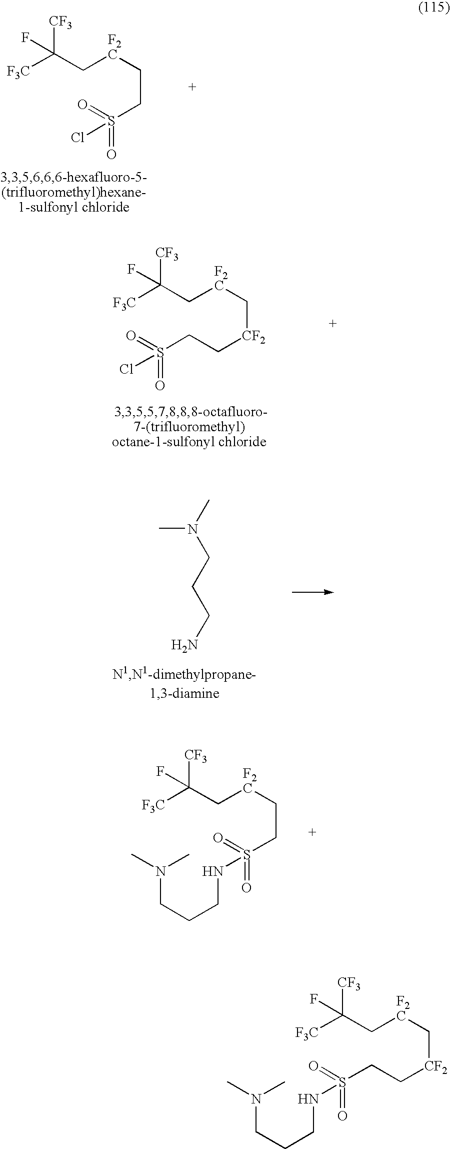

Referring to scheme (115) above, in a flask that can be equipped with an agitator, thermocouple, ice water bath, and an addition funnel, 41.42 mL (0.33 mole) of 3-(dimethylamino)propylamine and about 75 mL of chloroform can be placed to form a first mixture. The first mixture can be cooled to about 0° C. In the addition funnel, 14.8 grams (0.03 mole) of 3,3,5,5,7,8,8,8-octafluoro-7-(trifluoromethyl)octane-1-sulfonyl chloride (refer to scheme (114) above), 27 grams (0.07 mole) of 3,3,5,6,6,6-hexafluoro-5-(trifluoromethyl)hexane-1-sulfonyl chloride (refer to scheme (114) above) and about 75 mL of chloroform can be placed to form a second mixture. The second mixture can be added drop wise to the first mixture over a period of about an hour to form a reaction mixture. The peak temperature during addition can be about 5.9° C. The reaction mixture can be allowed to warm to room temperature and stir overnight. The reaction mixture can be successively washed with two 300 mL portions of a saturated NaHCO₃ solution, one 300 mL portion of a saturated solution of NaCl and one 300 mL portion of water wherein each step can produce a multiphase mixture from which an organic phase can be separated from an aqueous phase and each organic phase can be collected and transferred to the next step. The final organic phase can be dried and concentrated to afford 38.5 grams of the

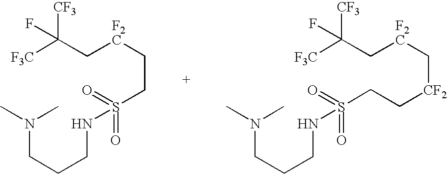

product mixture which can be observed as a brown oil that solidified upon standing. The product structure can be confirmed by NMR and/or LCMS analysis.

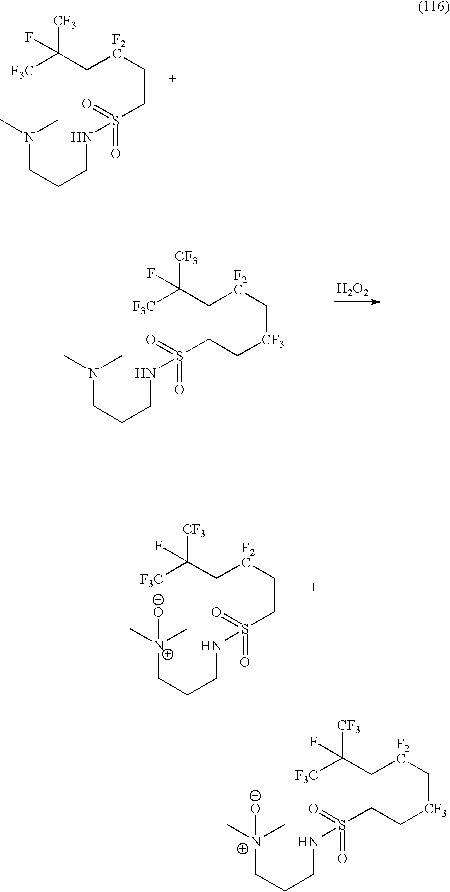

In conformity with scheme (116) above, in a flask that can be equipped with an agitator, thermocouple, ice water bath and an addition funnel, 10 grams of a mixture comprising

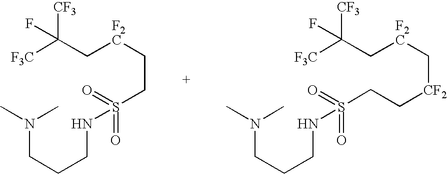

(refer to scheme (115) above) and about 25 mL of ethanol and about 3.5 mL of water to form a mixture. The mixture can be chilled to about 0° C. using the bath. To the mixture, 11 mL of a 50% (wt/wt) solution of hydrogen peroxide in water can be added over a period of about 15 minutes to form a reaction mixture. The reaction mixture can be observed to have peak temperature during addition of 23° C. The reaction mixture can be heated to and maintained at 35° C. for about 48 hours. To the reaction mixture, about 25 mL of ethanol and 6 grams of decolorizing carbon can be added over a period of about 20 minutes to form a slurry. A slight exotherm can be observed along with some foaming during the addition. The slurry can be heated to and maintained at about 50° C. for about 8 hours. The slurry can be allowed to cool to room temperature and maintained over the weekend. The slurry can be filtered through celite and the filter cake washed with about 100 mL of ethanol to afford a filtrate that can be observed as clear and brown. The filtrate can be concentrated to afford about 7.4 grams of the

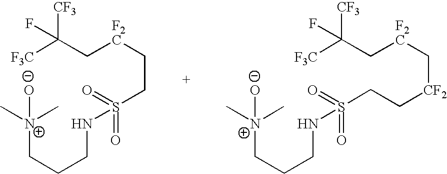

product mixture that can be observed as a brown oil. The product structure can be confirmed by NMR and/or LCMS analysis.

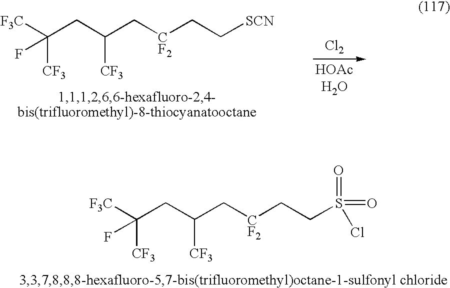

In accordance with scheme (117) above, in a flask that can be equipped with an agitator, a gas sparging apparatus and a thermocouple, 18.4 grams (0.04 mole) of 1,1,1,2,6,6-hexafluoro-2,4-bis(trifluoromethyl)-8-thiocyanatooctane (refer to scheme (52) above) and 60 mL of acetic acid can be placed to form a mixture. The mixture can be heated to about 50° C. and vigorously sparged with chlorine gas for about 6 hours to form a reaction mixture. The gas and the heat can be turned off and the mixture can be stirred at room temperature for about 72 hours. The reaction mixture can be sparged with chlorine gas at 50° C. for about 7 hours. The reaction mixture can be allowed to cool and about 2 mL of water can be added. To the reaction mixture, about 100 mL of chloroform and about 100 mL of water can be added to form a multiphase mixture from which an organic phase can be separated from an aqueous phase. The organic phase can be collected and washed three times with 100 mL portions of saturated bicarbonate solution, 100 mL portion of saturated brine, dried over sodium sulfate, filtered, and concentrated to afford 18 grams of the 3,3,7,8,8,8-hexafluoro-5,7-bis(trifluoromethyl)octane-1-sulfonyl chloride product that can be observed as a yellow oil. The product structure can be confirmed by NMR and GC/MS analysis.

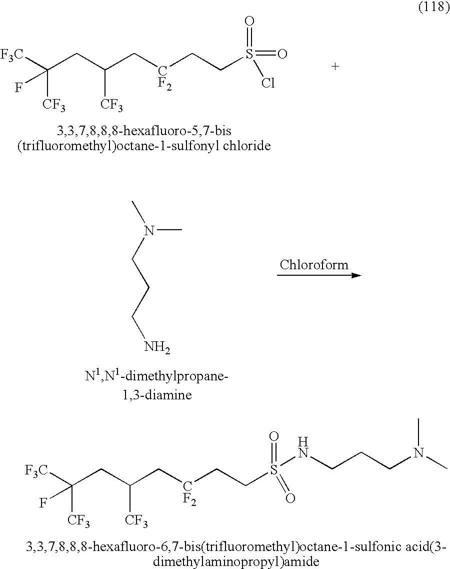

Referring to scheme (118) above, in a flask that can be equipped with an agitator, thermocouple, an ice water bath, and an addition funnel, about 15 mL (0.12 mole) of 3-(dimethylamino)propylamine and about 40 mL of chloroform can be added to form a first mixture. The first mixture can be chilled to about 0° C. using the ice water bath. In the addition funnel, 18 grams (0.04 mole) of 3,3,7,8,8,8-hexafluoro-5,7-bis-trifluoromethyl-octanesulfonyl chloride (refer to scheme (117) above) and 40 mL of chloroform can be combined to form a second mixture. The second mixture can be added dropwise to the first mixture over a one hour period to form a reaction mixture. During the addition, the reaction mixture can be maintained at a temperature of about below 5° C. The reaction mixture can be allowed to warm to room temperature and held overnight. The reaction mixture can be washed by successively adding two 200 mL portions of a saturated NaHCO₃ solution, one 200 mL portion of a saturated NaCl solution and one 200 mL portion of water wherein each step can produce a multiphase mixture from which an organic phase can be separated from an aqueous phase and treated in the successive step. The organic phase can be dried and concentrated to afford 19.5 grams of the 3,3,7,8,8,8-hexafluoro-5,7-bis(trifluoromethyl)octane-1-sulfonic acid(3-dimethylaminopropyl)amide product. The product structure can be confirmed by NMR and LCMS analysis.

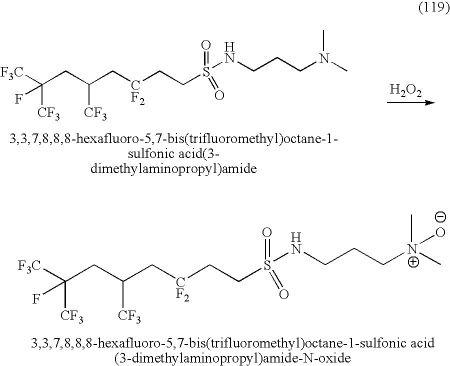

In reference to scheme (119) above, in a flask that can be equipped with an agitator, thermocouple, and an addition funnel, 9 grams (0.017 mole) of 3,3,7,8,8,8-hexafluoro-5,7-bis(trifluoromethyl)octane-1-sulfonic acid(3-dimethylaminopropyl)amide (refer to scheme (118) above), and about 20 mL of ethanol and about 3 mL of water can be placed to form a mixture. To the mixture, 8.5 mL (0.13 mole) of a 50% (wt/wt) solution of hydrogen peroxide in water can be added over a period of about 15 minutes to form a reaction mixture. The peak temperature of the reaction mixture during the addition can be about 2.5° C. The reaction mixture can be heated to and maintained at about 35° C. for about 4 hours. To the reaction mixture, about 20 mL of ethanol and 6.3 grams of decolorizing carbon can be added over a period of 20 minutes to quench the peroxides. A slight exotherm and reaction mixture foaming can be observed. The reaction mixture can be agitated at room temperature over the weekend. The reaction mixture can be filtered through celite which can be washed with 100 mL of ethanol to afford what can be observed as a clear and colorless filtrate. The filtrate can be concentrated to afford 7.35 grams of the 3,3,7,8,8,8-hexafluoro-5,7-bis(trifluoromethyl)octane-1-sulfonic acid(3-dimethylaminopropyl)amido-N-oxide product. The product structure can be confirmed by NMR and LCMS analysis.

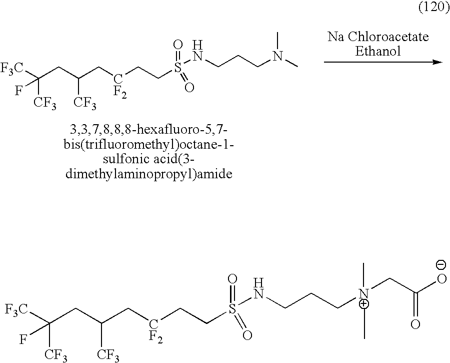

According to scheme (120) above, in a flask that can be equipped with an agitator, thermocouple, and a reflux condenser, 43 mL of ethanol, 2.01 grams (0.017 mole) of sodium chloroacetate and 9 grams (0.017 mole) of 3,3,7,8,8,8 hexafluoro-5,7-bis(trifluoromethyl)octane-1-sulfonic acid (3-dimethylamino-propyl)-amide (refer to scheme (119) above) can be placed to form a mixture. The mixture can be heated to reflux and maintained for about 5 days. The mixture can be cooled and filtered through celite to form a filtrate. The filtrate concentrated to afford 7.5 grams of the

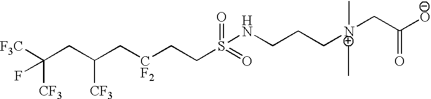

product that can be observed as a brown colored fryable foam. The product structure can be confirmed by NMR and LCMS analysis.

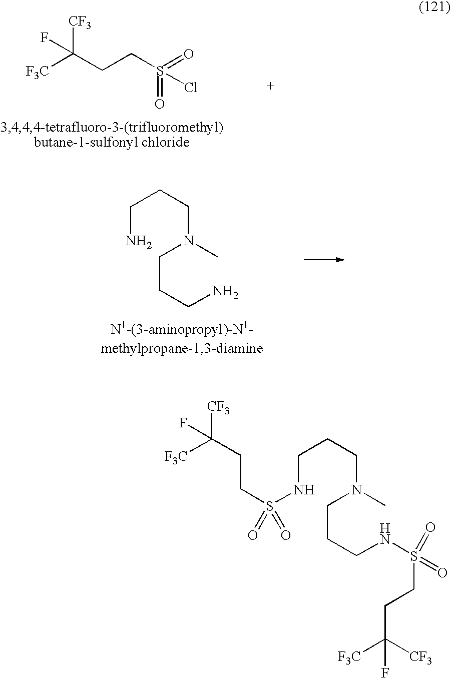

According to scheme (121) above, in a flask that can be equipped with an agitator, thermocouple, and an dry-ice/acetone bath, 10.0 grams (0.069 mole) of 3,3 diamino N methyl dipropylamine and about 60 mL chloroform can be placed to form a first mixture. The first mixture can be chilled to about 0° C. by using the dry-ice/acetone bath. In the addition funnel, 14.6 grams (0.049 mole) of 3,4,4,4-tetrafluoro-3-trifluoromethyl-butane-1-sulfonyl chloride (see, e.g. Published International Applications) and about 40 mL of chloroform can be added to form a second mixture. The second mixture can be added to the first mixture drop wise over a period of about 35 minutes to form a reaction mixture. The reaction mixture can be kept at a temperature at or below about 5° C. The peak temperature during addition can be about −2.5° C. The reaction mixture can be allowed to warm to room temperature and maintained for about two hours. The reaction mixture can be washed with three 100 mL portions of water wherein each can form a multiphase mixture from which an organic phase can be separated from an aqueous phase. The organic phase can be dried and concentrated to afford 16.8 grams of a crude product mixture that contained starting material. The product mixture can be placed on a Kugelrohr apparatus at 80° C. and 0.03 mmHg for about 30 minutes to afford 13.9 grams of a second crude product mixture that contained starting material. The second product mixture can be triturated with two 200 mL portions of water to afford 6.9 grams of the

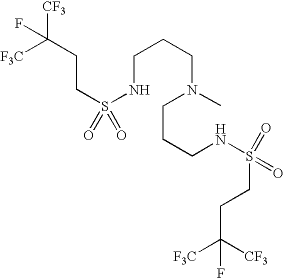

product. The product structure can be confirmed by NMR and/or LCMS analysis.

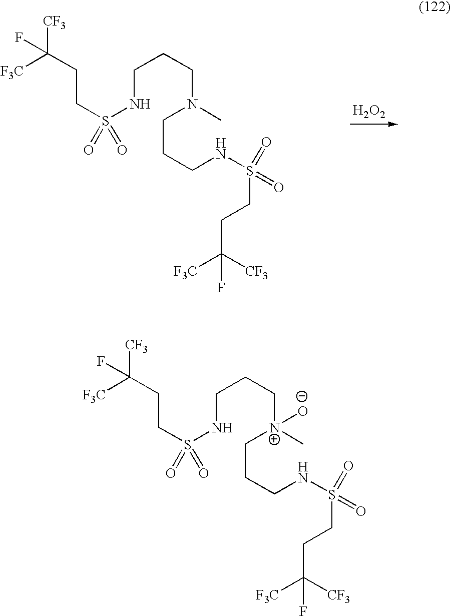

In accordance with scheme (122), in a flask that can be equipped with an agitator, thermocouple and an addition funnel, 7.8 grams (0.012 mole) of

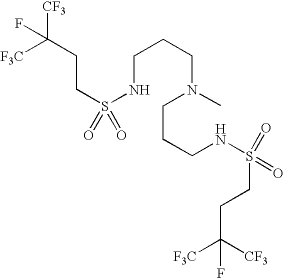

and about 23 mL of ethanol and about 3.6 mL of water can be placed to form a mixture. To the mixture, 6.14 grams of a 50% (wt/wt) solution of hydrogen peroxide in water can be added slowly over a period of 15 minutes at room temperature to form a reaction mixture. The peak temperature of the reaction mixture during addition can be about 20.8° C. The reaction mixture can be observed as a cloudy orange solution which can clarify upon heating. The reaction mixture can be heated to and maintained at about 35° C. for about 3 hours. The reaction mixture can be allowed to cool to room temperature and maintained for overnight. The reaction mixture can be heated to and maintained at about 35° C. for about 2 hours. To the reaction mixture, 5 grams of carbon can be added slowly to quench the peroxides over a period of about 20 minutes to form a slurry. To the slurry, about 30 mL of ethanol can be also added to facilitate uniform stirring. The mixture was left to stir overnight at room temperature. The slurry can be heated to about 50° C. for about four hours. The slurry can be filtered through celite and the filter cake washed with about 300 mL of ethanol to provide a filtrate that can be observed as clear and colorless. The filtrate can be concentrated to afford 4.8 grams of the

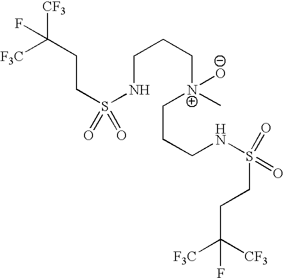

product. The product structure can be confirmed by NMR and/or LCMS analysis.

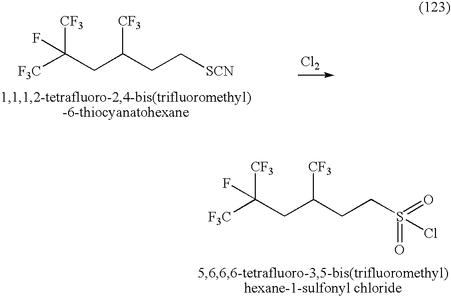

Referring to scheme (123) above, in a flask that can be equipped with an agitator, thermocouple, reflux condenser, and a chlorine (Cl₂ gas) sparging apparatus, 28 grams (79.7 mmol) of 1,1,1,2-tetrafluoro-2,4-bis(trifluoromethyl)-6-thiocyanatohexane (refer to scheme (53) above) and 40 ml of HOAc (glacial) can be placed to form a mixture. The mixture can be heated to 50° C. and sparged via a dispersion tube with chlorine and maintained for four hours to form a reaction mixture. The reaction mixture can be observed to change in color from amber to yellow and turbid and a 5° C. exotherm. The reaction mixture can be stirred at 50° C. for overnight. The reaction mixture can be chlorinated for about 8.5 hours. Conversion can be observed to be about 31.2%. The reaction mixture can be cooled to <20° C. with an ice bath and 125 ml of water added drop wise. The reaction mixture can be sparged with chlorine for a few minutes and sealed with a septum. The reaction mixture can be heated to 50° C. and maintained for overnight. The reaction mixture can be observed to be about 49.1% complete. The reaction mixture can be sparged with chlorine and maintained for about 8.5 hours whereupon the reaction mixture can be observed to be about 63.8% complete. To the reaction mixture, chlorine can be sparged for a period of time and stopped whereupon the reaction mixture can be stirred at 50° C. and maintained for about 16 hours. The reaction mixture can be cooled to room temperature and maintained for about 8 hours. Conversion of the reaction mixture can be observed to be about 82.5%. The reaction mixture can be heated to 60° C. and sparged with chlorine for a period of about 8.5 hours whereupon the conversion can be observed to be 94.0%. Sparging can be continued for overnight and the conversion can be observed to be about 99.5%. Sparging can be halted and the reaction mixture cooled to <10° C. in an ice bath. To the reaction mixture, 40 mL of water can be added drop wise to form a multiphase mixture from which an organic phase can be separated from an aqueous phase and allowed to warm to room temperature. To the multiphase mixture can be added 50 ml of CHCl₃ and 50 ml of water. The aqueous phase can be collected and extracted with 50 ml of CHCl₃ and the combined extracts can be washed three times with 75 ml portions of water. The organic phase can be collected and dried over Na₂SO₄, filtered and concentrated in vacuo to afford 30.15 grams of the 5,6,6,6-tetrafluoro-3,5-bis(trifluoromethyl)hexane-1-sulfonyl chloride product (96.3% yield) that can be observed as a cloudy light yellow oil. The product structure can be confirmed by NMR and/or chromatographic analysis.

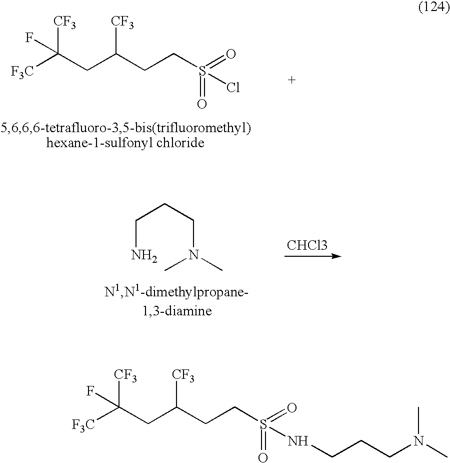

According to scheme (124) above, in a flask that can be equipped with an agitator, thermocouple, reflux condenser, and an addition funnel, 22.0 g of 3-dimethylaminopropylamine and 175 ml of CHCl₃ can be placed to form a mixture. The mixture can be chilled via dry-ice/acetone bath. To the mixture, a mixture of 30.0 grams (76.4 mmol) of 5,6,6,6-tetrafluoro-3,5 bis(trifluoromethyl)hexane-1-sulfonyl chloride (refer to scheme (123) above) and 175 ml of CHCl₃ can be added drop wise over a period of about 30 minutes to form a reaction mixture. The reaction mixture temperature can be observed to be between about 0° C. and −5° C. The reaction mixture can be allowed to warm to room temperature and maintained for overnight. The mixture can be washed once with 300 ml of water, twice with 300 ml portions of a saturated solution of sodium bicarbonate in water and one 300 ml portion of a saturated brine solution to form a multiphase mixture from which an organic phase can be separated from an aqueous phase. The organic phase can be dried over MgSO₄, filtered and concentrated in vacuo to afford 32.18 grams of the

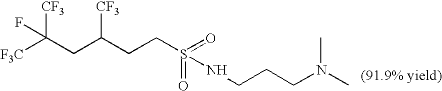

of what can be observed as a colorless liquid. The product structure can be confirmed by NMR and/or chromatographic analysis.

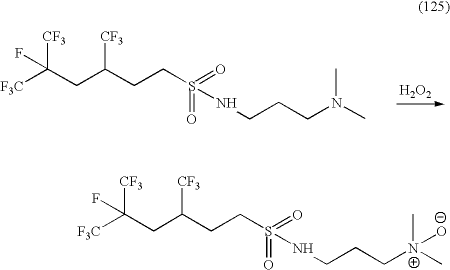

In accordance with scheme (125) above, in a flask that can be equipped with an agitator, thermocouple, reflux condenser, and an addition funnel, 10 grams (0.02 mole) of

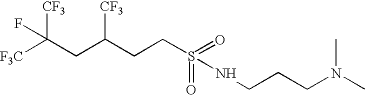

(refer to scheme (124) above), 22 mL of ethanol and 3.5 mL of water can be placed to form a mixture. To the mixture, 10.5 mL of a 50% (wt/wt) solution of hydrogen peroxide in water can be added over a period of 15 minutes to form a reaction mixture. The peak temperature during addition can be about 25.1° C. The reaction mixture can be heated to 35° C. and maintained for overnight. The reaction mixture can be cooled to room temperature and 20 mL of ethanol and 6 grams of decolorizing carbon can be added over 20 minutes to form a slurry. During the addition an exotherm can be observed along with some mild foaming. The slurry can be stirred at 50° C. and maintained for about five hours. The slurry can be filtered through celite to afford a filtrate which can be observed as being clear and colorless. The filtrate can be stripped to afford 8.8 grams of the

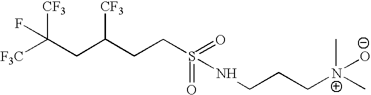

product which can be observed as a white solid (85.4% yd). The product structure can be confirmed by NMR and/or chromatographic analysis.

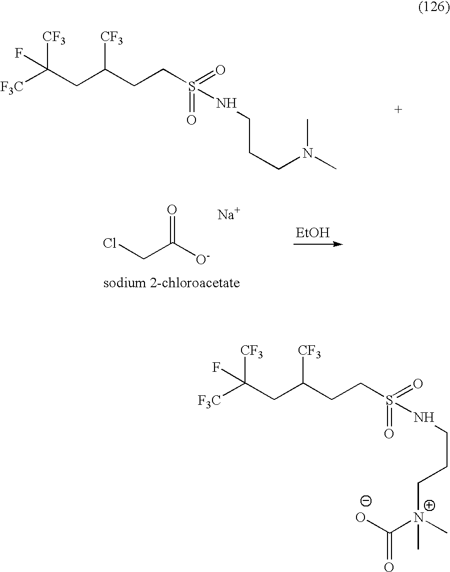

In conformity with scheme (126) above, in a flask that can be equipped with an agitator, thermocouple, reflux condenser, and an addition funnel, 55 mL of ethanol, 2.54 grams of sodium chloroacetate and 10 grams (0.22 mole) of

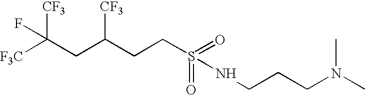

(refer to scheme (125) above) can be placed to form a mixture. The mixture can be heated to reflux and maintained for six days. The mixture can be cooled and filtered through celite to afford a filtrate. The filtrate can be stripped of solvent to afford 8 grams of the

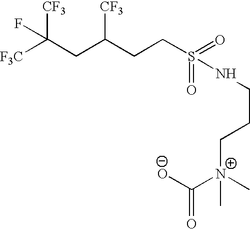

product that can be observed as a tan fryable foam (70.8% yd.). The product structure can be confirmed by

NMR and/or chromatographic analysis.

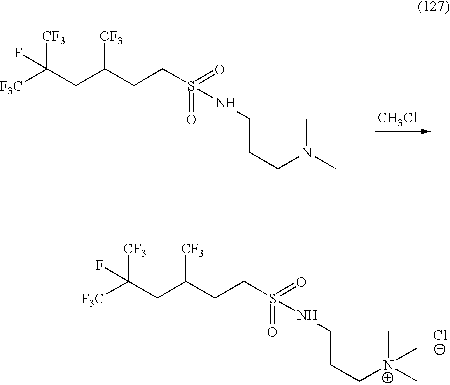

Conforming to scheme (127) above, in a flask that can be equipped with an agitator, thermocouple, reflux condenser, and an addition funnel, 10 grams (0.02 mole) of

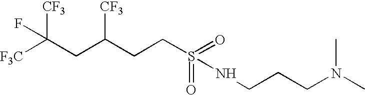

(refer to scheme (125) above) and 115 mL of tert-butyl methyl ether can be placed to form a mixture and chilled in a dry ice acetone bath. To the mixture, 11.6 grams (0.09 mole) of a 2M solution of chloromethane in tert-butyl methyl ether can be added to form a reaction mixture. The reaction mixture can be sealed and heated to 55° C. and maintained for 4 days. The reaction mixture can be observed to become a white slurry. The reaction mixture can be cooled, vented and filtered to afford 7.25 grams of the

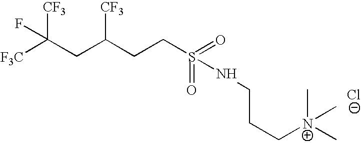

product (65.3% yd). The product can be washed with ether and dried to afford what can be observed as an off white solid. The product structure can be confirmed by NMR and/or chromatographic analysis.

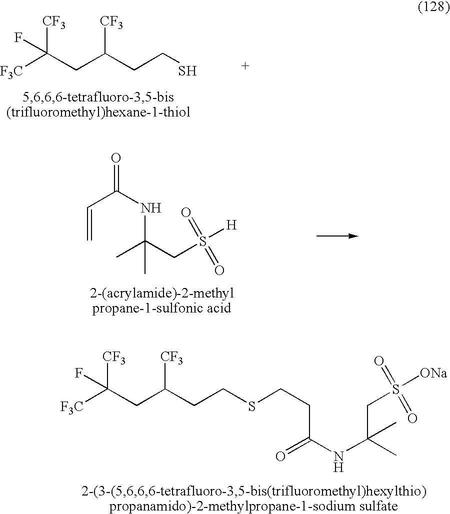

In accordance with scheme (128) above, in a flask that can be equipped with an agitator, thermocouple, reflux condenser, and an addition funnel, 30 mL of ethanol and 0.64 grams (0.03 mole) of cut sodium metal can be placed to form a mixture. To the mixture, 5 grams (0.02 mole) of 5,6,6,6-tetrafluoro-3,5-bis(trifluoromethyl)hexane-1-thiol (refer to scheme (54) above) can be added slowly to form a first reaction mixture and allowed to stir for 30 minutes at room temperature. To the first reaction mixture, 2.9 grams (0.01 mole) of 2-(acrylamido)-2-methylpropane-1-sulfonic acid can be added slowly at room temperature to form a second reaction mixture and allowed to stir at room temperature for overnight. To the second reaction mixture, 4.6 mL of a 6N solution of HCl in water can be added to form what can be observed as a white slurry. The white slurry can be filtered to afford a first filtrate and stripped of ethanol and titrated with two 100 mL portions of ether and filtered to afford a second filtrate. The second filtrate can be stripped and placed on a Kugelrohr apparatus (40 C, 45 min, 0.03 mmHg) to afford 7.25 grams of what can be observed as a yellow solid. The yellow solid can be dried and 20 mL of ethanol and 0.54 grams of NaOH can be added to afford a third reaction mixture and allowed to stir for two hours. The ethanol can be stripped to afford 5.4 grams of the 2-(3-(5,6,6,6-tetrafluoro-3,5-bis(trifluoromethyl)hexylthio)propanamido)-2-methylpropane-1-sodium sulfate (66.75 yd.) product. The product structure can be confirmed by NMR and/or chromatographic analysis.

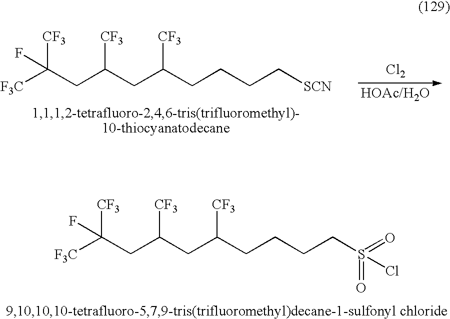

Referring to scheme (129) above, in a flask that can be equipped with an agitator, thermocouple, reflux condenser, and an dispersion tube, 34.6 grams (72.8 mmol) of 1,1,1,2-tetrafluoro-2,4,6-tris(trifluoromethyl)-10-thiocyanatodecane (refer to scheme (57) above) and 40 ml of glacial acetic acid can be placed to form a mixture. The mixture can be heated to 60° C. and sparged via a dispersion tube with chlorine gas to form a reaction mixture and maintained for overnight. The reaction mixture can be observed to change in color from amber to yellow and become turbid with time. The reaction mixture can be cooled to about 10° C. and 50 ml of water added drop wise to form a multiphase mixture from which an organic phase can be separated from an aqueous phase. The multiphase mixture can be warmed to room temperature and diluted with 200 ml of CHCl₃ and 100 ml of water. The aqueous phase can be separated and extracted with 200 ml of CHCl₃ to afford an extract. The extract and organic phase can be combined and washed three times with 300 ml portions of water to form a multiphase mixture from which an organic phase can be separated from an aqueous phase. The organic phase can be washed with 300 ml of brine and then dried over Na₂SO₄. Filtration and concentration in vacuo can result in 31.99 grams of the 9,10,10,10-tetrafluoro-5,7,9-tris(trifluoromethyl)decane-1-sulfonyl chloride product (98.1% yield) which can be observed as a cloudy colorless oil. The product structure can be confirmed by NMR and/or chromatographic analysis.

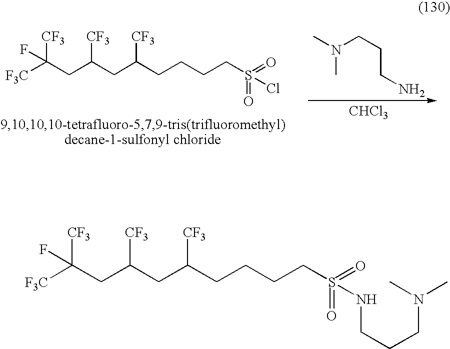

According to scheme (130) above, in a flask that can be equipped with an agitator, thermocouple, ice/acetone bath, and an addition funnel, 32.00 grams (61.9 mmol) of 9,10,10,10-tetrafluoro-5,7,9-tris(trifluoromethyl)decane-1-sulfonyl chloride (refer to scheme (129) above), and 150 ml of CHCl₃ can be placed to form a mixture. To the cooled mixture, 17.70 grams (174 mmol) of 3-dimethylaminopropylamine and 150 ml of CHCl₃ can be added drop wise to form a reaction mixture over a 60 minute period while maintaining the reaction temperature between 0° C. and −5° C. The reaction can be allowed to warm to room temperature and stir over the weekend. The reaction mixture can be washed once with 400 ml of water, twice with 300 ml portions of a saturated solution of sodium bicarbonate, 300 ml of water, and 300 ml of brine. The organic phase can be dried over Na₂SO₄ and filtered to afford a filtrate. The filtrate can be concentrated in vacuo to afford 34.44 gram of the

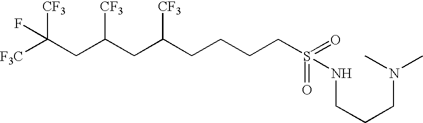

(95.5% yield) of what can be observed as a light yellow liquid. The product structure can be confirmed by NMR and/or chromatographic analysis.

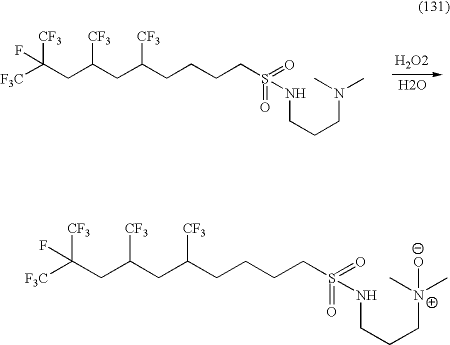

In accord with scheme (131) above, in a flask that can be equipped with an agitator, thermocouple, reflux condenser, and an addition funnel, 10.0 grams (17.2 mmol) of

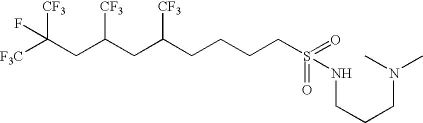

(refer to scheme (130) above) and 20 ml of absolute ethanol and 2.5 ml of water to form a mixture at room temperature. To the mixture, 8.0 ml of a 50% solution of H₂O₂ in water over a 1 minute period to form a reaction mixture. The reaction mixture can be heated to 35° C. and maintained for overnight. The reaction mixture can be cooled to room temperature, diluted with 20 ml of EtOH, and treated portionwise with 5 g of decolorizing carbon (neutral) over a 90 minute period to form a slurry. The temperature can be observed to increase to 30° C. with foaming. The slurry can be heated to 50° C. and stirred for 3 hours. The slurry can be cooled to room temperature and stirred for overnight. A filtered sample of the black slurry was tested negative for any unquenched peroxide with Kl/Starch paper. The slurry can be filtered through celite and concentrated in vacuo, and co-stripped three times with CHCl₃ to afford a semi-concentrate. The semi-concentrate can be further concentrated under high vacuum at 50° C. to afford 10.4 grams of the

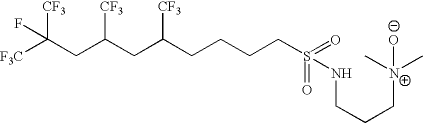

product that can be observed as a viscous amber oil. The product structure can be confirmed by NMR and/or chromatographic analysis.

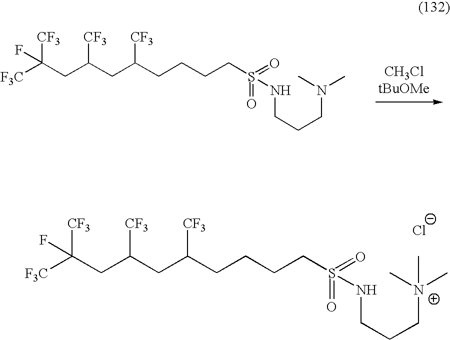

In conformity with scheme (132), in a sealable tube, 10.0 grams (17.2 mmol)

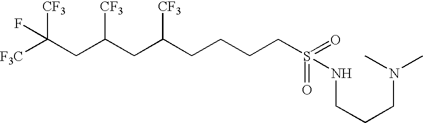

of (refer to scheme (130) above) and 12.1 grams (34.3 mmol) of chloromethane and 60 ml of methyl-tert-butyl ether can be placed in a sealed tube and heated to 55° C. for an extended period of time to form a mixture. Stirring can be halted and an oil can be observed to settle to the bottom. The tube can be cooled below about 0° C. whereupon the oil can be observed to solidify into a pale yellow waxy solid, vented, and the liquid was decanted from the solid. The solid can be dissolved in dichloromethane, transferred, and concentrated to afford 10.9 grams of the

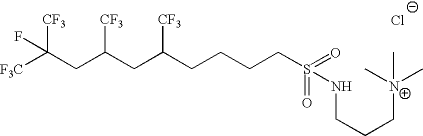

product. The product structure can be confirmed by NMR and/or chromatographic analysis.

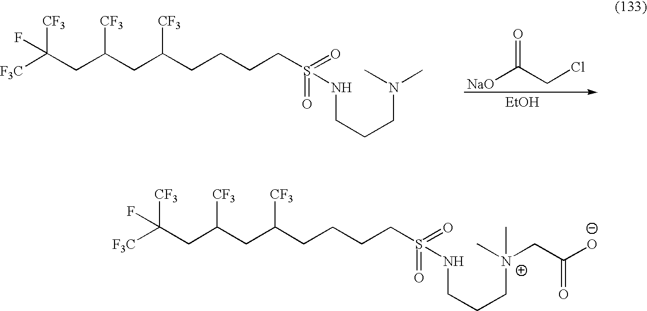

Conforming to scheme (133) above, in a flask that can be equipped with an agitator, thermocouple, reflux condenser, and an addition funnel, 10.0 grams (17.2 mmol) of

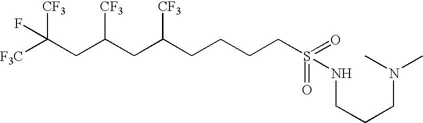

(refer to scheme (130) above), 40 ml of absolute ethanol and 2.0 grams (17.2 mmol) of sodium chloroacetate can be placed to form a mixture. The mixture can be heated to reflux (79° C.) and stirred for about three days to afford what can be observed as viscous off-white slurry. The slurry can be filtered to afford a wet-cake. The wet-cake can be dried in a vacuum oven at 45° C. for overnight to afford a solid. Solvent can be observed in the solid, the solid can be pulverized and dried at high vacuum at 45° C. for 3 hours to afford 7.72 grams (70.2% yield) of the

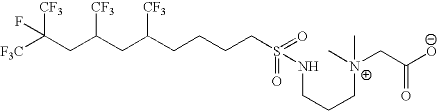

product that can be observed as a white powder. The product structure can be confirmed by NMR and/or chromatographic analysis.

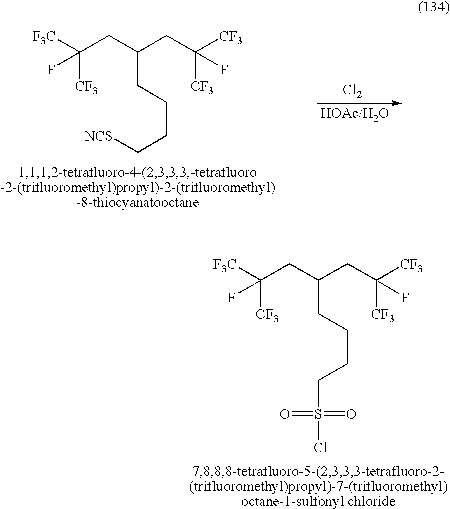

In accordance with scheme (134) above, in a flask that can be equipped with an agitator, thermocouple and a chlorine gas dispersion tube, 34.6 grams (70.1 mmol) of 1,1,1,2-tetrafluoro-4-(2,3,3,3-tetrafluoro-2-(trifluoromethyl)propyl)-2-(trifluoromethyl)-8-thiocyanatooctane (refer to scheme (46) above) and 40 ml of glacial acetic acid to form a mixture and can be heated to about 60° C. The heated mixture can be sparged via the dispersion tube with Cl₂ gas to form a reaction mixture and can be maintained for overnight. The reaction mixture, can be observed to change from an amber solution to a yellow solution and turbid over time. The reaction mixture can be cooled to about 10° C. and 40 ml of water can be added drop wise to form a multiphase mixture from which an organic phase can be separated from an aqueous phase. The multiphase mixture can be warmed to room temperature and diluted twice with 50 ml and 100 ml of CHCl₃ and 60 ml of water, respectively, in order to facilitate phase separation. The aqueous phase (about 400 ml) can be extracted with 200 ml of CHCl₃. The extracts can be washed three times with 300 ml portions of water. The cloudy organic phase can be washed with 300 ml of brine and dried over Na₂SO₄. Filtration and concentration in vacuo to afford 36.72 g (97.9% yield) of the 7,8,8,8-tetrafluoro-5-(2,3,3,3-tetrafluoro-2-(trifluoromethyl)propyl)-7-(trifluoromethyl)octane-1-sulfonyl chloride as what can be observed as a cloudy colorless oil. The product structure can be confirmed by NMH and/or chromatographic analysis.

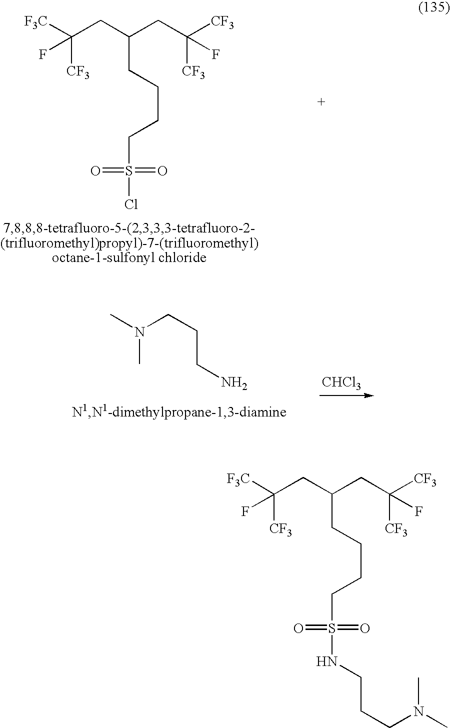

In reference to scheme (135) above, in a flask that can be equipped with an agitator, thermocouple, ice/acetone bath, and an addition funnel, 36.5 grams (68.3 mmol) of 7,8,8,8-tetrafluoro-5-(2,3,3,3-tetrafluoro-2-(trifluoromethyl)propyl)-7-(trifluoromethyl)octane-1-sulfonyl chloride (refer to scheme (134) above) and 150 ml of CHCl₃ can be placed to form a mixture. The mixture can be chilled to 0° C. and a solution of 19.50 grams (191.3 mmol) of 3-dimethylaminopropylamine in 150 ml of CHCl₃ can be added drop wise over a 30 minute period while maintaining the reaction temperature between 0° C. and −5° C. to form a reaction mixture. The reaction mixture can be allowed to warm to room temperature and maintained stirring overnight. The reaction mixture can be washed once with 300 ml of water, twice with 300 ml portions of bicarbonate, 300 ml of water, and 300 ml of brine. The extracts can be checked by GC to ensure all of the dimethylaminopropylamine (8.15 min.) was removed. The organic layer can be dried over Na₂SO₄. Filtration and concentration in vacuo can afford 39.30 grams (95.9% yield) of the

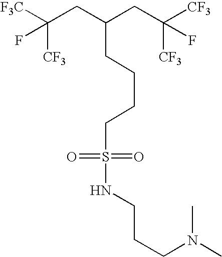

product that can be observed as a pale yellow liquid. The product structure can be confirmed by NMR and/or chromatographic analysis.

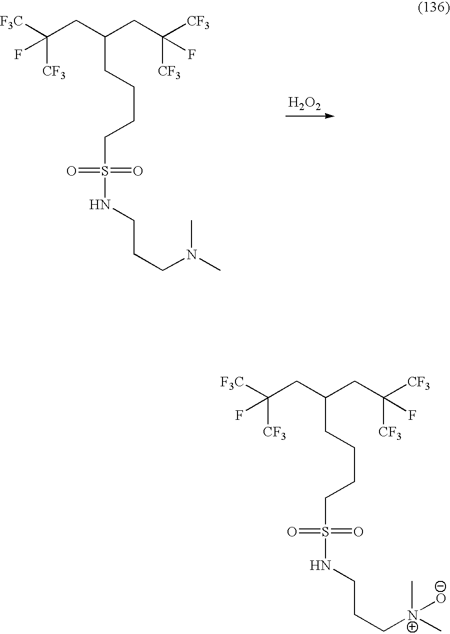

Referring to scheme (136) above, in a flask that can be equipped with an agitator, thermocouple, reflux condenser, and an addition funnel, 10.0 grams (16.7 mmol) of

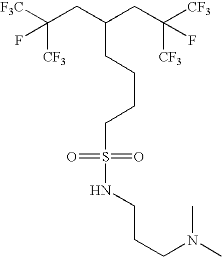

(refer to scheme (135) above); 20 ml of absolute ethanol and 2 ml of water can be placed to form a mixture. To the mixture, about 7.75 ml of a 50% solution of H₂O₂ in water can be added drop wise over a 2 minute period at room temperature to form a reaction mixture. The reaction mixture can be heated to 35° C. and maintained for overnight. The reaction mixture can be cooled to room temperature, diluted with 20 ml of ethanol, and treated portion-wise with 5 grams of decolorizing carbon (neutral) over a 90 minute period to form a slurry. The temperature can increase from room temperature to a maximum of 30° C. and foaming occurred. The slurry can be stirred overnight at room temperature. A filtered sample of the slurry can be tested for any unquenched peroxide with Kl/Starch paper. The test can result as positive for peroxide and the slurry can be heated to 50° C. and stirred for 3 hours. The slurry, after testing negative, can be filtered through celite and the filtrate concentrated in vacuo to afford 10.4 grams the

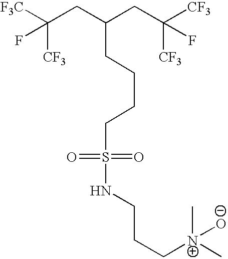

product. The concentrate can be dissolved in and stripped three times each dichloromethane and CHCl₃ to remove the ethanol. Concentration under high vacuum at 50° C. resulted in 10.3 grams of the product which can be observed as a viscous amber oil. The product structure can be confirmed by NMR and/or chromatographic analysis.

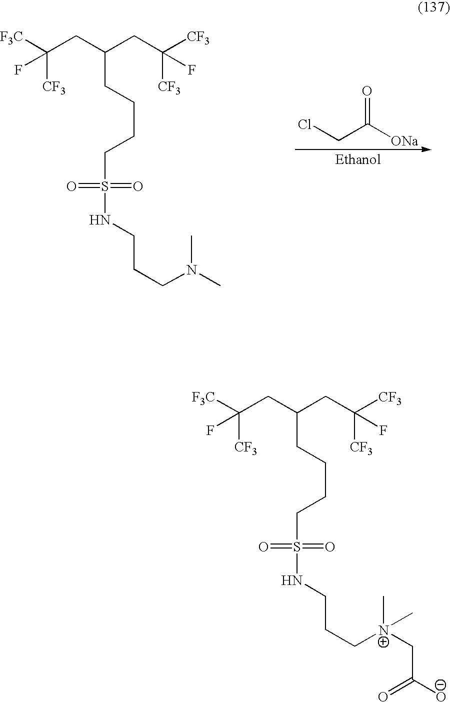

In accordance with scheme (137) above, in a flask that can be equipped with an agitator, thermocouple, reflux condenser, and an addition funnel, 10.0 grams (16.7 mmol) of

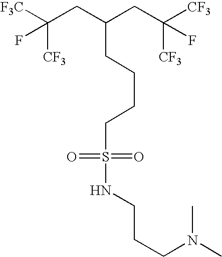

(refer to scheme (135) above), 40 ml of absolute ethanol and 1.95 gram (16.7 mmol) of sodium chloroacetate can be placed to form a mixture. The mixture can be heated to reflux (79° C.) and stirred for 32 to 48 hours and can be observed as an off-white slurry. The slurry can be filtered and the wet cake collected. The wet-cake can be dried in a vacuum at 45° C. for overnight to afford what can be observed as a white solid. Solvent can be present and the white solid pulverized and dried at high vacuum at 45° C. for 3 hours resulting in 7.72 grams of the

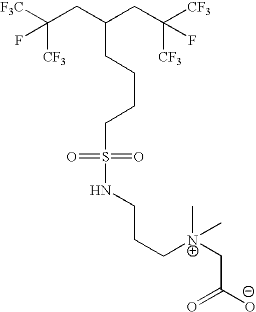

product (70.2% yield) of what can be observed as a white powder. The product structure can be confirmed by NMR and/or chromatographic analysis.

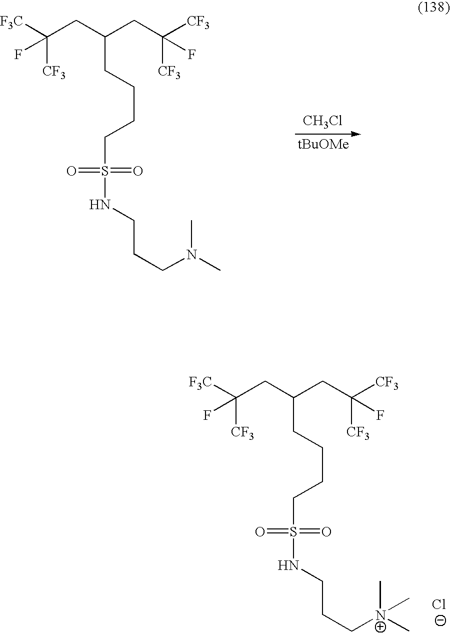

In conformity with scheme (138) above, in a sealed tube, 10.0 grams (16.7 mmol) of

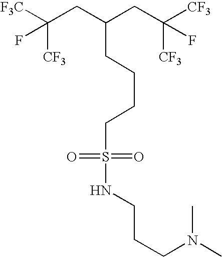

(refer to scheme (135) above) and 85 ml of a 1N solution of chloromethane in methyl-tert-butyl ether can be placed to form a mixture. The mixture can be heated to 55° C. for an extended period of time. The mixture can be cooled in a dry ice acetone bath and 12 grams of chloromethane can be added. The mixture can be heated to 55° C. and maintained for about 3 days. The mixture can be cooled below 0° C. and vented, and multiphase mixture can be observed wherein a solid phase can be separated from a solid phase. The liquid phase can be separated from the solid phase. The solid can be purified by placing under vacuum at 50° C. to afford 9.35 grams of the

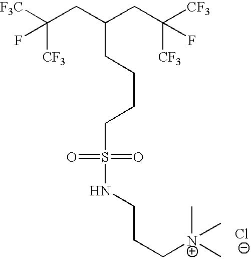

product that can be observed as a white waxy solid. The product structure can be confirmed by NMR and/or chromatographic analysis.

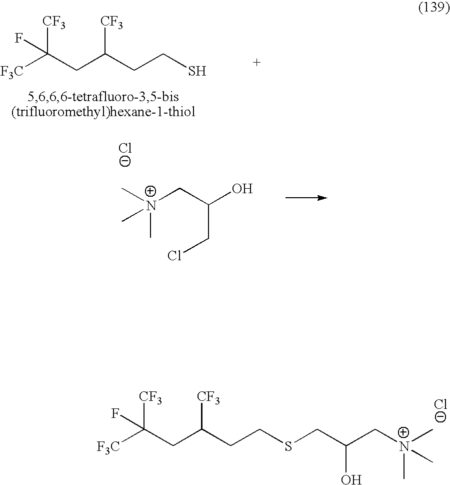

Referring to scheme (139) above, in a flask that can be equipped with an agitator, thermocouple, reflux condenser, and an addition funnel, 4.16 grams of water, 5 grams (0.015 mole) of 5,6,6,6-tetrafluoro-3,5-bis (trifluoromethyl)hexane-1-thiol (refer to scheme (54) above), 4.81 grams (0.015 mole) of 3-chloro-2 hydroxypropyl trimethyl ammonium chloride (60% in water) and 0.61 grams (0.015 mole) of sodium hydroxide can be placed to form a mixture. The mixture can be stirred at room temperature after about 15 minutes the mixture can be observed to warm significantly and thicken to form what can be observed as a white semisolid. To the semisolid, 5 mL of ethanol can be added to facilitate stirring. The semisolid can be stirred at room temperature and maintained for about 5 hours. To the semisolid, 100 mL of ethanol can be added and filtered to afford a filtrate and a wet cake. The filtrate can be stripped and three 150 mL portions of ethanol can be added and an azeotropic distillation performed to afford what can be observed as a yellow residue. The yellow residue can be dissolved in 150 mL of chloroform and filtered to afford a filtrate and a wet-cake. The filtrate can be concentrated to afford what can be observed as a yellow oil and placed on a Kugelrohr apparatus (50° C., 60 minutes, 0.03 mmHg) to afford 7.1 grams of the

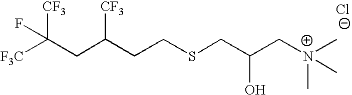

product that can be observed as a yellow oil (95.6% yd.). The product structure can be confirmed by NMR and/or chromatographic analysis.

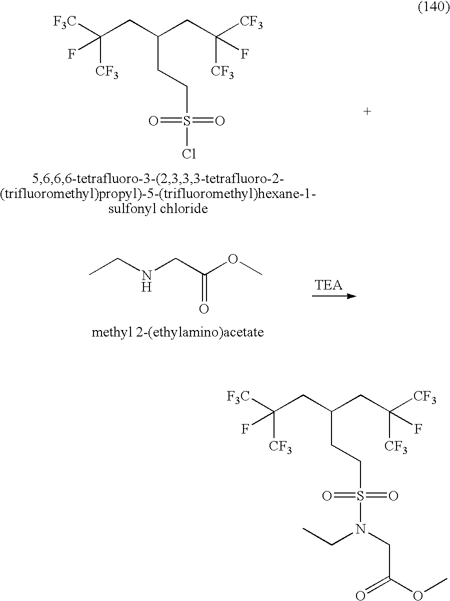

Referring to scheme (140) above, in a flask that can be equipped with an agitator, thermocouple, reflux condenser, and an addition funnel, 5 grams (0.01 mole) of 5,6,6,6-tetrafluoro-3-(2,3,3,3-tetrafluoro-2-(trifluoromethyl)propyl)-5-(trifluoromethyl)hexane-1-sulfonyl chloride (refer to scheme (74) above), 1.2 grams (0.01 mole) of methyl 2-(ethylamino)acetate and 10 mL of chloroform can be placed to form a mixture and chilled to 0° C. To the mixture, 3 mL of triethylamine (TEA) and 10 mL of chloroform can be added drop wise to form a reaction mixture. The peak temperature during addition can be about 3.9° C. The reaction mixture can be allowed to warm to room temperature while stirring and maintained for overnight. To the reaction mixture, 20 mL of cholorform can be added and washed with two 25 mL portions a saturated solution NaHCO₃ in water, two 25 mL portions of water and 25 mL of a saturated NaCl solution in water to form multiphase mixtures from which an organic phase can be separated from an aqueous phase. The organic phase can be collected, dried and concentrated to form a concentrate. To the concentrate, 25 mL of chloroform can be added to form a diluant. To the diluant, 25 mL of a 5% (wt/wt) solution of HCl in water can be added to afford an acidified diluant. To the acidified diluant, 25 mL of a 1N solution of NaOH can be added to form a neutral multiphase mixture from which an organic phase can be separated from an aqueous phase. The organic phase can be dried and concentrated to afford 3.45 grams of the

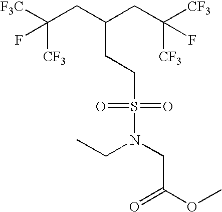

product that can be observed as a yellow oil (59.5% yd.). The product structure can be confirmed by NMR and/or chromatographic analysis.

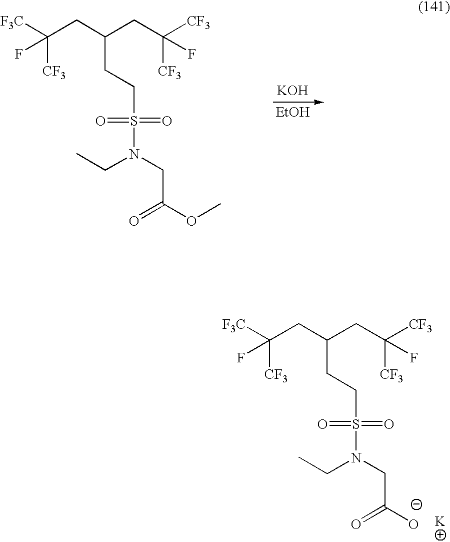

In reference with scheme (141) above, in a flask that can be equipped with an agitator, thermocouple, reflux condenser, and an addition funnel, 3.45 grams (0.006 mole) of

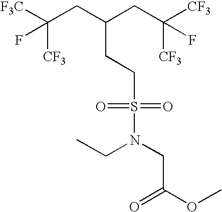

(refer to scheme (140) above) and 11.7 mL of ethanol and 0.39 grams (0.006 mole) of KOH can be added to form a reaction mixture. The reaction mixture can be allowed to stir at room temperature for overnight. The reaction mixture can be stripped to afford 2.75 grams of the

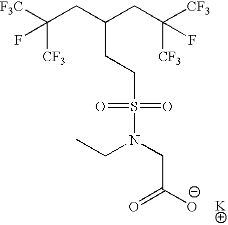

product (76.4% yd.) which can be observed as a solid. The product structure can be confirmed by NMR and/or chromatographic analysis.

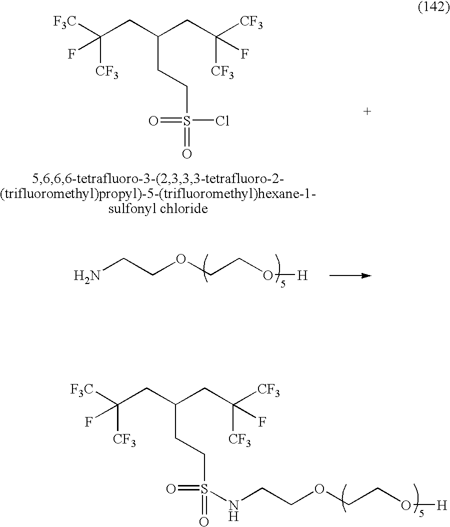

In reference to scheme (142) above, in a flask that can be equipped with an agitator, thermocouple, reflux condenser, and an addition funnel, 2 grams (0.004 mole) of 5,6,6,6-tetrafluoro-3-(2,3,3,3-tetrafluoro-2-(trifluoromethyl)propyl)-5-(trifluoromethyl)hexane-1-sulfonyl chloride (refer to scheme (74) above), 1.11 grams (0.004 mole) of 2-(2-(2-(2-(2-(2-aminoethoxy)ethoxy)ethoxy)ethoxy)ethoxy)ethanol and 7.9 mL of chloroform can be placed to form a mixture and chilled to about 0° C. To the mixture, 0.4 gram (0.004 mole) of triethylamine (TEA) and chloroform can be added drop wise to form a reaction mixture. The reaction mixture can be allowed to warm to room temperature. The reaction mixture can be washed with 20 mL of a 5% (wt/wt) solution of HCl in water and 20 mL of a 1N solution of NaOH in water and 20 mL of a saturated brine solution wherein each step in the washing procedure can form a multiphase mixture from which an organic phase can be separated from an aqueous phase. The organic phase can be dried, filtered and stripped of solvent to afford 1.6 grams of what can be observed as a brown oil containing residual TEA. To the oil, chloroform, 20 mL of a 5% (wt/wt) solution of HCl in water, 20 mL of a saturated solution of bicarbonate solution, and 20 mL of a saturated brine solution wherein each step in the washing procedure can form a multiphase mixture from which an organic phase can be separated from an aqueous phase. The organic phase can be dried, filtered and stripped of solvent to afford 0.65 grams of the

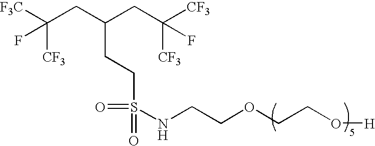

product which can be observed as a brown oil. The product structure can be confirmed by NMR and/or chromatographic analysis.

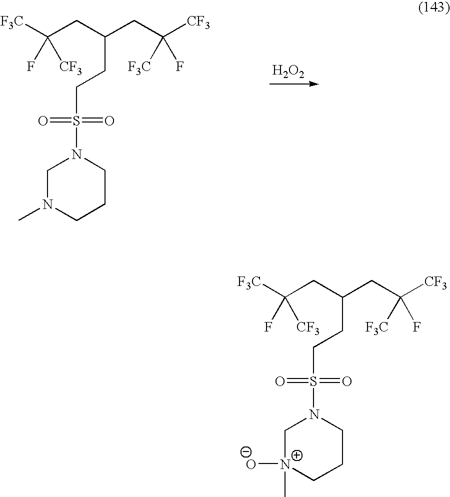

The starting material

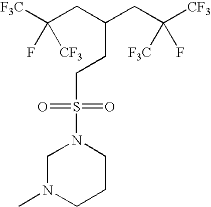

can be formed as a by-product during the preparation of

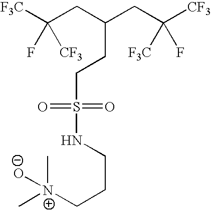

(see, e.g. Published International Applications).

According to scheme (143) above, in a flask that can be equipped with an agitator, thermocouple, reflux condenser, and an addition funnel, 20.0 grams (0.036 mole) of the

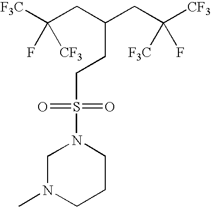

5.1 ml of water at room temperature can be placed to form a mixture. To the mixture, 16.3 ml of 50% solution of H₂O₂ in water can be added over a 1 minute period to form a reaction mixture. The reaction mixture can be heated to 35° C. and maintained for over the weekend. The reaction mixture can be treated portion-wise with 5 grams of decolorizing carbon (neutral) over a 30 minute period to form a slurry. The slurry can be heated to 50° C. and maintained for overnight. To the slurry, 4 grams of the carbon can be added and heated at 50° C. for about two hours. The slurry can be filtered through celite and stripped of EtOH on a rotary evaporator to afford a concentrate. Trace amounts of EtOH remaining in the concentrate can be removed by co-stripping three times with CHCl3 and concentration in vacuo at 45° C. under high vacuum to afford 20.13 grams of the

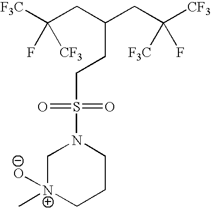

product. The product structure can be confirmed by NMR and/or chromatographic analysis.

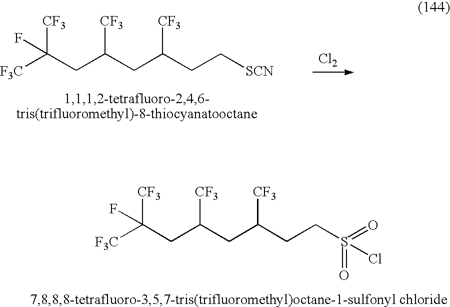

In accordance with scheme (144), in a flask that can be equipped with an agitator, thermocouple, reflux condenser, and a chlorine gas sparger, 28.8 grams (0.06 mole) of 1,1,1,2-tetrafluoro-2,4,6-tris(trifluoromethyl)-8-thiocyanatooctane (refer to scheme (61) above) and 75 mL of acetic acid can be placed to form a mixture. The mixture can be heated to 50° C. and vigorously sparged with chlorine gas for at least 16 hours to form a reaction mixture. The reaction mixture can be allowed to cool to room temperature and maintained for at least 24 hours. The sparging and heating can be resumed for at least about 8 hours. The reaction mixture can be allowed to cool and 2.5 mL of water, 150 mL of chloroform and 150 mL of water can be added to form a multiphase mixture from which an organic phase can be separated from an aqueous phase. The organic phase can be washed with three 150 mL portions of a saturated bicarbonate solution and one 150 mL portion of brine. The organic phase can be dried over sodium sulfate and stripped of solvent to afford 24.8 grams of the 7,8,8,8-tetrafluoro-3,5,7-tris(trifluoromethyl)octane-1-sulfonyl chloride that can be observed as a pale yellow oil (78.7% yd). The product structure can be confirmed by NMR and/or chromatographic analysis.

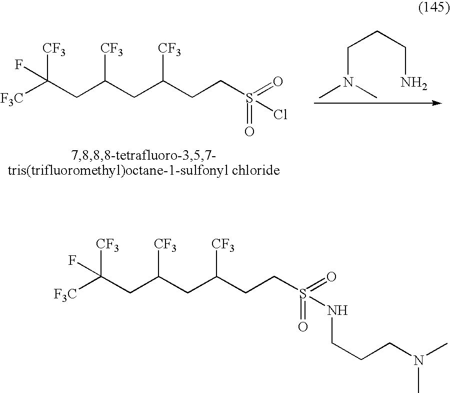

Referring to scheme (145) above, in a flask that can be equipped with an agitator, thermocouple, reflux condenser, and an addition funnel, 14 mL of 3-(dimethylamino)propylamine and 40 mL of chloroform can be placed to form a mixture. The mixture can be chilled to about 0° C. and 18 grams (0.04 mole) of 7,8,8,8-tetrafluoro-3,5,7-tris(trifluoromethyl)octane-1-sulfonyl chloride (refer to scheme (144) above) and 40 mL of chloroform can be added drop wise over a period of about 15 minutes to form a reaction mixture. The reaction mixture can be maintained at a temperature below about 10° C. with a peak temperature during addition can be about 10.1° C. The reaction mixture can be allowed to warm to room temperature and maintained for about four hours. The reaction mixture can be washed twice with 150 mL portions of a saturated solution of NaHCO₃ in water, 150 mL portion of a saturated solution of NaCl in water and 150 mL portion of water. The organic phase can be dried and stripped to afford 18.8 grams of the

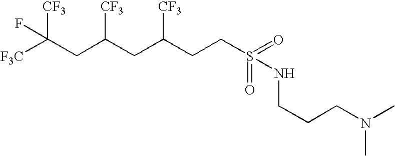

product which can be observed as a yellow oil (92.2% yd.). The product structure can be confirmed by NMR and/or chromatographic analysis.

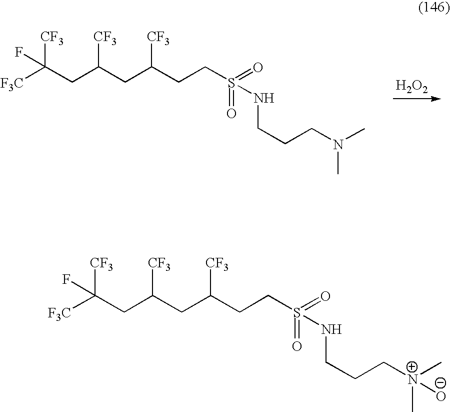

In reference to scheme (146) above, in a flask that can be equipped with an agitator, thermocouple, reflux condenser, and an addition funnel, 6 grams (0.01 mole) of

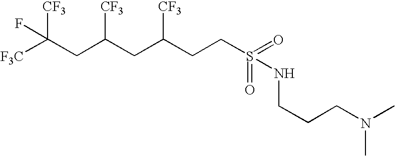

(refer to scheme (145) above), 11 mL of ethanol and 1.6 mL of water can be placed to form a mixture. To the mixture, 5.5 mL of a 50% (wt/wt) solution of hydrogen peroxide in water can be added over a period of about 15 minutes to form a reaction mixture. The peak temperature during addition can be observed to be about 29.2° C. The reaction mixture can be heated to about 35° C. and maintained for overnight. The reaction mixture can be cooled to room temperature and 20 mL ethanol and 3.6 grams of decolorizing carbon can be added over 20 minutes to quench the peroxides and form a reaction mixture. A slight exotherm can be observed along with some mild foaming. The slurry can be stirred at room temperature and maintained for overnight. Once the mixture tested negative for peroxides, it can be filtered through celite which can be washed with 100 mL of ethanol to afford a filtrate. The filtrate can be observed as clear and colorless and can be stripped to afford 3.6 grams of the

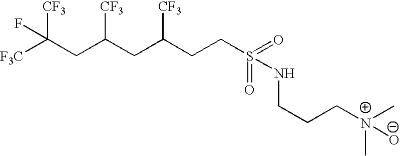

product and can be observed as a yellow oil (58.1% yd). The product structure can be confirmed by NMR and/or chromatographic analysis.

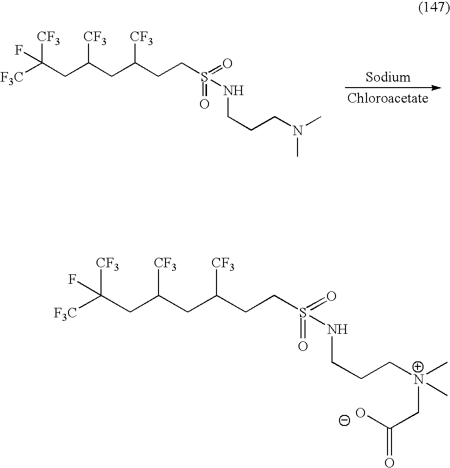

Conforming to scheme (147) above, in a flask that can be equipped with an agitator, thermocouple, reflux condenser, and an addition funnel, 27 mL of ethanol, 1.26 grams (0.011 mole) of sodium chloroacetate and 6 grams (0.011 mole) of

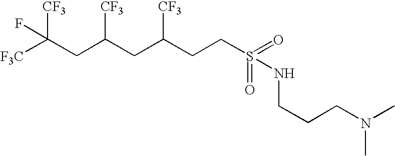

(refer to scheme (145) above) can be placed to form a mixture. The mixture can be heated to reflux for and maintained for about six days. The mixture can be cooled and filtered through celite to afford a filtrate. The filtrate can be stripped of solvent to afford 5.45 grams of the

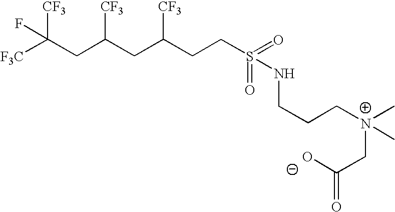

product that can be observed as a yellow fryable foam (82.6% yd.). The product structure can be confirmed by NMR and/or chromatographic analysis.

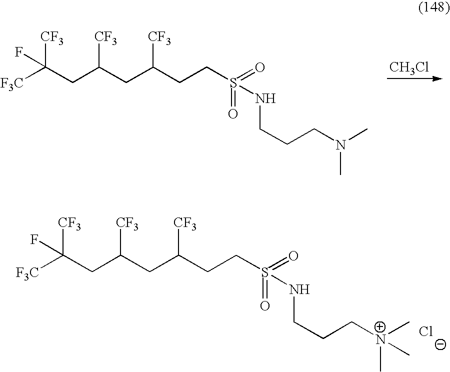

In conformity with scheme (148) above, in a sealable flask that can be equipped with an agitator and a thermocouple, 6 grams (0.11 mole) of

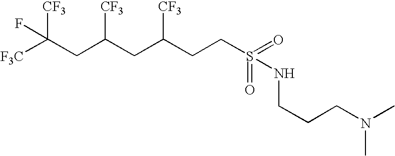

(refer to scheme (145) above) and 22 mL of a 1M solution of chloromethane in diethyl ether can be placed to form a mixture. The mixture can be heated to 50° C. and maintained for overnight. The mixture can be observed to change from a clear tan color to a white slurry. The slurry can be cooled and vented and filtered to afford what can be observed as a white gummy solid (1.3 gram) and a filtrate. The filtrate can be analyzed and observed to contain only starting material. The filtrate can be placed back in the reaction flask along with 25 mL of a 1M solution of chloromethane in diethyl ether to form a reaction mixture and reheated to 50° C. and maintained for 5 days. The reaction mixture can be cooled and vented and filtered to afford what can be observed as a white gummy solid (1.0 gram) and a filtrate. The filtrate can be concentrated to afford what can be observed as a yellow oil (1.0 g) and characterized by 1HNMR (MO6013-63F) and found to be the starting sulfonamide. The sulfonamide can be set aside. The two portions of gummy solids can be combined to afford 2.3 grams of the

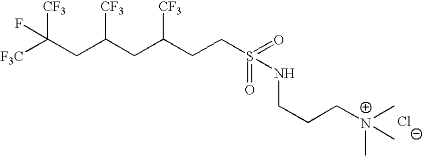

product (34.8% yd.). The product structure can be confirmed by NMR and/or chromatographic analysis.

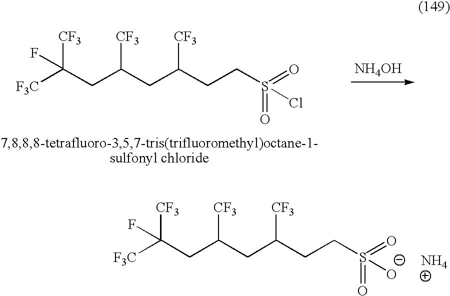

In reference to scheme (149) above, in a flask that can be equipped with an agitator, thermocouple, reflux condenser, and an addition funnel, 6.8 grams (0.01 mole) of 7,8,8,8-tetrafluoro-3,5,7-tris(trifluoromethyl)octane-1-sulfonyl chloride (refer to scheme (144) above) and 7.52 mL of a 2.5 M solution of NH4OH in water can be placed to form a mixture. To the mixture, 30 mL of 1,4 dioxane can be added to form a reaction mixture which can be observed as clear and colorless. The reaction mixture can be allowed to at room temperature for overnight. The reaction mixture can be stripped of dioxane and about 2 L of chloroform can be added and an azeotropic distillation performed in an attempt to remove the water to afford what can be observed as a yellow oil and stripped solvent. The stripped solvent can be placed on a rotoevaporator to afford what can be observed as an off-white semisolid. This semisolid can be combined with the yellow oil. The combination can be placed on a Kugelrohr apparatus (0.03 mmHg, 45° C.) to afford the

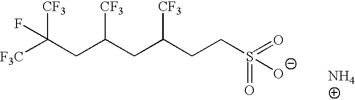

product that can be observed as an off-white semisolid. The product structure can be confirmed by NMR and/or chromatographic analysis.

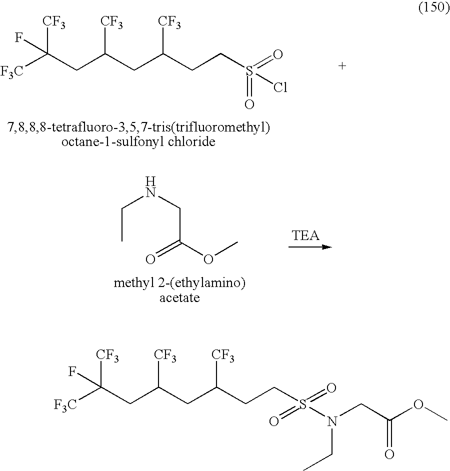

According to scheme (150) above, in a flask that can be equipped with an agitator, thermocouple, reflux condenser, and an addition funnel, 0.5 grams (0.001 mole) of 7,8,8,8-tetrafluoro-3,5,7-tris(trifluoromethyl)octane-1-sulfonyl chloride (refer to scheme (144) above) and 0.12 gram (0.001 mole) of methyl 2-(ethylamino)acetate and 2 mL of chloroform can be placed to form a mixture and chilled to about 0° C. To the mixture, 0.3 mL of triethylamine (TEA) can be added drop-wise to form a reaction mixture. The peak temperature during the addition can be observed to be about 3.0° C. The reaction mixture can be allowed to warm to room temperature and maintained for overnight. To the reaction mixture, 5 mL of cholorform can be added to form a diluent. The diluent can be sequentially washed with two 5 mL portions of a saturated solution of NaHCO3 in water, 5 mL of water and 5 mL of a saturated solution of NaCl to form a multiphase mixture from which an organic phase can be separated from an aqueous phase. The organic phase can be dried and stripped of solvent to afford a concentrate. The concentrate can be observed to contain TEA. To the concentrate, 10 mL of chloroform and washed with 10 mL of a 5% (wt/wt) solution of HCl in water and 10 mL of a 1N solution of NaOH in water to form a multiphase mixture from which an organic phase can be separated from an aqueous phase. The organic phase can be dried and stripped of solvent to afford 0.25 grams of the

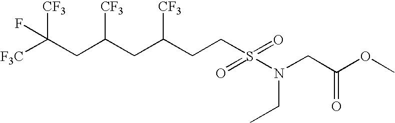

product (43.1% yd.). The product structure can be confirmed by NMR and/or chromatographic analysis.

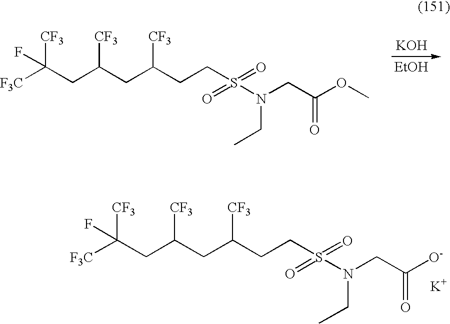

In accordance with scheme (151) above, in a flask that can be equipped with an agitator and a thermocouple, 0.25 gram of

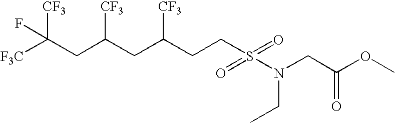

(refer to scheme (150) above), 0.9 mL of ethanol and 0.03 gram of KOH can be placed to form a mixture. The mixture can be allowed to stir at room temperature for overnight.

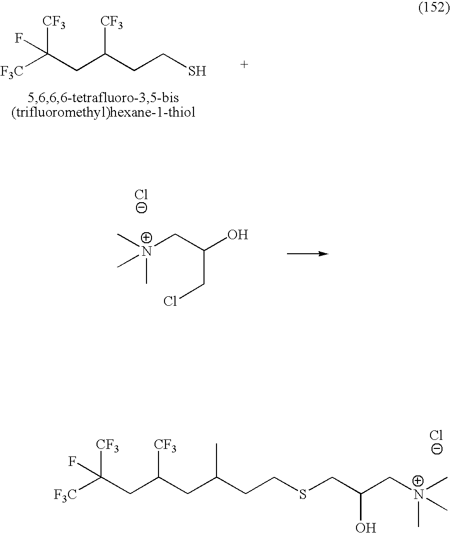

Referring to scheme (152) above, in a flask that can be equipped with an agitator, thermocouple, reflux condenser, and an addition funnel, 3.22 grams of water, 5 grams (0.012 mole) of 5,6,6,6-tetrafluoro-3,5-bis(trifluoromethyl)hexane-1-thiol (refer to scheme (54) above), 3.71 grams (0.012 mole) of 3-chloro-2 hydroxypropyl trimethyl ammonium chloride (60% in water) and 0.47 grams (0.012 mole) of sodium hydroxide can be placed to form a mixture. The mixture can be stirred at room temperature after about 15 minutes the mixture can be observed to warm significantly and thicken into a white semisolid. To the mixture, 5 mL of ethanol can be added to facilitate stirring. The mixture can be stirred at room temperature and maintained for about 5 hours. To the mixture, 100 mL of ethanol can be added and filtered to afford a filtrate and a wet cake. The filtrate can be stripped and three 150 mL portions of ethanol can be added and an azeotropic distillation conducted to afford what can be observed as a yellow residue. The yellow residue can be dissolved in 150 mL of chloroform and filtered to afford a filtrate and a wet-cake. The filtrate can be concentrated to afford what can be observed as a yellow oil and placed on a Kugelrohr apparatus (50° C., 60 minutes, 0.03 mmHg) to afford 6.5 grams of the

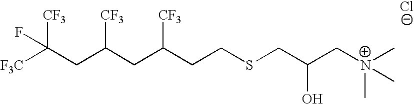

product that can be observed as a yellow oil (95.6% yd.). The product structure can be confirmed by NMR and/or chromatographic analysis.

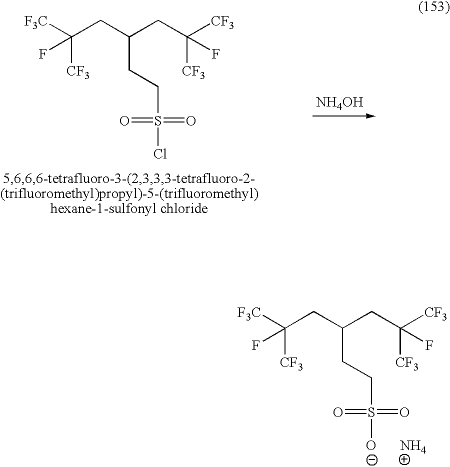

According to scheme (153) above, in a flask that can be equipped with an agitator, thermocouple and an addition funnel, 5 grams (0.01 mole) of 5,6,6,6-Tetrafluoro-3-(2,3,3,3-tetrafluoro-2-(trifluoromethyl)propyl)-5-(trifluoromethyl)hexane-1-sulfonyl chloride (refer to scheme (74) above) and about 9 mL of methanol can be placed to form a mixture. To the mixture, 2.4 mL of a 7.4 M solution of ammonium hydroxide in water can be added drop wise at room temperature to form a first reaction mixture. To the first reaction mixture, addition methanol can be added. The first reaction mixture can be observed as a clear and colorless solution and stirred overnight at room temperature. The first reaction mixture can be concentrated and treated with five 200 mL portions of ethanol to afford a second reaction mixture. The second reaction mixture can be subjected to an azeotropic distillation in effort to remove water to afford a concentrate. The concentrate can be triturated once more in about 200 mL of ethanol (200 mL) and the salts filtered off and discarded to afford a first filtrate. The first filtrate can be concentrated and dissolved in a 100 mL of a 80:20 mixture of chloroform/ethanol and filtered to afford a second filtrate. The second filtrate can be concentrated.

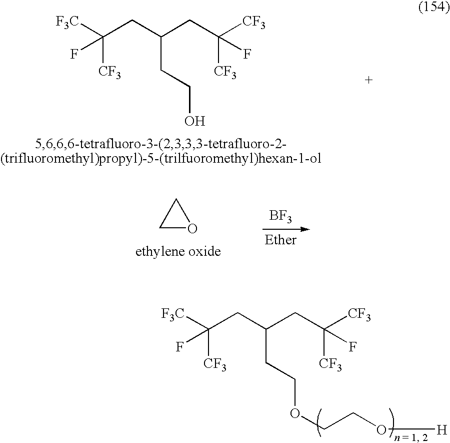

According to scheme (154) above, in a 60 mL autoclave, 18 grams (44.3 mmol) of 5,6,6,6-tetrafluoro-3-(2,3,3,3-tetrafluoro-2-(trifluoromethyl)propyl)-5-(trifluoromethyl)hexan-1-ol (refer to scheme (45) above) can be placed. To the autoclave, 22 grams (4.43 mole) of separately condensed ethylene oxide can be added to form a mixture. To the mixture, 0.15 mL of boron trifluoride etherate can be added to form a reaction mixture and the autoclave can be sealed. The reaction mixture can be slowly heated to 50° C. and maintained for an hour to afford a product mixture having the generalized structure

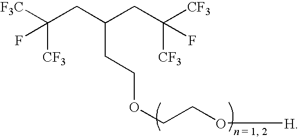

The product structure can be confirmed by NMR and/or chromatographic analysis.

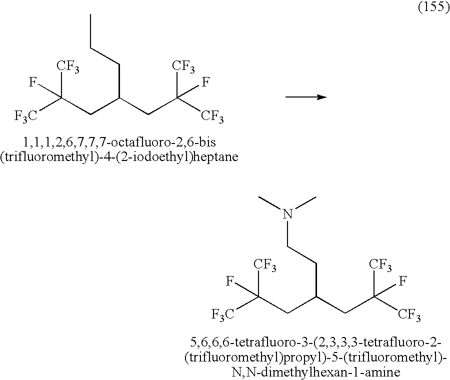

According to scheme (155) above, in a sealed tube 10 grams (0.019 mole) of 1,1,1,2,6,7,7,7-octafluoro-2,6-bis(trifluoromethyl)-4-(2-iodoethyl)heptane (refer to scheme (29) above) and 47 mL of a 2M solution of dimethyl amine in tetrahydrofuran can be placed to form a mixture. The mixture can be heated to 60° C. and maintained for about 2.5 hours. The mixture can be allowed to cool to room temperature and maintained overnight. To the mixture, about 200 mL of ethyl acetate and 200 mL of a saturated solution of NaHCO₃ in water can be added to form a multiphase mixture from which an organic phase can be separated from an aqueous phase. To the aqueous phase, about 200 mL of ethyl acetate can be added to form a multiphase mixture from which an organic phase can be separated from an aqueous phase. The organic phases can be combined, dried, filtered and concentrated to afford 7.2 grams of the 5,6,6,6-tetrafluoro-3-(2,3,3,3-tetrafluoro-2-(trifluoromethyl)propyl)-5-(trifluoromethyl)-N,N-dimethylhexan-1-amine product. The product structure can be confirmed by NMR analysis.

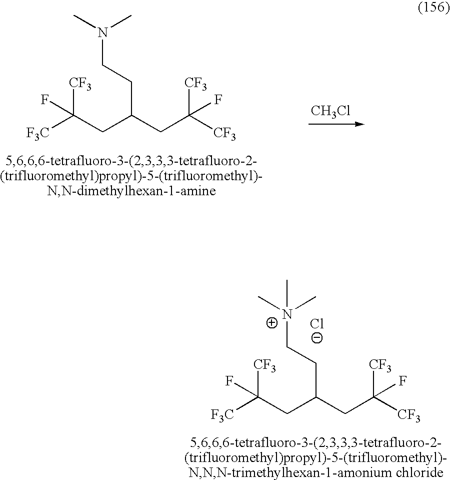

According to scheme (156) above, in a sealed reaction flask, 3.5 grams. (0.008 mole) of 5,6,6,6-tetrafluoro-3-(2,3,3,3-tetrafluoro-2-(trifluoromethyl)propyl)-5-(trifluoromethyl)-N,N-dimethylhexan-1-amine (refer to scheme (155) above) and 8 mL of a 1M solution of chloromethane in t-butyl methyl ether can be placed to form a mixture. The mixture can be heated to 55° C. and maintained overnight. The mixture can be allowed to cool to room temperature and maintained over the weekend. The mixture can be heated to 55° C. and maintained for 2 days. To the mixture, about 8 mL of a 1M solution of chloromethane can be added and maintained overnight. The mixture can be allowed to cool to room temperature and vented. To the mixture, about 50 mL of ethyl acetate can be added to form a diluted mixture. The diluted mixture can be concentrated to afford about 200 mg of the 5,6,6,6-tetrafluoro-3-(2,3,3,3-tetrafluoro-2-(trifluoromethyl)propyl)-5-(trifluoromethyl)-N,N,N-trimethylhexan-1-amonium chloride product that can be observed as a tan solid. The product structure can be confirmed by NMR and/or LCMS analysis.

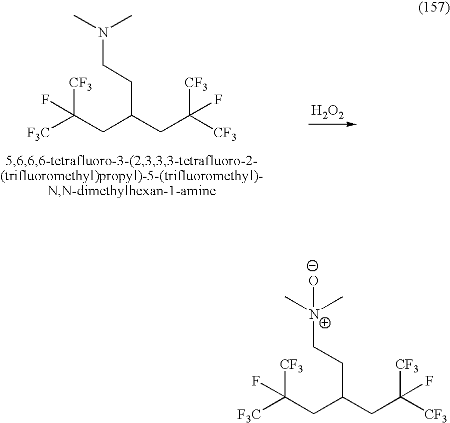

Referring to scheme (157) above, in a flask that can be equipped with an agitator, thermocouple and an addition funnel, 10 grams (0.02 mole) of 5,6,6,6-tetrafluoro-3-(2,3,3,3-tetrafluoro-2-(trifluoromethyl)propyl)-5-(trifluoromethyl)-N,N-dimethylhexan-1-amine (refer to scheme (155) above), about 35 mL of ethanol and 5.5 mL of water can be placed to form a mixture. To the mixture, 21.7 mL of a 50 (wt/wt) percent solution of hydrogen peroxide in water can be added slowly over a period of about 30 minutes to form a reaction mixture. The reaction mixture can be maintained at room temperature overnight. To the reaction mixture, about 35 mL of ethanol and 14 grams of carbon can be added to form a slurry. The slurry can be filtered through celite and the filter cake washed with ethanol to form a filtrate. The filtrate can be concentrated to afford 6.5 grams of the

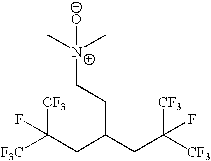

product. The product structure can be confirmed by NMR and/or chromatographic analysis.

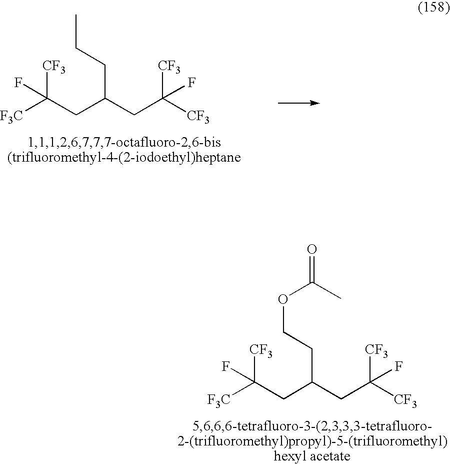

In reference to scheme (158) above, in a flask that can be equipped with an agitator and a thermocouple, 30 grams (0.056 mole) of 1,1,1,2,6,7,7,7-octafluoro-2,6-bis(trifluoromethyl)-4-(2-iodoethyl)heptane (refer to scheme (29) above), 13.82 gram (0.169 mole) of sodium acetate and about 185 mL of dimethylformamide can be placed to form a mixture. The mixture can be heated to 80° C. and maintained for about four hours. The mixture can be poured Into about 250 mL of water and extracted with three portions of 300 mL of ether to form a multiphase mixture from which an organic phase can be separated from an aqueous phase. The organic phases can be combined and washed with about 300 mL of a saturated brine solution to form a multiphase mixture from which an organic phase can be separated from an aqueous phase. The organic phase can be collected, dried, and concentrated by employing a Kugelrohr distillation apparatus at 40° C. for about one hour. The product structure can be confirmed by NMR and/or chromatographic analysis.

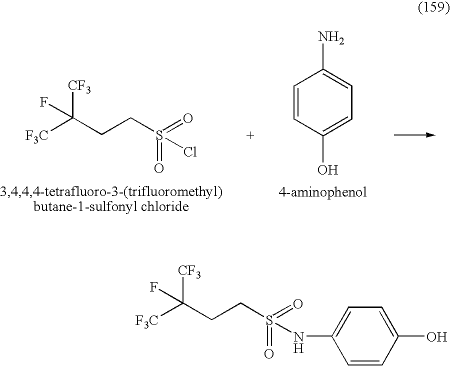

According to scheme (159) above, in a flask that can be equipped with an agitator, thermocouple and an ice water bath, 235.4 grams (2.16 moles) of 4-aminophenol and about 1350 mL of dimethylformamide (DMF) can be combined to form a mixture. The mixture can be warmed until observed as homogeneous. The mixture can be cooled to about 5° C. using the ice water bath. To the mixture, 160 grams (0.54 mole) of 3,4,4,4-tetrafluoro-3-trifluoromethyl-butane-1-sulfonyl chloride (see, e.g., Published International Applications) in about 675 mL of DMF can be added drop wise over the period of about an hour to form a reaction mixture, keeping the temperature below 5° C. The reaction mixture can be allowed to warm to room temperature and maintained for about one hour. The reaction mixture can be poured into about 2100 mL of a 1N solution of hydrochloric acid in water and extracted three 10 mL portions of methylene chloride to form a multiphase mixture from which an organic phase can be separated from an aqueous phase. The organic phases can be combined and washed with about 6 L of water (6 L) to form a multiphase mixture from which an organic phase can be separated from an aqueous phase. The organic phase can be collected, dried, concentrated and placed on the Kugelrohr apparatus at 50° C. and 0.03 mmHg for 10 hours to afford 193 grams of the crude

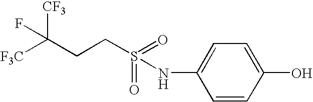

product that can be observed as a viscous dark red oil. The product structure can be confirmed by NMR and/or chromatographic analysis.

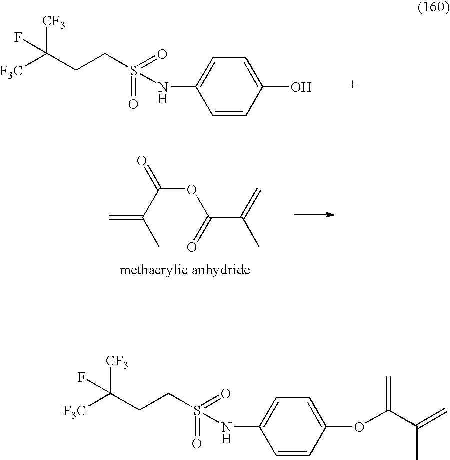

In reference to scheme (160) above, in a flask that can be equipped with an agitator, thermocouple, ice water bath, a nitrogen feed and an addition funnel, 164 grams (0.44 mole) of

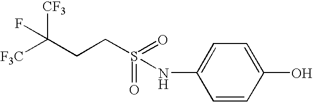

(refer to scheme (159) above), about 1280 mL of methylene chloride and 49.44 grams (0.49 mole) of triethylamine can be placed to form a mixture. The mixture can be chilled to about 0° C. using the ice water bath. To the mixture, 75.3 grams (0.49 mole) of methacrylic anhydride and about 855 mL of methylene chloride (855 mL) can be added drop wise over a period of about one hour to form a reaction mixture. The reaction mixture can be allowed to warm to room temperature and maintained over the weekend. To the reaction mixture, about 15 mL of methylacrylic anhydride can be added and the reaction mixture held at room temperature overnight. To the reaction mixture, about 8 mL of methylacrylic anhydride can be added and maintained overnight. The reaction mixture can be washed successively with about 2200 mL of a 2N solution of HCl in water, two 2200 mL portions of a saturated solution of NaHCO₃ in water, and about 2200 mL of a saturated solution of NaCl in water, wherein each washing step can produce a multiphase mixture from which an organic phase can be separated from an aqueous phase and the organic phase collected and continued to the next step. The organic phase can be collected and concentrated to afford an oil that can be observed as having a dark red color. The oil can be placed on a Kugelrohr apparatus at 75° C./0.03 mmHg for about one hour to afford 219.9 grams of the

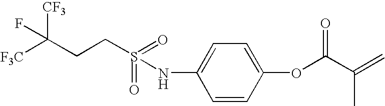

product. The product structure can be confirmed by NMR and/or chromatographic analysis.

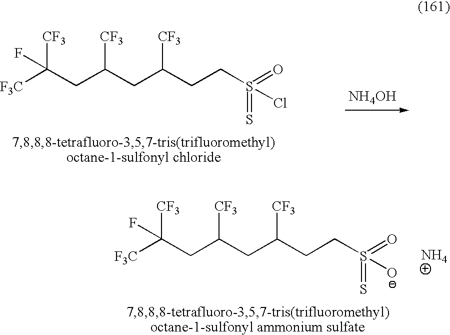

In accordance with scheme (161) above, in a flask that can be equipped with an agitator, thermocouple, reflux condenser, and an addition funnel, 6.8 grams (0.01 mole) of 7,8,8,8-tetrafluoro-3,5,7-tris(trifluoromethyl)octane-1-sulfonyl chloride (refer to scheme (144) above) and 7.52 mL of a 2.5 M solution of NH₄OH in water can be placed to form a mixture. To the mixture, 30 mL of 1,4-dioxane can be added to form a reaction mixture. The reaction mixture can be allowed to stir overnight at room temperature. The reaction mixture can be stripped of dioxane and an azeotropic distillation performed by added about 2 L of chloroform to afford what can be observed as a yellow oil. The yellow oil can be concentrated on a Kugelrohr apparatus (0.03 mmHg, 45° C.) to afford the 7,8,8,8-tetrafluoro-3,5,7-tris(trifluoromethyl)octane-1-sulfonyl ammonium sulfate product that can be observed as an off-white semisolid. The product structure can be confirmed by NMR and/or chromatographic analysis.

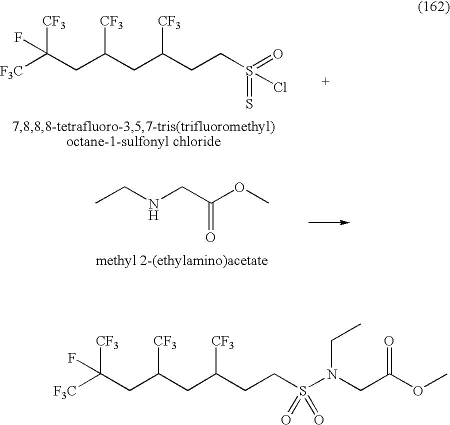

Referring to scheme (162) above, in a flask that can be equipped with an agitator, thermocouple, reflux condenser, and an addition funnel, 0.5 grams (0.001 mole) of 7,8,8,8-tetrafluoro-3,5,7-tris(trifluoromethyl) octane-1-sulfonyl chloride (refer to scheme (144) above), 0.12 grams (0.001 mole) of methyl 2-(ethylamino)acetate and 2 mL of chloroform can be placed to form a mixture and chilled to 0° C. To the mixture, 0.3 mL of triethylamine (TEA) can be added drop wise to form a reaction mixture. The peak temperature during the addition can be observed to be about 3.0° C. The reaction mixture can be allowed to warm to room temperature and maintained for overnight to afford what can be observed as a clear yellow solution. To the clear yellow solution, 5 mL of cholorform can be added to form a diluent. The diluent can be washed with two 5 mL portions of a saturated solution of NaHCO₃ in water, 5 mL of water and 5 mL of a saturated solution of NaCl in water wherein each step in the washing procedure can afford a multiphase mixture from which an organic phase can be separated from an aqueous phase. The organic phase can be dried and stripped of solvent to afford an oil. To the oil 10 mL of chloroform and washed with 10 mL of a 5% (wt/wt) solution of HCl in water and 10 mL of a 1N solution of NaOH in water to afford a multiphase mixture from which an organic phase can be separated from an aqueous phase. The organic phase can be dried and stripped of solvent to afford 0.25 grams of the

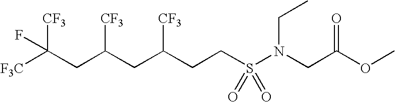

product (43.1% yd.). The product structure can be confirmed by NMR and chromatographic analysis.

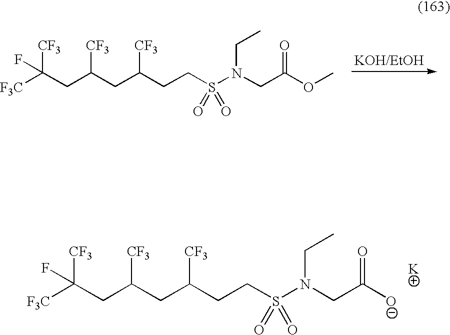

In reference to scheme (163) above, in a flask that can be equipped with an agitator, thermocouple, reflux condenser, and an addition funnel, 0.25 grams of

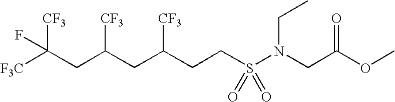

(refer to scheme (162) above) and 0.9 mL of ethanol and 0.03 grams of KOH can be added to form a mixture. The mixture can be allowed to stir at room temperature for overnight. The mixture can be stripped to afford 80 milligrams of the

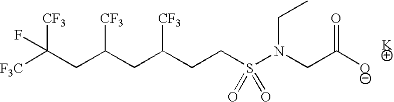

product (30.7% yd.). The product structure can be confirmed by NMR and/or chromatographic analysis.

According to exemplary embodiments, Q_{S} portions can include N-oxide functionality. For example straight-chain R_{F} groups can be coupled to Q_{S} portions having N-oxide functionality to provide useful surfactants.

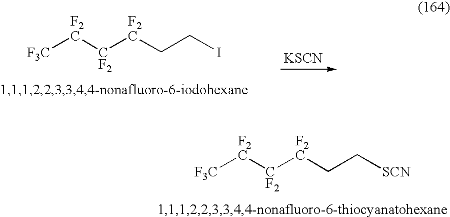

Referring to scheme (164) above, in a flask that can be equipped with an agitator, thermocouple and a reflux condenser, 75 grams (0.2 mole) of solution of 1,1,1,2,2,3,3,4,4-nonafluoro-6-iodohexane (SynQuest Laboratories, INC. Alachua, Fla. 32616-0309), about 150 mL of ethanol, 29.23 grams (0.3 mole) of potassium thiocyanate and 0.75 mL of glacial acetic acid to form a mixture. The mixture can be heated to reflux and maintained for about six hours. The mixture can be observed as a heterogeneous mixture of white salts and yellow liquid. The mixture can be concentrated and about 200 mL of water and about 200 mL of ether can be added to form a multiphase mixture from which an organic phase can be separated from an aqueous phase. The phases can be separated and the aqueous phase twice more extracted with about 200 mL of ether. The organic phases can be combined, dried over sodium sulfate, filtered and concentrated to afford 54 grams of the 1,1,1,2,2,3,3,4,4-nonafluoro-6-thiocyanatohexane product which can be observed as a brown oil. The product structure can be confirmed by NMR and/or GC analysis.

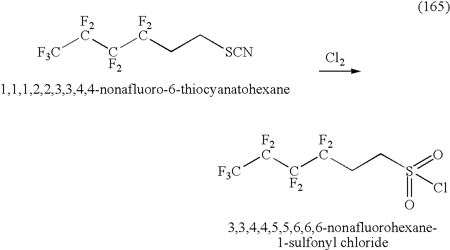

In accordance with scheme (165) above, in a flask that can be equipped with an agitator, thermocouple and a sparging apparatus, 54 grams (0.18 mole) of 1,1,1,2,2,3,3,4,4-nonafluoro-6-thiocyanatohexane (refer to scheme (164) above) and about 175 mL of acetic acid can be placed to form a mixture. The mixture can be heated to about 50° C. and vigorously sparged with chlorine gas for about 3 days to form a reaction mixture. The gas and heat can be discontinued each night and resumed the following morning. The reaction mixture can be allowed to cool and about 6.4 mL of water added. To the reaction mixture, about 200 mL of chloroform and about 200 mL of water to form a multiphase mixture from which an organic phase can be separated from an aqueous phase. The organic phase can be successively washed with two 200 mL portions of a saturated bicarbonate solution one 200 mL portion of a saturated brine solution. The organic phase can be collected and dried over sodium sulfate, filtered and concentrated to afford 58.6 grams of the 3,3,4,4,5,5,6,6,6-nonafluorohexane-1-sulfonyl chloride product that can be observed as a pale oil. The product structure can be confirmed by NMR and/or GC/MS and/or GC analysis.

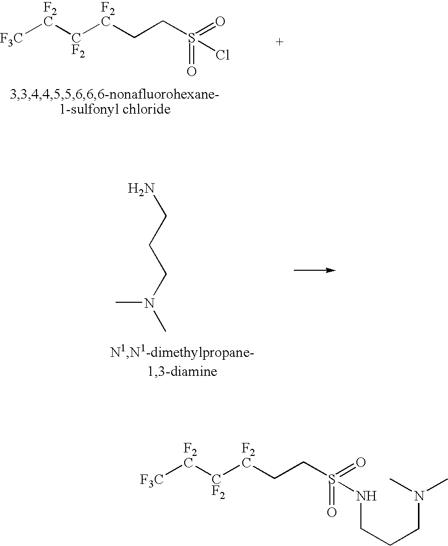

Referring to scheme (166) above, in a flask that can be equipped with an agitator, thermocouple, ice water bath, and an addition funnel, 51.83 mL (0.51 mole) of 3-(dimethylamino)propylamine and about 200 mL of chloroform can be placed to form a first mixture. The first mixture can be cooled to about 0° C. In the addition funnel, 58.6 grams (0.17 mole) of 3,3,4,4,5,5,6,6,6-nonafluorohexane-1-sulfonyl chloride (refer to scheme (165) above) and about 200 mL of chloroform can be placed to form a second mixture. The second mixture can be added drop wise to the first mixture over a period of about an hour to form a reaction mixture. The reaction mixture can be maintained at a temperature below about 5° C. The peak temperature during addition can be about −1.1° C. The reaction mixture can be allowed to warm to room temperature and stir over a period of about one hour. The reaction mixture can be successively washed with two 500 mL portions of a saturated NaHCO₃ solution, one 500 mL portion of a saturated solution of NaCl and one 500 mL portion of water wherein each step can produce a multiphase mixture from which an organic phase can be separated from an aqueous phase and each organic phase can be collected and transferred to the next step. The final organic phase can be dried and concentrated to afford 61.6 grams of the

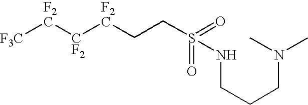

product which can be observed as a brown oil. The product structure can be confirmed by NMR and/or LCMS analysis.

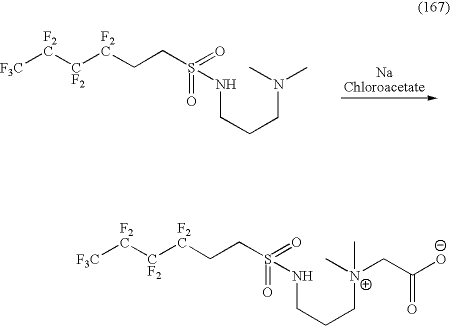

In reference to scheme (167) above, in a flask that can be equipped with an agitator, thermocouple, and a reflux condenser, about 91 mL of, ethanol, 4.24 grams (0.036 mole) of sodium chloroacetate and 15 grams (0.036 mole) of

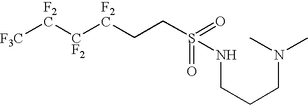

(refer to scheme (166) above) can be placed to form a mixture. The mixture can be heated to reflux for and maintained for about 3 days. The mixture can be allowed to cool, filtered and concentrated to afford 13.5 grams of the

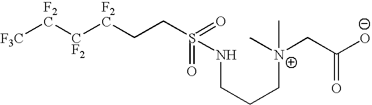

product. The product structure can be confirmed by NMR and/or LCMS analysis.

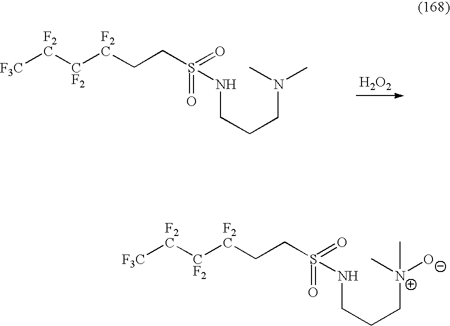

In conformity with scheme (168) above, in a flask that can be equipped with an agitator, thermocouple, ice water bath and an addition funnel, 15 grams (0.036 mole) of

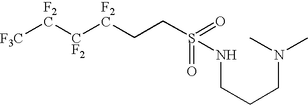

(refer to scheme (166) above) and about 30.4 mL of ethanol and about 4.5 mL of water to form a mixture. To the mixture, 17.2 mL of a 50% (wt/wt) solution of hydrogen peroxide in water can be added over a period of about 30 minutes to form a reaction mixture. The reaction mixture can be observed to have peak temperature during addition of 32° C. The reaction mixture can be heated to and maintained at 35° C. for about 5 hours. The reaction mixture can be allowed to cool to room temperature. To the reaction mixture, about 30 mL of ethanol and 11.25 grams of decolorizing carbon can be added over a period of about 20 minutes to form a slurry. A slight exotherm can be observed along with some foaming during the addition. The slurry can be heated to about 50° C. and maintained for about three hours. The slurry can be filtered through celite and the filter cake washed with about 200 mL of ethanol to afford a filtrate that can be observed as clear and colorless. The filtrate can be concentrated to afford 10.3 grams of the

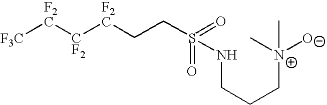

product that can be observed as a white solid. The product structure can be confirmed by NMR and/or LCMS analysis.

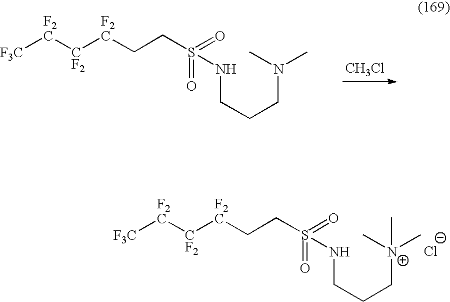

Referring to scheme (169) above, in a flask that can be equipped with an agitator and a thermocouple, 15 grams (0.036 mole) of

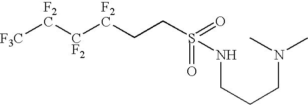

(refer to scheme (166) above) in about 37 mL of a 1M solution of chloromethane in tert-butyl methyl ether to form a mixture. The mixture can be heated to about 55° C. and maintained overnight. The mixture can be observed as a white semi-solid. To the mixture, a sufficient portion of ethyl acetate can be added to form a reaction mixture. The reaction mixture can be concentrated, diluted with about 300 mL of ether and filtered to afford 10.3 grams of the

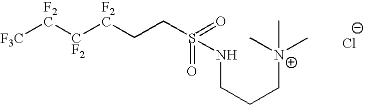

product. The product structure can be confirmed by NMR and/or LCMS analysis.

According to another embodiment, a mercaptan R_{F}-intermediate may also be produced by reacting a iodine R_{F}-intermediate with thiourea to make the isothiuronium salt and treating the isothiuronium salt with sodium hydroxide to give the mercaptan R_{F}-intermediate plus sodium iodide, as described in U.S. Pat. No. 3,544,663 herein incorporated by reference.

In an exemplary aspect of the disclosure, the mercaptan R_{F}-intermediate may be attached to a Qs portion such as group 2-acrylamido-2-methyl-1 propane sulfonic acid available from Lubrizol as AMPS 2403, as generally described in U.S. Pat. No. 4,000,188 herein incorporated by reference.

Aminoxides of the R_{F}-surfactants can be produced according to processes that include those generally described in U.S. Pat. No. 4,983,769, herein incorporated by reference. Accordingly, sulfoamidoamines can be combined with ethanol and water and 70% (wt/wt) hydrogen peroxide and heated to at least 35° C. for 24 hours. Activated carbon can be added and the mixture and refluxed for about 2 hours. The reaction mixture can be filtered and the filtrate evaporated to dryness to provide the amine oxide of the R_{F}-surfactant.

In accordance with another embodiment of the disclosure, processes are provided that can be used to alter the surface tension of a part of a system having at least two parts. The system can include liquid/solid systems, liquid/gas systems, gas/solid systems, and/or liquid/liquid systems. In an exemplary embodiment, the liquid/liquid systems can have one part that includes water and another part that includes a liquid that is relatively hydrophobic when compared to water. According to another example, the liquid/liquid system can contain one part that is relatively hydrophobic when compared to water and/or relatively hydrophobic when compared to another part of the system. R_{F}-surfactants can be used to alter the surface tension of a part of the system, for example, by adding the R_{F}-surfactant to the system.

R_{F}-surfactants may be used as relatively pure solutions or as mixtures with other components. For example, and by way of example only, the R_{F}-surfactants can be added to a system and the surface tension of the system determined by the Wilhelmy plate method and/or using the Kruss Tensiometer method.

As another example, the surface tensions of

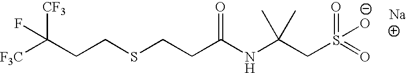

at various concentrations can be determined and the data as indicated in Plot #1 below.

### Surface Tension Plot #1

As another example, the surface tension of

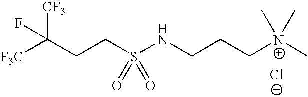

at two (wt/wt) percent in deionized water can be determined to be an average of about 29.9 mN/m.

As another example,

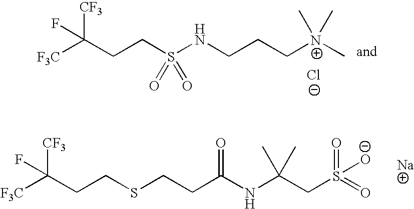

can be combined in substantially equal proportions and formulated in water at various concentrations can be determined and the data as indicated in Plot #2 below.

As another example, the surface tension of

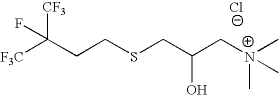

at 2 (wt/wt) percent in deionized water can be observed to afford a surface tension average value of about 31.2 mN/m.

As another example, the surface tension of

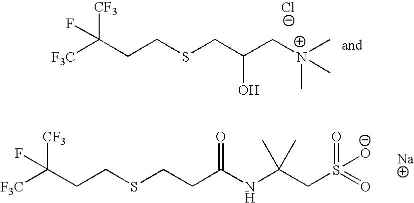

can be combined in substantially equal proportions in water at various concentrations can be determined and the data as indicated in Plot #3 below. Combinatorial effect can be illustrated by the data in the table below.
Combinatorial effect can be illustrated by the data in table 11 below.

As another example, the surface tensions of

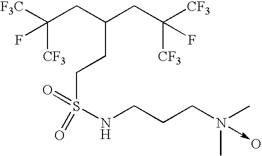

at various concentrations at a pH of about 5, can be determined and the data as indicated in Plot #4 below.

As another example, the surface tensions of,

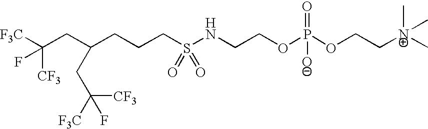

at about pH 7.2 to about pH 8.3, various concentrations can be determined and the data as indicated in Plot #5 below.

As another example the surface tensions of,

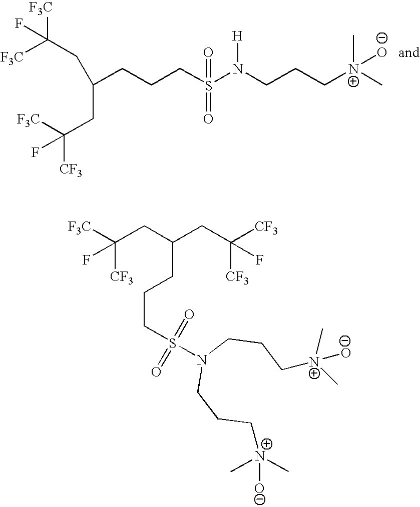

can be combined in substantially equal proportions and formulated in water at various concentrations can be determined and the data as indicated in Plot #6 below.

As another example the surface tensions,

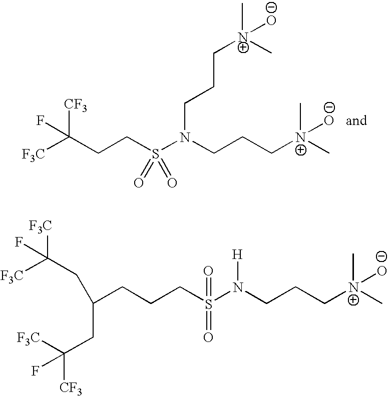

combined in substantially equal proportions and formulated in water at various concentrations can be determined and the data as indicated in Plot #7 below.
As another example,

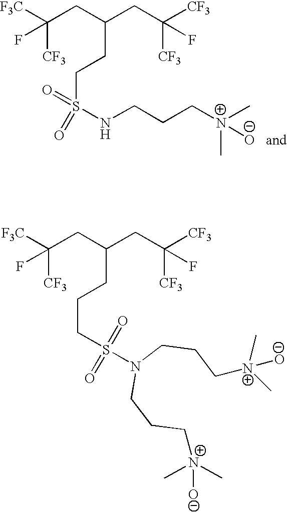

can be combined in substantially equal proportions and formulated in water at various concentrations can be determined and the data as indicated in Plot #8 below.

As another example,

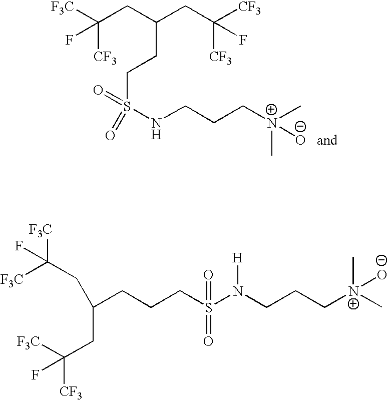

can be combined in substantially equal molar proportions and formulated in water at various concentrations can be determined and the data as indicated in Plot #9 below.

As another example, the surface tensions of,

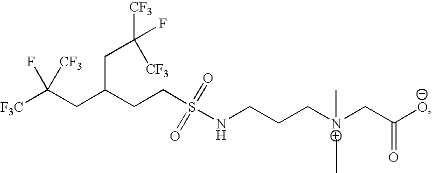

at various concentrations can be determined and the data as indicated in Plot #10 below.

As another example, the surface tensions of

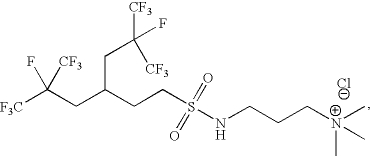

at various concentrations can be determined and the data as indicated in Plot #11 below.

As another example, the surface tensions of

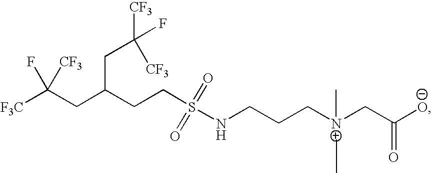

at various concentrations can be determined and the data as indicated in Plot #12 below.

As another example, the surface tensions of

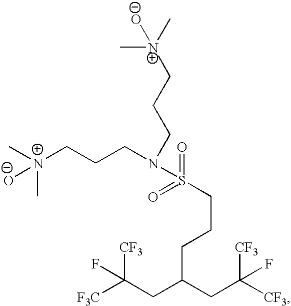

at various concentrations can be determined and the data as indicated in Plot #13 below.

As another example, the surface tensions of

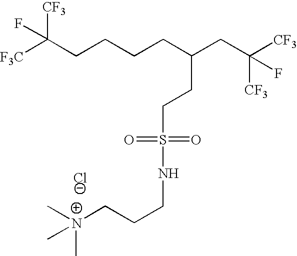

at various concentrations can be determined and the data as Indicated in Plot #14 below.

As another example, the surface tensions of

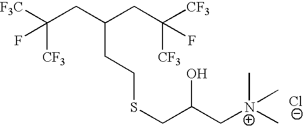

at various concentrations can be determined and the data as indicated in Plot #15 below.

As another example, the surface tensions of

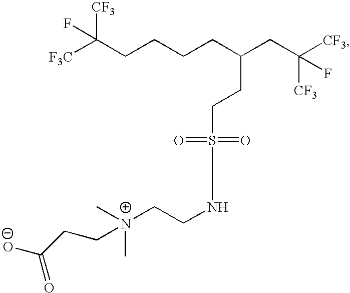

at various concentrations can be determined and the data as indicated in Plot #16 below.

As another example,

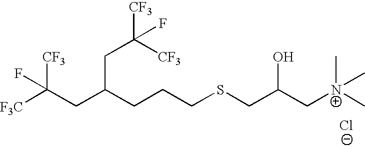

at various concentrations can be determined and the data as indicated in Plot #17 below.

As another example, the surface tensions of

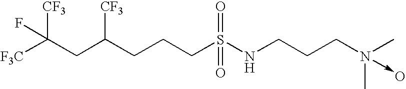

at various concentrations can be determined and the data as indicated in Plot #18 below.

As another example, the surface tensions of

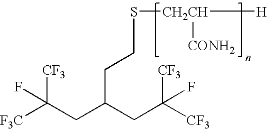

at various concentrations can be determined and the data as indicated in Plot #19 below.

As another example, the surface tensions of

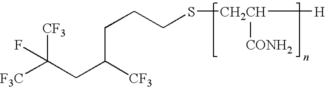

at various concentrations can be determined and the data as indicated in Plot #20 below.

As another example, the surface tensions of

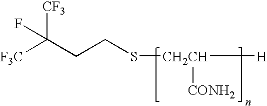

at various concentrations can be determined and the data as indicated in Plot #21 below.

As another example, the surface tensions of

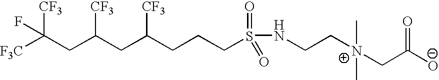

at various concentrations can be determined and the data as indicated in Plot #22 below.

As another example, the surface tensions of

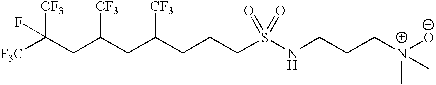

at various concentrations can be determined and the data as indicated in Plot #23 below.

As another example, the surface tensions of

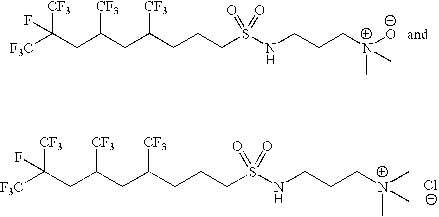

combined in substantially equal proportions and formulated in water at various concentrations can be determined and the data as indicated in Plot #24 below.

As another example, the surface tensions of

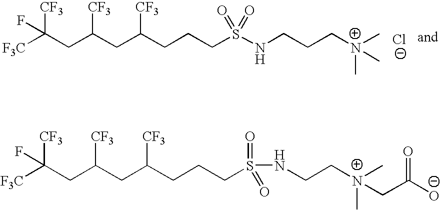

combined in substantially equal proportions and formulated in water at various concentrations can be determined and the data as indicated in Plot #25 below.

As another example, the surface tensions of

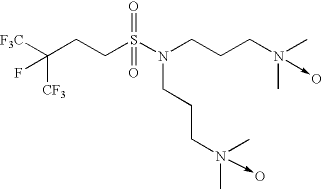

at various concentrations can be determined and the data as indicated in Plot #26 below.

As another example, the surface tensions of

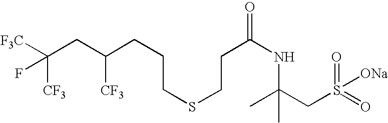

at various concentrations can be determined and the data as indicated in Plot #27 below.

As another example, the surface tensions of

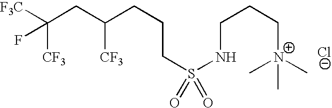

at various concentrations can be determined and the data as indicated in Plot #28 below.

As another example, the surface tensions of

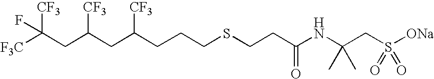

at various concentrations can be determined and the data as indicated in Plot #29 below.

As another example, the surface tensions of

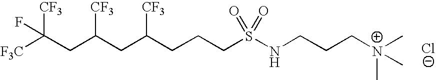

at various concentrations can be determined and the data as indicated in Plot #30 below.

As another example, the surface tensions of

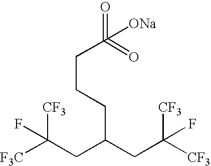

at various concentrations can be determined and the data as indicated in Plot #31 below.

As another example, the surface tensions of

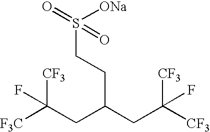

at various concentrations can be determined and the data as indicated in Plot #32 below.

As another example, the surface tensions of

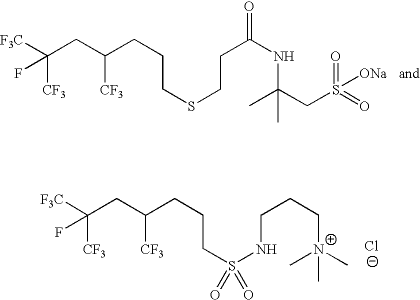

combined in substantially equal proportions and formulated in water at various concentrations can be determined and the data as indicated in Plot #33 below.

As another example, the surface tensions of

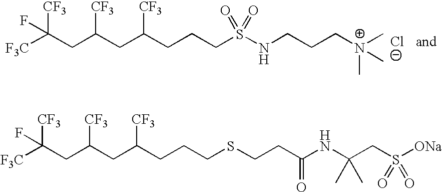

combined in substantially equal proportions and formulated in water at various concentrations can be determined and the data as indicated in Plot #34 below.

As another example, the surface tensions of

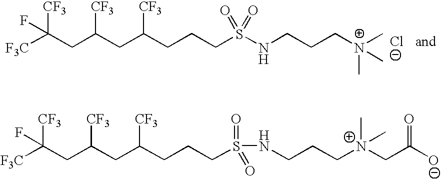

combined in substantially equal proportions and formulated in water at various concentrations can be determined and the data as indicated In Plot #35 below.

As another example, the surface tensions of

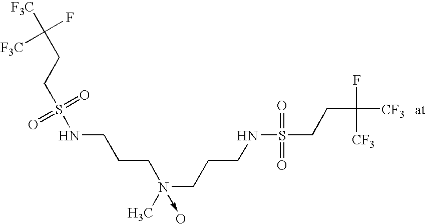

various concentrations can be determined and the data as indicated in Plot #36 below.

As another example, the surface tensions of

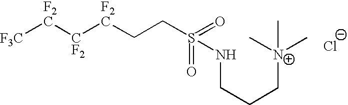

at various concentrations can be determined and the data as indicated in Plot #37 below.

As another example, the surface tensions of

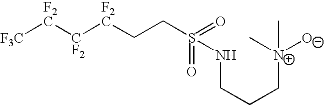

at various concentrations can be determined and the data as indicated in Plot #38 below.

As another example, the surface tensions of

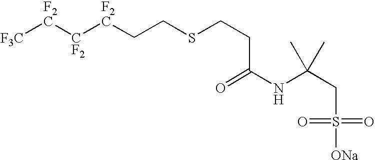

at various concentrations can be determined and the data as indicated in Plot #0.39 below.

As another example, the surface tensions of

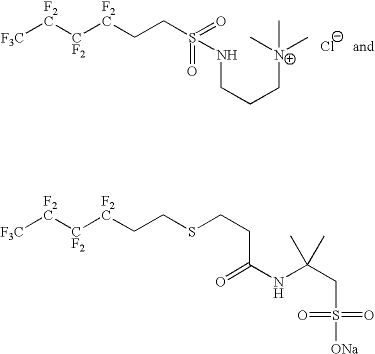

combined in substantially equal proportions and formulated in water at various concentrations can be determined and the data as indicated in Plot #40 below.

As another example, the surface tensions of

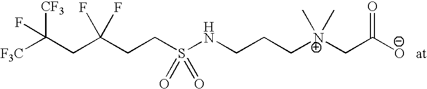

various concentrations can be determined and the data as indicated in Plot #41 below.

As another example, the surface tensions of

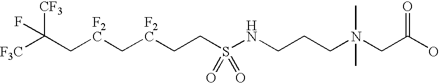

at various concentrations can be determined and the data as indicated in Plot #42 below.

As another example, the surface tensions of

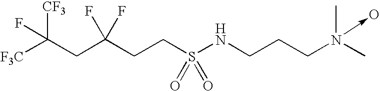

at various concentrations can be determined and the data as indicated in Plot #43 below.

As another example, the surface tensions of

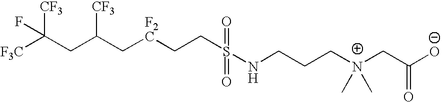

at various concentrations can be determined and the data as indicated in Plot #44 below.

As another example, the surface tensions of

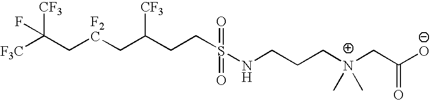

at various concentrations can be determined and the data as indicated in Plot #45 below.

As another example, the surface tensions of

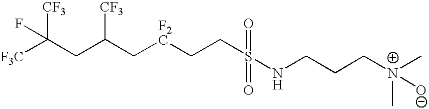

at various concentrations can be determined and the data as indicated In Plot #46 below.

As another example, the surface tensions of

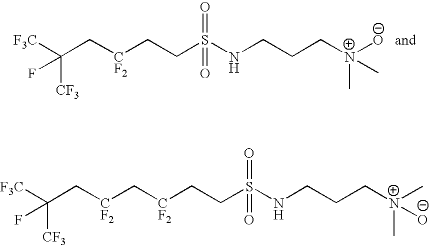

at various concentrations can be determined and the data as indicated in Plot #47 below.

As another example, the surface tensions of

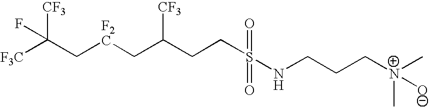

at various concentrations can be determined and the data as indicated in Plot #48 below.

As another example, the surface tensions of

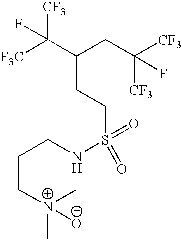

at various concentrations can be determined and the data as indicated in Plot #49 below.

As another example, the surface tensions of

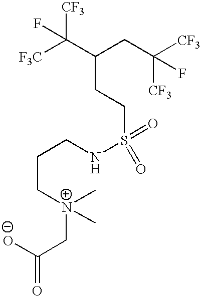

at various concentrations can be determined and the data as indicated in Plot #50 below.

As another example, the surface tensions of

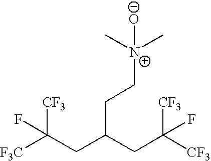

at various concentrations can be determined and the data as indicated in Plot #51 below.

As another example, the surface tensions of

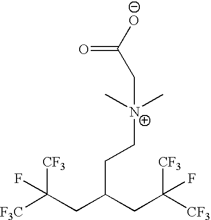

at various concentrations can be determined and the data as indicated in Plot #52 below.

As another example, the surface tensions of

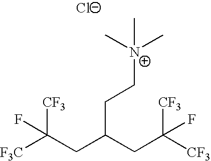

at various concentrations can be determined and the data as indicated in Plot #53 below.

As another example, the surface tensions of

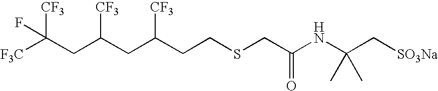

at various concentrations can be determined and the data as indicated in Plot #54 below.

As another example, the surface tensions of

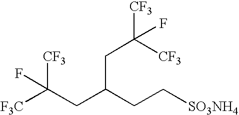

at various concentrations can be determined and the data as indicated in Plot #55 below.

As another example, the surface tensions of

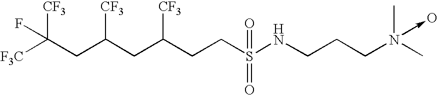

at various concentrations can be determined and the data as indicated in Plot #56 below.

As another example, the surface tensions of

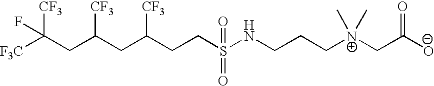

at various concentrations can be determined and the data as indicated in Plot #57 below.

As another example, the surface tensions of

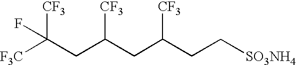

at a concentration of 0.25% (wt/wt) in deionized water can be determined to be about 24 (mN/m).

As another example, the surface tensions of

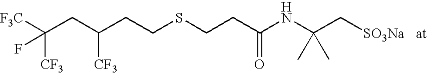

various concentrations can be determined and the data as indicated in Plot #58 below.

As another example, the surface tensions of

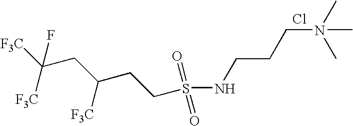

at various concentrations can be determined and the data as indicated in Plot # 59 below.

As another example, the surface tensions of

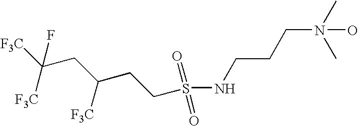

at various concentrations can be determined and the data as indicated in Plot #60 below.

As another example, the surface tensions of

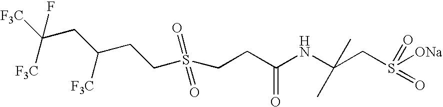

at various concentrations can be determined and the data as indicated in Plot #61 below.

As another example, the surface tensions of

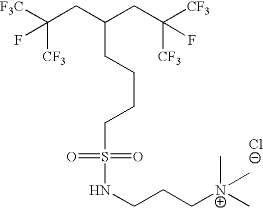

at various concentrations can be determined and the data as indicated in Plot #62 below.

As another example, the surface tensions of

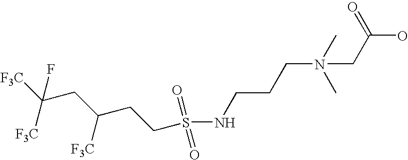

at various concentrations can be determined and the data as indicated in Plot #63 below.

As another example, the surface tensions of

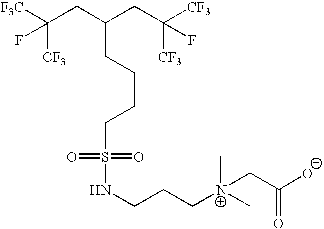

at various concentrations can be determined and the data as indicated in Plot #64 below.

As another example, the surface tensions of

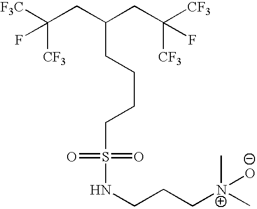

at various concentrations can be determined and the data as indicated in Plot #65 below.

As another example, the surface tensions of

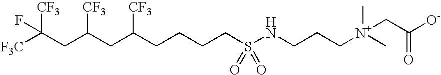

at various concentrations can be determined and the data as indicated in Plot #66 below.

As another example, the surface tensions of

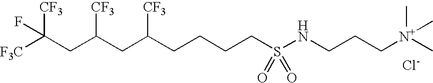

at various concentrations can be determined and the data as indicated in Plot #67 below.

As another example, the surface tensions of

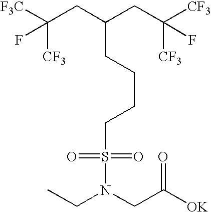

at various concentrations can be determined and the data as indicated in Plot #68 below.

As another example, the surface tensions of

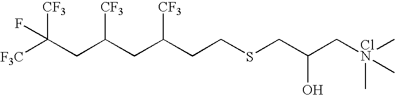

at various concentrations can be determined and the data as indicated in Plot #69 below.

As another example, the surface tensions of

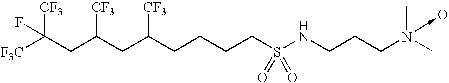

at various concentrations can be determined and the data as indicated in Plot #70 below.

As another example, the surface tensions of

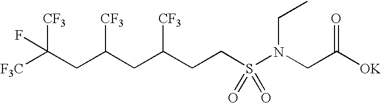

at various concentrations can be determined and the data as indicated in Plot #71 below.

As another example, the surface tensions of

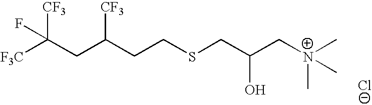

at various concentrations can be determined and the data as indicated in Plot #72 below.

As another example, the surface tensions of

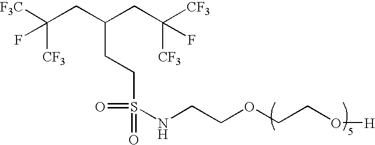

at various concentrations can be determined and the data as indicated in Plot #73 below.

As another example, the surface tensions of

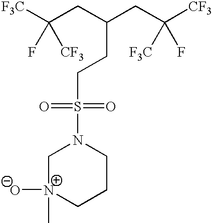

at various concentrations can be determined and the data as indicated in Plot #74 below.

TABLE 11

| 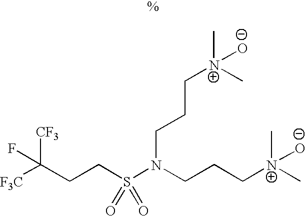 | Test solution gram | 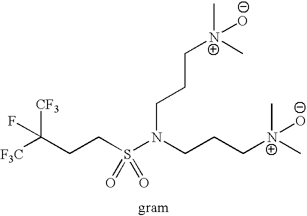 | DI Water gram |
|---|---|---|---|
| 2.0 | 100 | 2.0 gm | 98 |
| 1.0 | 100 | 50 grams of 2% Solution | 50 |
| 0.5 | 100 | 50 grams of 2% Solution | 50 |
| 0.25 | 100 | 50 grams of 2% Solution | 50 |
| 0.125 | 100 | 50 grams of 2% Solution | 50 |

TABLE 12

| Sample # | 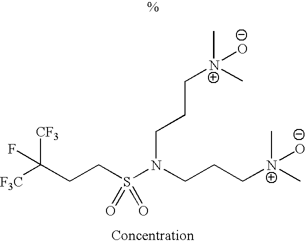 | Avg. Surface Tension Dynes/cm (mN/m) | Sample pH |
|---|---|---|---|
| 1 | 2.0 | 20.7 | 5.5 |
| 2 | 1.0 | 21.9 | 5.5 |
| 3 | 0.5 | 28.8 | 5.5 |
| 4 | 0.25 | 31.2 | 5.5 |
| 5 | 0.125 | 33.1 | 5.5 |

TABLE 13

| 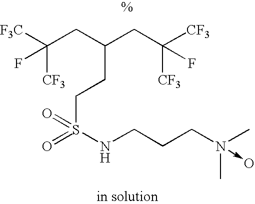 | Test solution gram | 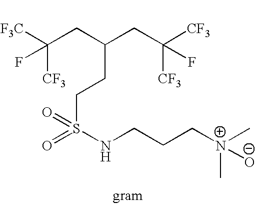 | DI Water gram |
|---|---|---|---|
| 2.0 | 100 | 2.0 gm | 98 |
| 1.0 | 100 | 50 grams of 2% Solution | 50 |
| 0.5 | 100 | 50 grams of 1% Solution | 50 |
| .05 | 100 | 2.5 grams of 2% solution | 97.5 |
| .025 | 100 | 50 grams of .05% solution | 50 |
| .015 | 100 | 3 grams of 0.5% Solution | 97 |
| 0.0125 | 100 | 50 grams of .025% Solution | 50 |

TABLE 14

| Sample # | 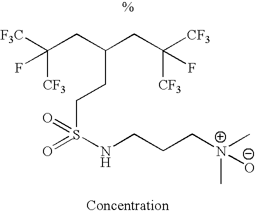 | Avg. Surface Tension Dynes/cm (mN/m) |
|---|---|---|
| 1 | 2.0 | 19.9 |
| 2 | 1.0 | 19.8 |
| 3 | 0.5 | 19.9 |
| 4 | 0.05 | 20.07 |
| 5 | 0.025 | 20.0 |
| 6 | 0.015 | 23.7 |
| 7 | 0.0125 | 24.5 |

An exemplary surfactant testing formulation can be prepared by the following example. In a flask, 2.0 grams of 6,7,7,7-tetrafluoro-4-(2,3,3,3-tetrafluoro-2-(trifluoromethyl)propyl)-6-(trifluoromethyl)heptane-1-sulfonic acid bis-(3-dimethylamino-propyl)amide can be dissolved in about 98 mL of deionized water to prepare a testing solution that can be observed to be clear and have a pH of about 5.1. Additional testing solutions of varying concentrations can be made according to table 15 below.

TABLE 15
List of Components in Testing Solutions

| % Surfactant | | |
|---|---|---|
| in Testing | Surfactant | Deionized Water |
| Solution | (gram) | (gram) |
| 2.0 | 2.0 | 98 |
| 1.0 | 50 grams of the 2 % Solution | 50 |
| 0.5 | 50 grams of the 1 % Solution | 50 |
| 0.25 | 50 grams of the 0.5 % Solution | 50 |
| 0.125 | 50 grams of the 0.25 % Solution | 50 |
| 0.01 | 0.5 grams of the 2.0 % Solution | 99.5 |

TABLE 16
Effect of Surfactant Concentration on Surface Tension

| Sample # | 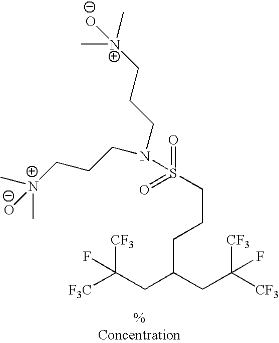 | Avg. Surface Tension Dynes/cm (mN/m) | Sample pH |
|---|---|---|---|
| 1 | 2.0 | 20.7 | 5.1 |
| 2 | 1.0 | 20.7 | 5.1 |
| 3 | 0.5 | 22.1 | 5.1 |
| 4 | 0.25 | 20.8 | 5.2 |
| 5 | 0.125 | 20.3 | 5.4 |
| 6 | 0.01 | 26.3 | 5.3 |

Surface tensions and corresponding concentrations of R_{F}-surfactants are denoted in Tables 17-19 below.

TABLE 17
R_{F}-Surfactant Surface Tensions

| | Surface | |
|---|---|---|
| | Tension | Concentration |
| R_{F}-surfactant | (mN/m) | % (wt/wt) |
| 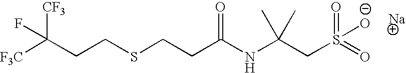 | 33.1 | 2 |
| 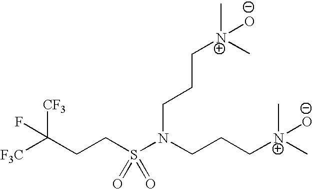 | 20.7 | 2 |
| 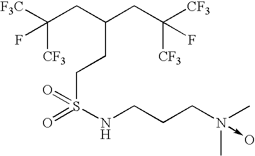 | 19.8 | 1.0 |
| 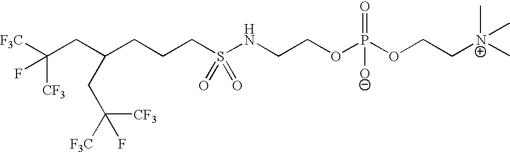 | 20.2 | 0.06 |
| 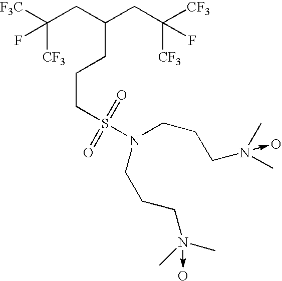 | 20.3 | 0.125 |
| 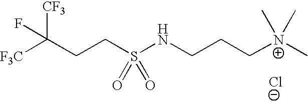 | 23.6 | 3.5 |
| 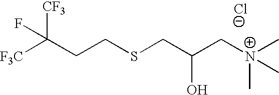 | 31.2 | 2.0 |
| 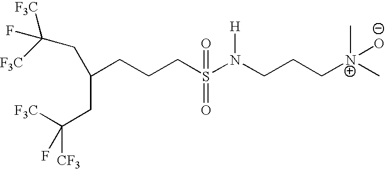 | 19.8 | 0.05 |
| 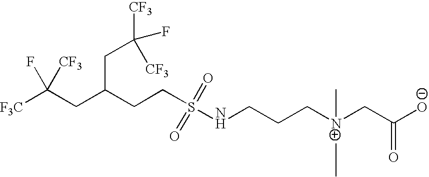 | 20.0 | 0.06 |
| 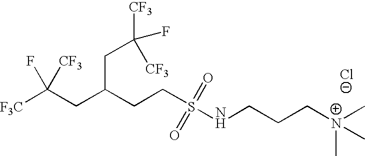 | 21.2 | 1.0 |
| 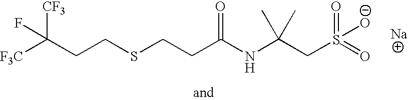 | 20.3 | 2.0 |
| 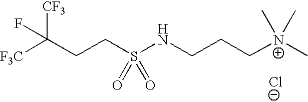 | | |
| 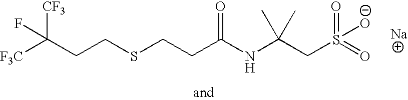 | 21.0 | 2.0 |
| 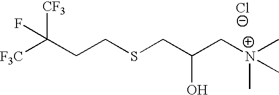 | | |
| 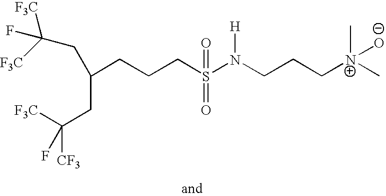 | 20.2 | 0.25 |
| 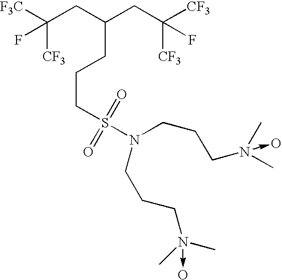 | | |
| 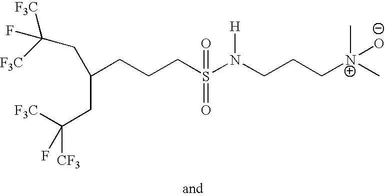 | 19.9 | 0.03 |
| 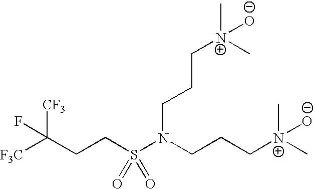 | | |
| 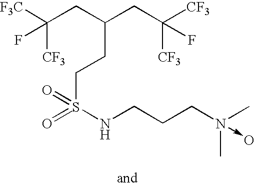 | 19.8 | 0.25 |
| 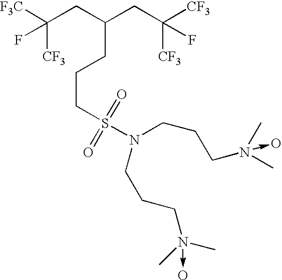 | | |
| 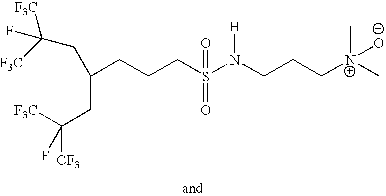 | 19.3 | 0.25 |
| 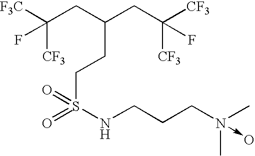 | | |
| 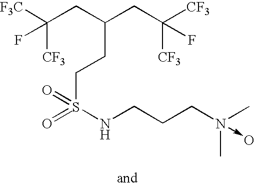 | 19.3 | 0.125 |
| 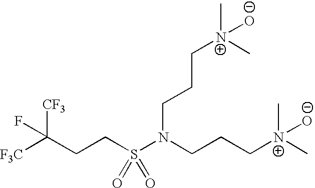 | | |

R_{f}-Surfactant Surface Tension

| | | Surface | |
|---|---|---|---|
| | | Tension | Concentration |
| | R_{f}-Surfactant | (mN/m) | % (wt/wt) |
| 1 | 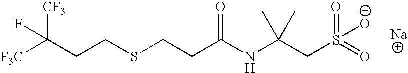 | 33.1 | 2.0 |
| 2 | 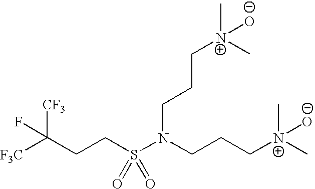 | 20.7 | 2.0 |
| 3 | 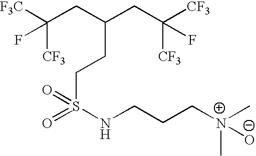 | 19.8 | 1.0 |
| 4 | 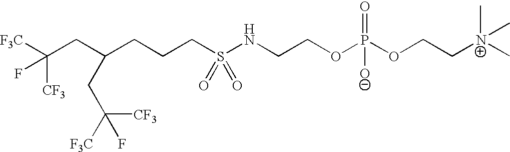 | 20.2 | 0.06 |
| 5 | 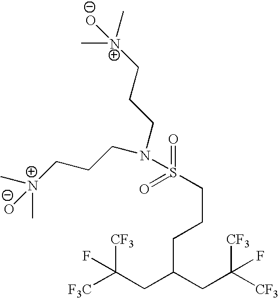 | 20.3 | 0.125 |
| 6 | 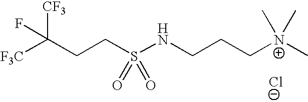 | 23.6 | 3.5 |
| 7 | 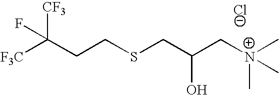 | 31.2 | 2.0 |
| 8 | 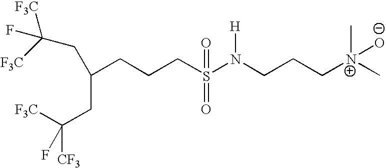 | 19.8 | 0.05 |
| 9 | 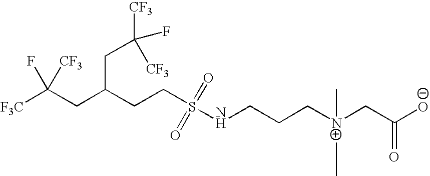 | 20.0 | 0.06 |
| 10 | 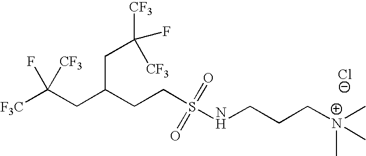 | 21.2 | 1.0 |
| 11 | 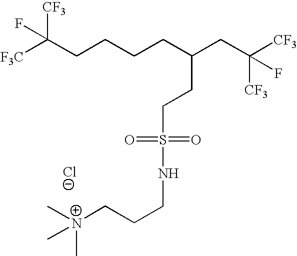 | 21.4 | 2.0 |
| 12 | 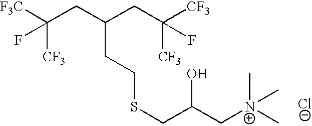 | 22.2 | 1.0 |
| 13 | 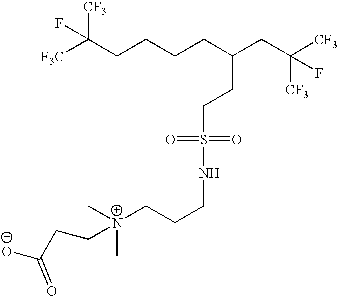 | 21.6 | 0.016 |
| 14 | 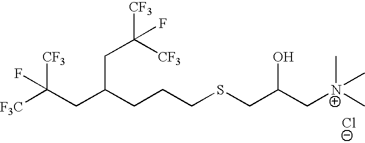 | 20.2 | 2.0 |

TABLE 18
Summary of Combinatorial Surface Tension Values

| | | Surface Tension |
|---|---|---|
| | | (mN/m)/Concentration |
| | | (wt/wt) in |
| R_{f}-Surfactant Combination | | Deionized Water |
| A | 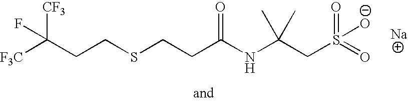 | 20.3/2.0 |
| | 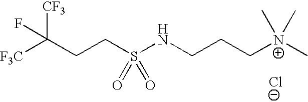 | |
| B | 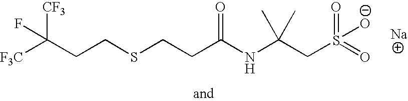 | 21.0/2.0 |
| | 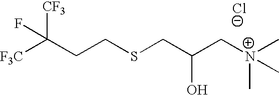 | |
| C | 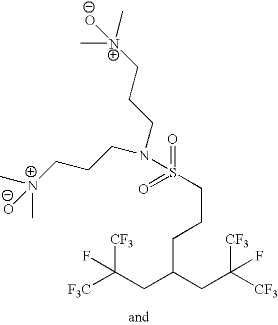 | 20.2/0.25 |
| | 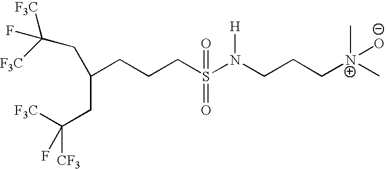 | |
| D | 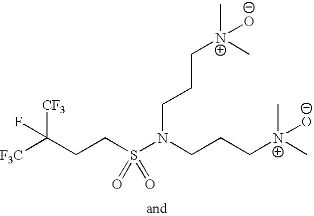 | 19.9/0.03 |
| | 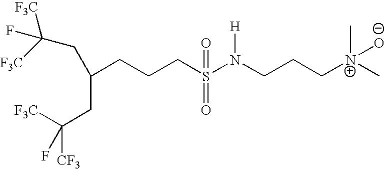 | |
| E | 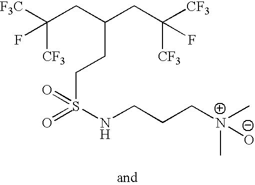 | 19.8/0.25 |
| | 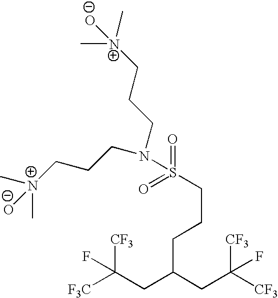 | |
| F | 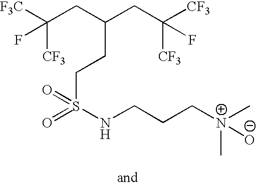 | 19.3/0.25 |
| | 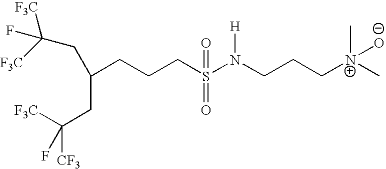 | |
| G | 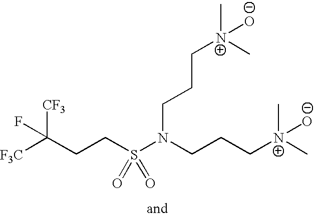 | 19.3/0.125 |
| | 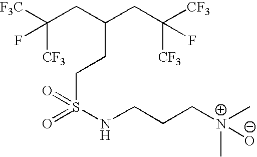 | |

TABLE 19
R_{F}-Surfactant Surface Tensions

| | Surface | |
|---|---|---|
| | Tension | Concentration |
| R_{F}-Surfactant | (mN/m) | % (wt/wt) |
| 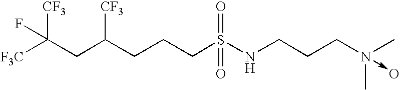 | 18.9 | 2 |
| 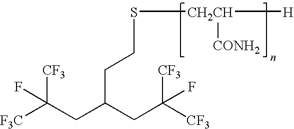 | 18 | 0.25 |
| 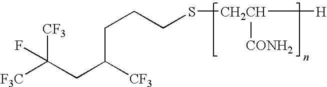 | 19 | 1 |
| 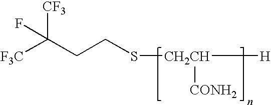 | 23.4 | 2 |
| 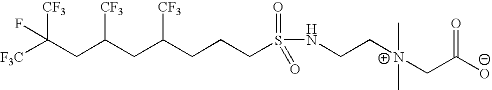 | 18.9 | 1 |
| 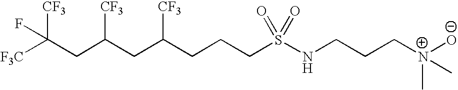 | 18.9 | 0.125 |
| 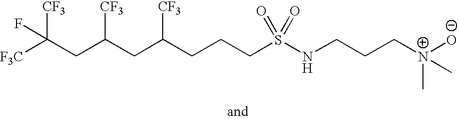 | 19.5 | 0.025 |
| 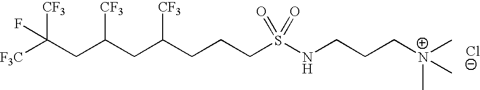 | | |
| 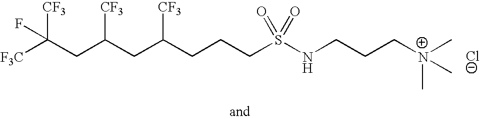 | 20.5 | 0.02 |
| 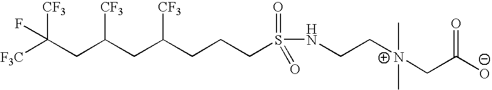 | | |
| 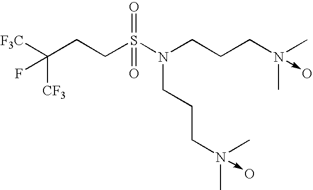 | 20.3 | 2 |
| 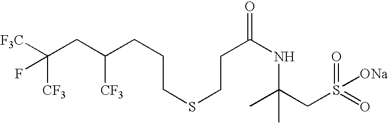 | 26 | 2 |
| 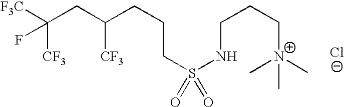 | 20.2 | 1 |
| 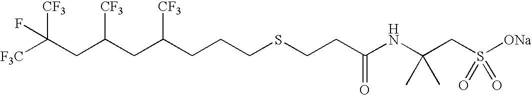 | 23 | 2 |
| 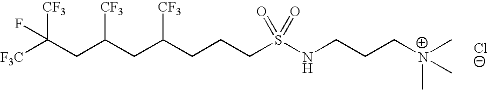 | 19.1 | 0.25 |
| 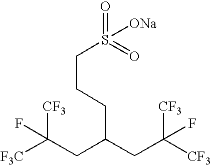 | 19.8 | 2 |
| 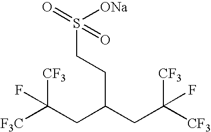 | 20.6 | 2 |
| 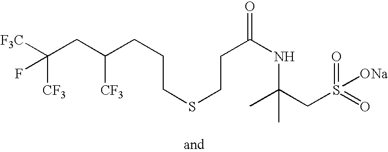 | 19.6 | 0.2 |
| 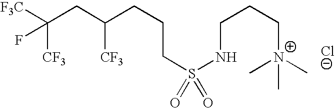 | | |
| 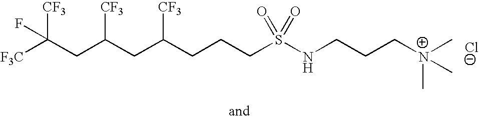 | 21.5 | 0.005 |
| 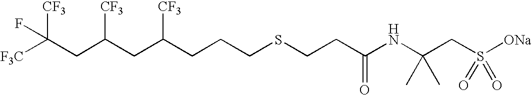 | | |
| 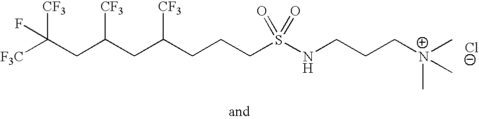 | 20.5 | 0.02 |
| 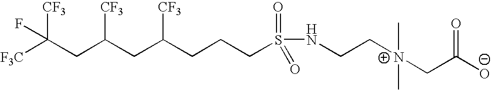 | | |
| 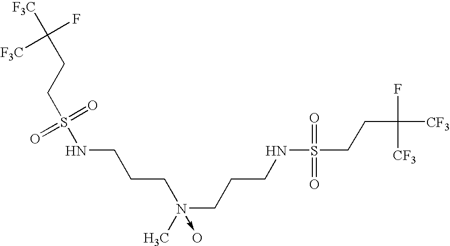 | 22 | 1 |
| 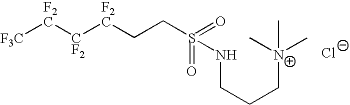 | 19.1 | 1 |
| 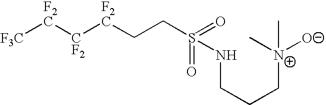 | 18 | 0.25 |
| 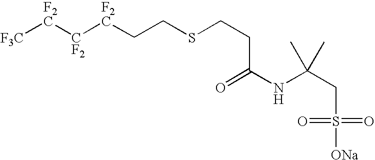 | 27 | 2 |
| 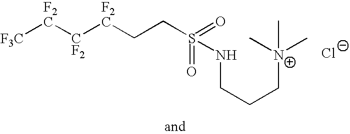 | 16.9 | 0.2 |
| 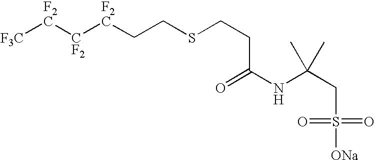 | | |
| 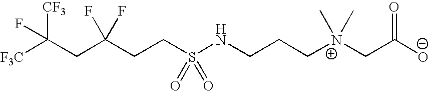 | 19.8 | 1 |
| 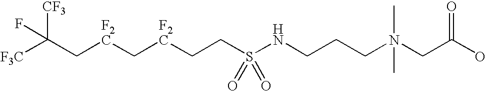 | 19.3 | 0.25 |
| 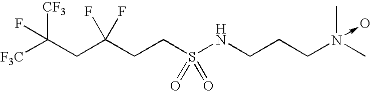 | 19.1 | 1 |
| 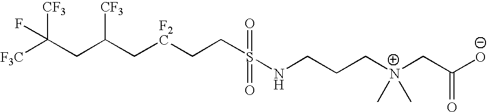 | 19.9 | 0.125 |
| 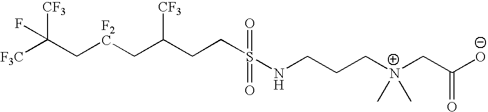 | 19.5 | 0.125 |
| 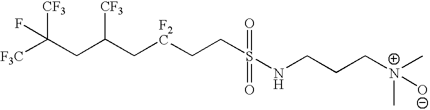 | 19.7 | 0.125 |
| 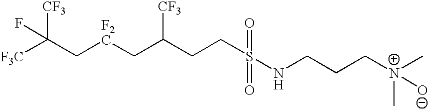 | 20.8 | 0.125 |
| 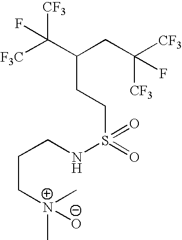 | 21 | 0.05 |
| 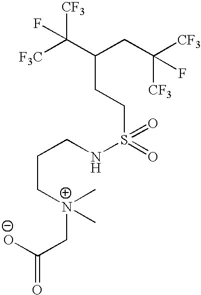 | 21.3 | 0.05 |
| 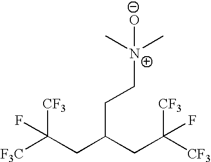 | 19.3 | 0.25 |
| 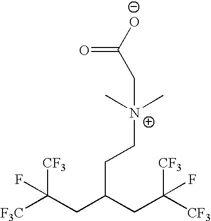 | 20 | 0.5 |
| 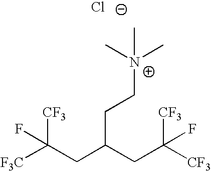 | 22.4 | 1.0 |
| 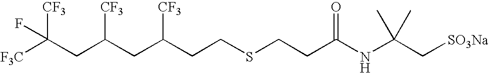 | 24.4 | 2.0 |
| 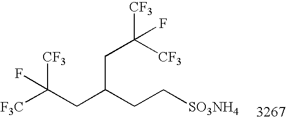 | 23.2 | 0.05 |
| 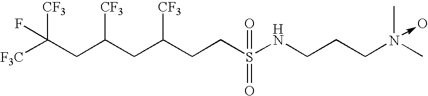 | 18.7 | 0.05 |
| 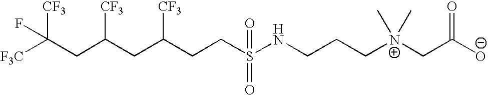 | 19.5 | 0.05 |
| 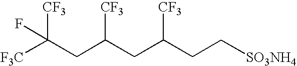 | 24 | 0.25 |
| 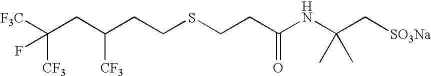 | 16.7 | 0.5 |
| 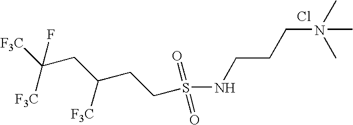 | 28 | 1.0 |
| 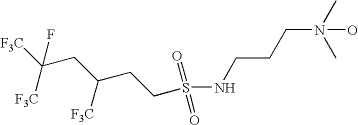 | 19.1 | 0.5 |
| 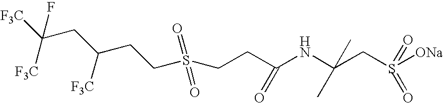 | 28.3 | 1.0 |
| 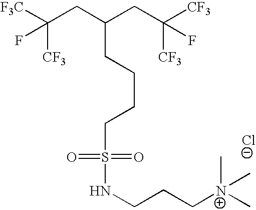 | 19.7 | 0.5 |
| 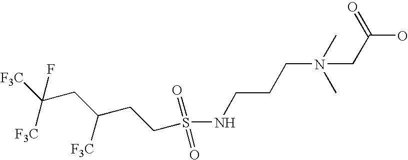 | 19.6 | 0.5 |
| 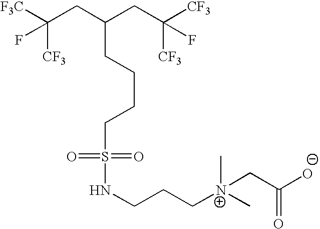 | 19.6 | 0.025 |
| 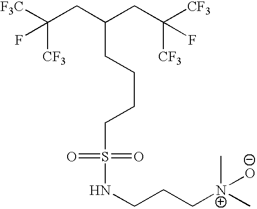 | 19.3 | 0.01 |
| 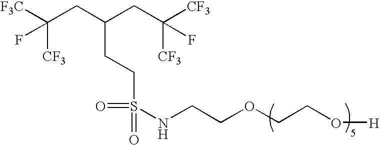 | 20.6 | 0.05 |
| 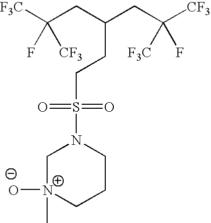 | 19.3 | 0.05 |

R_{F}-surfactants described above may be incorporated into detergents, emulsifiers, paints, adhesives, inks, wetting agents, foamers, and/or defoamers, for example.

R_{F}-surfactants can be incorporated into AFFF formulations and these formulations can be used as fire-fighting foams, to prevent, and/or extinguish combustion. An exemplary use of AFFFs that include an R_{F}-surfactant includes the addition of the AFFF to high pressure misting systems, the misting systems being used to prevent and/or extinguish combustion. AFFF formulations can be provided to a substrate, for example. The substrate can include liquid and/or solid compositions. The AFFF formulations can also be dispersed into an atmosphere including gaseous atmospheres, such air to prevent and/or extinguish combustion.

The formulations can include other components such as water soluble solvents. These solvents may facilitate the solubilization of the RF-surfactants and other surfactants. These solvents can also act as foam stabilizers and/or freeze protection agents. Exemplary solvents can include ethylene glycol, diethylene glycol, glycerol, ethyl Cellusolve®, butyl Carbitol®, Dowanol DPM®, Dowanol TPM®, Dowanol PTB®, propylene glycol, and/or hexylene glycol. Additional components to the formulation, such as polymeric stabilizers and thickeners, can be incorporated into the formulation to enhance the foam stability property of a foam produced from aeration of the aqueous solution of the formulation. Exemplary polymeric stabilizers and thickeners include partially hydrolyzed protein, starches, polyvinyl resins such as polyvinyl alcohol, polyacrylamides, carboxyvinyl polymers, and/or poly(oxyethylene)glycol. Polysaccharide resins, such as xanthan gum, can be included in the formulation as a foam stabilizer in formulations for use in preventing or extinguishing polar solvent combustion, such as alcohol, ketone, and/or ether combustion, for example. The formulation can also include a buffer to regulate the pH of the formulation, for example, tris(2-hydroxyethyl) amine or sodium acetate, and a corrosion inhibitor such as toluoltriazole or sodium nitrite may be included. Water soluble electrolytes such as magnesium sulphate may be included and can improve film-spreading characteristics of the formulation.

For example and by way of example only, the following formulations can be prepared using R_{F}-surfactants.

TABLE 20
Exemplary AFFF Mix Formulation

| | Concentration | |
|---|---|---|
| Material | % (wt/wt) | g/150 g |
| 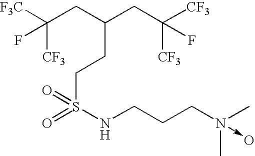 | 0.013 | 1.875 |
| SDS-30 (30% sodium decyl sulfate) | 0.105 | 15.750 |
| CA-40 (Colonial Chemical Colateric | 0.129 | 19.350 |
| CA-40, an imidazoline dicarboxylate | | |
| amphoteric surfactant) | | |
| Sequestrene 30 (EDTA disodium salt | 0.055 | 8.250 |
| 30% active) | | |
| BC (butyl carbitol) | 0.143 | 21.480 |
| EG (ethylene glycol) | 0.121 | 18.105 |
| APG 325N (50% active alkyl | 0.006 | 0.930 |
| polyglycoside from Cognis) | | |
| Water | 0.428 | 64.200 |

Foam quality
Fresh - Expansion 8.3
QDT 3:27 (quarter drain time)
Sea - Expansion 3.9 QDT 2:28

TABLE 21
Exemplary AFFF Mix Formulation

| | Concentration | |
|---|---|---|
| Material | % (wt/wt) | g/150 g |
| 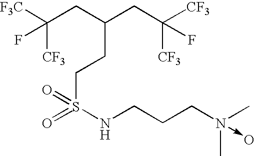 | 0.013 | 1.875 |
| SDS-30 | 0.108 | 16.200 |
| APG | 0.114 | 17.100 |
| EG | 0.038 | 5.700 |
| BC | 0.072 | 10.800 |
| Water | 0.655 | 98.500 |
| 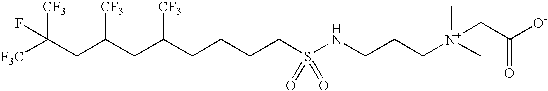 | 21.3 | 0.01 |
| 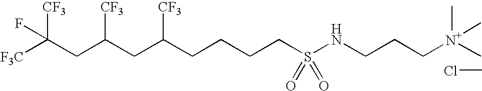 | 20.8 | 0.5 |
| 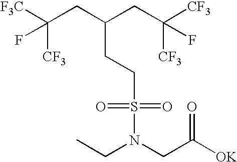 | 18.9 | 1.0 |
| 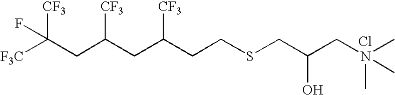 | 22.1 | 0.5 |
| 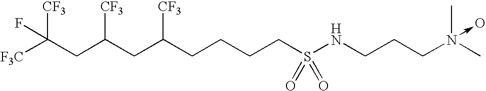 | 19.8 | 0.01 |
| 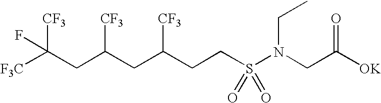 | 23.7 | 0.25 |
| 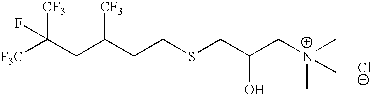 | 32.9 | 1.0 |

Foam quality
Fresh - Expansion
8.1 QDT 3:43
Sea - Expansion
6.2 QDT 3:22

TABLE 22
Exemplary AFFF Mix Formulation

| | Concentration | |
|---|---|---|
| Material | % (wt/wt) | g/150 g |
| 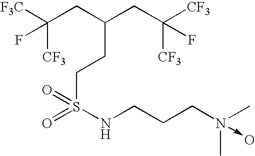 | 0.025 | 3.000 |
| SDS-30 (30% sodium decyl sulfate) | 0.105 | 12.600 |
| CA-40 (Colonial Chemical Colateric | 0.129 | 15.480 |
| CA-40, an imidazoline dicarboxylate | | |
| amphoteric surfactant) | | |
| Sequestrene 30 (EDTA disodium salt | 0.055 | 6.600 |
| 30% active) | | |
| BC (butyl carbitol) | 0.143 | 17.184 |
| EG (ethylene glycol) | 0.121 | 14.484 |
| APG 325N (50% active alkyl | 0.006 | 0.744 |
| polyglycoside from Cognis) | | |
| Water | 0.416 | 49.920 |

Foam quality
Fresh - Expansion
8.3 QDT 3:10
Sea - Expansion
4.2 QDT 2:44

TABLE 23
Exemplary AFFF Mix Formulation

| | Concen- |
|---|---|
| | tration |
| Material | % (wt/wt) |
| 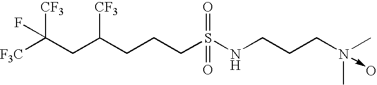 | 15.0 |
| Alpha Foamer | 2.8 |
| SDS-30 (30% sodium decyl sulfate) | 6.0 |
| APG 325N (50% active alkyl polyglycoside from Cognis) | 2.7 |
| HG (hexylene glycol) | 9.0 |
| Magnesium Sulfate | 2.0 |
| Water | Balance |

Expansion Ratio 9.0, QDT 4:21

TABLE 24
Exemplary AFFF Mix Formulation

| | Concen- |
|---|---|
| | tration |
| Material | % (wt/wt) |
| 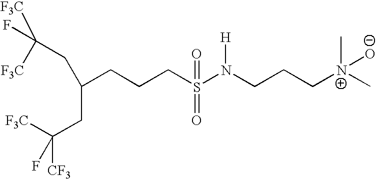 | 2.5 |
| Witconate 3203 | 10.0 |
| HG (hexylene glycol) | 9.0 |
| Magnesium Sulfate | 2.0 |
| Water | Balance |

No Foam

TABLE 25
Exemplary AFFF Mix Formulation

| | Concen- |
|---|---|
| | tration |
| Material | % (wt/wt) |
| 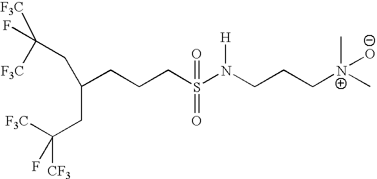 | 2.5 |
| Witconate 3203 | 10.0 |
| APG 325N (50% active alkyl polyglycoside from Cognis) | 2.7 |
| HG (hexylene glycol) | 9.0 |
| Magnesium Sulfate | 2.0 |
| Water | Balance |

Expansion Ratio 4.1, QDT 2:50

TABLE 26
Exemplary AFFF Mix Formulation

| | Concen- |
|---|---|
| | tration |
| Material | % (wt/wt) |
| 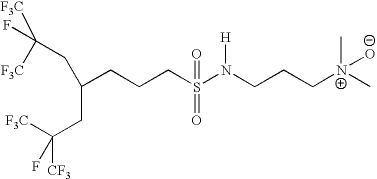 | 2.5 |
| HS-100 | 5.0 |
| HG (hexylene glycol) | 9.0 |
| Magnesium Sulfate | 2.0 |
| Water | Balance |

Expansion Ratio 4.7, QDT 2:40

TABLE 27
Exemplary AFFF Mix Formulation

| | Concen- |
|---|---|
| | tration |
| Material | % (wt/wt) |
| 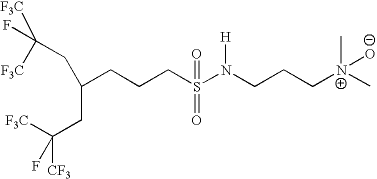 | 2.5 |
| HS-100 | 2.5 |
| Witconate 3203 | 5.0 |
| HG (hexylene glycol) | 9.0 |
| Magnesium Sulfate | 2.0 |
| Water | Balance |

Expansion Ratio = 4.3, QDT = 2:34

TABLE 28
Exemplary AFFF Mix Formulation

| | Concen- |
|---|---|
| | tration |
| Material | % (wt/wt) |
| 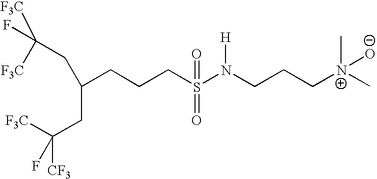 | 2.5 |
| Deriphat 160C | 4.0 |
| SDS-30 | 0.8 |
| HG (hexylene glycol) | 9.0 |
| Magnesium Sulfate | 2.0 |
| Water | Balance |

no foam (expansion ratio is less than 4.0)

TABLE 29
Exemplary AFFF Mix Formulation

| | Concen- |
|---|---|
| | tration |
| Material | % (wt/wt) |
| 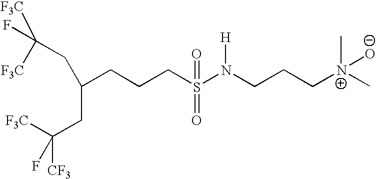 | 2.5 |
| Witconate 3203 | 10.0 |
| APG 325N | 6.0 |
| HG (hexylene glycol) | 9.0 |
| Magnesium Sulfate | 2.0 |
| Water | Balance |

Expansion Ratio = 4.6, QDT = 2:58

TABLE 30
Exemplary AFFF Mix Formulation

| | Concen- |
|---|---|
| | tration |
| Material | % (wt/wt) |
| 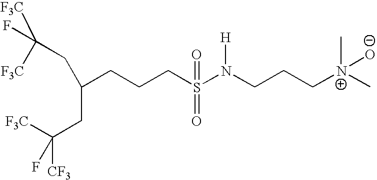 | 2.5 |
| HS-100 | 5.0 |
| APG 325N | 6.0 |
| HG (hexylene glycol) | 9.0 |
| Magnesium Sulfate | 2.0 |
| Water | Balance |

Expansion Ratio 5.8, QDT = 3:04

TABLE 31
Exemplary AFFF Mix Formulation

| | Concen- |
|---|---|
| | tration |
| Material | % (wt/wt) |
| 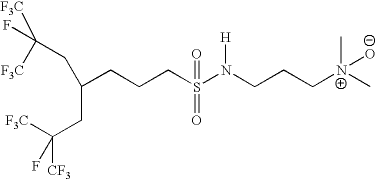 | 2.5 |
| HS-100 | 5.0 |
| APG 325N | 7.7 |
| Alpha Foamer | 2.3 |
| SDS-30 | 2.8 |
| HG (hexylene glycol) | 9.0 |
| Magnesium Sulfate | 2.0 |
| Water | Balance |

Expansion Ratio = 7.3, QDT = 3:27

The R_{F}-surfactants can also be useful in formulations that include other surfactants such as alkyl sulfate, alkylethersulfates, alphaolefinsulfonates, alkyl sulfobetaines, alkyl polyglycerides, alkylamidopropylbetaines, alkylimidazolinedicarboxylates, 2-alkylthiopropionamido-2 methyl-propanesulfonoic acid sodium salt, alkyliminodipropinates, alkylsulfonates, ethoxylated alkylphenols, dialkylsulfosuccinates, and/or alkyltrimethyl ammonium chloride.

A variation of AFFF, ARAFFF, an acronym for Alcohol Resistant Aqueous Film Forming Foam(s), can be used to extinguish hydrocarbon fires in much the same manner that AFFF foams are used and may also be used to extinguish fires involving water soluble solvents such as acetone and isopropanol which conventional AFFF foams will not extinguish.

ARAFFF formulations can contain the same ingredients as conventional AFFF formulations plus a polysaccharide such as xanthan gum and, in some formulations, a polymeric foam stabilizer. Polymeric foam stabilizers are offered by DuPont® and Dynax®, Inc. An exemplary DuPont product, Forafac® 1268, is a water soluble acrylic polymer. An exemplary Dynax product, DX5011®, is an ethyleneimine polymer. Xanthan gum is offered by several suppliers, including Kelco CP (Kelzan) and Rhodia North America (Rhodopol).

Polysaccharide alone can be sufficient to make ARAFFF formulations alcohol resistant, but the amount required produces a foam concentrate that can be quite viscous. The use of a polymeric foam stabilizer can permit a reduction in the amount of polysaccharide required to give useful alcohol resistance.

Because of the possibility of microbial attack on polysaccharide solutions, ARAFFF concentrates can contain an effective amount of a biocide such as Kathon CG ICP, manufactured by Rohm & Haas. Many other biocides such as Acticide, Nipacide and Dowicil can also be effective.

Some ARAFFF formulations can be designed to be proportioned at different percentages depending on whether the substrate to be extinguished is a hydrocarbon or an alcohol type substrate, for example. Alcohol type can include any fuel having a hydroxyl group.

Exemplary ARAFFF formulations utilizing the R_{F}-surfactants can be provided and/or formulated in accordance with the methods described in the Published International Applications. Water can be the balance of the formulation. Foam stabilizers, such as R_{F}-stabilizers that include R_{F} groups described above, for example, can be prepared. R_{F}-stabilizers can include R_{F}-Q_{FS} compositions. According to exemplary embodiments the R_{F} portion can at least partially include an R_{F}(R_{T})n portion as described above. The R_{F}(R_{T})n portion of the surfactant can also include the R_{S} portion described above. In accordance with exemplary implementations the R_{S} portion can be incorporated to provide additional carbon between the R_{F} and/or R_{F}(R_{T})n portions and the Q_{FS} portion of the surfactant. Exemplary R^{s} portions include —CH₂—CH₂-Q_{FS} can include portions that have a greater hydrophilic character than R_{F}. Exemplary Q_{FS} portions include the Qs portions described herein as well as those having polyalkoxylated amines. Exemplary QFS portions of foam stabilizers can be those utilized in U.S. Pat. Nos. 5,750,043, 5,491,261, 5,218,021, 4,606,973, 4,460,480, and/or 3,769,307, the entirety of which are incorporated by reference herein.

Exemplary R_{F}-Foam Stabilizers include, but are not limited to those in Table 32 below.

TABLE 32
Exemplary R_{F}-Foam Stabilizers

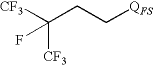

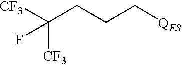

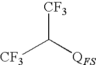

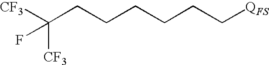

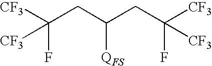

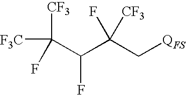

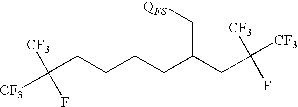

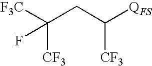

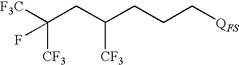

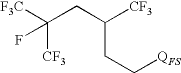

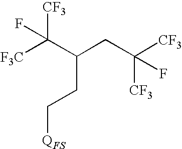

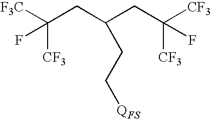

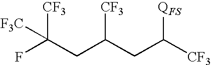

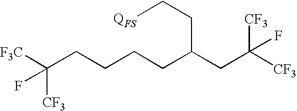

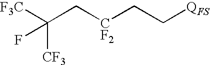

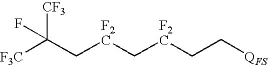

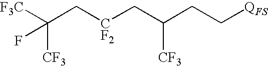

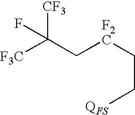

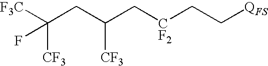

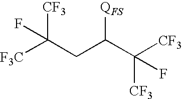

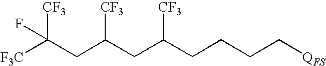

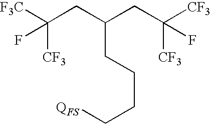

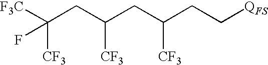

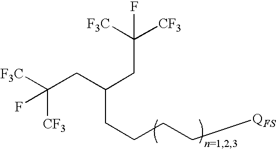

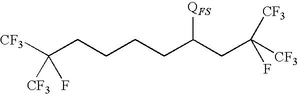

R_{F}-metal complexes such as R_{F}-Q_{MC} incorporating the R_{F} portions are also provided. The R_{F} portions can be incorporated as acid halides or carboxylic acids, for example, with the acid halide including, but not limited to, acid fluorides, for example. According to exemplary embodiments the R_{F} portion can at least partially include an R_{F}(R_{T})n portion as described above. The R_{F}(R_{T})n portion of the complex can also include the R_{S} portion described above. In accordance with exemplary implementations the R_{S} portion can be incorporated to provide additional carbon between the R_{F} and/or R_{F}(R_{T})n portions and the Q_{MC} portion of the complex. Exemplary Rₛ, portions include —CH₂—CH₂—. R_{F}-metal complexes can include R_{F}-intermediates and, as such, Q_{g} can be interchangeable with Q_{MC} in certain instances. O_{MC} can include the portion of a ligand of a metal complex that is coordinated with the complexed metal, for example. According to exemplary embodiments, the Q_{MC} group can include a charged group such as an unprotonated carboxylic acid group. The Q_{MC} group can be configured to complex one or more metal ions such as Cr^{3t}. The Q_{MC} group can be referred to as a chelating group. Exemplary R_{F}-metal complexes include, but are not limited to, those in Table 33 below.

TABLE 33
R_{F}-Metal Complexes

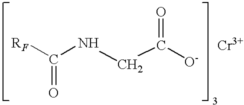

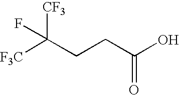

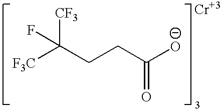

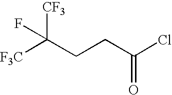

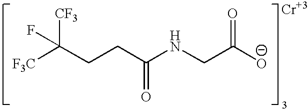

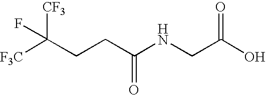

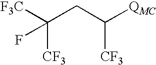

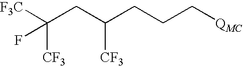

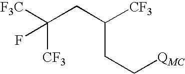

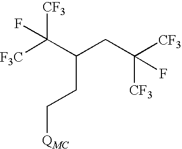

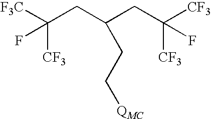

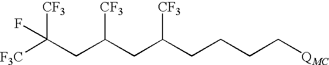

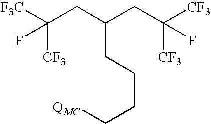

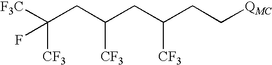

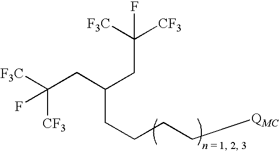

Exemplary R_{F}-metal complexes can be prepared by way of the following exemplary synthetic steps.

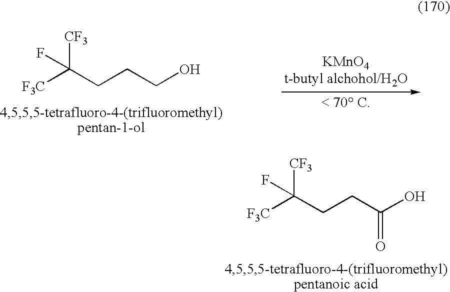

According to scheme (170) above, in a flask that can be equipped with an agitator, thermocouple, reflux condenser, and an addition funnel, 27.8 grams (0.12 mole) of 4,5,5,5-tetrafluoro-4-(trifluoromethyl)pentan-1-ol (see, e.g., Published International Patents) can be added. To the addition funnel, 117.3 grams (0.74 mole) of potassium permanganate, 117.2 grams of tert-butyl alcohol, and about 89 mL of water can be added to form a mixture. To the flask, the mixture can be added drop wise to form a reaction mixture at a rate such that the reaction mixture temperature is maintained at about 70° C. The reaction mixture can be slowly heated to reflux and held for about three hours. The reaction mixture can be cooled, diluted with water and filtered. The filter residue can be washed thoroughly with water. The washings and filtrate can be combined and acidified with concentrated HCl to provide a lower organic layer. The organic layer can be separated, washed with water and concentrated by distillation to afford the 4,5,5,5-tetrafluoro-4-(trifluoromethyl)pentanoic acid product. The product structure can be confirmed by NMR and/or chromatographic analysis.

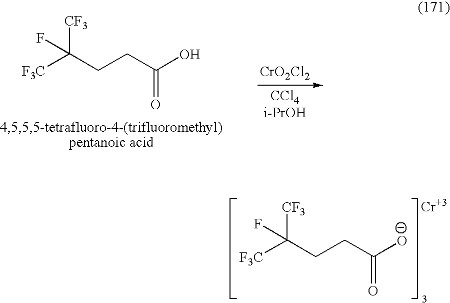

In reference to scheme (171) above, in a flask that can be equipped with an agitator, thermocouple, reflux condenser, and an addition funnel, 12.01 grams (0.05 mole) of 4,5,5,5-tetrafluoro-4-(trifluoromethyl)pentanoic acid (see, e.g., Published International Patents), about 70 mL of dry isopropanol (i-PrOH) can be added to form a mixture. To the addition funnel, 25 grams (0.161 mole) of chromyl chloride and about 70 mL of carbon tetrachloride (CCl₄) can be added and thoroughly mixed to form an addition mixture. To the mixture, the addition mixture can be slowly added

TABLE 33
R_{F}-Metal Complexes

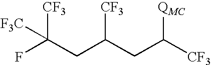

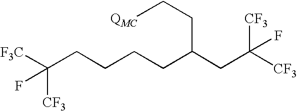

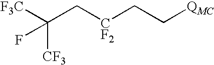

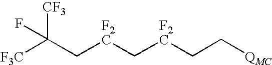

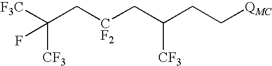

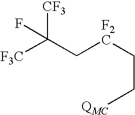

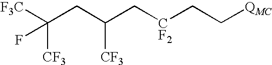

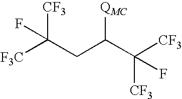

to form a reaction mixture at such a rate as to maintain the reaction mixture temperature between about 40° C. and about 60° C. The reaction mixture can be heated to reflux and held for about one hour then cooled and filtered. The filtrate can be concentrated by rotary evaporator to afford a mixture about 30 (wt/wt) percent of the product

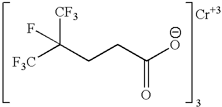

To the mixture, about 1 mL of water can be added as a stabilizer.

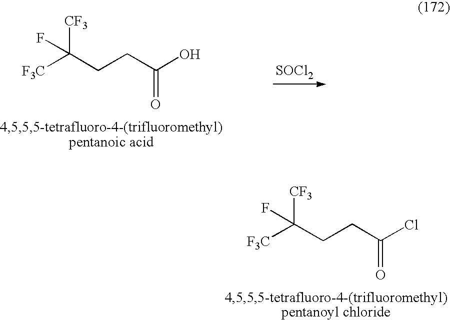

In reference to scheme (172) above, in a flask that can be equipped with an agitator, thermocouple, reflux condenser, and an addition funnel, 3.6 grams (0.03 mole) of thionyl chloride can be added and gently warmed. To the warmed thionyl chloride, 6.05 grams (0.025 mole) of 4,5,5,5-tetrafluoro-4-(trifluoromethyl)pentanoic acid can be added drop wise over a period of about 3 minutes to 30 minutes to form a reaction mixture. The reaction mixture can be distilled to afford the 4,5,5,5-tetrafluoro-4-(trifluoromethyl)pentanoyl chloride product. The product structure can be confirmed by NMR and/or chromatographic analysis.

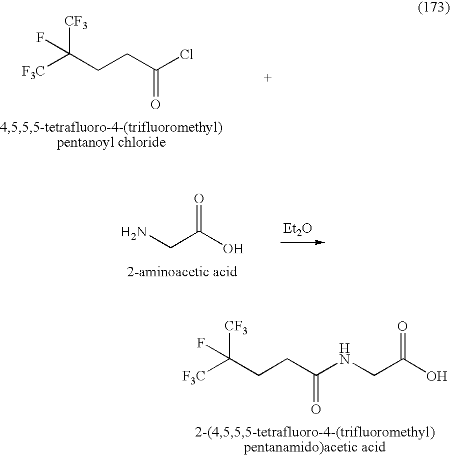

In accordance with scheme (173) above, in a flask that can be equipped with an agitator, thermocouple, reflux condenser, and an addition funnel, 9.8 grams (0.13 mole) of 2-aminoacetic acid and 70 mL of anhydrous diethyl ether can be added to form a mixture. To the mixture, 26.05 grams (0.1 mole) of 4,5,5,5-tetrafluoro-4-(trifluoromethyl)pentanoyl chloride (see scheme (166) above) and about 30 mL of anhydrous diethyl ether can be added drop wise to form a reaction mixture. The reaction mixture can be heated to reflux under a nitrogen atmosphere and held for about three hours. The reaction mixture can be filtered and the filtrate concentrated in vacuo to afford a residue. To the residue, about 50 mL of anhydrous diethyl ether can be added and washed with water to form a multiphase mixture from which an organic phase can be separated from an aqueous phase. The organic phase can be dried over magnesium sulfate, filtered, and concentrated in vacuo to afford the 2-(4,5,5,5-tetrafluoro-4-(trifluoromethyl)pentamido)acetic acid product. The product structure can be confirmed by NMR and/or chromatographic analysis.

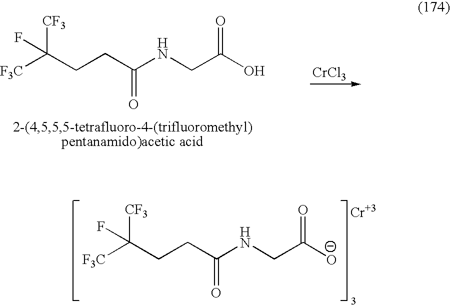

In conformity with scheme (174) above, in a flask that can be equipped with an agitator, thermocouple, reflux condenser, and an addition funnel, 54.4 grams (0.204 mole) of chromic chloride hexahydrate, about 50 mL of methanol can be added to form a mixture and subjected to moderate heat. Separately, 9.6 grams (0.24 mole) of sodium hydroxide can be added to about 40 mL of methanol at about 50° C. to form an addition mixture. To the vigorously agitated mixture, the addition mixture can be added drop wise over about one hour to form a new mixture. The new mixture can be heated to reflux and maintained for about one hour subsequently heating to reflux and maintaining there for about one hour. To the new mixture, 8.86 grams (0.034 mole) of 2-(4,5,5,5-tetrafluoro-4-(trifluoromethyl)pentamido)acetic acid (refer to scheme (167) above) can be added drop wise to form a reaction mixture and heated to reflux and maintained for about one hour. The reaction mixture can be cooled to from about 18° C. to about 24° C., and/or about 21° C., filtered and adjusted to 30 (wt/wt) percent solid concentration by the addition of methanol to afford a chromium complex that can have a molar ratio of chromium to fluorocarbon of about 6:1 and the molar ratio of sodium hydroxide to fluorocarbon of about 7:1.

The above described chrome complex solutions can be applied as a surface treatment of a variety of materials including, but not limited to, leather by the employment of the methods described in U.S. Pat. No. 3,351,643 and U.S. Pat. No. 3,948,887, herein incorporated by reference.

An exemplary method for preparing the R_{F}-metal complexes includes reacting the RF-intermediate having halogen functionality, such as Q_{g} is I, disclosed above, with fuming sulfuric acid to produce an R_{F}-intermediate having acid fluoride functionality, for example. R_{F}-metal complexes can be prepared with reference to scheme (175) below.

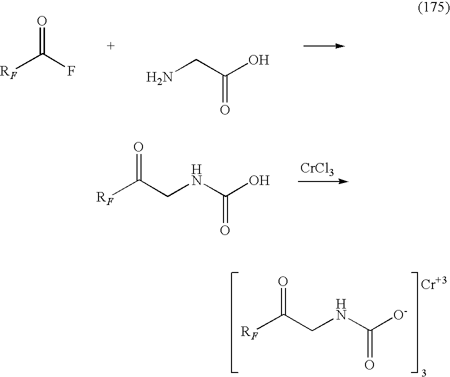

An acid fluoride R_{F}-intermediate can be reacted with an amino acid such as glycine to produce an amine ester. The amine ester may then be reacted with chromic chloride in an alcohol such as methanol or isopropanol to produce an exemplary R_{F}-metal complex such as a R_{F} chrome complex. Exemplary acid R_{F}-intermediates for use In preparation of R_{F}-metal complexes can include ethylene carboxylic acid RF-intermediates and/or mixtures of ethylene carboxylic acid R_{F}-intermediates and carboxylic acid R_{F}-intermediates. Exemplary preparations can be performed in accordance with U.S. Pat. Nos. 3,351,643, 3,574,518, 3,907,576, 6,525,127, and 6,294,107, herein incorporated by reference. R_{F}-metal complexes can include a ligand having a R_{F} portion and a Q_{MC} portion associated with the metal of the complex. In exemplary embodiments the Q_{MC} portion can have a greater affinity for the metal of the complex than the R_{F} portion. R_{F}-metal complexes can be used to treat substrates such as paper, leather, textiles, yarns, fabrics, glass, ceramic products, and/or metals. In some cases treating substrates with the complexes render the substrates less permeable to water and/or oil.

An embodiment of the present invention also provides for incorporation of the R_{F} portions into phosphate esters which, in exemplary embodiments, can be used to treat substrates and/or be used as dispersing agents during the preparation of polymers. Exemplary R_{F}-phosphate esters include R_{F}-Q_{PE}, with the Q_{PE} portion being the phosphate portion of the R_{F}-composition. According to exemplary embodiments the R_{F} portion can at least partially include an R_{F}(R_{T})n portion as described above. The R_{F}(R_{T})n portion of the ester can also include the R_{S} portion described above. In accordance with exemplary implementations the R_{S} portion can be incorporated to provide additional carbon between the R_{F} and/or R_{F}(R_{T})n portions and the Q_{PE} portion of the ester. Exemplary Rₛ portions include —CH₂—CH₂—. R_{F}-phosphate esters, include, but are not limited to, those in Table 34 below.

TABLE 34
R_{F}-Phosphate Esters

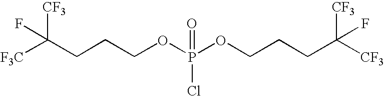

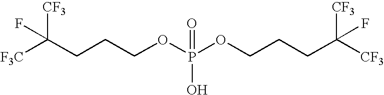

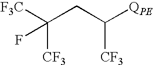

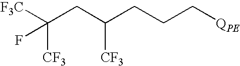

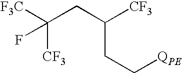

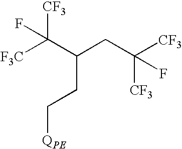

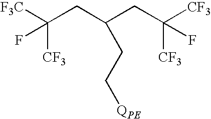

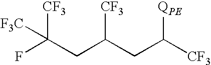

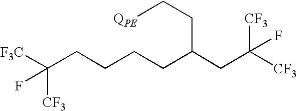

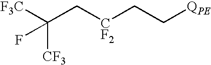

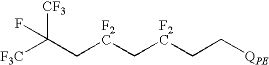

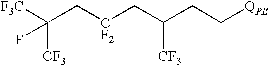

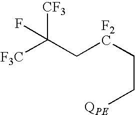

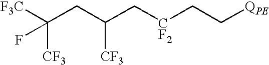

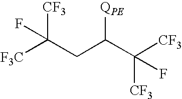

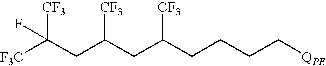

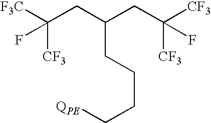

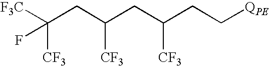

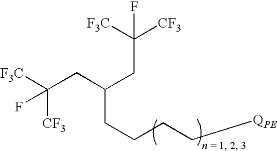

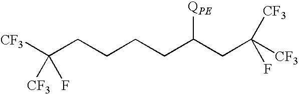

R_{F}-phosphates can be used as dispersing agents in the preparation of polymers or they can be diluted and used to treat substrate materials in aqueous bathes, for example, by ordinary means such as padding, dipping, impregnating, spraying, etc. These compositions can be incorporated into or used to treat such materials as textile fabric, textile yarns, leather, paper, plastic, sheeting, wood, ceramic clays, as well as, manufactured articles prepared therefrom such as articles of apparel, wallpaper, paper bags, cardboard boxes, porous earthenware, etc. U.S. Pat. No. 3,112,241 describes methods for treating materials using phosphate esters and is herein Incorporated by reference. R_{F}-phosphoric acid ester can be used to treat substrates such as wood pulp products, including paper products such as packaging products including food packaging products.

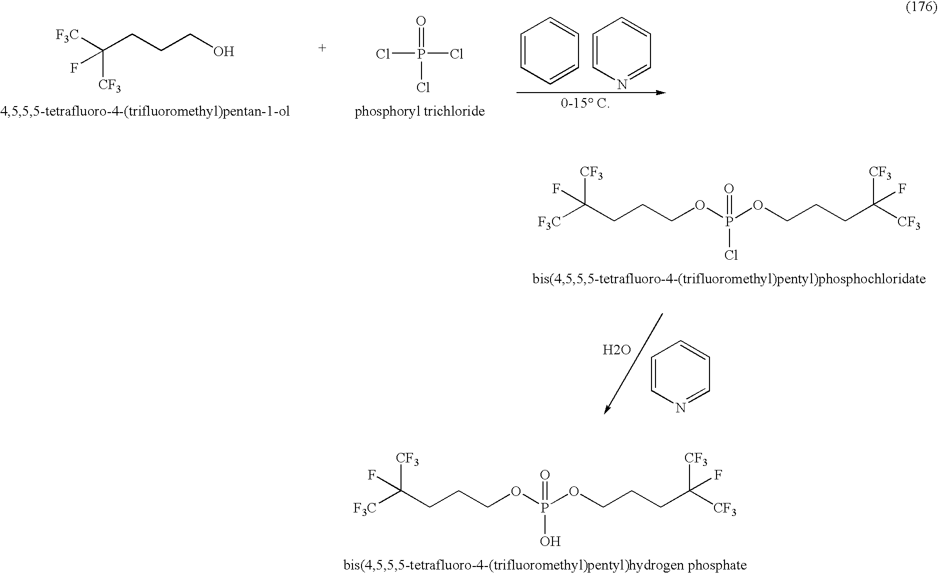

According to scheme (176) above, about 28.2 gram (0.361 mole) benzene, about 5.45 gram (0.069 mole) pyridine, and about 8.12 gram (0.053 mole) phosphoryl trichloride can be added to a 125 mL three neck round bottom flask that can be equipped with a thermocouple, a 50 mL pressure equalizing addition funnel, and an agitator to form mixture A which may be observed as pale brown in color. About 23.86 gram (0.105 mole) 4,5,5,5-tetrafluoro-4-(trifluoromethyl)pentan-1-ol (see, e.g. Published International Applications), about 22.44 gram (0.305 mole) benzene, and about 5.6 gram (0.071 pyridine) can be added to the pressure equalizing funnel to form mixture B which can be observed as colorless. Mixture A can be chilled to about 7° C., from about 0° C. to about 15° C., and/or about 2° C. followed by the addition of mixture B over about two hours to form a new mixture. During the addition of mixture B to mixture A an exotherm and a white precipitate can be observed. The ice bath can be removed and the new mixture gradually warmed to from about 18° C. to about 24° C., and/or about 21° C. and then heated to reflux and held for about 45 minutes, from about 30 minutes to 60 minutes, and/or about 40 minutes to about 50 minutes to afford a mixture that can contain the bis(4,5,5,5-tetrafluoro-4-(trifluoromethyl)pentyl hydrogen phosphate product. m/Z 519 (M⁺+H), 499 (M⁺-F), 449 (M⁺-CF₃).

An embodiment includes the R_{F} portions incorporated into glycols, such as R_{F}-glycols, including R_{F}-Qₕ, with Qₕ representing the ether portion of the glycol after conjugation or, as hydroxyl functionality before conjugation as the ether. According to exemplary embodiments the R_{F} portion can at least partially include an R_{F}(R_{T})n portion as described above. The R_{F}(R_{T})n portion of the glycol can also include the R_{S} portion described above. In accordance with exemplary implementations the R_{S} portion can be Incorporated to provide additional carbon between the R_{F} and/or R_{F}(R_{T})n portions and the Qₕ portion of the glycol. Exemplary Rₛ portions include —CH₂—CH₂—. Exemplary R_{F}-glycols include, but are not limited to, those in Table 35 below.

TABLE 35
Exemplary R_{F}-Glycols

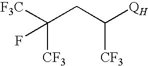

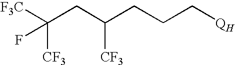

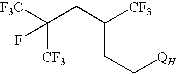

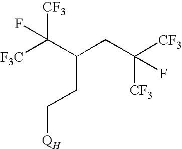

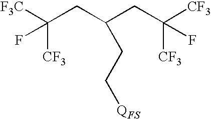

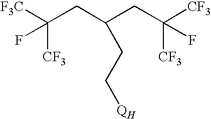

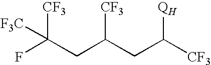

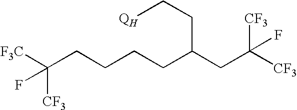

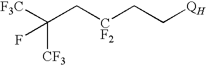

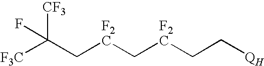

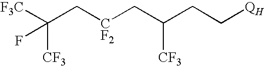

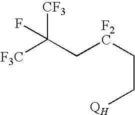

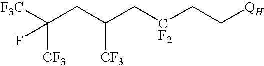

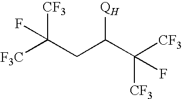

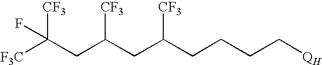

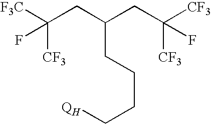

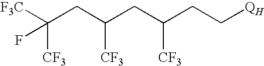

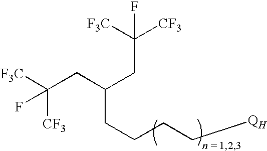

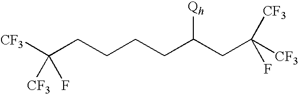

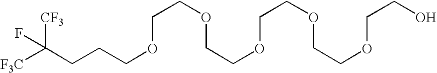

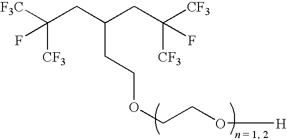

R_{F}-glycols can be incorporated into polymers such as urethanes including polyurethane elastomers, films and coatings, for example. R_{F}-glycols can also be converted to phosphoric acids or phosphate esters of those glycols as well. Referring to scheme (177) below, R_{F} portions can be incorporated Into glycols.

Methods for preparing glycols are described in U.S. Pat. No. 4,898,981, U.S. Pat. No. 4,491,261, U.S. Pat. No. 5,091,550, U.S. Pat. No. 5,132,445, and Dupau, et. al., Adv. Synth. Catal. 2002.344. No. 3&4, Procedure B, all of which are herein incorporated by reference. For example, and by way of example only, a RF-intermediate (Q_{g}=SH) can be reacted with a sulfide diol or 2,6 diox-aspiro (3,3) heptane to produce exemplary R_{F}-gycols (Qₕ=H₂CH₂CSH₂CH₂ . . . ) The R_{F}-glycol can then be used directly or indirectly to prepare a RF condensation product such as polyesters, polyureas, polycarbonates, and polyurethanes. This glycol functionality can also be incorporated into block polymers using R_{F}-glycols. U.S. Pat. No. 5,491,261 discloses several other glycols that can benefit from the R_{F} portion of the present invention and is herein incorporated by reference.

R_{F}-glycols may also be converted to phosphoric acid functionality or phosphate esters (not shown). U.S. Pat. Nos. 5,091,550, 5,132,445, 4,898,981, and 5,491,261 all disclose methods of preparing diols and converting diols to phosphate esters and are herein incorporated by reference. In an exemplary implementation, the diols can be converted to phosphoric acid or phosphate esters by reacting the diols in the presence of phosphoric acid. These compositions can be incorporated into compounds which can act as oil and grease proofing for paper, as well as, soil release agents for textile fibers.

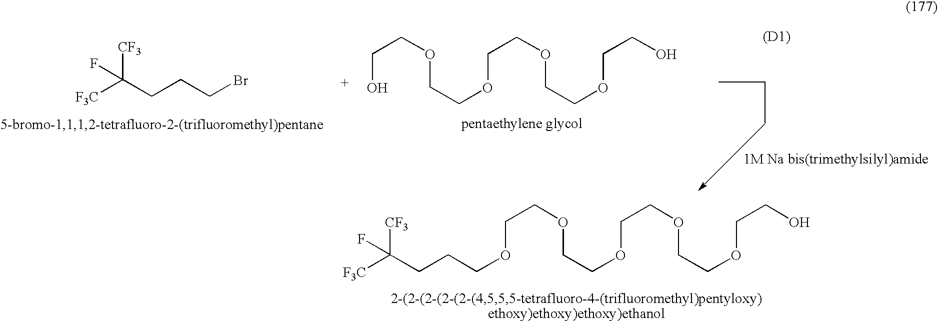

According to scheme (177) above, in a flask that can be equipped with a thermocouple, addition funnel, heating mantle, and a nitrogen feed line, about 1.2 grams (0.005 mole) of pentaethylene glycol in about 10 mL anhydrous tetrahydrofuran (THF) can be placed to form a mixture under a nitrogen atmosphere. The mixture can be cooled to from about 0° to about 5° C. in an ice/acetone bath. To the mixture, about 5.15 mL of a 1M solution of sodium bis(trimethylsilyl)amide in THF can be added to form a second mixture. The second mixture can be stirred at from about 0° to about 5° C. for about 15 minutes, followed by the drop wise addition of 1.5 grams (0.005 mole) of 5-Bromo-1,1,1,2-tetrafluoro-2-trifluoromethyl-pentane (see, e.g. Published International Applications) dissolved in about 10 mL THF to form a reaction mixture. The reaction mixture can be allowed to warm to from about 18° C. to about 24° C., and/or about 21° C. and held for about two hours. The reaction mixture can be heated to about 40° C. and held for from about 15 hours to about 21 hours, and/or about 18 hours. The reaction mixture can be allowed to cool to from about 18° C. to about 24° C., and/or about 21° C. and about 17 mL of a 5 percent (wt/wt) solution of HCl can be added to afford a multiphase mixture with a pH of about seven from which an organic phase can be separated from an aqueous phase. The organic layer can be concentrated in vacuo to afford about 0.8 gram of 2-(2-(2-(2-(2-(4,5,5,5-tetrafluoro-4-(trifluoromethyl)pentyloxy)ethoxy)ethoxy)ethoxy)ethoxy)ethanol product. The product structure can be confirmed by NMR and/or chromatographic analysis.

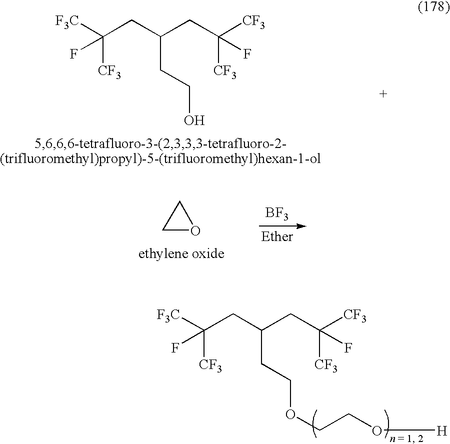

According to scheme (178) above, in a 60 mL autoclave, 18 grams (44.3 mmol) of 5,6,6,6-tetrafluoro-3-(2,3,3,3-tetrafluoro-2-(trifluoromethyl)propyl)-5-(trifluoromethyl)hexan-1-ol (refer to scheme (45) above) can be placed. To the autoclave, 22 grams (4.43 mole) of separately condensed ethylene oxide can be added to form a mixture. To the mixture, 0.15 mL of boron trifluoride etherate can be added to form a reaction mixture and the autoclave can be sealed. The reaction mixture can be slowly heated to 50° C. and maintained for an hour to afford a product mixture having the generalized structure

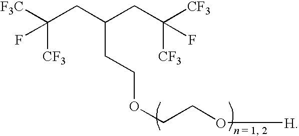

The product structure can be confirmed by NMR and/or chromatographic analysis.

According to another embodiment of the present invention oligomers, polymers, copolymers, acrylics, and/or resins, for example, can be prepared that include an R_{F}-monomer unit, such as R_{F}-Q_{MU}. The monomer unit portion, Q_{MU}, can be a single unit within a complex of units and the monomer unit need not repeat within the complex. In an exemplary embodiment, the monomer unit can be a single unit within the complex or it may be one of many identical units linked together, such as a homopolymer, for example. The complex can also include block polymers and/or polyurethane resins. The R_{F} of the unit can include a pendant group of the monomer unit. The monomer unit may be associated with a complex, perhaps even bonded to the complex, for example, and Q_{MU} can include the portion of the monomer unit that is associated with the complex. The complex may be coated onto a substrate or it may be chemically bonded to the substrate. For example, a preparation of R_{F}-intermediates can be provided to the substrate and groups such as hydroxyl groups common to substrates like cotton, may provide sites that allow the R_{F}-intermediate to chemically bond to the substrate when forming part of, or being associated with a complex. In an exemplary embodiment, Q_{MU} can represent the acrylate functionality of an acrylic and R_{F} can be a pendant group from the acrylics chain and/or backbone. According to exemplary embodiments the R_{F} portion can at least partially include an R_{F}(R_{T})n portion as described above. The R_{F}(R_{T})n portion of the monomer unit can also include the R_{S} portion described above. In accordance with exemplary implementations the R_{S} portion can be incorporated to provide additional carbon between the R_{F} and/or R_{F}(R_{T})n portions and the Qₛ portion of the monomer unit. Exemplary Rₛ portions include —CH₂—CH₂—. Exemplary R_{F}-monomer units include but are not limited to those in Table 36 below.

TABLE 36
Exemplary R_{F}-Monomer Units

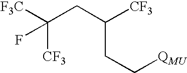

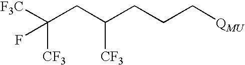

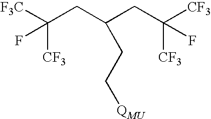

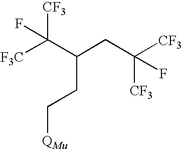

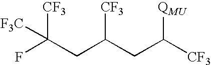

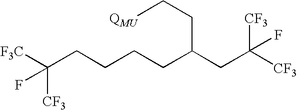

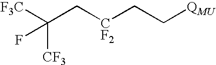

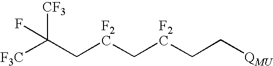

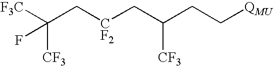

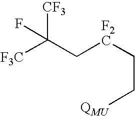

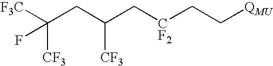

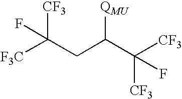

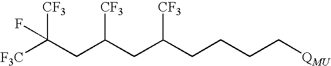

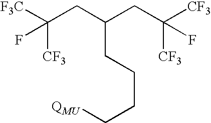

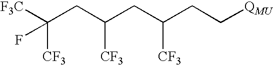

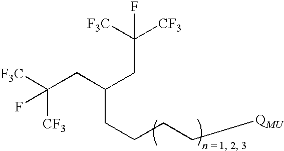

In exemplary embodiments oligomers containing a R_{F}-monomer unit can be prepared from R_{F}-monomers (R_{F}Q_{M}). R_{F}-monomers can include R_{F}-intermediates above, but may contain functionality that allows for their conjugation with another monomer, but not necessarily the same R_{F}-monomer. According to exemplary embodiments the R_{F} portion can at least partially include an R_{F}(R_{T})n portion as described above. The R_{F}(R_{T})n portion of the monomer can also include the R_{S} portion described above. In accordance with exemplary implementations the R_{S} portion can be incorporated to provide additional carbon between the R_{F} and/or R_{F}(R_{T})n portions and the Q_{M} portion of the monomer. Exemplary Rₛ portions include —CH₂—CH₂—. Exemplary R_{F}-monomers include, but are not limited to those in Table 37 below.

TABLE 37
Exemplary R_{F}-Monomers

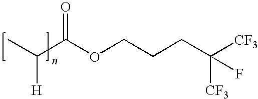

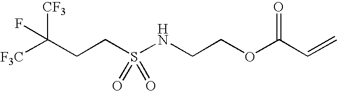

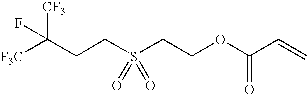

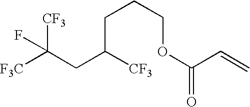

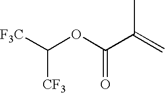

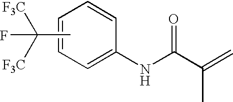

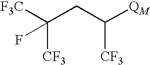

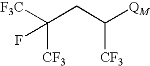

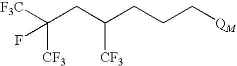

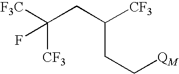

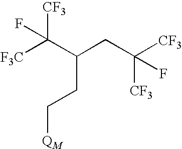

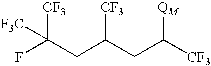

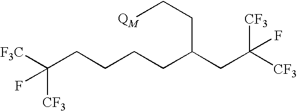

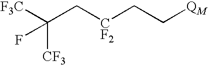

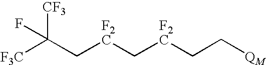

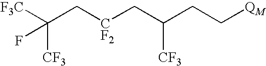

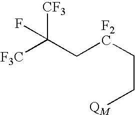

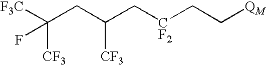

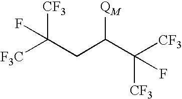

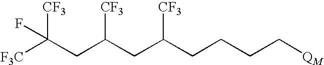

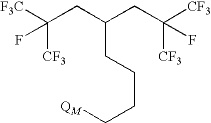

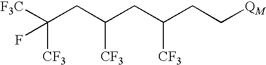

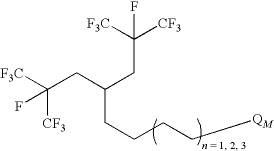

Referring to scheme (179) below, multiple reactions sequences are shown for the preparation of R_{F}-monomers having the R_{F} group.

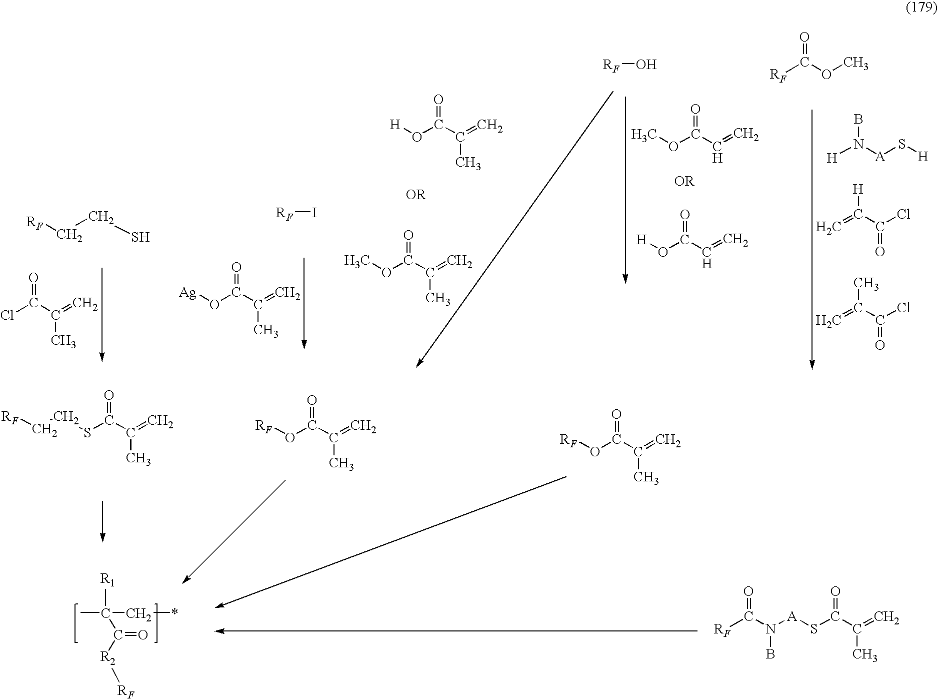

U.S. Pat. Nos. 3,491,169, 3,282,905, 3,497,575, 3,544,663, 6,566,470, 4,147,851, 4,366,299, 4,439,329, and 5,439,998 all relate to the use and preparation of acrylic emulsion polymers that can benefit from the R_{F} groups and, are herein incorporated by reference. Thiol R_{F}-intermediates, Iodine R_{F}-intermediates, hydroxyl R_{F}-intermediates, and/or acetate R_{F}-intermediates can be converted to R_{F}-monomers according to scheme (179) above, and these R_{F}-monomers can be used to prepare a composition containing an R_{F}-monomer unit.

For example, and by way of example only, the R_{F} portion can be incorporated into a R_{F}-monomer as described in U.S. Pat. No. 6,566,470 represented as R_{F}—W—X—C(═O)—C(R₁)═CH₂, with the R_{F} portion as described above. W can be an alkylene with 1 to 15 carbons, hydroxyalkylene with 3 to 15 carbons, —(CₙH₂ₙ)(OCₘH₂ₘ)_{q}—, —SO₂NR₂—(CₙH₂ₙ)—, or —CONR₂—(CₙH₂ₙ)—, with n is 1 to 12, m is 2 to 4, q is 1 to 10, and R₁ is an alkyl group with 1 to 4 carbon atoms, for example, X can be O, S and/or N(R₂), where R₂ is as R₁.

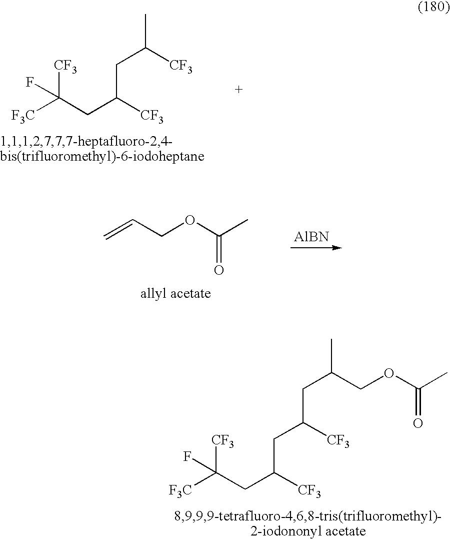

According to scheme (180) above, in a flask that can be equipped with an agitator, thermocouple, reflux condenser that can be equipped with a dry ice/acetone trap, and an addition funnel, 222 grams (0.46 mole) of 1,1,1,2,5,5,5-heptafluoro-2,4-bis(trifluoromethyl)-6-iodoheptane (i.e., telomers of F71, TFP and ethylene) can be placed and heated to about 95° C. In the addition funnel, 46.3 grams (0.46 mole) of allyl acetate and 5.0 grams (0.03 mole) of 2,2′-azobisisobutrylonitrile (AIBN) can be added to form a mixture. The mixture heated to drive the AIBN into solution and added to the flask drop wise over about 80 minutes to form a reaction mixture wherein an exotherm and color change from purplish-pink to clear to pale yellow can be observed. The reaction mixture can be held at from about 90° C. to about 110° C. for from about four hours to about five hours. The reaction mixture can be allowed to cool to from about 18° C. to about 24° C., and/or about 21° C. and held from about 15 hours to about 21 hours, and/or about 18 hours. To the reaction mixture, 1.0 gram (0.006 mole) of AIBN can be added and heated to about 95° C. and held for about 7 hours whereupon an addition 1 gram (0.006 mole) of AIBN can be added and heated to about 150° C. and held for about 2 hours. The reaction mixture can be allowed to cool to from about 18° C. to about 24° C., and/or about 21° C. and held from about 15 hours to about 21 hours, and/or about 18 hours. To the reaction mixture, 0.6 gram (0.004 mole) AIBN can be added and heated to reflux and held for about 3 hours. The reaction mixture can be distilled under vacuum to afford 128.44 grams of an isomeric mixture of the 8,9,9,9-tetrafluoro-4,6,8 tris(trifluoromethyl)-2-iodononyl acetate which can be about 97 (wt/wt) percent pure by gas chromatography. m/z: 528 (M⁺-C₂H₃O₂), 461 (M⁺-I)

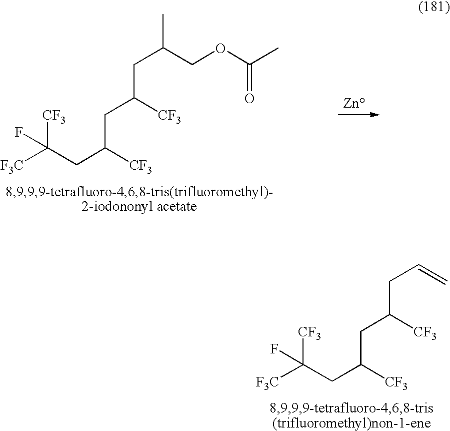

According to scheme (181) above, in a flask that can be equipped with an agitator, thermocouple, and a simple vacuum distillation unit, 39.6 grams (0.09 mole) of an isomeric mixture of 8,9,9,9-tetrafluoro-4,6,8-tris(trifluoromethyl)-2-iodononyl acetate (refer to scheme (180) above) and 9.0 grams (0.14 mole) of zinc can be placed to form a mixture. The mixture can be heated to from about 100° C. to about 105° C. at about 15 mmHg whereupon 9.96 grams of the 8,9,9,9-tetrafluoro-4,6,8-tris(trifluoromethyl)non-1-ene product can be collected in the receiver flask. The product structure can be confirmed by NMR and/or chromatographic analysis.

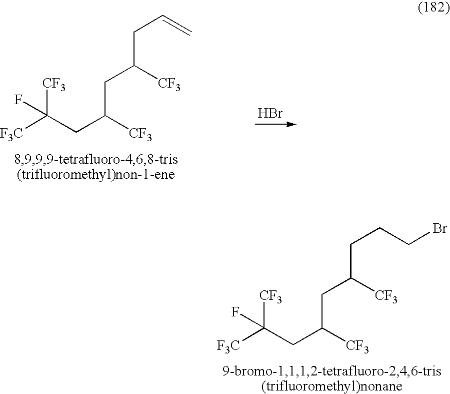

In reference to scheme (182) above, in a 1 L photochemical reaction vessel that can be equipped with a threaded nylon bushing and an agitator. The threaded nylon bushing can be equipped with a nine inch Pen-Ray® 5.5 watt ultraviolet (UV) lamp with corresponding power supply, pressure gauge, gaseous anhydrous hydrobromic acid feeding tube (feeding tube) set at a depth to feed the gaseous anhydrous hydrobromic acid (HBr) subsurface relative to the olefin, and a venting valve, 894.7 grams (2.22 moles) of 8,9,9,9-tetrafluoro-4,6,8-tris(trifluoromethyl)non-1-ene (see, e.g. Published International Applications) can be placed. Gaseous anhydrous HBr can be continuously fed and/or semi-continuously fed into the reactor with the UV light activated for from about six hours to about 16 hours to form a mixture. The mixture can be washed with saturated sodium bicarbonate solution and twice with water wherein each step a multiphase mixture can be formed from which an organic phase can be separated from an aqueous phase. The organic phases can be combined and dried over magnesium sulfate, filtered, and distilled (b.p. 90° C.-95° C.) to afford the 9-bromo-1,1,1,2-tetrafluoro-2,4,6-tris(trifluoromethyl)nonane product. m/z: 403 (M⁺-Br)

A 3M aqueous solution of sodium hydroxide (7.8 grams) can be added to the mixture via an addition funnel over a 15 minute period after which the mixture can be chilled to 0° C. using an ice bath. Hydrogen peroxide (23.6 grams, 35% (wt/wt) aqueous solution) can be added drop-wise over a 15 minute period to the mixture and then the mixture can be washed in H₂O (three times). The organic layer can be removed and transferred into a 100 mL three-neck round bottom flask and distilled to produce an 85% area percent pure (by gas chromatography 4,5,5,5-Tetrafluoro-4-(trifluoromethyl)pentan-1-ol.

An exemplary R_{F}-Q_{M} such as

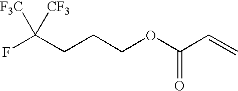

can be provided in solution and conjugated and/or polymerized with another

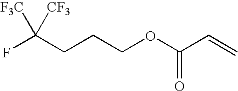

or another compound to form a complex, such as an oligomer, that can include

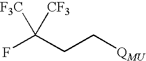

with Q_{MU} representing a remainder of the complex.

Exemplary homopolymers and copolymers can be prepared from Rf-monomers and are illustrated in the examples set forth below.

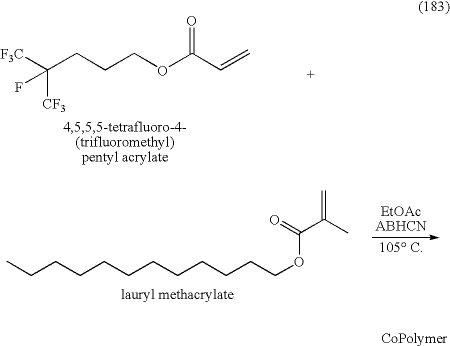

With reference to scheme (183) above, 1.25 grams (0.004 mole) of 4,5,5,5-tetrafluoro-4-(trifluoromethyl)pentyl acrylate, 3.75 grams (0.015 mole) of lauryl methacrylate, 6.0 grams of ethyl acetate, and 0.025 gram (1.02×10−4 mole) of azobis(cyclohexanecarbonitrile) (ABHCN) can be placed into a 500 mL glass lined reactor which can be equipped with an agitator, thermocouple, and an ability to heat the reactor, to form a mixture. The Parr bottle can be flushed with oxygen free nitrogen for about 30 seconds, sealed, and stirred for about 4 hours at a temperature of about 105° C. The resulting copolymer can have a molecular weight of about 51,000 by gas permeation chromatography and a percent non-volatile material of about 34.4. The percent non-volatile material value can be arrived at by weighing out about 0.5 gram of copolymer solution and placing it into an oven at about 110° C. for about 20 minutes and then measuring the weight difference.

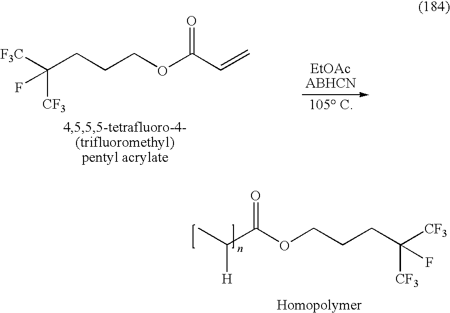

According to scheme (184) above, about 5.0 grams (0.018 mole) of 4,5,5,5-tetrafluoro-4-(trifluoromethyl)pentyl acrylate, about 6.0 grams of ethyl acetate, and about 0.025 gram (1.02×10⁴ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 4 hours at a temperature of about 105° C. The resulting copolymer can have a molecular weight of about 11,700 by gas permeation chromatography and a percent non-volatile material of about 25.2.

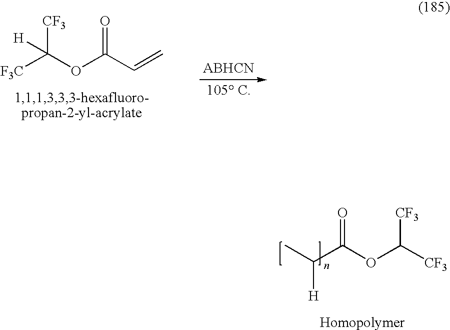

According to scheme (185) above, about 5.0 grams (0.021 mole) of 1,1,1,3,3,3-hexafluoropropan-2-yl acrylate, about 6.0 grams of ethyl acetate, and about 0.025 gram (1.02×10⁻⁴ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 4 hours at a temperature of about 105° C. The resulting copolymer can have a molecular weight of about 13,875 by gas permeation chromatography and a percent non-volatile material of about 29.3.

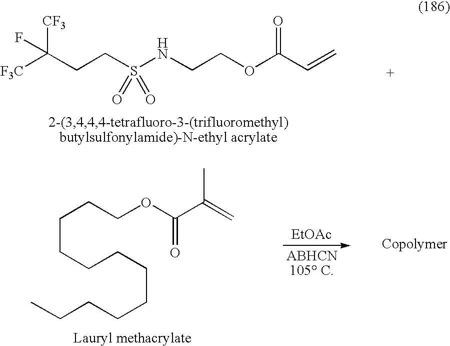

According to scheme (186) above, about 3.5 grams (0.009 mole) of 2-(3,4,4,4-tetrafluoro-3-(trifluoromethyl)butylsulfonylamide)-N-ethylmethacrylate, about 6.0 grams of ethyl acetate, 1.5 grams (0.006 mole) of lauryl methacrylate, and about 0.025 gram (1.02×10⁻⁴ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 4 hours at a temperature of about 105° C. The resulting copolymer can have a molecular weight of about 19,800 by gas permeation chromatography and a percent non-volatile material of about 40.7.

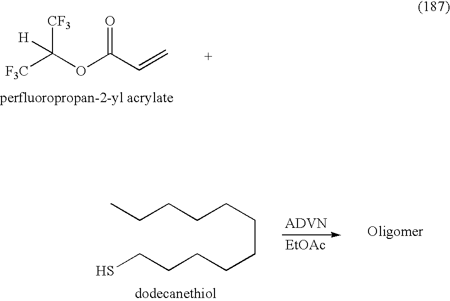

According to scheme (187) above, about 5.0 grams (0.018 mole) of 4,5,5,5-tetrafluoro-4-(trifluoromethyl)pentyl acrylate about 13.0 grams of ethyl acetate, 0.3 gram (0.0016 mole) of dodecanethiol, and about 0.01 gram (6.05×10⁻⁵ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 12 hours at about 80° C. The resulting copolymer can have a molecular weight of about 4330 by gas permeation chromatography and a percent non-volatile material of about 25.5.

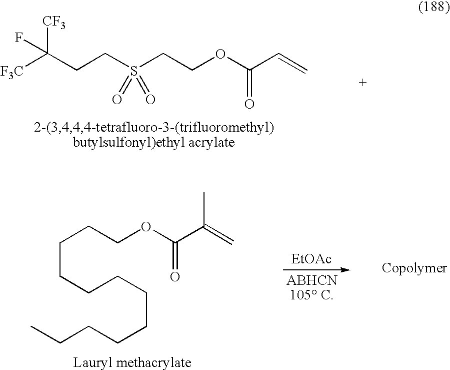

According to scheme (188) above, about 3.5 grams (0.01 mole) of 2-(3,4,4,4-tetrafluoro-3-(trifluoromethyl)butylsulfonyl)ethyl acrylate, about 6.0 grams of ethyl acetate, 1.5 grams (0.006 mole) of lauryl methacrylate, and about 0.025 gram (1.02×10⁻⁴ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 4 hours at a temperature of about 105° C. The resulting copolymer can have a molecular weight of about 53,100 by gas permeation chromatography and a percent non-volatile material of about 30.7

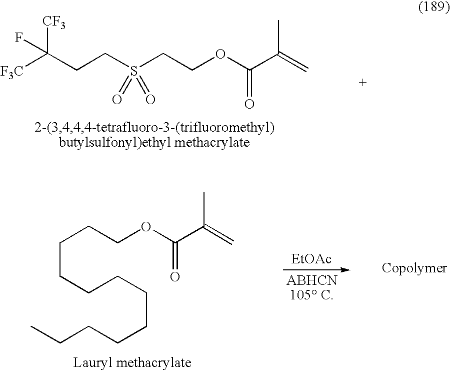

According to scheme (189) above, about 3.5 grams (0.01 mole) of 2-(3,4,4,4-tetrafluoro-3-(trifluoromethyl)butylsulfonyl)ethyl methacrylate, about 6.0 grams of ethyl acetate, 1.5 grams (0.006 mole) of lauryl methacrylate, and about 0.025 gram (1.02×10⁻⁴ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 4 hours at a temperature of about 105° C. The resulting copolymer can have a molecular weight of about 50,900 by gas permeation chromatography and a percent non-volatile material of about 26.4.

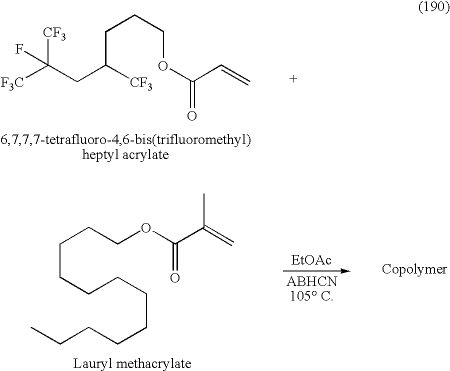

According to scheme (190) above, about 3.5 grams (0.009 mole) of 6,7,7,7-tetrafluoro-4,6-bis(trifluoromethy)heptyl acrylate, 1.5 grams (0.006 mole) of lauryl methacrylate, about 6.0 grams of ethyl acetate, and about 0.025 gram (1.02×10⁻⁴ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 4 hours at a temperature of about 105° C. The resulting copolymer can have a molecular weight of about 41,900 by gas permeation chromatography and a percent non-volatile material of about 33.7.

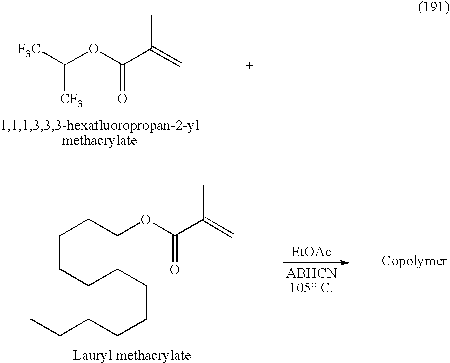

According to scheme (191) above, about 3.5 grams (0.015 mole) of 1,1,1,3,3,3-hexafluoropropan-2-yl methacrylate, 1.5 grams (0.006 mole) of lauryl methacrylate, about 6.0 grams of ethyl acetate, and about 0.025 gram (1.02×10⁻⁴ mole) of azobis(cyclohexanecarbonitrile) can be placed into a 500 mL glass lined Parr bottle which can be equipped with an agitator, thermocouple, and a means of heating the reactor to form a mixture. The reactor can be flushed with oxygen free nitrogen for about 30 seconds, sealed, and stirred for about 4 hours at a temperature of about 105° C. The resulting copolymer can have a percent non-volatile material of about 31.6.

As set forth above, the Rf-Diacrylate monomer can be prepared and polymerized by various methods and put to use in various applications as described in U.S. Pat. Nos. 4,137,139, 4,533,710, and 6,881,858.

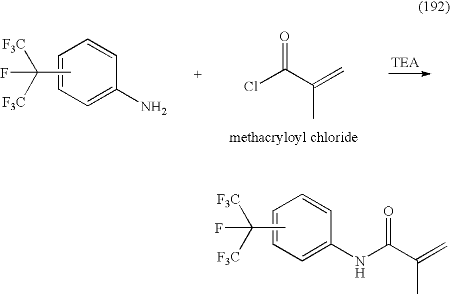

In accordance with scheme (192) above, in a flask that can be equipped with an agitator, thermocouple, reflux condenser, and an addition funnel, 50 ml of diethyl ether, 20.5 grams (0.0785 mole) of

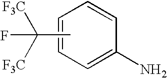

(can be prepared according to the procedure(s) set forth in EP 1 006 102 A2 the entirety of which is incorporated by reference) and 9.53 grams (0.0942 mole) of triethylamine can be placed to form a mixture. The mixture can be chilled to about 15° C. and 9.5 grams (0.086 moles) of methacryloyl chloride can be added drop wise at a rate sufficient to maintain a reaction temperature below about 18° C. to form a reaction mixture. The reaction mixture can be allowed to warm to room temperature over a period of about 1 hour while stirring. To the reaction mixture, 100 mL of water can be added to form a multiphase mixture from which an organic phase can be separated from an aqueous phase. The organic phase can be collected and dried over MgSO₄, filtered and concentrated under vacuum to afford what can be observed as a thick oil which solidified upon sitting. The solids can be recrystallized in a 35 mL ether and 50 mL hexane mixture to afford a slurry. The slurry can be filtered and dried to afford 10.5 grams of the

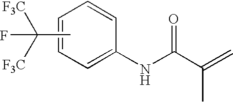

product. The product structure can be confirmed by NMR and/or chromatographic analysis.

Using the same general procedures found in examples E1 through E15, the polymerizations listed in table 38 below can be carried out using the concentrations shown.

TABLE 38
Polymer Composition and Properties

| | | % | % | |
|---|---|---|---|---|
| | % | (wt/wt) | (wt/wt) | MW (GPC) |
| Monomer | Monomer | LMA | NVM | (× 1000) |
| 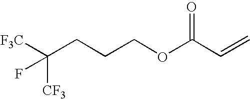 | 100 | 0 | 25.2 | 11.7 |
| 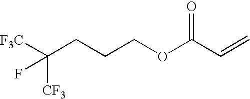 | 70 | 30 | 41.0 | 43 |
| 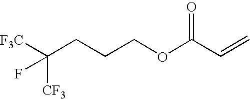 | 30 | 70 | 28.1 | 43.2 |
| 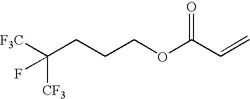 | 25 | 75 | 34.4 | 51 |
| 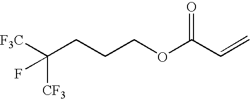 | 20 | 80 | 57.1 | 33.4 |
| 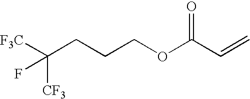 | 15 | 85 | 34.3 | 40 |
| 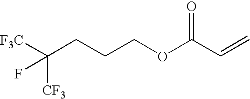 | 10 | 90 | 34.0 | 41 |
| 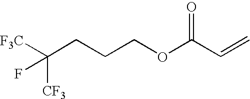 | 5 | 95 | 30.5 | 36.5 |
| 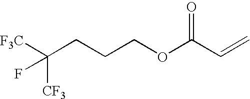 | 4 | 96 | 32.9 | 19 |
| 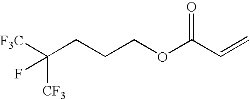 | 3 | 97 | 34.6 | 20.8 |
| 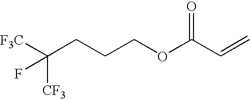 | 2 | 98 | 33.6 | 35.7 |
| 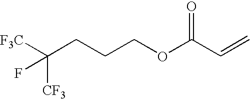 | 1 | 99 | 34.8 | 37 |
| 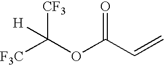 | 100 | 0 | 29.3 | 13.9 |
| 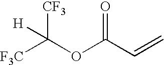 | 70 | 30 | 33.8 | 23.3 |
| 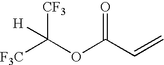 | 30 | 70 | 34.2 | 12 |
| 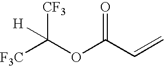 | 4 | 96 | 32.9 | 19 |
| 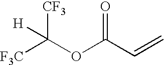 | 3 | 97 | 34.6 | 20.8 |
| 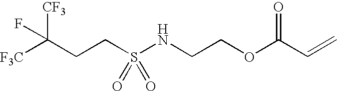 | 70 | 30 | 40.7 | 19.8 |
| 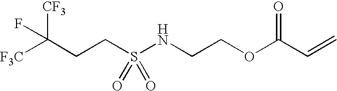 | 30 | 70 | 39.4 | 25.6 |
| 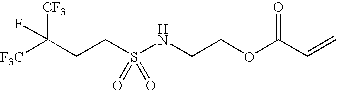 | 0.5 | 99.5 | 41.8 | 34.5 |
| 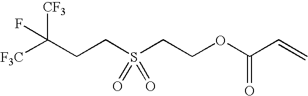 | 70 | 30 | 30.7 | 53.1 |
| 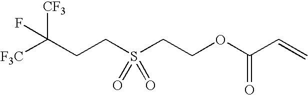 | 30 | 70 | 37.6 | 38.1 |
| 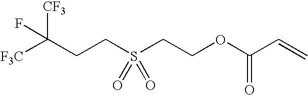 | 0.5 | 99.5 | 35.2 | 30.2 |
| 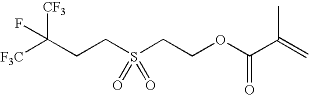 | 70 | 30 | 26.4 | 50.9 |
| 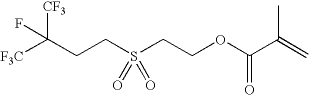 | 30 | 70 | 37.6 | 52.7 |
| 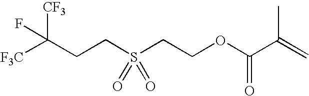 | 0.5 | 99.5 | 39.1 | 31.9 |
| 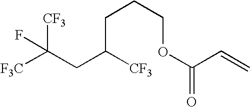 | 70 | 30 | 33.7 | 41.9 |
| 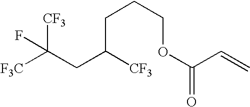 | 30 | 70 | 33.6 | 40.3 |
| 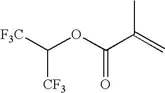 | 70 | 30 | 31.6 | NA |
| 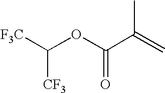 | 30 | 70 | 32.1 | NA |
| 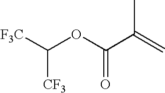 | 0.5 | 99.5 | 30.8 | NA |

NVM = Non-volatile material
GPC MW = Weight average molecular weight
NA = not available at present date
LMA = lauryl methacrylate

Gel Permeation Chromotography (GPC) Instrument Parameters
Waters 515 HPLC Pump
Waters 410 Differential Refractometer Detector
Phenomenex Phenogel 5 Columns
Polystyrene Standards having molecular weights of: 162, 580, 920, 1300, 2090, 2960, 3790, 5000, 7000, 9860, 43000, 76600, 117000, 135000, 186000, 210000, 275200, 488400

For example and by way of example only, solutions of R_{F}-monomers can be provided to a substrate and allowed to complex, for example, via evaporating the solvent of the solution to form a complex that includes a R_{F}-monomer unit. Providing these solutions to a substrate such as glass, nylon, and/or cotton and allowing the R_{F}-monomer to become part of a complex, such as coating the substrate.

The surface energy of the complex can be determined using the standard Fowkes method using diiodomethane and water as probe liquids, and the Zisman method of surface energy analysis using octane, decane, tetradecane, and hexadecane as probe liquids. Contact angle of drops of Zisman probe liquids, as well as, the Fowkes probes can be determined, using a Kruss prop Shape Analysis System. Surface energy data of complexes that include R_{F}-Q_{P} monomer units are recited in the following Tables 40-42.

R_{F}-monomers can be incorporated with other monomers and then incorporated into the construction of paper materials or used to treat paper materials. R_{F}-monomers can also be used to prepare polymer solutions. Polymeric solutions can be diluted to a percentage aqueous or non-aqueous solution and then applied to substrates to be treated, such as paper plates.

R_{F}-monomers can also be incorporated Into copolymers with comonomers such as the dialkyl amino alkyl acrylate or methacrylate or acrylamide or methacrylamide monomer and its amine salt quaternary ammonium or amine oxide form, as described in U.S. Pat. No. 4,147,851, herein incorporated by reference. The general formula for R_{F}-monomers can be R_{F}qO₂CC(R)═CH₂, with R being H or CH₃, q being an alkylene of 1 to 15 carbon atoms, hydroxyalkylene of 3 to 15 carbon atoms, or CₙH₂ₙ(OC_{q}H_{2q})ₘ—, —SO₂NR₁(CₙH₂ₙ)—, or —CONR₁(CₙH₂ₙ)—, n is 1 to 15, q is 2 to 4, and m is 1 to 15. Monomers used to form copolymers with acrylates and the R_{F}-monomers include those having amine functionality. These copolymers can be diluted in a solution and applied or incorporated directly into or on substrates to be treated, such as paper.

R_{F}-monomers can also be used to form acrylate polymers or other acrylate monomers consistent with those described in U.S. Pat. No. 4,366,299, herein incorporated by reference. As described, R_{F}-monomers can be incorporated into paper products or applied thereon.

R_{F}-monomers, acrylates and/or acrylics, for example, can be applied to finished carpet or incorporated into the finished carpet fiber before it is woven into carpet. R_{F}-monomers can be applied to carpet by a normal textile finishing process known as padding, in which the carpet is passed through a bath containing the R_{F}-monomer and, for example, latex, water, and/or other additives such as non-rewetting surfaces. The carpet can then be passed through nip rollers to control the rate of the add-on before being dried in a tenter frame.

R_{F}-monomers may also be incorporated into the fiber by reacting the fiber with R_{F}-intermediates having isocyanate functionality, R_{F}-isocyanate, for example.

R_{F} portions can also be incorporated into materials used to treat calcitic and/or siliceous particulate materials. For example, R_{F}-monomers can be incorporated into a copolymer where the copolymer can either be part of a formulation to treat these materials or used by itself to treat these materials as described in U.S. Pat. No. 6,383,569, herein incorporated by reference. The R_{F}-monomer can have the general formula R_{F}-Q-A-C(O)—C(R)═CH₂ wherein R_{F} is described above, R is H or CH₃, A is O, S, or N(R₁), wherein R₁ is H or an alkyl of from 1 to 4 carbon atoms, Q is alkylene of 1 to about 15 carbon atoms, hydroxyalkylene of 3 to about 15 carbon atoms, —(CₙH₂ₙ)(OC_{q}H_{2q})ₘ—, —SO₂—NR₁(CₙH₂ₙ)—, or —CONR₁(CₙH₂ₙ)—, wherein R₁ is H or an alkyl of 1 to 4 carbon atoms, n is 1 to 15, q is 2 to 4, and m is 1 to 15.

R_{F}-compositions and mixtures containing the R_{F} portion can be used to treat substrates including hard surfaces like construction materials such as brick, stone, wood, concrete, ceramics, tile, glass, stucco, gypsum, drywall, particle board, and chipboard. These compositions and mixtures can be used alone or in combination with penetration assistance such as non-ionic surfactants. These compositions can be applied to the surface of calcitic and/or siliceous architectural construction material by known methods, for example, by soaking, impregnation, emersion, brushing, rolling, or spraying. The compositions can be applied to the surface to be protected by spraying. Suitable spraying equipment is commercially available. Spraying with a compressed air sprayer is an exemplary method of application to the particular substrate. U.S. Pat. Nos. 6,197,382 and 5,674,961 also describe methods for applying and using polymer solutions and are herein incorporated by reference.

In an exemplary process of producing solutions having components with R_{F}, an R_{F}-intermediate having a methyl-epoxide functionality may be condensed with a monocarboxylic alkenoic acid to prepare an unsaturated R_{F}-ester (not shown). Exemplary methods for producing these kinds of unsaturated esters are described in U.S. Pat. No. 5,798,415, herein incorporated by reference. Additional esters may be prepared according to U.S. Pat. No. 4,478,975, herein incorporated by reference. Components of these solutions can also include dimethyl amino ethyl methacrylate, and these components can be applied in organic and inorganic solvents, as described in U.S. Pat. No. 6,120,892 herein incorporated by reference. R_{F}-monomers can also be combined with other monomers to produce copolymers or in solutions with amido and sulfur monomers as described by U.S. Pat. No. 5,629,372 herein incorporated by reference.

R_{F}-intermediates having amine functionality can also be reacted with tetrachlorophthalic anhydride using U.S. Pat. No. 4,043,923 as an exemplary reaction scheme (not shown). U.S. Pat. No. 4,043,923 is herein incorporated by reference. The reaction product can be mixed with a carpet cleaning solution to provide soil repellency.

In exemplary embodiments urethanes containing a R_{F}Q_{U} (R_{F}-Urethanes) can be prepared from R_{F}-Intermediates. R_{F}-Urethanes can include R_{F}-intermediates above, but may contain functionality that allows for their conjugation with another R_{F}Q_{U} compounds, but not necessarily the same R_{F}Q_{U} compound. According to exemplary embodiments the R_{F} portion of the urethane can at least partially include an R_{F}(R_{T})n portion as described above. The R_{F}(R_{T})n portion of the urethane can also include the R_{S} portion described above. In accordance with exemplary implementations the R_{S} portion can be incorporated to provide additional carbon between the R_{F} and/or R_{F}(R_{T})n portions and the Q_{U} portion of the urethane. Exemplary R_{S} portions include —CH₂—CH₂—. Exemplary RF-urethanes, such as R_{F}-Q_{U}, can include, but are not limited to those listed in Table 39 below.

TABLE 39
Exemplary R_{F}-Urethanes

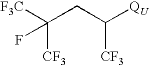

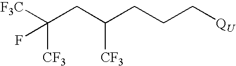

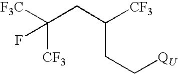

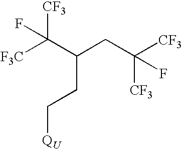

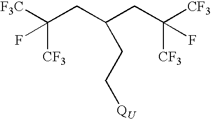

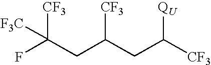

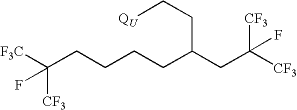

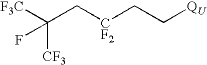

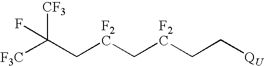

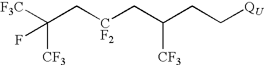

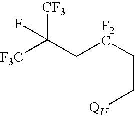

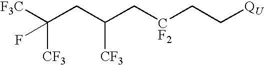

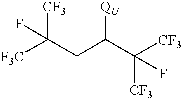

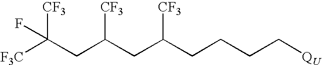

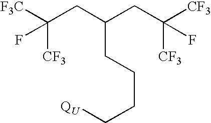

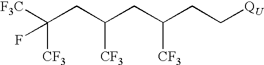

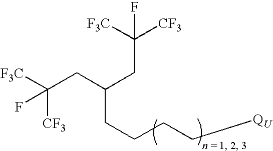

Referring to scheme (193) below, urethanes, including R_{F} portions can be prepared from R_{F}-intermediates.

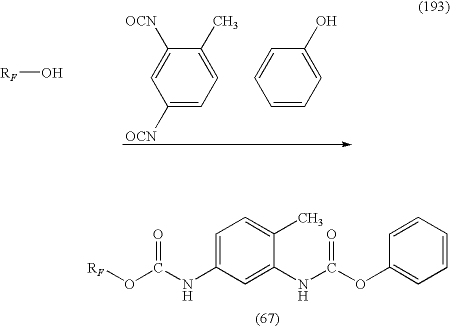

An R_{F}-intermediate (R_{F}—OH) can be combined with hexamethylene diisocyanate polymers (DESMODUR N-100) following the general reaction sequence described in U.S. Pat. No. 5,827,919, herein incorporated by reference, to produce a urethane. Another method for preparing urethanes includes reacting a R_{F}-intermediate (R_{F}—SCN) with epichlorohydrin to produce a “twin tailed” R_{F}-intermediate which can be reacted with diisocyanate and/or a urethane prepolymer as described in U.S. Pat. No. 4,113,748, herein incorporated by reference (not shown). Urethanes having the RF group can then be incorporated as an additive to compositions such as latex paint. U.S. Pat. No. 5,827,919 describes methods for utilizing these urethanes and is herein incorporated by reference. R_{F}-urethanes and polyurethanes can be used to treat substrates such as carpet, drapery, upholstery, automotive, awning fabrics, and rainwear.

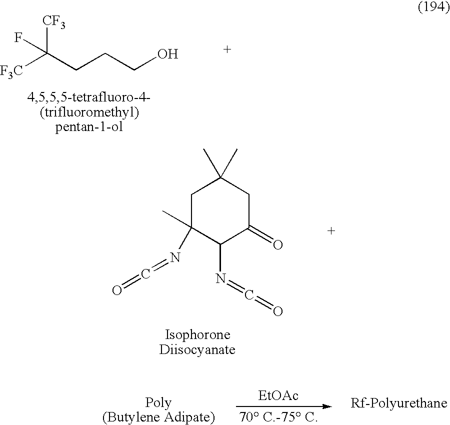

According to scheme (194) above, into a 500 mL flask that can be equipped with an agitator, thermocouple, and an addition funnel, 20.24 grams of Poly(butylene adipate), 9.83 grams (0.044 mole) of isophorone diisocyanate, and 66.3 grams of ethyl acetate can be added to form a mixture. The mixture can be heated to from about 70° C. to about 75° C. while stirring for from about three hours to about four hours. To the mixture, 1 drop of dibutyl tin dilaurate and 3.66 grams (0.016 mole) of 4,5,5,5-tetrafluoro-4-(trifluoromethyl)pent-1-ol can be added to form a reaction mixture. The reaction mixture can be held at said temperature range for about two hours. The resulting fluoropolyurethane can have a fluorine content of about 10.24 (wt/wt) percent.

In accordance with scheme (194) above, into a 500 mL flask that can be equipped with an agitator, thermocouple, and an addition funnel, 15.8 grams of Poly(butylene adipate), 13.5 grams (0.061 mole) of isophorone diisocyanate, and 67.2 grams of ethyl acetate can be added to form a mixture. The mixture can be heated to from about 70° C. to about 75° C. while stirring for from about three hours to about four hours. To the mixture, 1 drop of dibutyl tin dilaurate and 3.61 grams (0.016 mole) of 4,5,5,5-tetrafluoro-4-(trifluoromethyl)pent-1-ol can be added to form a reaction mixture. The reaction mixture can be held at said temperature range for about two hours. The resulting fluoropolyurethane can have a fluorine content of about 9.96 (wt/wt) percent.

According to scheme (194) above, into a 500 mL flask that can be equipped with an agitator, thermocouple, and an addition funnel, 14.2 grams of Poly(butylene adipate), 14.5 grams (0.065 mole) of isophorone diisocyanate, and 67.5 grams of ethyl acetate can be added to form a mixture. The mixture can be heated to from about 70° C. to about 75° C. while stirring for from about three hours to about four hours. To the mixture, 1 drop of dibutyl tin dilaurate and 3.87 grams (0.017 mole) of 4,5,5,5-tetrafluoro-4-(trifluoromethyl)pent-1-ol can be added to form a reaction mixture. The reaction mixture can be held at said temperature range for about two hours. The resulting fluoropolyurethane can have a fluorine content of about 10.67 (wt/wt) percent.

Referring to scheme (194) above, into a 500 mL flask that can be equipped with an agitator, thermocouple, and an addition funnel, 12.8 grams of Poly(butylene adipate), 15.5 grams (0.07 mole) of isophorone diisocyanate, and 67.8 grams of ethyl acetate can be added to form a mixture. The mixture can be heated to from about 70° C. to about 75° C. while stirring for from about three hours to about four hours. To the mixture, 1 drop of dibutyl tin dilaurate and 3.9 grams (0.017 mole) of 4,5,5,5-tetrafluoro-4-(trifluoromethyl)pent-1-ol can be added to form a reaction mixture. The reaction mixture can be held at said temperature range for about two hours. The resulting fluoropolyurethane can have a fluorine content of about 10.67 (wt/wt) percent.

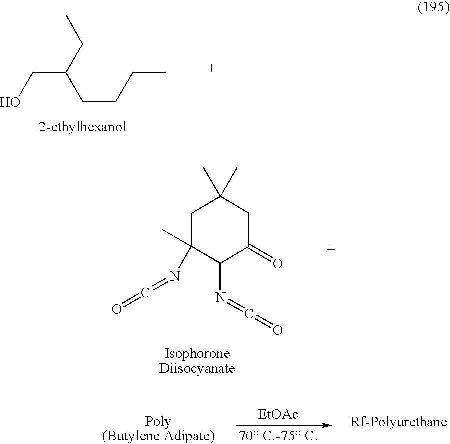

In reference to scheme (195) above, into a 500 mL flask that can be equipped with an agitator, thermocouple, and an addition funnel, 13.46 grams of Poly(butylene adipate), 16.24 grams (0.073 mole) of isophorone diisocyanate, and 67.9 grams of ethyl acetate can be added to form a mixture. The mixture can be heated to from about 70° C. to about 75° C. while stirring for from about three hours to about four hours. To the mixture, 1 drop of dibutyl tin dilaurate and 2.34 grams (0.018 mole) of 2-ethylhexanol can be added to form a reaction mixture. The reaction mixture can be held at said temperature range for about two hours.

In a flask, about 6.5 (wt/wt) percent of 1,2,3,4-butanetetracarboxylic acid, 6.0 (wt/wt) percent of sodium hypophosphite, and the balance comprising the fluoropolyurethane to form a coating mixture.

On a section of 100% cotton fabric, about 25 microliters of the coating mixture can be placed using a calibrated pipette to form a spot. A total of six spots can be placed on the fabric followed by placement into an oven at about 180° C. for about two minutes to promote crosslinking then can be allowed to set in air for about 24 hours.

On a section of Nylon 66 mesh fabric (PN CMN-0005 from Small Parts Incorporated), about 25 microliters of the coating mixture can be placed using a calibrated pipette to form a spot. A total of six spots can be placed on the fabric followed by placement into an oven at about 180° C. for about two minutes to promote crosslinking then can be allowed to set in air for about 24 hours.

On a clean glass slide, about 25 microliters of the coating mixture can be placed using a calibrated pipette to form a spot. The spot can be allowed to spread along the glass slide surface. A total of six slides can be prepared followed by placement into an oven at about 180° C. for about two minutes to promote crosslinking then can be allowed to set in air for about 24 hours.

Two methods can be employed to obtain surface energy values, the standard Fowkes method using diiodomethane and water as probe liquids, and the Zisman method of surface energy analysis. The Zisman method can use the liquid set decane, dodecane, tertadecane, and hexadecane as four probe liquids—which can also provide contact angle data for hydrophobic oils on fluorourethane coatings. Each of the six liquids tested can employ a method wherein five drops of liquid were placed on each dried coating and measured for contact angle using a Kruss prop Shape Analysis System DSA10. Drop sizes were controlled to be about 1.0 microliter.

The surface energy values are summarized in the tables below:

TABLE 40
Polyfluorourethane Surface Energy Properties on Cleaned Glass

| | Zisman | Fowkes | | | |
|---|---|---|---|---|---|
| | Surface | Surface | Polar | Dispersive | Surface |
| | Energy | Energy | Component | Component | Polarity |
| Coating | (mJ/m²) | (mJ/m²) | (mJ/m²) | (mJ/m²) | (%) |
| 40-62 | 26.02 | 26.35 | 4.06 | 22.29 | 15.41 |
| 40-60 | 23.48 | 23.77 | 2.67 | 21.10 | 11.24 |
| 40-60B | 22.51 | 22.82 | 2.23 | 20.59 | 9.78 |
| 40-61 | 22.29 | 22.63 | 2.12 | 20.51 | 9.38 |
| 40-61B | 21.87 | 22.16 | 1.93 | 20.23 | 8.70 |

TABLE 41
Polyfluorourethane Surface Energy Properties on Nylon Fabric

| | Zisman | Fowkes | | | |
|---|---|---|---|---|---|
| | Surface | Surface | Polar | Dispersive | Surface |
| | Energy | Energy | Component | Component | Polarity |
| Coating | (mJ/m²) | (mJ/m²) | (mJ/m²) | (mJ/m²) | (%) |
| 40-62 | 25.88 | 26.19 | 3.95 | 22.24 | 15.09 |
| 40-60 | 22.93 | 23.25 | 2.41 | 20.84 | 10.36 |
| 40-60B | 21.97 | 22.25 | 1.98 | 20.27 | 8.91 |
| 40-61 | 21.77 | 22.07 | 1.88 | 20.19 | 8.50 |
| 40-61B | 21.34 | 21.62 | 1.72 | 19.90 | 7.95 |

TABLE 42
Polyfluorourethane Surface Energy Properties on Cotton Fabric

| | Zisman | Fowkes | | | |
|---|---|---|---|---|---|
| | Surface | Surface | Polar | Dispersive | Surface |
| | Energy | Energy | Component | Component | Polarity |
| Coating | (mJ/m²) | (mJ/m²) | (mJ/m²) | (mJ/m²) | (%) |
| 40-61B | 21.62 | 21.91 | 1.83 | 20.08 | 8.35 |

The R_{F} portion can also be complexed as an acid with amine and quaternary ammonium polymers as described In U.S. Pat. No. 6,486,245, herein incorporated by reference (not shown).

What is claimed is:

1. A surfactant composition comprising R_{F}(R_{T})ₙQs, wherein:
the R_{F} group comprises at least two —CF₃ groups;
the R_{T} group comprises a group having at least two carbons;
n is at least 1; and
the Qs group is at least one atom of the periodic table of elements, wherein at least a portion of the R_{F} and R_{T} groups are hydrophobic relative to the Qs group, and at least a portion of the Qs group is hydrophilic relative to the R_{F} and R_{T} groups.

2. The composition of claim 1 wherein the R_{T} group comprises an R_{S} group, the R_{S} group comprising a C-2 group, the R_{S} group providing at least two carbons between the Qs group and the remainder of the R_{T} and R_{F} groups.

3. The composition of claim 2 wherein the C-2 group comprises —CH₂—CH₂—.

4. The composition of claim 1 wherein the R_{F} group comprises —CF(CF₃)₂.

5. The composition of claim 1 wherein the R_{F} group is one of ((CF₃)₂CFCH₂)₂CH—, ((CF₃)₂CFCH₂)₂CH₂CH₂—, (CF₃)₂CFCH₂((CF₃)₂CF)CH—, (CF₃)₂CFCH₂CH(CF₃)CH₂CH(CF₃)—, or (CF₃)₂CFCH₂CH₂CH₂CH₂((CF₃)₂CFCH)CH—.

6-9. (canceled)

10. The composition of claim 1 wherein the R_{T} group comprises one or more of 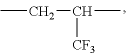
—CH₂CF₂—, —CH₂—(CH₂CF(CF₃)₂)CH—, and —CH₂—CH₂—.

11-13. (canceled)

14. The composition of claim 1 wherein n is at least 2 and the composition comprises

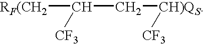

15. The composition of claim 1 wherein the Qs group comprises a sulfonyl group.

16. The composition of claim 1 wherein R_{F}(R_{T})ₙQs is

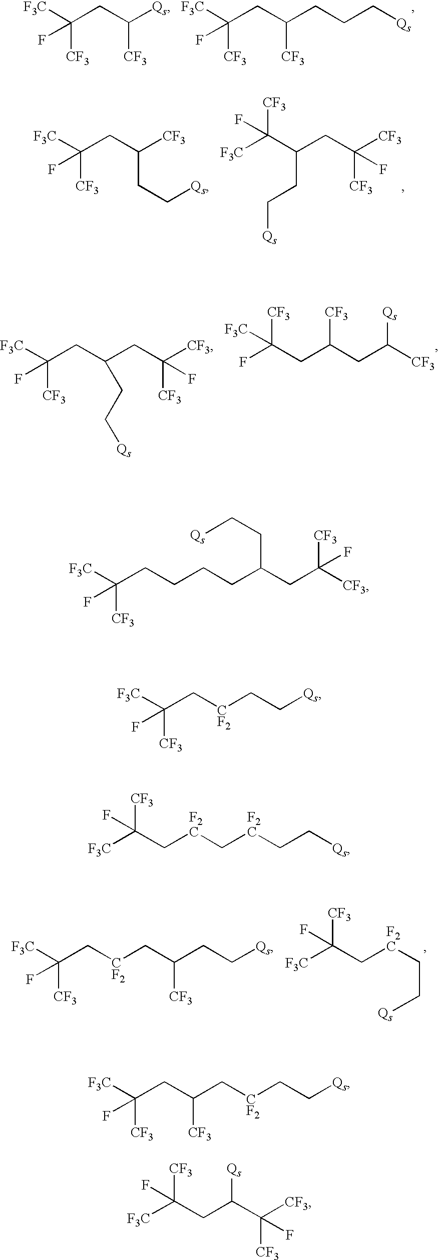

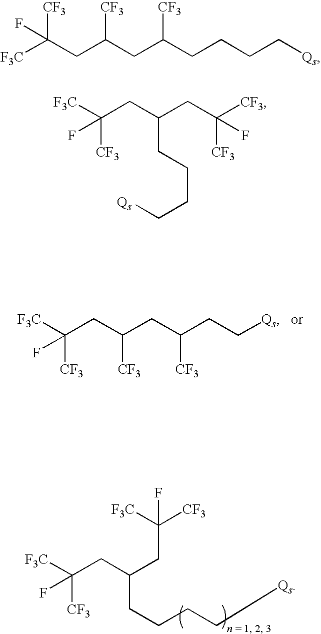

17-32. (canceled)

33. A surfactant composition comprising R_{F}-Q_{S}, wherein:
R_{F} comprises at least one fluorine atom; and
Q_{S} comprises and n-oxide group.

34. The composition of claim 33 wherein the R_{F}-Q_{S} is

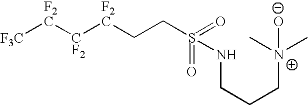

35. A foam stabilizer composition comprising R_{F}(R_{T})ₙQ_{FS}, wherein:
the R_{F} group comprises at least two —CF₃ groups;
the R_{T} group comprises a group having at least two carbons;
n is at least 1; and
the Q_{FS} group is at least one atom of the periodic table of elements, wherein at least a portion of the R_{F} and R_{T} groups are hydrophobic relative to the Q_{FS} group, and at least a portion of the Q_{FS} group is hydrophilic relative to the R_{F} and R_{T} groups.

36. The composition of claim 35 wherein the R_{T} group comprises an R_{S} group, the R_{S} group comprising a C-2 group, the R_{S} group providing at least two carbons between the Q_{FS} group and the remainder of the R_{T} and R_{F} groups.

37. The composition of claim 36 wherein the C-2 group comprises —CH₂—CH₂—.

38. The composition of claim 35 wherein the R_{F} group comprises —CF(CF₃)₂.

39. The composition of claim 35 wherein the R_{F} group is one of ((CF₃)₂CFCH₂)₂CH—, (CF₃)₂CFCH₂CH₂CH₂CH₂((CF₃)₂CFCH)CH—, (CF₃)₂CFCH₂((CF₃)₂CF)CH—, (CF₃)₂CFCH₂CH(CF₃)CH₂CH(CF₃)—, or ((CF₃)₂CFCH₂)₂CH₂CH₂—.

40-43. (canceled)

44. The composition of claim 35 wherein the R_{T} group comprises one or more of 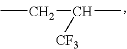
—CH₂—CF₂—, —CH₂—(CH₂CF(CF₃)₂)CH—, or —CH₂—CH₂—.

45-47. (canceled)

48. The composition of claim 35 wherein n is at least 2 and the composition comprises

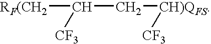

49. The composition of claim 35 wherein the Q_{FS} group comprises a sulfonyl group.

50. The composition of claim 35 wherein R_{F}(R_{T})ₙQ_{FS}

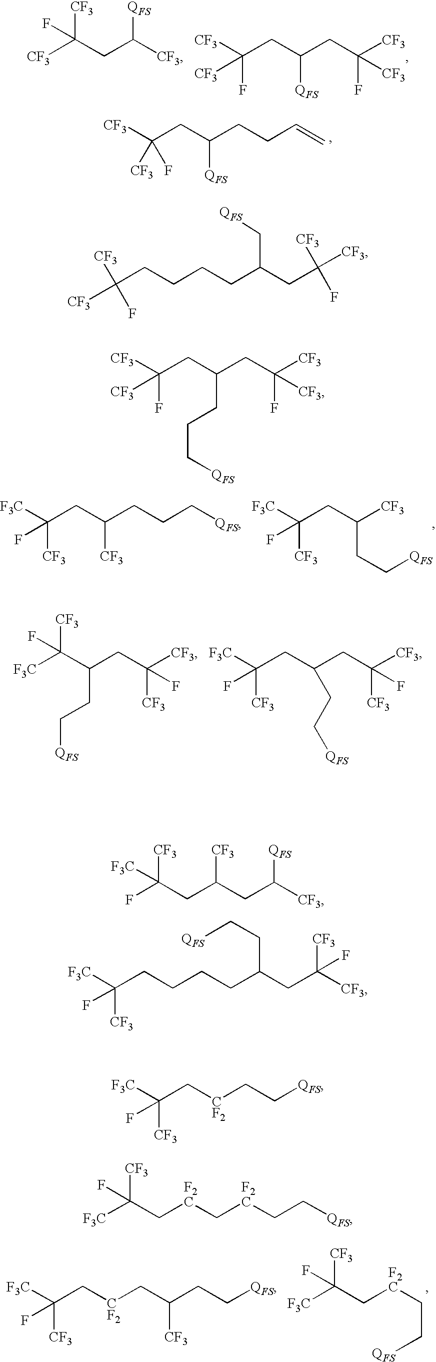

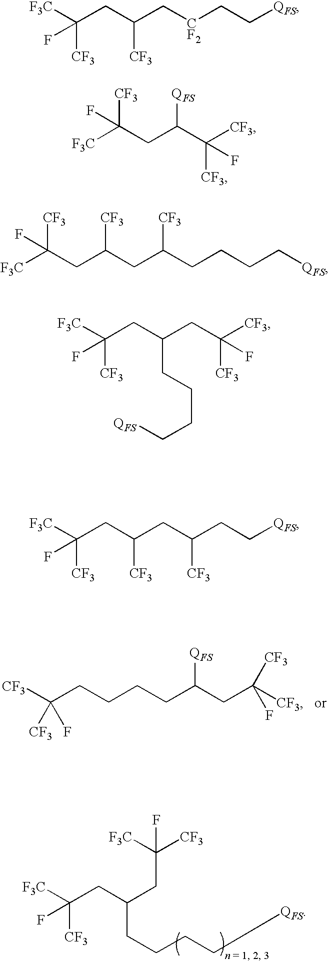

51-71. (canceled)

72. A monomer comprising R_{F}(R_{T})ₙQ_{M}, wherein:
the R_{F} group comprises at least two —CF₃ groups;
the R_{T} group comprises a group having at least two carbons;
n is at least 1; and
the Q_{M} group is at least one atom of the periodic table of elements.

73. The composition of claim 72 wherein the RT group comprises an R_{S} group, the R_{S} group comprising a C-2 group, the R_{S} group providing at least two carbons between the Q_{M} group and the remainder of the R_{T} and R_{F} groups.

74. The composition of claim 72 wherein the C-2 group comprises —CH₂—CH₂—.

75. The monomer of claim 72 wherein the R_{F} group comprises —CF(CF₃)₂.

76. The monomer of claim 72 wherein the R_{F} group is one of ((CF₃)₂CFCH₂)₂CH—, ((CF₃)₂CFCH₂)₂CH₂CH₂—, (CF₃)₂CFCH₂((CF₃)₂CF)CH—, (CF₃)₂CFCH₂CH(CF₃)CH₂CH(CF₃)—, or (CF₃)₂CFCH₂CH₂CH₂CH₂((CF₃)₂CFCH)CH—.

77-79. (canceled)

80. The monomer of claim 72 wherein the R_{F} group is (CF₃)₂CFCH₂CH₂CH₂CH₂((CF₃)₂CFCH)CH—.

81. The monomer of claim 72 wherein the R_{T} group comprises one or more of 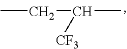
—CH₂—CF₂—, —CH₂—(CH₂CF(CF₃)₂)CH—, or CH₂—CH₂—.

82-84. (canceled)

85. The monomer of claim 72 wherein n is at least 2 and the monomer comprises

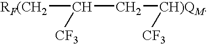

86. The monomer of claim 72 wherein the Q_{M} group comprises an olefinic group.

87. The monomer of claim 72 wherein R_{F}(R_{T})ₙQ_{M} is

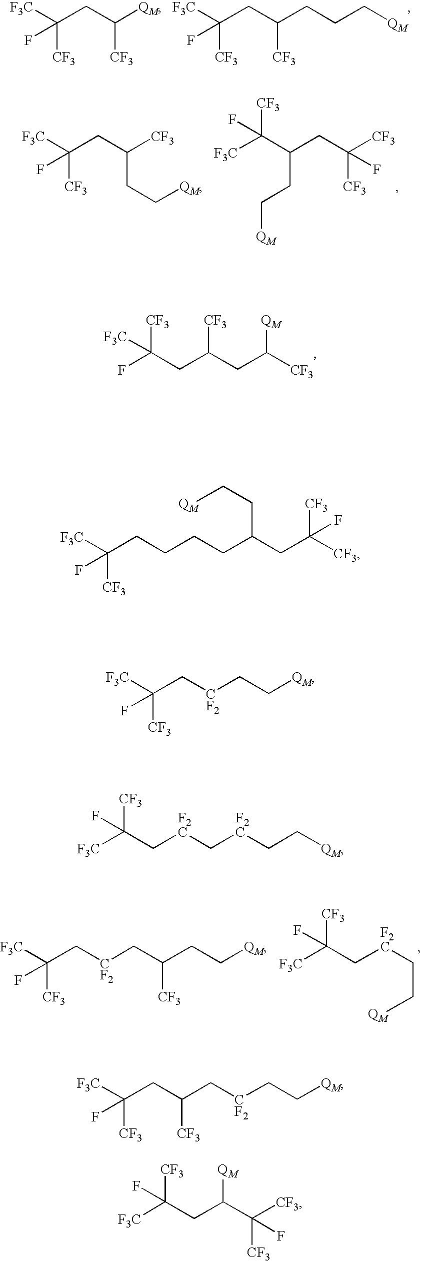

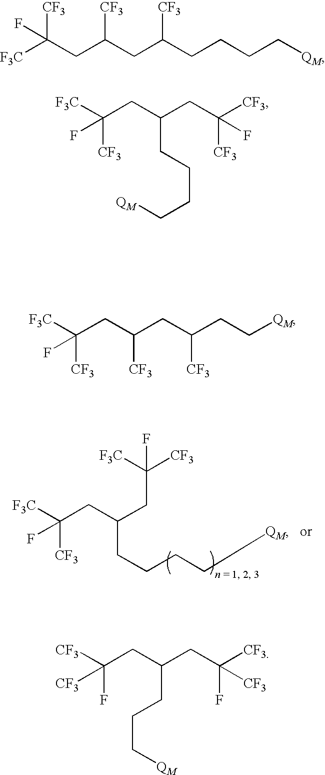

88-103. (canceled)

104. A polymer comprising R_{F}(R_{T})ₙQ_{MU}, wherein:
the R_{F} group comprises at least two —CF₃ groups;
the R_{T} group comprises a group having at least two carbons;
n is at least 1; and
the Q_{MU} group is a portion of a polymer chain backbone.

105. The composition of claim 104 wherein the R_{T} group comprises an R_{S} group, the R_{S} group comprising a C-2 group, the R_{S} group providing at least two carbons between the Q_{MU} group and the remainder of the R_{T} and R_{F} groups.

106. The composition of claim 104 wherein the C-2 group comprises —CH₂—CH₂—

107. The polymer of claim 104 wherein the R_{F} group comprises —CF(CF₃)₂.

108. The polymer of claim 104 wherein the R_{F} group is one of ((CF₃)₂CFCH₂)₂CH—, ((CF₃)₂CFCH₂)₂CH₂—, (CF₃)₂CFCH₂((CF₃)₂CF)CH—, (CF₃)₂CFCH₂CH(CF₃)CH₂CH(CF₃)—, or (CF₃)₂CFCH₂CH₂CH₂CH₂((CF₃)₂CFCH)CH—.

109-112. (canceled)

113. The polymer of claim 104 wherein the R_{T} group comprises one or more of 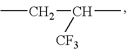
—CH₂—CF₂—, —CH₂—(CH₂CF(CF₃)₂)CH—, and —CH₂—CH₂—.

114-116. (canceled)

117. The polymer of claim 104 wherein n is at least 2 and the polymer comprises

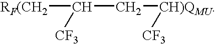

118. The polymer of claim 104 wherein the Q_{FS} group comprises a sulfonyl group.

119. The polymer of claim 104 wherein R_{F}(R_{T})ₙQ_{MU} is

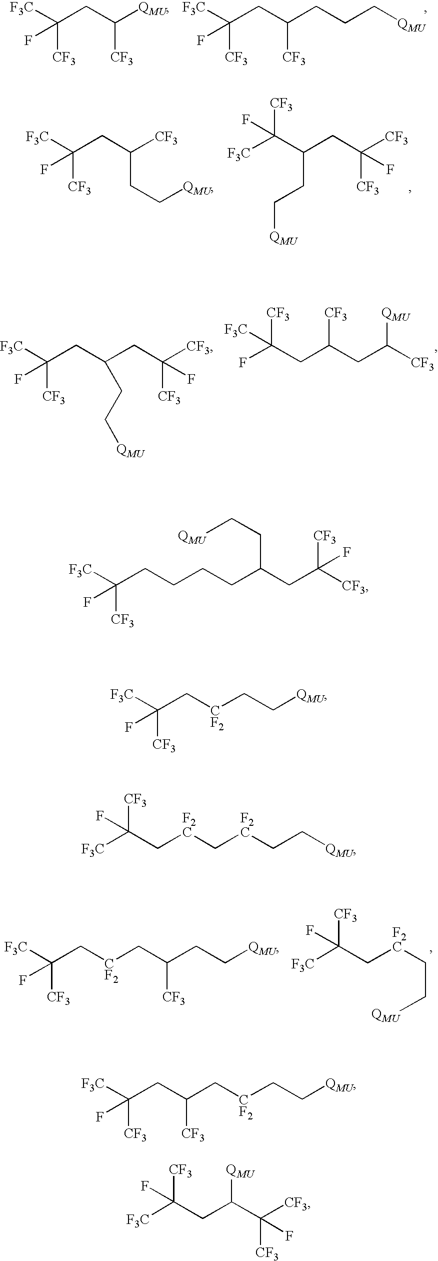

120-137. (canceled)

138. A urethane comprising R_{F}(R_{T})ₙQ_{U}, wherein:
the R_{F} group comprises at least two —CF₃ groups;
the R_{T} group comprises a group having at least two carbons;
n is at least 1; and
the Q_{U} group is at least one atom of the periodic table of elements.

139. The urethane of claim 138 wherein the R_{T} group comprises an R_{S} group, the R_{S} group comprising a C-2 group, the R_{S} group providing at least two carbons between the Q_{U} group and the remainder of the R_{T} and R_{F} groups.

140. The urethane of claim 139 wherein the C-2 group comprises —CH₂—CH₂—.

141. The urethane of claim 138 wherein the R_{F} group comprises —CF(CF₃)₂.

142. The urethane of claim 138 wherein the R_{F} group is one of ((CF₃)₂CFCH₂)₂CH—, ((C₃)₂CFCH₂)₂CH₂CH₂—, (CF₃)₂CFCH₂((CF₃)₂CF)CH—(CF₃)₂CFCH₂CH(CF₃)CH₂CH(CF₃)—, or (CF₃)₂CFCH₂CH₂CH₂CH₂((CF₃)₂CFCH)CH—.

143-146. (canceled)

147. The urethane of claim 138 wherein the R_{T} group comprises one or more of 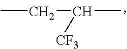
—CH₂—CF₂—, —CH₂—(CH₂CF(CF₃)₂CH—, and —CH₂CH₂—.

148-150. (canceled)

151. The urethane of claim 138 wherein n is at least 2 and the urethane comprises

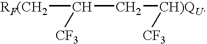

152. The urethane of claim 138 wherein the Q_{U} group comprises an cyclic aromatic group.

153. The urethane of claim 138 wherein R_{F}(R_{T})ₙQ_{U} is

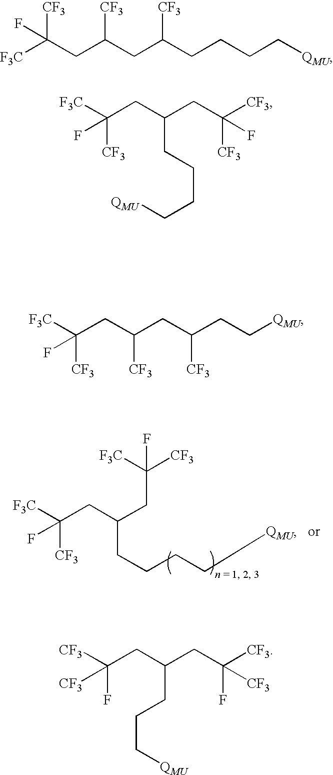

154-170. (canceled)

171. A glycol comprising R_{F}(R_{T})ₙQ_{H}, wherein:
the R_{F} group comprises at least two —CF₃ groups;
the R_{T} group comprises a group having at least two carbons;
n is at least 1; and
the Q_{H} group is a portion of a glycol chain backbone.

172. The glycol of claim 171 wherein the R_{T} group comprises an R_{S} group, the R_{S} group comprising a C-2 group, the R_{S} group providing at least two carbons between the Q_{H} group and the remainder of the R_{T} and R_{F} groups.

173. The glycol of claim 172 wherein the C-2 group comprises —CH₂—CH₂—

174. The glycol of claim 171 wherein the R_{F} group comprises —CF(CF₃)₂.

175. The glycol of claim 171 wherein the R_{F} group is one of ((CF₃)₂CFCH₂)₂CH—, ((CF₃)₂CFCH₂)₂CH₂CH₂—, (CF₃)₂CFCH₂((CF₃)₂CF)CH—, (CF₃)₂CFCH₂CH(CF₃)CH₂CH(CF₃)—, or (CF₃)₂CFCH₂CH₂CH₂CH₂((CF₃)₂CFCH)CH—.

176-179. (canceled)

180. The glycol of claim 171 wherein the RT group comprises one or more of 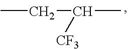
—CH₂—CF₂—, —CH₂—(CH₂CF(CF₃)₂)CH—, and —CH₂—CH₂—.

181-183. (canceled)

184. The glycol of claim 171 wherein n is at least 2 and the glycol comprises

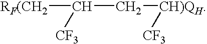

185. The glycol of claim 171 wherein the Q_{H} group comprises at least one hydroxyl group.

186. The glycol of claim 171 wherein R_{F}(R_{T})ₙQ_{H} is one

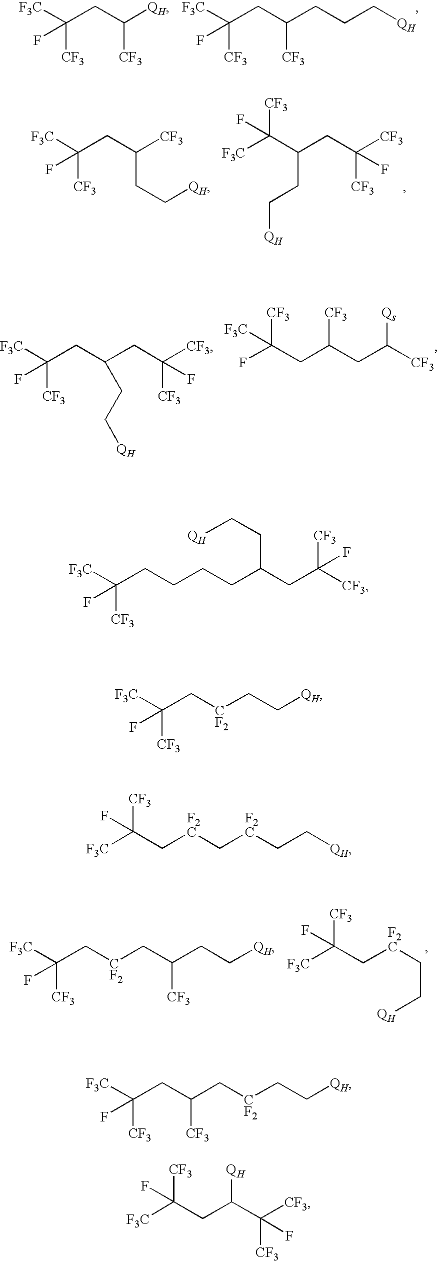

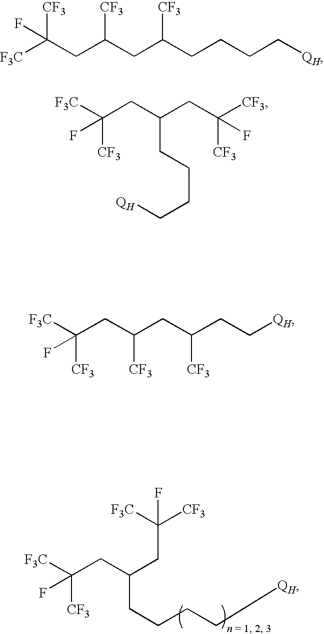

187-204. (canceled)

205. A metal complex comprising R_{F}(R_{T})ₙQ_{MC}, wherein:
the R_{F} group comprises at least two —CF₃ groups;
the R_{T} group comprises a group having at least two carbons;
n is at least 1; and
the Q_{MC} group comprises a charged group configured to complex one or more metal ions.

206. The metal complex of claim 205 wherein the R_{T} group comprises an R_{S} group, the R_{S} group comprising a C-2 group, the R_{S} group providing at least two carbons between the Q_{U} group and the remainder of the R_{T} and R_{F} groups.

207. The metal complex of claim 206 wherein the C-2 group comprises —CH₂—CH₂—.

208. The metal complex of claim 205 wherein the R_{F} group comprises —CF(CF₃)₂.

209. The metal complex of claim 205 wherein the R_{F} group is one of ((CF₃)₂CFCH₂)₂CH—, ((CF₃)₂CFCH₂)₂CH₂C₂—, (CF₃)₂CFCH₂((CF₃)₂CF)CH—, (CF₃)₂CFCH₂CH(CF₃)CH₂CF₃)—, or (CF₃)CF₂CFCH₂CH₂CH₂CH₂((CF₃)₂CFCH)CH—.

210-213. (canceled)

214. The metal complex of claim 205 wherein the R_{T} group comprises one or more of 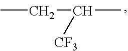
—CH₂—CF₂—, —CH₂—(CH₂CF(CF₃)₂)CH—, and —CH₂—CH₂—.

215-217. (canceled)

218. The metal complex of claim 205 wherein n is at least 2 and the metal complex comprises

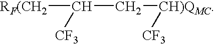

219. The metal complex of claim 205 wherein the Q_{MC} group comprises a chelating group.

220. The metal complex of claim 205 wherein R_{F}(R_{T})ₙQ_{MC} is one of

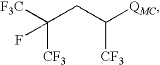

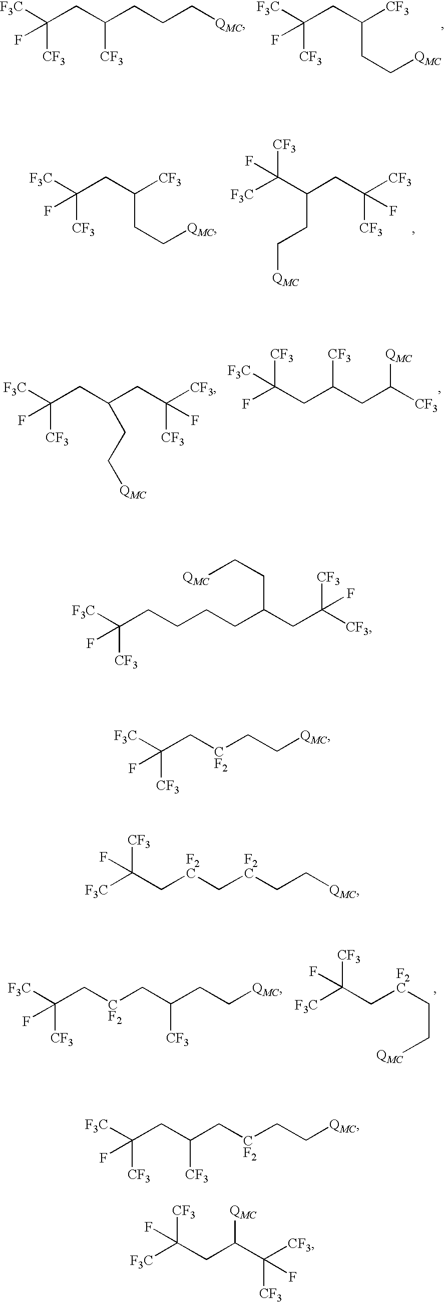

221-236. (canceled)

237. A phosphate ester composition comprising R_{F}(R_{T})ₙQ_{PE}, wherein:
the R_{F} group comprises at least two —CF₃ groups;
the R_{T} group comprises a group having at least two carbons;
n is at least 1; and
the Q_{PE} group is a portion of a phosphate ester.

238. The composition of claim 237 wherein the R_{T} group comprises an R_{S} group, the R_{S} group comprising a C-2 group, the R_{S} group providing at least two carbons between the Q_{PE} group and the remainder of the R_{T} and R_{F} groups.

239. The composition of claim 238 wherein the C-2 group comprises —CH₂—CH₂—

240. The composition of claim 237 wherein the R_{F} group comprises —CF(CF₃)₂.

241. The composition of claim 237 wherein the R_{F} group is one of ((CF₃)₂CFCH₂)₂CH—, ((CF₃)₂CFCH₂)₂CH₂CH₂—, (CF₃)₂CFCH₂((CF₃)₂CF)CH—, (CF₃)₂CFCH₂CH(CF₃)CH₂CH(CF₃)—, or (CF₃)₂CFCH₂CH₂CH₂CH₂((CF₃)₂CFCH)CH—.

242-245. (canceled)

246. The composition of claim 237 wherein the R_{T} group comprises one or more of 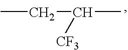
—CH₂—CF₂—, —CH₂—(CH₂CF(CF₃)₂)CH—, and —CH₂—CH₂—.

247-249. (canceled)

250. The composition of claim 237 wherein n is at least 2 and the composition comprises

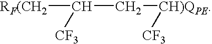

251. The composition of claim 237 wherein the Q_{PE} group comprises at least one carbonyl group.

252. The composition of claim 237 wherein R_{F}(R_{T})ₙQ_{PE}

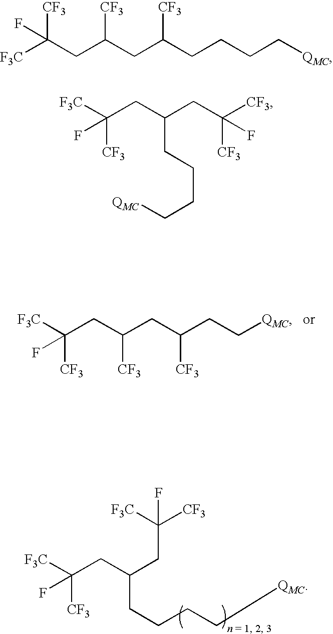

253-269. (canceled)

270. A composition comprising R_{F}(R_{T})ₙQ, wherein:
the R_{F} group comprises at least two —CF₃ groups;
the R_{T} group comprises a group having at least two carbons;
n is at least 1; and
the Q group comprises one or more atoms of the periodic table of elements.

271. The composition of claim 270 wherein the R_{T} group comprises an R_{S} group, the R_{S} group comprising a C-2 group, the R_{S} group providing at least two carbons between the Qs group and the remainder of the R_{T} and R_{F} groups.

272. The composition of claim 271 wherein the C-2 group comprises —CH₂—CH₂—.

273. The composition of claim 270 wherein the R_{F} group comprises —CF(CF₃)₂.

274. The composition of claim 270 wherein the R_{F} group is one of —C₆F₁₃, ((CF₃)₂CFCH₂)₂CH—, ((CF₃)₂CFCH₂)₂CH₂CH₂—, (CF₃)₂CFCH₂((CF₃)₂CF)CH—, (CF₃)₂CFCH₂CH(CF₃)CH₂CH(CF₃)—, or (CF₃)₂CFCH₂CH₂CH₂CH₂((CF₃)₂CFCH)CH—.

275-279. (canceled)

280. The composition of claim 270 wherein the R_{T} group comprises one or more of 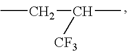
—CH₂—CF₂—, —CH₂—(CH₂CF(CF₃)₂)CH—, and —CH₂—CH₂—.

281-283. (canceled)

284. The composition of claim 270 wherein n is at least 2 and the composition is

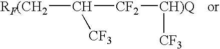

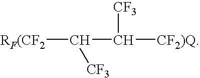

285. (canceled)

286. The composition of claim 270 wherein the Q group comprises a halogen.

287. A composition comprising one or both of 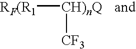 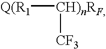
wherein:
the R_{F} group comprises at least two fluorine groups;
the R₁ group comprises at least one carbon atom and a halogen;
n is at least 1; and
the Q group comprises one or more atoms of the periodic table of elements.

288. The composition of claim 287 wherein the R_{F} group comprises at least two —CF₃ groups.

289. The composition of claim 287 wherein the R₁ group consists of —CF₂—.

290. The composition of claim 287 wherein n is equal to 1 and the composition comprises

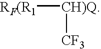

291. The composition of claim 287 wherein the Q group comprises at least one halogen.

292. A composition comprising:
R_{Cl}(R_{T})ₙH, wherein:
the R_{Cl} group comprises at least —CCl₃;
the R_{T} group comprises at least one C-2 group, the C-2 group comprising a —CF₂— group and at least one pendant —CF₃ group; and
n is at least 1.

293. The composition of claim 292 wherein n is at least 2 and the composition comprises

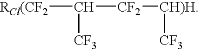

294. The composition of claim 292 wherein n is at least 2 and the composition comprises

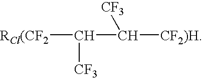

295. A composition comprising R_{F}(R_{T})ₙQg, wherein:
the R_{F} group comprises at least two —CF₃ groups;
the R_{T} group comprises a group having at least two carbons;
n is at least 1; and
the Qg group comprises one or more atoms of the periodic table of elements.

296. The composition of claim 295 wherein R_{F}(R_{T})ₙQg

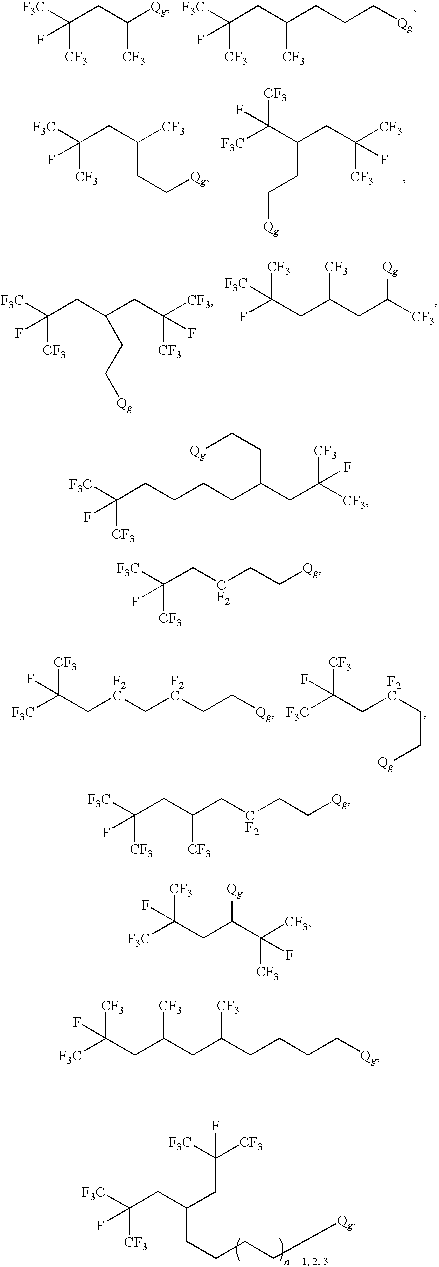

297-311. (canceled)
